SEQUENCE LISTING (30 sequences)

SEQ ID NO: 1 - protein, 20 aa, Artificial Sequence
  chemically synthesized
TFFYGGSRGKRNNFKTEEYC

SEQ ID NO: 2 - protein, 20 aa, Artificial Sequence
  chemically synthesized
  MISC_FEATURE (20)..(20): can be Lys or Cys or absent
TWPKHFDKHTFYSILKLGKH

SEQ ID NO: 3 - protein, 13 aa, Artificial Sequence
  chemically synthesized
  MISC_FEATURE (13)..(13): terminal Lys residue optionally Cys, each with C(=O)-NH2 terminus
EAKIEKHNHYQKK

SEQ ID NO: 4 - protein, 23 aa, Artificial Sequence
  chemically synthesized
  MISC_FEATURE (23)..(23): terminal residue is Lys or Cys or absent, when present terminus is C(=O)-NH2 or C(=O)-OH terminus
EAKIEKHNHYQKQLEIAHEKLRK

SEQ ID NO: 5 - protein, 13 aa, Artificial Sequnce
  chemically synthesized
  MISC_FEATURE (1)..(1): Xaa = (R)-2-(7-octenyl) Ala
  MISC_FEATURE (1)..(8): hydrocarbon bridge
  MISC_FEATURE (8)..(8): Xaa = (S)-2-(4-pentenyl)Ala
  MISC_FEATURE (13)..(13): terminal residue is Lys or Cys or absent, when present terminus is C(=O)-NH2 or C(=O)-OH
XAKIEKHXHYQKK

SEQ ID NO: 6 - protein, 30 aa, Artificial Sequence
  chemically synthesized
  MISC_FEATURE (30)..(30): terminal Leucine has terminal C(=O)-NH2 or (C=O)-OH
LRKLRKRLLRDADDLLRKLRKRLLRDADDL

SEQ ID NO: 7 - protein, 20 aa, Artificial Sequence
  chemically synthesized
  MISC_FEATURE (20)..(20): terminus is C(=O)-OH or C(=O)NH2
TEELRVRLASHLRKLRKRLL

SEQ ID NO: 8 - protein, 17 aa, Artificial Sequence
  chemically synthesized
  MISC_FEATURE (1)..(1): acetylated
  MISC_FEATURE (11)..(11): Xaa = norleucine
VKFNKPFVFLXIEQNTK

SEQ ID NO: 9 - protein, 17 aa, Artificial Sequence
  chemically synthesized
VKFNKPFVFLMIEQNTK

SEQ ID NO: 10 - protein, 20 aa, Artificial Sequence
  chemically synthesized
  MISC_FEATURE (20)..(20): Cys optionally absent, when present terminus is C(=O)-OH or C(=O)-NH2
TFFYGGCRGKRNNFKTEEYC

SEQ ID NO: 11 - protein, 20 aa, Artificial Sequence
  chemically synthesized
  MISC_FEATURE (20)..(20): Cys optionally absent, when present terminus is C(=O)-OH or C(=O)-NH2
TFFYGGSRGRRNNFRTEEYC

SEQ ID NO: 12 - protein, 20 aa, Artificial Sequence
  chemically synthesized
  MISC_FEATURE (20)..(20): Cys optionally absent, when present terminus is C(=O)-OH or C(=O)-NH2
TFFYGGSRGRRNNFRTEEYC

SEQ ID NO: 13 - protein, 20 aa, Artificial Sequence
  chemically synthesized
  MISC_FEATURE (20)..(20): Cys optionally absent, when present terminus is C(=O)-OH or C(=O)-NH2
CYEETKFNNRKGRSGGYFFT

SEQ ID NO: 14 - protein, 19 aa, Artificial Sequence
  chemically synthesized
TFFYGGCRAKRNNFKRAKY

SEQ ID NO: 15 - protein, 19 aa, Artificial Sequence
  chemically synthesized
TFFYGGCRGKKNNFKRAKY

SEQ ID NO: 16 - protein, 19 aa, Artificial Sequence
  chemically synthesized
PFFYGGCRGKRNNFKTEEY

SEQ ID NO: 17 - protein, 19 aa, Artificial Sequence
  chemically synthesized
TFFYGGKRGKRNNFKTKEY

SEQ ID NO: 18 - protein, 19 aa, Artificial Sequence
  chemically synthesized
TFFYGGCRGKRNNFKTKRY

SEQ ID NO: 19 - protein, 19 aa, Artificial Sequence
  chemically synthesized
TFFYGGKRGKRNNFKTAEY

SEQ ID NO: 20 - protein, 19 aa, Artificial Sequence
  chemically synthesized
TFFYGGKRGKRNNFKREKY

SEQ ID NO: 21 - protein, 19 aa, Artificial Sequence
  chemically synthesized
RFKYGGCLGNKNNFLRLKY

SEQ ID NO: 22 - protein, 19 aa, Artificial Sequence
  chemically synthesized
RFKYGGCLGNKNNYLRLKY

SEQ ID NO: 23 - protein, 7 aa, Artificial Sequence
  chemically synthesized
SVWIWYE

SEQ ID NO: 24 - protein, 10 aa, Artificial Sequence
  chemically synthesized
DVWIINKKLK

SEQ ID NO: 25 - protein, 19 aa, Artificial Sequence
  chemically synthesized
  MISC_FEATURE (19)..(19): terminus is C(=O)-OH or C(=O)NH2
MLRTKDLIWTLFFLGTAVS

SEQ ID NO: 26 - protein, 25 aa, Artificial Sequence
  chemically synthesized
  MISC_FEATURE (25)..(25): terminus is C(=O)-OH or C(=O)-NH2
MLRTKDLIWTLFFLGTAVSKKRPKP

SEQ ID NO: 27 - protein, 25 aa, Artificial Sequence
  chemically synthesized
  MISC_FEATURE (25)..(25): terminus is C(=O)-OH or C(=O)-NH2
MLRTKDLIWTLFFLGTAVSKKLVFF

SEQ ID NO: 28 - protein, 5 aa, Artificial Sequence
  chemically synthesized
  REPEAT (1)..(5): sequence repeats 1, 2, 3, 4, 5, or 6 times
GGGGS

SEQ ID NO: 29 - protein, 5 aa, Artificial Sequence
  chemically synthesized
  REPEAT (1)..(5): sequence repeats 1, 2, 3, 4, 5, or 6 times
SSSSG

SEQ ID NO: 30 - protein, 13 aa, Artificial Sequence
  chemically synthesized
SGSSSSGSSSSGS

(19) United States
(12) Patent Application Publication
(10) Pub. No.: US 2024/0181067 A1
(43) Pub. Date: Jun. 6, 2024

(54) TARGETED BIFUNCTIONAL DEGRADERS AND METHODS USING SAME

(71) Applicant: YALE UNIVERSITY, New Haven, CT (US)

(72) Inventors: David Spiegel, New Haven, CT (US); Rebecca Howell, New Haven, CT (US); David McDonald, New Haven, CT (US)

(21) Appl. No.: 18/547,436

(22) PCT Filed: Feb. 22, 2022
(86) PCT No.: PCT/US22/17334

§ 371 Date: Aug. 22, 2023

Related U.S. Application Data
(60) Provisional application No. 63/152,110, filed on Feb. 22, 2021.

(51) Int. Cl. A61K 47/55; A61K 47/54; A61K 47/60; A61K 47/65; A61P 25/00

(52) U.S. Cl. CPC A61K 47/55; A61K 47/545; A61K 47/60; A61K 47/65; A61P 25/00

(57) ABSTRACT

The present disclosure provides, in one aspect, bifunctional compounds that can be used to promote or enhance degradation of an extracellular or cell surface protein. In certain embodiments, the extracellular protein mediates a disease and/or disorder in a subject, and treatment or management of the disease and/or disorder requires degradation, removal, and/or reduction in concentration of the protein in the subject. In some embodiments, the disease and/or disorder is a neurological disease and/or disorder. Thus, in certain embodiments, administration of a compound of the disclosure to the subject removes or reduces the amount of the extracellular or cell surface protein in the brain, thus treating, ameliorating, or preventing the disease and/or disorder.

### CROSS-REFERENCE TO RELATED APPLICATION

This application claims the benefit of priority to U.S. Provisional Patent Application Ser. No. 63/152,110 entitled “TARGETED BIFUNCTIONAL DEGRADERS,” filed Feb. 22, 2021, the disclosure of which is incorporated herein by reference in its entirety.

### STATEMENT REGARDING FEDERALLY SPONSORED RESEARCH OR DEVELOPMENT

This invention was made with government support under GM067543 awarded by National Institutes of Health. The government has certain rights in the invention.

### BACKGROUND OF THE DISCLOSURE

Several neurological diseases arise from the accumulation and aggregation of pathogenic proteins in the brain. However, current treatment options, particularly for Alzheimer's disease, aim to improve symptoms without addressing the underlying pathogenic protein causation or slowing disease progression. For example, potential Alzheimer's disease treatment could involve modulation of various brain-located pathogenic proteins, such as but not limited to inflammatory cytokines, extracellular tau, and beta-amyloid.

There is a need in the art for novel compounds and methods that allow for inhibition, removal, and/or degradation of certain extracellular or cell surface proteins that mediate a disease and/or disorder in a subject. The present disclosure addresses this need.

### BRIEF SUMMARY OF THE DISCLOSURE

In one aspect, a compound of formula (I), or a salt, geometric isomer, stereoisomer, or solvate thereof is provided. The compound of formula (I) has the structure:

[TBM]ₙ-[Linker]ₘ-[LRP1BM]ₒ (I),

wherein
m is an integer from 0 to 15;
n and o are each independently an integer from 1 to 15;
[TBM] represents a Target binding motif comprising or consisting of:
   (a) a compound selected from:

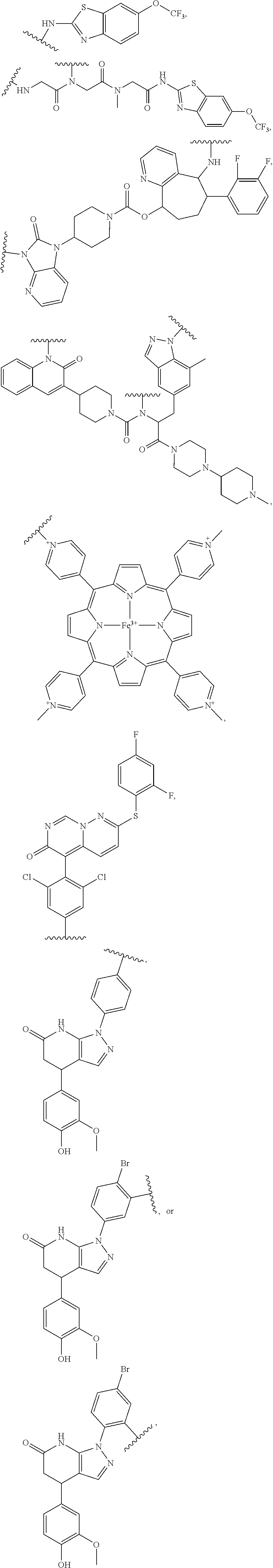

or a derivative or prodrug thereof,
wherein
[symbol] indicates possible points of covalent attachment to a [Linker] or a [LRP1BM];
   (b) a compound of formula (I):

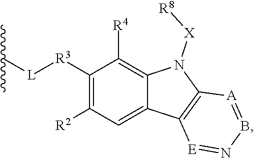

or a derivative or prodrug thereof,
wherein:
A is N or CR⁵;
B is N or CR⁶;
E is N or CR⁷;
L is a substituted or unsubstituted alkylene, substituted or unsubstituted alkenylene, substituted or unsubstituted alkynylene, substituted or unsubstituted carbocyclylene, substituted or unsubstituted heterocyclylene, substituted or unsubstituted arylene, substituted or unsubstituted heteroarylene, substituted or unsubstituted heteroalkylene, a bond, —O—, —NR^{A}—, —S—, —C(═O)—, —C(═O)O—, —C(═O)NR^{A}, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂—NR^{A}—, —NR^{A}S(O)₂—, or a combination thereof;
X is a bond or substituted or unsubstituted C₁₋₁₂ alkylene, wherein one or more carbon is optionally replaced with C(═O), O, S, SO₂, NH, or NC₁₋₆ alkyl optionally substituted with halogen, OH, or C₁₋₆ alkyl;
R⁸ is hydrogen, —N₃, alkynyl, OH, halogen, NH₂, N(C₁₋₆ alkyl)₂, aryl, heteroaryl, or a protecting group, wherein the aryl and heteroaryl are optionally substituted with halogen, SO₂, NH₂, or C₁₋₆ alkyl optionally substituted with halogen or C₃₋₈ cycloalkyl;
R³ is —(CH₂)ₙ—, —(CH₂)ₙ—C(═O), —(CH₂)ₙC(═O)—O—, (CH₂)ₙO—, -A-(CH₂)ₙ—O—, —(CH₂)ₙ-A-O—, -A-O—(CH₂)ₙ—(C═O)NR^{A}—, —(CH₂)ₙ—S—, -A-(CH₂)ₙS—, —(CH₂)ₙ-A-S—, -A-S—(CH₂)ₙ—(C═O)NR^{A}—, —(CH₂)ₙ—NR^{A}—, -A-(CH₂)ₙ—NR^{A}, —(CH₂)ₙ-A-NR^{A}—, —(CH₂)ₙ—(C═O)NR^{A}—, -A-(CH₂)ₙ—(C═O)NR^{A}—, —(CH₂)ₙ-A-(C═O)NR^{A}—, -A-NR^{A}—(CH₂)ₙ—(C═O)NR^{A}—, —(CH₂)ₙ—S(O)₂NR^{A}, -A-(CH₂)ₙ—S(O)₂NR^{A}—, or —(CH₂)ₙ-A-S(O)₂NR^{A}—;
each occurrence of R^{A} is independently selected from hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, substituted or unsubstituted aryl, substituted or unsubstituted heteroaryl, or a nitrogen protecting group when attached to a nitrogen atom, or two R^{A} groups are joined to form a substituted or unsubstituted heterocyclic ring;
each occurrence of A is independently selected from substituted or unsubstituted heterocyclylene, substituted or unsubstituted arylene, or substituted or unsubstituted heteroarylene;
R¹, R² and R₄-R⁸ are each independently hydrogen, OH, halogen, NH₂, CH₃, SO₂, NO₂, a leaving group, a protecting group, aryl, heteroaryl, NHR¹², N(R¹²)₂ C₃₋₈ cycloalkyl, N(R¹²)₂ heterocyclyl or —(CH₂)ₙ—R¹²;
R¹² is hydrogen, —CH₃, aryl, or heteroaryl; and
n is 0-12;
wherein one or more carbon of R¹-R⁷ is optionally replaced with C(═O)O, O, S, SO₂, NH, NH—C₁₋₆ alkyl, NC₁₋₆, alkyl, NH₂, or N(C₁₋₆ alkyl)₂; and
[symbol] indicates the point of covalent attachment to a [Linker] or a [LRP1BM];
   (c) a compound of formula (II):

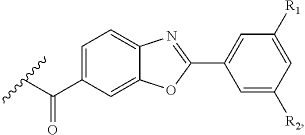

or a derivative or prodrug thereof,
wherein
R¹ and R² are each independently selected from hydrogen, N₃, alkynyl, OH, halogen, NH₂, N(C₁₋₆ alkyl)₂, C₁₋₆ alkyl, aryl, heteroaryl, NHR¹², N(R¹²)₂ C₃₋₈ cycloalkyl, N(R¹²)₂ heterocyclyl, or —(CH₂)ₙ—R¹²;
wherein the aryl and heteroaryl are optionally substituted with halogen, —SO₂, NO₂, —NH₂, or C₁₋₆ alkyl optionally substituted with halogen or C₃₋₈ cycloalkyl;
R¹² is hydrogen, —CH₃, aryl, or heteroaryl; and
n is 0-12;
wherein one or more carbon of R¹ or R² is optionally replaced with C(═O), O, S, SO₂, NH, NH—C₁₋₆ alkyl, NC₁₋₆ alkyl, NH₂, or N(C₁₋₆ alkyl)₂; and
[symbol] indicates the point of covalent attachment to a [Linker] or a [LRP1BM];
   (d) a compound of formula (III):

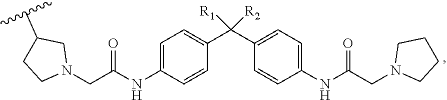

or a derivative or prodrug thereof,
wherein
R₁ is selected from benzene, phenyl, cyclohexyl, hydrogen, and CF₃;
R₂ is selected from hydrogen and CF₃; and
[symbol] indicates the point of covalent attachment to a [Linker] or a [LRP1BM];
   (e) a compound of formula (IV):

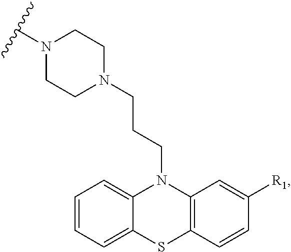

or a derivative or prodrug thereof,
wherein
R₁ is selected from hydrogen, Cl, OMe, SMe, and CF₃, and
[symbol] indicates the point of covalent attachment to a [Linker] or a [LRP1BM];
   (f) a compound of formula (V):

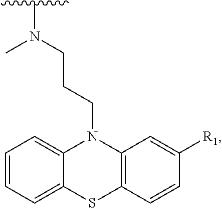

or a derivative or prodrug thereof,
wherein
R₁ is selected from hydrogen, Cl, OMe, SMe, and CF₃, and
[symbol] indicates the point of covalent attachment to a [Linker] or a [LRP1BM]; or
   (g) an amino acid sequence selected from:

SVWIWYE,
DVWIINKKLK,
MLRTKDLIWTLFFLGTAVS-NH₂,
MLRTKDLIWTLFFLGTAVS-KKRPKP-NH₂,
and
MLRTKDLIWTLFFLGTAVS-KKLVFF-NH₂;

[LRP1BM] represents a low density lipoprotein receptor-related protein 1 (LRP1) receptor binding motif comprising one of the following amino acid sequences:

TFFYGGSRGKRNNFKTEEYC-OH (or -NH₂),
TWPKHFDKHTFYSILKLGKH-OH,
EAKIEKHNHYQKK/C-NH₂,
EAKIEKHNHYQKQLEIAHEKLRK/C-NH₂,
R₈AKIEKHS₅HYQKK/C-NH₂,

wherein R₈ represents (R)-2-(7-octenyl)Ala-OH, S₅ represents (S)-2-(4-pentenyl)Ala-OH, and there is a hydrocarbon bridge between position 1 and 8,

LRKLRKRLLRDADDLLRKLRKRLLRDADDL-NH₂,
TEELRVRLASHLRKLRKRLL-NH₂,
Ac-VKFNKPFVFLNleIEQNTK-NH₂,

wherein Nle represents norleucine,

VKFNKPFVFLMIEQNTK,
TFFYGGCRGKRNNFKTEEYC-OH (or -NH₂),
TFFYGGSRGKRNNFRTEEYC-OH (or -NH₂),
TFFYGGSRGRRNNFRTEEYC-OH (or -NH₂),
cyeetkfnnrkGrsGGyfft-OH (or -NH₂),
TFFYGGCRAKRNNFKRAKY,
TFFYGGCRGKKNNFKRAKY,
PFFYGGCRGKRNNFKTEEY,
TFFYGGKRGKRNNFKTKEY,
TFFYGGCRGKRNNFKTKRY,
TFFYGGKRGKRNNFKTAEY,
TFFYGGKRGKRNNFKREKY,
RFKYGGCLGNKNNFLRLKY,
and
RFKYGGCLGNKNNYLRLKY,

wherein the underlined amino acids in the above sequences indicate that the amino acids may be present or absent and underlined K/C indicates that either K or C may be present; and
[Linker] represents a polyethylene glycol containing linker having 1-12 ethylene glycol residues, or [Linker] represents a Linking group comprising:
   (a) —CH₂CH₂(OCH₂CH₂)ₘOCH₂—, —(CH₂)ₘCH₂—, or —[N(R^{a})—CH(R^{b})(C═O)]ₘ—,
      or a polypropylene glycol or polypropylene-co-polyethylene glycol group containing 1-100 alkylene glycol units;
      wherein each R^{a} is independently H, C₁-C₃ alkyl, or C₁-C₆ alkanol, or combines with R^{b} to form a pyrrolidine or hydroxypyrroline group;
      wherein each R^{b} is independently selected from the group consisting of hydrogen, methyl, isopropyl, —CH(CH₃)CH₂CH₃, —CH₂CH(CH₃)₂, —(CH₂)₃-guanidine, —CH₂C(═O)NH₂, —CH₂C(═O)OH, —CH₂SH, —(CH₂)₂C(═O)NH₂, —(CH₂)₂C(═O)OH, —(CH₂)imidazole, —(CH₂)₄NH₂, —CH₂CH₂SCH₃, benzyl, —CH₂OH, —CH(OH)CH₃, —(CH₂)imidazole, or —(CH₂)phenol; and
      wherein m is an integer ranging from 1 to 15;
   (b) —[N(R′—(CH₂)₁₋₁₅—C(═O)]ₘ—, wherein R′ is H or a C₁-C₃ alkyl optionally substituted with 1-2 hydroxyl groups, and m is an integer ranging from 1 to 100;
   (c) —Z-D-Z′—, wherein:
      Z and Z′ are each independently a bond, —(CH₂)ᵢ—O—, —(CH₂)ᵢ—S—, —(CH₂)ᵢ—N(R)—,

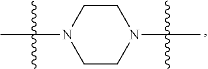 [SMILES: *N1CCN(*)CC1]

—(CH₂)ᵢ—C(R²)═C(R²)— (cis or trans), —(CH₂)ᵢ-≡-, or —Y—C(═O)—Y—,
each R is independently H, C₁-C₃ alkyl, or C₁-C₆ alkanol,
each R² is independently H or C₁-C₃ alkyl,
each Y is independently a bond, O, S, or N(R),
each i is independently 0 to 100,
D is a bond, —(CH₂)ᵢ—Y—C(═O)—Y—(CH₂)ᵢ—, —(CH₂)_{m′}—, or —[(CH₂)ₙ—X₁)]ⱼ—, with the proviso that Z, Z′, and D are not each simultaneously bonds;
X₁ is O, S, or N(R),
j is an integer ranging from 1 to 100,
m′ is an integer ranging from 1 to 100,
n is an integer ranging from 1 to 100;
(d) —CH₂—(OCH₂CH₂)ₙ—CH₂—, —(CH₂CH₂O)_{n′}CH₂CH₂—, or —(CH₂CH₂CH₂O)ₙ—, wherein each n and n′ is independently an integer ranging from 1 to 25;
(e) -PEG-CON-PEG-, wherein each PEG is independently a polyethylene glycol group containing from 1-12 ethylene glycol residues and CON is selected from

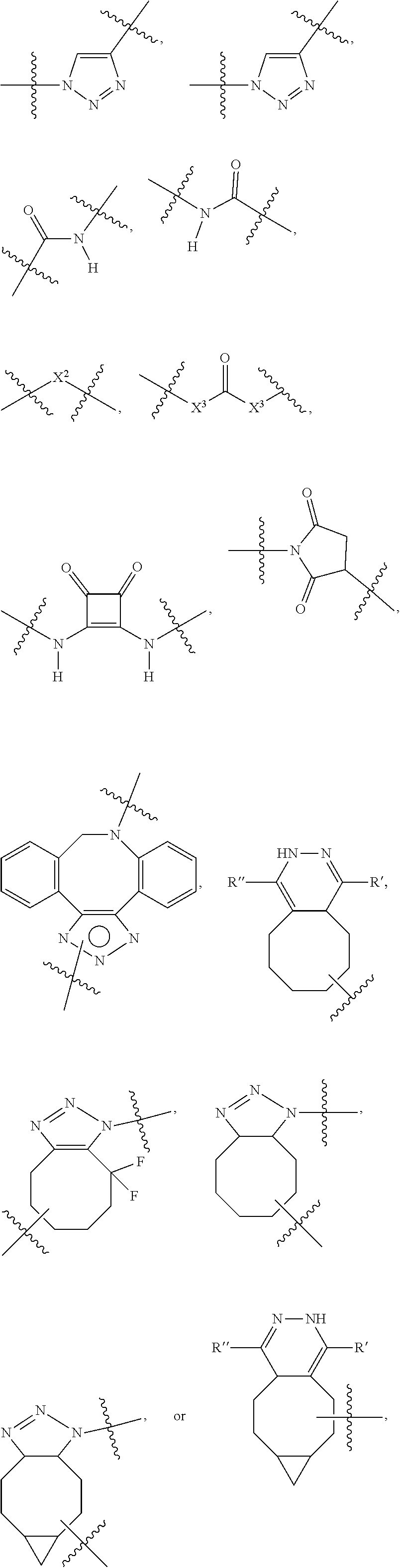

wherein R′ and R″ are each independently H, methyl, or a bond;
(f) -PEG-CON-PEG-, wherein each PEG is independently a polyethylene glycol group containing from 1-12 ethylene glycol residues and CON comprises a diamide structure selected from —C(═O)—N(R¹)—(CH₂)_{n″}—N(R¹)C(═O)—, —N(R¹)—C(═O)(CH₂)_{n″}—C(═O)N(R¹)—, or —N(R¹)—C(═O)(CH₂)_{n″}—N(R¹)C(═O)—, wherein each R¹ is independently H or C₁-C₃ alkyl, and n″ is independently an integer from 0 to 8;
(g) -PEG-CON-PEG-, wherein each PEG is independently a polyethylene glycol group containing from 1-12 ethylene glycol residues and CON comprises a structure

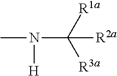

wherein:
   R^{1a}, R^{2a} and R^{3a} are each independently H, —(CH₂)_{M1}—, —(CH₂)_{M2}C(═O)_{M3}(NR⁴)_{M3}—(CH₂)_{M2}—, —(CH₂)_{M2}(NR⁴)_{M3}C(O)_{M3}—(CH₂)_{M2}—, or —(CH₂)_{M2}O—(CH₂)_{M1}—C(O)NR⁴—, with the proviso that R^{1a}, R^{2a} and R^{3a} are not simultaneously H;
   each M1 is independently 1, 2, 3, or 4; in certain embodiments, 1 or 2;
   each M2 is independently 0, 1, 2, 3, or 4; in certain embodiments, 0, 1 or 2;
   each M3 is independently 0 or 1; and
   each R⁴ is independently H, C₁-C₃ alkyl, C₁-C₆ alkanol, or —C(═O)(C₁-C₃ alkyl), with the proviso that M2, and M3 within the same R^{1a}, R^{2a} and R^{3a} cannot all be simultaneously 0;
(h) -PEG-CON-PEG-, wherein each PEG is independently a polyethylene glycol group containing from 1-12 ethylene glycol residues and CON comprises a structure:

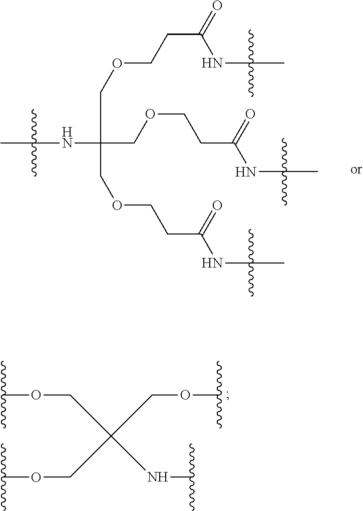 [SMILES: *NC(=O)CCOCC(COCCC(=O)N*)(COCCC(=O)N*)N* ; *NC(CO*)(CO*)CO*]

(i) a natural or an unnatural amino acid;
(j) [Gly-Gly-Gly-Gly-Ser]ₙ, where n is 1, 2, 3, 4, 5 or 6;
(k) [Ser-Ser-Ser-Ser-Gly]_{y}, where y is ≥1; or
(l) Ser-Gly-Ser-Ser-Ser-Ser-Gly-Ser-Ser-Ser-Ser-Gly-Ser.

In one aspect, the compounds of formula (I) are useful in methods of treating, ameliorating, and/or preventing a disease or disorder in a subject. Such methods include administering a therapeutically effective amount of at least one compound of formula (I), or a salt, geometric isomer, stereoisomer, or solvate thereof. In one aspect, the disease or disorder comprises a neurological disease or disorder.

Diseases or disorders that are treated, ameliorated, or prevented by compounds of formula (I) include Huntington's Disease (HD), Parkinson's Disease (PD), Amyotropic Lateral Sclerosis (ALS), multiple system atrophy (MSA), Alzheimer's Disease, Lewy body dementia, Multiple System Atrophy, spinal and bulbar muscular atrophy (Kennedy's disease), Tourette Syndrome, spinocerebellar ataxia (SCA), schizophrenia, age associated memory impairment, autism, migraines, Rett syndrome, complex regional pain syndrome (CRPS), obsessive-compulsive disorder (OCD), attention-deficit disorder, bipolar disorder, hereditary cerebral angiopathy, ATTR amyloidosis, or depression.

### BRIEF DESCRIPTION OF THE DRAWINGS

The following detailed description of exemplary embodiments of the invention will be better understood when read in conjunction with the appended drawings. For the purpose of illustrating the invention, non-limiting embodiments are shown in the drawings. It should be understood, however, that the invention is not limited to the precise arrangements and instrumentalities of the embodiments shown in the drawings.

FIG. 1 is a scheme depicting how illustrative disclosed bifunctional molecules remove target neurological pathogenic proteins.

FIG. 2 depicts low density lipoprotein receptor related protein 1 (LRP1) binding motifs.

FIG. 3 depicts non-limiting Target binding motifs.

FIG. 4 depicts structure of Angiopep-2, with non-limiting sites for possible modifications.

FIG. 5 depicts non-limiting Target binding motifs used for proof of concept studies.

FIG. 6 depicts saturable delivery of streptavidin AF647 by Angiopep-2.

FIG. 7 depicts non-limiting results of ELISA studies demonstrating that biotinylated Angiopep-2 binds streptavidin.

FIG. 8 depicts that biotinylated Angiopep-2 delivers streptavidin AF647 to murine brain endothelial cells.

FIG. 9 depicts illustrative Angiopep-2 mediated endocytosis of the noncovalent cargo protein streptavidin.

FIG. 10 depicts illustrative results of ELISA studies demonstrating that DNP-modified Angiopep-2 binds anti-DNP antibody.

FIG. 11 depicts that non-limiting biotinylated LRP1 targeting peptides (RAP Mimetics) bind streptavidin protein.

FIG. 12 depicts the Ac.Ac.Biotin Angiopep-2 mediated degradation of streptavidin AF488.

### DETAILED DESCRIPTION OF THE DISCLOSURE

The present disclosure provides, in one aspect, bifunctional compounds that can be used to promote or enhance degradation of an extracellular protein or cell surface protein. In certain embodiments, the extracellular or cell surface protein mediates a disease and/or disorder in a subject, and treatment or management of the disease and/or disorder requires degradation, removal, or reduction in concentration of the extracellular or cell surface protein in the subject. Thus, in certain embodiments, administration of a compound of the disclosure to the subject removes the extracellular or cell surface protein and/or reduces the circulation concentration of the extracellular or cell surface protein, thus treating, ameliorating, or preventing the disease and/or disorder in the subject. In some embodiments, the extracellular or cell surface protein is a neurological protein. In certain embodiments, the extracellular or cell surface protein mediates a neurological disease and/or disorder in a subject. In some embodiments, the extracellular or cell surface protein comprises a pathological protein which accumulates or aggregates in the brain of a subject suffering from a neurological disease or disorder. In some embodiments, the extracellular or cell surface protein comprises a pathological protein which accumulates or aggregates at the blood-brain barrier (BBB) of a subject suffering from a neurological disease or disorder. In certain embodiments, the cell surface protein comprises a pathological protein which accumulates or aggregates on endothelial cells at the BBB of a subject suffering from a neurological disease or disorder. In another embodiment, the bifunctional compounds of the disclosure induce the trafficking of a protein into and/or out of the central nervous system (CNS). In some embodiments, the bifunctional compounds can induce trafficking of a protein into and/or out of the CNS without degrading the protein.

In certain embodiments, the compound of the disclosure comprises a LRP1 binding motif which targets the low-density lipoprotein receptor-related protein 1 (LRP1). In certain embodiments, the LRP1 is found in the brain. In some embodiments, the LRP1 binding motif is covalently bonded, through an optional Linker group, to a Target binding motif. In some embodiments, the Target binding motif comprises a protein binding moiety. In some embodiments, the protein binding moiety binds noncovalently to a pathological protein. In some embodiments, the pathological protein comprises an extracellular protein. In other embodiments, the pathological protein comprises a cell surface protein. In certain embodiments, the pathological protein is found in the brain or at the BBB. In some embodiments, the disclosed bifunctional compound bonded to the extracellular or cell surface protein undergoes endocytosis, the extracellular or cell surface protein is eventually degraded, and the bifunctional compound can be degraded or recycled to the outside of the cell. The structure and function of LRP1 is described in, for example, Potere N., et al., “Low Density Lipoprotein Receptor-Related Protein-1 in Cardiac Inflammation and Infarct Healing,” Frontiers in Cardiovascular Medicine, vol. 6, 2019.

In accordance with the present disclosure, conventional chemical synthetic and pharmaceutical formulation methods, as well as pharmacology, molecular biology, microbiology, and recombinant DNA techniques within the skill of the art may be employed. Such techniques are well-known and are otherwise explained fully in the literature.

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀ alkyl. In certain embodiments, the heteroalkyl group is an unsubstituted heteroC₁₋₁₀ alkyl. In certain embodiments, the heteroalkyl group is a substituted heteroC₁₋₂₀ alkyl. In certain embodiments, the heteroalkyl group is an unsubstituted heteroC₁₋₂₀ alkyl.

The ten “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₁₋₁₀ alkynyl.

The term “carbocyclyl” or “carbocyclic” refers to a radical of a non-aromatic cyclic hydrocarbon group having from 3 to 14 ring carbon atoms (“C₃₋₄ carbocyclyl”) and zero heteroatoms in the non-aromatic ring system Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆) cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇) cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈) and the like. Exemplary C₃₋₁₀ carbocyclyl groups include, without limitation, the aforementioned C₃₋₈ carbocyclyl groups as well as cyclononyl (C9), cyclononenyl (C9), cyclodecyl (C₁₀), cyclodecenyl (C₁₀), octahydro-1H-indenyl ((C9), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds, “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl in certain embodiments, the carbocyclyl group is a substituted C₃₋₁₄ carbocyclyl.

In some embodiments, “carbocyclyl” is a monocyclic, saturated carbocyclyl group laving from 3 to 14 ring carbon atoms (“C₃₋₁₄ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₄ cycloalkyl. In certain embodiments, the cycloalkyl group is a substituted C₃₋₄ cycloalkyl.

“Heteroaralkyl” is a subset of “alkyl” and refers to an alkyl group substituted by a heteroaryl group, wherein the point of attachment is on the alkyl moiety.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl. “substituted” or “unsubstituted” alkynyl, “substituted” or “unsubstituted” heteroalkyl, “substituted” or “unsubstituted” heteroalkenyl, “substituted” or “unsubstituted” heteroalkynyl. “substituted” or “unsubstituted” carbocyclyl, “substituted” or “unsubstituted” heterocyclyl. “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH R, —ON(R^{bb})₂, —N(R^{bb})₂, —N(R^{bb})₃⁺X⁻, —N(OR^{cc})R^{bb}, —SH, —SR^{bb}, —SSR^{CC}, —C(═O)R^{aa}, —CO₂H, —CHO, —C(OR^{cc})₃, —COR^{aa}, —OC(═O)R^{aa}, —OCO₂R^{aa}, —C(═O)N(R^{bb})₂, —OC(═O)N(R^{bb})₂, —R^{bb}C(═O)R^{aa}, —R^{bb}CO₂R^{aa}, —NR^{bb}C(═O)N(R^{bb})₂, —C(═R^{bb})R^{aa}, —C(═R^{bb})OR^{aa}, —OC(═R^{bb})R^{aa}, —OC(═R^{bb})OR^{aa}, —C(═R^{bb})N(R^{bb})₂, O(═R^{bb})N(R^{bb})₂, —R^{bb}C(═NR^{bb})N(R^{bb})₂, —C(═O)R^{bb}SO₂R^{aa}, —NR\hat{ }SQzR^{bb}, —SO₂N(R^{bb})₂, —SO₂R^{bb}, —SO₂OR^{aa}, —OSO^{bb}, —S(═O)R^{aa}, —OS(═O)R^{aa}, —SiCR\hat{ }s, —OSiCR\hat{ }s, —C(═S)N(R^{bb})₂, —C(═O)SR^{aa}, —C(═S)SR^{aa}, —SC(═S)SR^{aa}, —SR^{aa}SR^{bb}, —OC(═O)SR^{aa}, —SC(═O)OR^{aa}, —SC(═O)R^{a}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —R^{bb}P(═O)(R^{aa})₂, —NR^{bb}P(═O)(OR^{cc})₂, —R^{bb}P(═O)(N(R)₂)₂, —P(R^{CC})₂, —P(OR^{cc})₂, —P(R^{CC})₃⁺X⁻, —P(OR^{cc})₃⁺X⁻, —P(R^{CC})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{aa})₂, —BR(OR^{cc}), C₂₋₄₀ alkyl, C₂₋₄₀ perhaloalkyl, C₂₋₁₀alkenyl, C₂₋₁₀alkynyl, heteroC₁₋₂₀ alkyl, heteroC₂₋₁₀ alkenyl, heteroC₂₋₁₀alkynyl, C₃₋₁₀ carbocyclyl, 3-14 membered heterocyclyl. C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb} or ═NOR^{cc}.
each instance of R^{aa} is, independently, selected from C₁₋₁₀ alkyl, C₁₋₁₀ perhaloalkyl, C₂₋₁₀ alkenyl, C₂₋₁₀alkynyl, heteroC₁₋₂₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R\hat{ } groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{cc}, —N(R^{CC})₂, —CN, —C(═O)R^{aa}, —C(═O)N(R^{aa})₂, —CO₂R^{aa}, —SO₂R^{aa}, —C(═R^{cc})OR^{aa}, —C(═R^{CC})N(R_{CC})₂, —SO₂N(R^{cc})₂, —SO₂R^{cc}, —SO₂OR^{cc}, —SOR^{cc}, —C(═S)N(R^{cc})₂, —C(═O)SR^{cc}, —C(═S)SR^{CC}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀alkenyl, C₂₋₁₀alkynyl, heteroC₁₋₁₀ alkyl, heteroC₂₋₁₀alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion,
each instance of R_{cc} is, independently, selected from hydrogen, C₁₋₁₀alkyl, C₁₋₁₀ perhaloalkyl, C₂₋₁₀alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 Rd groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —R^{ff}C(═O)Rₑₑ, —R^{ff}CO₂R^{ee}, —R^{ff}C(═O)N(R^{ff})₂, —C(═R^{ff})OR^{ee}, —OC(═R^{ff})R^{ee}, —OC(═R^{ff})OR^{ee}, —C(═R^{ff})N(R^{ff})₂, —OC(═R^{ff})N(R_{ff})₂, —R^{ff}C(═NR^{ff})N(R^{ff})₂, —R^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₂, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ v aryl, 5-10 membered heteroaryl, wherein each alkyl, alkenyl, alkynyl, heteroalkyl, heteroalkenyl, heteroalkynyl, carbocyclyl, heterocyclyl, aril, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R ff groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆alkyl)₂, —N(C₁₋₆ alkyl)₃⁺X⁻, —H(C₁₋₆ alkyl)₂⁺X⁻, —N₂(C₁₋₆ alkyl)⁺X⁻, —N₃⁺X⁻, —N(C₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆alkyl), —OC(═O)C₁₋₆ alkyl), —OCO₂(C₁₋₆ alkyl), —C(═O)NH₂, —C(═O)N(C₁₋₆ alkyl)₂, —OC(═O) NH(C₁₋₆alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O) H(C₁₋₆ alkyl), —NHC(═O)NH₂, —C(═O)O(C₁₋₆ alkyl), —OC(═O)(C₁₋₆ alkyl), —OC(═O)O C₁₋₆ alkyl, —C(═O)N(C₁₋₆ alkyl)₂, —C(═O)O(C₁₋₆ alkyl), —C(═O)NH, —OC(═O)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆alkyl), —C(═S)SC₁₋₆ alkyl, —SC(═S) C₁₋₆ alkyl, —P(═O)(O C₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH—, —OR^{aa}, —N(R^{CC})₂, —CN, —C(═O)R^{aa}, —C(═O)N(R^{cc})₂, —CO₂R^{aa}, —SO₂R^{aa}, —C(═NR^{bb})R^{aa}, —C(═NR^{CC})OR^{aa}, —C(═NR^{CC})N(R^{CC})₂, —SO₂N(R^{cc})₂, —SO₂R^{CC}, —SO₂OR^{cc}, —SOR\hat{ }, —C(═S)N(R^{CC})₂, —C(═O)SR^{CC}, —C(═S)SR^{CC}, —P(═O)(OR^{cc})₂, —P(═O)(R^{aa})₂, —P(═O)(N(R_{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₁₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc}, and R^{dd} are as defined herein.

In certain embodiments, the substituent present on the nitrogen atom is an nitrogen protecting group (also referred to herein as an “anno protecting group”). Nitrogen protecting groups include, but are not limited to, —OH, —OR^{aa}, —N(R^{CC})₂, —C(═O)R^{aa}, —C(═O)N(R^{CC})₂, —CO₂R^{aa}, —SO₂R^{aa}, —C(═NR^{cc})R^{aa}, —C(═NR^{CC})OR^{aa}, —C(═NR^{CC})N(R^{CC})₂, —SO₂N(R^{cc})₂, —SO₂R^{cc}, —SO₂OR^{cc}, —SOR^{aa}, —C(═S)N(R^{CC})₂, —C(═O)SR^{CC}, —C(═S)SR^{CC}, C_{MO} alkyl (e.g., aralkyl, heteroaralkyl), C₂₋₁₀ alkenyl, C₂₋₁₀ alkynyl, heteroC₁₋₁₀ alkyl, heteroC₂₋₁₀alkenyl, heteroC₂₋₁₀alkynyl. C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluorenylmethyl carbamate, 2,7-di-t-butyl-[9-(10,10-dioxo-10,10,10,10-tetrahydrothioxanthyl)] methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate. [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxy benzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pine), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fem), N-2-picolylamino N′-oxide, N-1,1-dimethylthiomethyleneamine, N-benzylideneamine, N-p-methoxy benzlideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl] amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobennzenesulfenamide, 2-nitro-4-methoxybenzenesulfenamide, triphenylmethylsulfenamide, and 3-nitropyridinesulfenamide (Npys). In certain embodiments, a nitrogen protecting group is benzyl (Bn), tert-butyloxycarbonyl (BOC), carbobenzyloxy (Cbz), 9-flturenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (Tf), or dansyl (Ds).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{a}, —C(O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{CC})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well know % n in the art and include those described in detail in Protecting Groups in Organic Synthesis. T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-metboxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, ρ,ρ′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxy phenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-naphthyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxy acetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, an oxygen protecting group is silyl. In certain embodiments, an oxygen protecting group is t-butyldiphenylsilyl (TBDPS), t-butyldimethylsilyl (TBDMS), triisopropylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxyethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB), t-butyl, benzyl (Bn), allyl, or pivaloyl (Piv).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —SiCR\hat{ }s, —P(R^{CC})₂, P(R^{xx})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or tnvalent. Exemplary counterions include halide ions (e.g., F⁻, Cl⁻, Br⁻, I⁻), NO₃⁻, C10₄⁻, OFT, H₂PO₄⁻, HCO₃⁻, HSO₄⁻, sulfonate ions (e.g., methansulfonate, trifluoromethanesulfonate, 7-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄-, Al(OC(CF₃)₃)₄⁻, and carborane anions (e.g., CBnH₁₂⁻ or (HCBnMe₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

The term “leaving group” is given its ordinary meaning in the art of synthetic organic chemistry and refers to an atom or a group capable of being displaced by a nucleophile. See, for example, Smith, March's Advanced Organic Chemistry 6th ed. (501-502). Examples of suitable leaving groups include, but are not limited to, halogen (such as F, CI, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy. N,O-dimethylhydroxylamino, pixyl, and haloformates. In some cases, the leaving group is a sulfonic acid ester, such as toluenesulfonate (tosylate, -OTs), methanesulfonate (mesylate, -OMs), >-bromobenzenesulfonyloxy (brosylate, -OBs), —OS(═O)₂(CF₂)₃CF₃ (nonaflate, -ONf), or trifluoromethanesulfonate (triflate, -OTf). In some cases, the leaving group is a brosylate, such as 7-bromobenzenesulfonyloxy. In some cases, the leaving group is a nosylate, such as 2-nitrobenzenesulfonyloxy. The leaving group may also be a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties. Further exemplary leaving groups include, but are not limited to, halo (e.g., chloro, bromo, iodo) and activated substituted hydroxyl groups (e.g., —OC(═O)SR^{aa}, —OC(═O)R^{aa}, —OCOR^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(O)(OR^{cc})₂, —OP(═O)N(R^{bb})₂, and —OP(═O)(NR^{bb})₂, wherein R^{aa}, R^{bb}, and R^{cc} are as defined herein).

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “aminoalkyl” as used herein refers to amine connected to an alkyl group, as defined herein. The amine group can appear at any suitable position in the alkyl chain, such as at the terminus of the alkyl chain or anywhere within the alkyl chain.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

As used herein, the term “C₆₋₁₀-C₆₋₁₀ biaryl” means a C₆₋₁₀ aryl moiety covalently bonded through a single bond to another C₆₋₁₀ aryl moiety. The C₆₋₁₀ aryl moiety can be any of the suitable aryl groups described herein. Non-limiting example of a C₆₋₁₀-C₆₋₁₀ biaryl include biphenyl and binaphthyl.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7.

Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Eₘₐₓ) achieved within an assay.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

As used herein, the term “C₆₋₁₀-5-6 membered heterobiaryl” means a C₆₋₁₀ aryl moiety covalently bonded through a single bond to a 5- or 6-membered heteroaryl moiety. The C₆₋₁₀ aryl moiety and the 5-6-membered heteroaryl moiety can be any of the suitable aryl and heteroaryl groups described herein. Non-limiting examples of a C₆₋₁₀-5-6 membered heterobiaryl include:

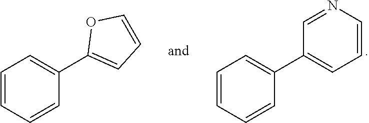 [SMILES: c1ccc(-c2ccco2)cc1 ; c1ccc(-c2cccnc2)cc1]

When the C₆₋₁₀-5-6 membered heterobiaryl is listed as a substituent (e.g., as an “R” group), the C₆₋₁₀-5-6 membered heterobiaryl is bonded to the rest of the molecule through the C₆₋₁₀ moiety.

As used herein, the term “5-6 membered-C₆₋₁₀ heterobiaryl” is the same as a C₆₋₁₀-5-6 membered heterobiaryl, except that when the 5-6 membered-C₆₋₁₀ heterobiaryl is listed as a substituent (e.g., as an “R” group), the 5-6 membered-C₆₋₁₀ heterobiaryl is bonded to the rest of the molecule through the 5-6-membered heteroaryl moiety.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, wherein X¹, X², and X³ are all different, wherein X¹ and X² are the same but X³ is different, and other analogous permutations.

The term “monovalent” as used herein refers to a substituent connecting via a single bond to a substituted molecule. When a substituent is monovalent, such as, for example, F or Cl, it is bonded to the atom it is substituting by a single bond.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀)hydrocarbyl, wherein R can be hydrogen (in examples that include other carbon atoms) or a carbon-based moiety, and wherein the carbon-based moiety can be substituted or unsubstituted.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The term “solvent” as used herein refers to a liquid that can dissolve a solid, liquid, or gas. Non-limiting examples of solvents are silicones, organic compounds, water, alcohols, ionic liquids, and supercritical fluids.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “therapeutic” treatment is a treatment administered to a subject who exhibits signs of pathology, for the purpose of diminishing or eliminating those signs.

The term “thioalkyl” as used herein refers to a sulfur atom connected to an alkyl group, as defined herein. The alkyl group in the thioalkyl can be straight chained or branched. Examples of linear thioalkyl groups include but are not limited to thiomethyl, thioethyl, thiopropyl, thiobutyl, thiopentyl, thiohexyl, and the like. Examples of branched alkoxy include but are not limited to iso-thiopropyl, sec-thiobutyl, tert-thiobutyl, iso-thiopentyl, iso-thiohexyl, and the like. The sulfur atom can appear at any suitable position in the alkyl chain, such as at the terminus of the alkyl chain or anywhere within the alkyl chain.

The terms “treat,” “treating” and “treatment,” as used herein, means reducing the frequency or severity with which symptoms of a disease or condition are experienced by a subject by virtue of administering an agent or compound to the subject.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds

In one aspect, the present disclosure relates to a bifunctional molecule of formula (I), or a salt, geometric isomer, stereoisomer, or solvate thereof:

[TBM]ₙ-[Linker]ₘ-[LRP1BM]ₒ (I),

wherein
[TBM] represents a Target binding motif,
[LRP1BM] represents a LRP1 binding motif,
m is an integer from 0 to 15, and
n and o are each independently an integer from 1 to 15.

In some embodiments, the Linker is a group having a valence ranging from 1 to 15. In certain embodiments, the valence of the Linker is 1 to 10. In certain embodiments, the valence of the Linker is 1 to 5. In certain embodiments, the valence of the Linker is 1, 2, or 3. In certain embodiments, the Linker covalently links one or more Target binding motifs to one or more LRP1 binding motifs.

In some embodiments, m an integer ranging from 0 to 15. In certain embodiments, m is an integer ranging from 1 to 15. In certain embodiments, m is an integer ranging from 1 to 10. In certain embodiments, m is an integer ranging from 1 to 5. In certain embodiments, m is an integer ranging from 1 to 3. In certain embodiments, m is 1, 2, or 3.

In some embodiments, n and o are each independently an integer ranging from 1 to 15. In certain embodiments, n and o are each independently an integer ranging from 1 to 10. In certain embodiments, n and o are each independently an integer ranging from 1 to 5. In certain embodiments, n and o are each independently an integer ranging from 1 to 3. In certain embodiments, each of n and o is independently 1, 2 or 3.

### LRP1 Binding Motif

In some embodiments, the LRP1 binding motif comprises a peptide that targets the low-density lipoprotein receptor-related protein 1 (LRP1). In certain embodiments, the LRP1 binding motif targets LRP1 in the brain and/or at the BBB. While not wishing to be limited by theory, it is believed that LRP1 is involved in endolysosomal trafficking, as well as receptor-mediated transcytosis across the blood brain barrier, indicating that peptides targeting this receptor can be capable of both transport and degradation of target neurological proteins.

In some embodiments, the LRP1 binding motif comprises one of the following amino acid sequences:
Angiopep-2: TFFYGGSRGKRNNFKTEEYC-OH (or —NH₂) (SEQ ID NO:1), Demeule, et al., J. Pharmacol. Exp. Ther. 324(3):1064-1072;
L57: TWPKHFDKHTFYSILKLGKH-OH (SEQ ID NO:2), Sakamoto, et al., 2017, Biochemistry and biophysics reports 12:135-139;
Rap12: EAKIEKHNHYQK/C-NH₂(SEQ ID NO:3), Ruan, et al., 2018, Journal of Controlled Release 279:306-315;
Rap22: EAKIEKHNHYQKQLEIAHEKLRK/C-NH₂ (SEQ ID NO:4), Ruan, et al., 2018, Journal of Controlled Release 279:306-315;
Stapled (ST)-RAP12: RSAKIEKHSSHYQKK/C—NH₂ (SEQ ID NO:5), wherein R₈ represents (R)-2-(7-octenyl)Ala-OH, S5 represents (S)-2-(4-pentenyl)Ala-OH, and there is a hydrocarbon bridge between position 1 and 8, Ruan et al., Chemical Engineering Journal, 2021, 403:126296;
ApoE (141-155): LRKLRKRLLRDADDLLRKLRKRLLRDADDL-NH₂ (SEQ ID NO:6), Croy, et al., 2004, Biochemistry 43.23:7328-7335;
ApoE (130-149): TEELRVRLASHLRKLRKRLL-NH₂ (SEQ ID NO:7), Croy, et al., 2004, Biochemistry 43.23:7328-7335;
Ac-VKFNKPFVFLNleIEQNTK-NH₂ (SEQ ID NO:8), wherein Nle represents norleucine, Toldo et al., 2017, JACC: Basic to Translational Science 2.5:561-574;
VKFNKPFVFLMIEQNTK (SEQ ID NO:9), Toldo et al., 2017, JACC: Basic to Translational Science 2.5:561-574;
Angiopep-1: TFFYGGCRGKRNNFKTEEYC—OH (or —NH₂) (SEQ ID NO:10), Demeule, et al., Journal Pharmacology and Experimental Therapeutics, 2008, 324(3):1064;
Angiopep-5: TFFYGGSRGKRNNFRTEEYC—OH (or —NH₂) (SEQ ID NO:11), Demeule, et al., Journal Pharmacology and Experimental Therapeutics, 2008, 324(3):1064;
Angiopep-7: TFFYGGSRGRRNNFRTEEYC—OH (or —NH₂) (SEQ ID NO: 12), Demeule, et al., Journal Pharmacology and Experimental Therapeutics, 2008, 324(3):1064;
Retroinverso Angiopep-2: cyeetkfnnrkGrsGGyfft-OH (or —NH₂) (SEQ ID NO:13), Wei et al., Molecular Pharmaceutics, 2014, 11(10): 3261;
sequences derived from the C-terminal sequence of aprotinin including but not limited to:

(SEQ ID NO: 14)
TFFYGGCRAKRNNFKRAKY;
(SEQ ID NO: 15)
TFFYGGCRGKKNNFKRAKY;
(SEQ ID NO: 16)
PFFYGGCRGKRNNFKTEEY;
(SEQ ID NO: 17)
TFFYGGKRGKRNNFKTKEY;
(SEQ ID NO: 18)
TFFYGGCRGKRNNFKTKRY;
(SEQ ID NO: 19)
TFFYGGKRGKRNNFKTAEY;
(SEQ ID NO: 20)
TFFYGGKRGKRNNFKREKY;
(SEQ ID NO: 21)
RFKYGGCLGNKNNFLRLKY;
and
(SEQ ID NO: 22)
RFKYGGCLGNKNNYLRLKY,

### (Demeule et al., Journal Pharmacology and Experimental Therapeutics, 2008, 324(3):1064);

wherein the underlined amino acids in the above sequences indicate that the amino acids may be present or absent and underlined K/C indicates that either K or C may be present.

In certain embodiments, the amino end of any of SEQ ID NOs 1-22 binds to the Linker group or the Target binding motif. In other embodiments, the carboxylic acid end of any of SEQ ID NOs 1-22 binds to the Linker group or the Target binding motif. In yet other embodiments, the carboxylic acid terminus of any of SEQ ID NOs 1-22 is a non-reactive carboxamide group and the amine terminus is covalently linked to the Linker group or the Target binding motif.

### Target Binding Motif

In some embodiments, the Target binding motif comprises a protein binding moiety. In certain embodiments, the protein binding moiety binds to a pathological protein. In one embodiment, the protein binding moiety binds to an exosome comprising the pathological protein. In some embodiments, the pathological protein is found in the brain. In some embodiments, the protein binding moiety binds noncovalently to the pathological protein. In some embodiments, the pathological protein is an extracellular protein. In other embodiments, the pathological protein is a cell surface protein. In other embodiments, the pathological protein is a CNS protein. In some embodiments, the protein binding moiety binds a protein which is accumulates and/or aggregates in a subject suffering from a neurological disease or disorder. In some embodiments, the protein binding moiety binds a protein which is accumulates and/or aggregates in the brain of a subject suffering from a neurological disease or disorder.

The pathological protein can be any pathological protein known to a person of skill in the art. Exemplary pathological proteins include, but are not limited to, Complement Factor B, Complement Factor D, DPP4, Complement component C3b, IgG, TNF alpha, Lysyl Oxidase 2 (LOXL2), IL-17, Amyloid beta, Tau, Hormone-sensitive lipase, Lipoprotein-associated Phospholipase A2, Factor Xa, Matrix metalloproteinase IX (MMP-9), Thrombin, Elastase, Factor XI, PKK (pre-kallikrein), BLyS, B cell activating factor (BAFF), FGF23 (fibroblast growth factor 23), Anti-DNA antibodies, extracellular Myeloperoxidase (MPO), IL-18, Transthyretin (misfolded), Myostatin, CD40 (soluble), CXCL12, CD40 Ligand (soluble), Plasminogen activator inhibitor type 1 (PAI-1), PABA (protective antigen of Bacillus anthracis); edema factor, suPAR (soluble urokinase plasminogen activator receptor), PF4, Tetanus toxin, IL-6, VEGF, Beta2-m, IgA, SAA (serum amyloid A), Soluble PSMA, MIF, ApoB-100, Protein arginine deiminase (PAD, PAD4), C. difficile toxin B, CJD-associated prion, Hemolysin, IL-2, Botulinum toxin Antibodies to citrullinated protein antibody (ACPA), HTT, Anti-ganglioside IgG, Antibodies to Klebsiella dipeptidase protein, Antibodies to anionic phospholipids, beta-2-glycoprotein-I, IgM, Anticardiolipin antibodies, lupus anticoagulant, IgG autoantibodies, Anti-vWF antibodies, Amyloid light chains, IgA, IgE, IgG autoantibodies to thyroid peroxidase, thyroglobulin, TSH receptors, sFlt1, IL-21, IL-13, IL-5, Serum amyloid P component, amyloid precursor protein, C reactive protein (CRP), an inflammatory cytokine, a calcitonin gene-related peptide (CGRP), a CGRP receptor, an N-methyl-D-aspartate (NMDA) receptor, α-synuclein, IAPP, transthyretin, and combinations thereof. In some embodiments, the pathological protein is selected from an inflammatory cytokine, a calcitonin gene-related peptide (CGRP), a CGRP receptor, an N-methyl-D-aspartate (NMDA) receptor, myeloperoxidase (MPO), IAPP, transthyretin, extracellular tau, beta-amyloid, amyloid precursor protein, prion protein, and α-synuclein. In some embodiments, the Target binding motif binds to extracellular tau, beta-amyloid, amyloid precursor protein, prion protein, α-synuclein, or a combination thereof.

In some embodiments, the Target binding motif comprises formula (I):

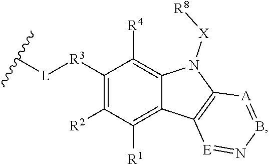

or a derivative or prodrug thereof,
wherein:
A is N or CR⁵;
B is N or CR⁶;
E is N or CR⁷;
L, is a substituted or unsubstituted alkylene, substituted or unsubstituted alkenylene, substituted or unsubstituted alkynylene-substituted or unsubstituted carbocyclylene, substituted or unsubstituted heterocyclylene, substituted or unsubstituted arylene, substituted
L is a substituted or unsubstituted alkylene, substituted or unsubstituted alkenylene, substituted or unsubstituted alkynylene, substituted or unsubstituted carbocyclylene, substituted or unsubstituted heterocyclylene, substituted or unsubstituted arylene, substituted or unsubstituted heteroarylene, substituted or unsubstituted heteroalkylene, a bond, —O—, —NR^{A}—, —S—, —C(═O)—, —C(═O)O—, —C(═O)NR^{A}, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂—NR^{A}—, —NR^{A}S(O)₂—, or a combination thereof;
X is a bond or substituted or unsubstituted C₁₋₁₂ alkylene, wherein one or more carbon is optionally replaced with C(═O), O, S, SO₂, NH, or NC₁₋₆ alkyl optionally substituted with halogen, OH, or C₁₋₆ alkyl;
R⁸ is hydrogen, —N₃, alkynyl, OH, halogen, NH₂, N(C₁₋₆ alkyl)₂, aryl, heteroaryl, or a protecting group, wherein the aryl and heteroaryl are optionally substituted with halogen, SO₂, NH₂, or C₁₋₆ alkyl optionally substituted with halogen or C₃₋₈ cycloalkyl;
R³ is —(CH₂)ₙ—, —(CH₂)ₙ—C(═O), —(CH₂)ₙC(═O)—O—, (CH₂)ₙO—, -A-(CH₂)ₙ—O—, —(CH₂)ₙ-A-O—, -A-O—(CH₂)ₙ—(C═O)NR^{A}—, —(CH₂)ₙ—S—, -A-(CH₂)ₙS—, —(CH₂)ₙ-A-S—, -A-S—(CH₂)ₙ—(C═O)NR^{A}—, —(CH₂)ₙ—NR^{A}—, -A-(CH₂)ₙ—NR^{A}, —(CH₂)ₙ-A-NR^{A}—, —(CH₂)ₙ—(C═O)NR^{A}—, -A-(CH₂)ₙ—(C═O)NR^{A}—, —(CH₂)ₙ-A-(C═O)NR^{A}—, -A-NR^{A}—(CH₂)ₙ—(C═O)NR^{A}—, —(CH₂)ₙ—S(O)₂NR^{A}, -A-(CH₂)ₙ—S(O)₂NR^{A}—, or —(CH₂)ₙ-A-S(O)₂NR^{A}—;
each occurrence of R^{A} is independently selected from hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, substituted or unsubstituted aryl, substituted or unsubstituted heteroaryl, or a nitrogen protecting group when attached to a nitrogen atom, or two R^{A} groups are joined to form a substituted or unsubstituted heterocyclic ring;
each occurrence of A is independently selected from substituted or unsubstituted heterocyclylene, substituted or unsubstituted arylene, or substituted or unsubstituted heteroarylene;
R¹, R², and R⁴-R⁸ are each independently hydrogen, OH, halogen, NH₂, CH₃, SO₂, NO₂, a leaving group, a protecting group, ail, heteroaryl, NHR¹², N(R¹²)₂ C₃₋₈cycloalkyl N(R¹²)₂ heterocyclyl, or —(CH₂)ₙ—R¹²;
R¹² is hydrogen, —CH₃, aryl, or heteroaryl; and
n is 0-12;
wherein one or more carbon of R¹-R⁷ is optionally replaced with C(═O), O, S, SO₂, NH, NH—C₁₋₆ alkyl, NC₁₋₆ alkyl, NH₂ or N(C₁₋₆ alkyl)₂.

In one embodiment, wherein [symbol] in formula (I) indicates possible points of covalent attachment to a Linker group or a LRP1 binding motif.

In one embodiment, A is CR⁵, B is CR⁶ and E is CR⁷. In another embodiment, each of A, B, and E are N.

In one embodiment, the Target binding motif of formula (I) or a derivative or prodrug thereof binds extracellular tau.

In one embodiment, the Target binding motif of formula (I) is

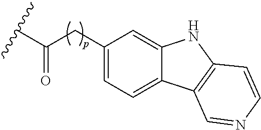

or a derivative or prodrug thereof, wherein p is an integer from 1-6. In certain embodiments, p is 2. In some embodiments,

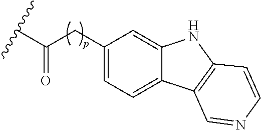

and derivatives or prodrugs thereof bind extracellular tau. In another embodiment, the Target binding motif of formula (I) is

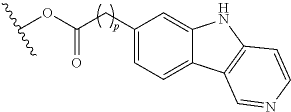

or a derivative or prodrug thereof, wherein p is an integer from 1-6. In certain embodiments, p is 2. In some embodiments,

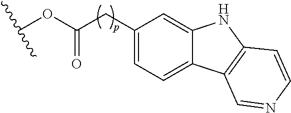

and derivatives or prodrugs thereof bind extracellular tau.

In other embodiments, the Target binding motif comprises the following structure:

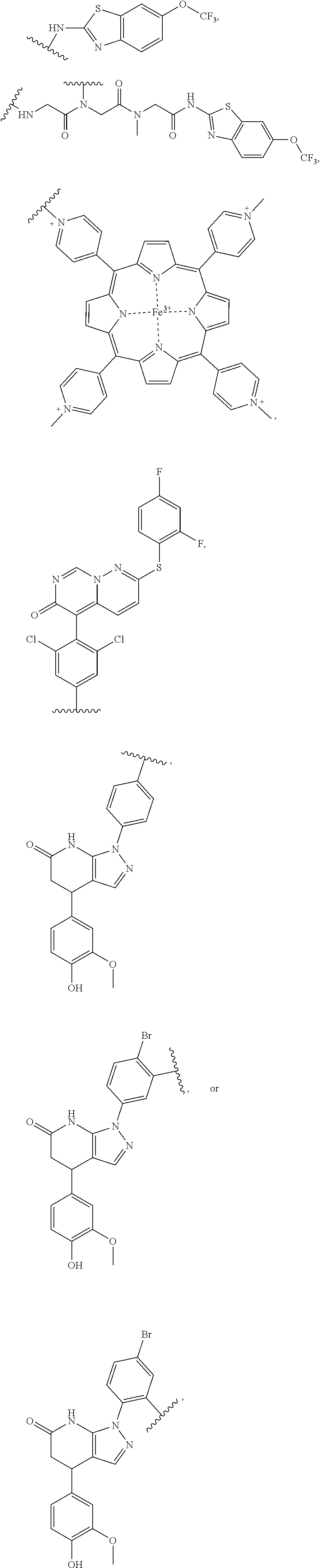

or a derivative or prodrug thereof, wherein [symbol] indicates possible points of covalent attachment to a Linker group or a LRP1 binding motif. In some embodiments

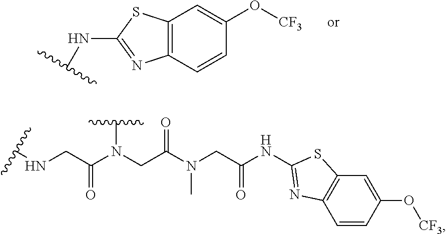 [SMILES: *Nc1nc2ccc(OC(F)(F)F)cc2s1 ; *NCC(=O)N(*)CC(=O)N(C)CC(=O)Nc1nc2ccc(OC(F)(F)F)cc2s1]

or a derivative or prodrug thereof acts as a glutamate modulator. In one embodiment,

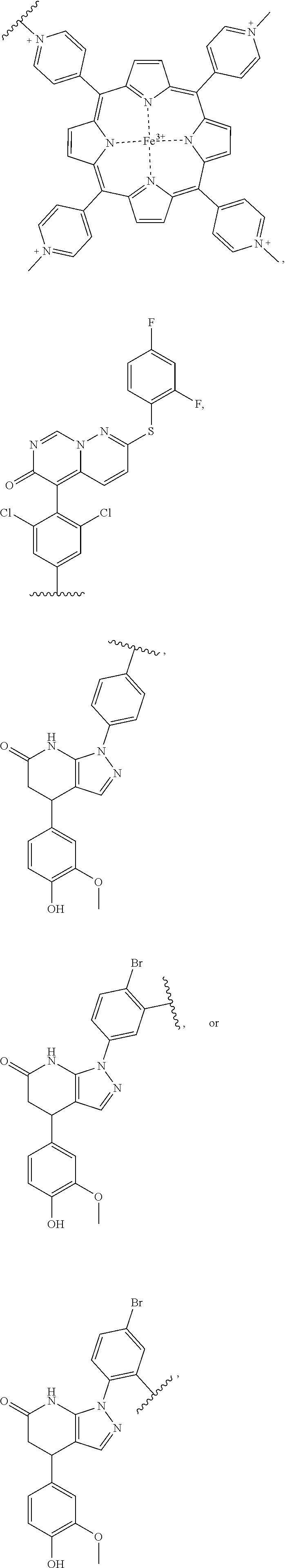

or a derivative or prodrug thereof, acts to target and/or bind a prion protein. In other embodiments, the Target binding motif comprises the following structure:

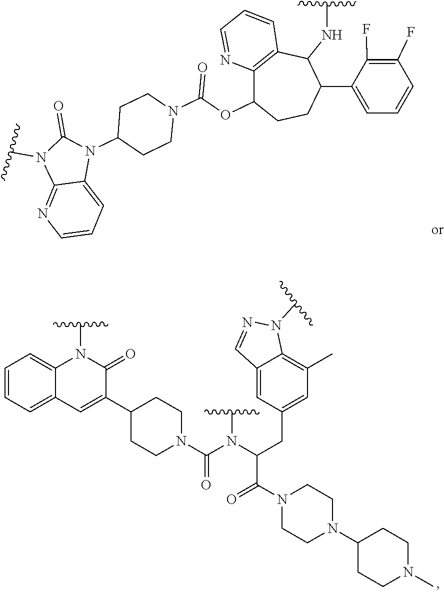 [SMILES: *NC1c2cccnc2C(OC(=O)N2CCC(n3c(=O)n(*)c4ncccc43)CC2)CCC1c1cccc(F)c1F ; *N(C(=O)N1CCC(c2cc3ccccc3n(*)c2=O)CC1)C(Cc1cc(C)c2c(cnn2*)c1)C(=O)N1CCN(C2CCN(C)CC2)CC1]

or a derivative or prodrug thereof, wherein [symbol] indicates possible points of covalent attachment to a Linker group or a LRP1 binding motif. In some embodiments,

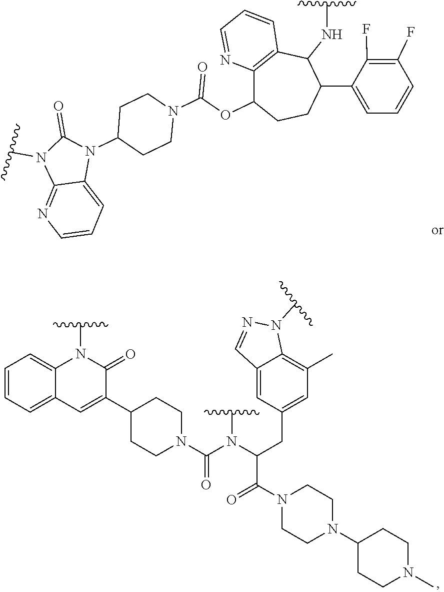 [SMILES: *NC1c2cccnc2C(OC(=O)N2CCC(n3c(=O)n(*)c4ncccc43)CC2)CCC1c1cccc(F)c1F ; *N(C(=O)N1CCC(c2cc3ccccc3n(*)c2=O)CC1)C(Cc1cc(C)c2c(cnn2*)c1)C(=O)N1CCN(C2CCN(C)CC2)CC1]

or a derivative or prodrug thereof, binds CGRP or a CGRP receptor.

In yet other embodiments, the Target binding motif comprises formula (II).

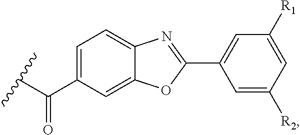

or a derivative or prodrug thereof,
wherein
R¹ and R² are each independently selected from hydrogen, —N₃, alkynyl, —OH, halogen, —NH₂, —N(C₁₋₆ alkyl)₂, C₁₋₆ alkyl, aryl, heteroaryl, NHR¹², N(R¹²)₂C₃₋₈ cycloalkyl, N(R¹²)₂heterocyclyl, or —(CH₂)ₙ—R¹²;
wherein the aryl and heteroaryl are optionally substituted with halogen, —SO₂, NO₂, —NH₂, or C₁₋₆ alkyl optionally substituted with halogen or C₃₋₈ cycloalkyl;
R¹² is hydrogen, —CH₃, aryl, or heteroaryl, and
n is 0-12;
wherein one or more carbon of R¹ or R² is optionally replaced with C(═O), O, S, SO₂, NH, NH—C₁₋₆alkyl, NC₁₋₆ alkyl, NH₂, or N(C₁₋₆alkyl)₂.

In one embodiment, [symbol] in formula (II) indicates possible points of covalent attachment to a Linker group or a LRP1 binding motif.

In one embodiment, the Target binding motif of formula (II) or a derivative or prodrug thereof binds transthyretin.

In one embodiment, each of R₁ and R₂ of formula (II) are independently F, Cl, Br, or I. In certain embodiments, R₁ and R₂ of formula (II) are each Cl.

In other embodiments, the Target binding motif comprises formula (III):

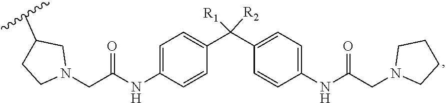

or a derivative or prodrug thereof,
wherein
R₁ is selected from benzene, phenyl, cyclohexyl, hydrogen, and CF₃,
R₂ is selected from hydrogen and CF₃, and
[symbol] indicates the point of covalent attachment to a Linker group or a LRP1 binding motif.

In one embodiment, the Target binding motif of formula (III), or a derivative or prodrug thereof, acts to target and/or bind a prion protein.

In other embodiments, the Target binding motif comprises formula (IV):

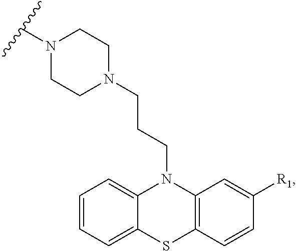

or a derivative or prodrug thereof,
wherein
R₁ is selected from hydrogen, Cl, OMe, SMe, and CF₃, and
[symbol] indicates the point of covalent attachment to a Linker group or a LRP1 binding motif.

In one embodiment, the Target binding motif of formula (IV), or a derivative or prodrug thereof, acts to target and/or bind a prion protein.
In other embodiments, the Target binding motif comprises formula (V):

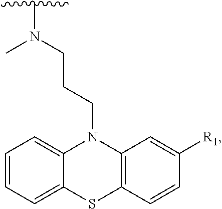

or a derivative or prodrug thereof,
wherein
R₁ is selected from hydrogen, Cl, OMe, SMe, and CF₃, and
[symbol] indicates the point of covalent attachment to a Linker group or a LRP1 binding motif.

In one embodiment, the Target binding motif of formula (V), or a derivative or prodrug thereof, acts to target and/or bind a prion protein.

In certain embodiments, a derivative of the above structures comprises one or more functional groups described elsewhere herein.

In other embodiments, the Target binding motif comprises one of the following amino acid sequences that targets extracellular tau:
VY-WIW: SVWIWYE (SEQ ID NO:23), (Seidler, P. M. et al., Journal of Biological Chemistry, 2019, 29:16451-16464); or
IN-M4: DVWIINKKLK (SEQ ID NO:24), (Seidler, P. M. et al., Journal of Biological Chemistry, 2019, 29:16451-16464), wherein SEQ ID NOs 23 and 24 can be attached to the Linker or LPR1 binding motif through the C or N terminus.

In other embodiments, the Target binding motif comprises one of the following amino acid sequences that targets amyloid beta:
NCAM1 (N): MLRTKDLIWTLFFLGTAVS-NH₂ (SEQ ID NO:25), (Henning-Knechtel, A. et al., Cell Reports Physical Science, 2020, 26:100014);
N-Pr: MLRTKDLIWTLFFLGTAVS-KKRPKP-NH₂ (SEQ ID NO:26), (Henning-Knechtel, A. et al., Cell Reports Physical Science, 2020, 26:100014); or
N-Aβ: MLRTKDLIWTLFFLGTAVS-KKLVFF-NH₂ (SEQ ID NO:27), (Henning-Knechtel, A. et al., Cell Reports Physical Science, 2020, 26:100014),
   wherein SEQ ID NOs 25-27 can be attached to the Linker or LPR1 binding motif through the C or N terminus. In some embodiments, the bolded portion of the N-Pr or N-Aβ sequence comprises the amino acids that target amyloid beta.

In certain embodiments, the amino end of any of SEQ ID NOs: 23-27 binds to the Linker group or the LPR1 binding motif. In other embodiments, the carboxylic acid end of any of SEQ ID NOs: 23-27 binds to the Linker group or the LPR1 binding motif. In yet other embodiments, the carboxylic acid terminus of any of SEQ ID NOs: 23-27 is a non-reactive carboxamide group and the amine terminus is covalently linked to the Linker group or the LPR1 binding motif.

In some embodiments, the TBM (Target binding motif) can be any of the ASGPR binding moieties described in: Reshitko, G. S., et al., “Synthesis and Evaluation of New Trivalent Ligands for Hepatocyte Targeting via the Asialoglycoprotein Receptor,” Bioconjugate Chem, doi: 10.1021/acs.bioconjchem.0c00202; Majouga, A. G., et al., “Identification of Novel Small-Molecule ASGP-R Ligands,” Current Drug Delivery, 2016, 13, 1303-1312, doi: 10.2174/1567201813666160719144651; Olshanova, A. S., et al., “Synthesis of a new betulinic acid glycoconjugate with N-acetyl-D-galactosamine for the targeted delivery to hepatocellular carcinoma cells,” Russian Chemical Bulletin, International Edition, Vol. 69, No. 1, pp. 158-163, January 2020; Yamansarov, E. Yu., et al., “New ASGPR-targeted ligands based on glycoconjugated natural triterpenoids,” Russian Chemical Bulletin, International Edition, Vol. 68, No. 12, pp. 2331-2338, December 2019; Congdon, M. D., et al., “Enhanced Binding and Reduced Immunogenicity of Glycoconjugates Prepared via Solid-State Photoactivation of Aliphatic Diazirine Carbohydrates,” Bioconjugate Chem, doi: 10.1021/acs.bioconjchem.0c00555; and Dhawan, V., et al., “Polysaccharide conjugates surpass monosaccharide ligands in hepatospecific targeting—Synthesis and comparative in silico and in vitro assessment,” Carbohydrate Research 509 (2021) 108417, doi: 10.1016/j.carres.2021.108417.

### Linker

In certain embodiments, m of formula (I) is 0, the Linker is absent, and the Target binding motif is covalently bonded to the LRP1 binding motif.

In certain embodiments, the Linker is an amino acid, wherein the amino acid is any natural or unnatural amino acid. In one embodiment, the amino acid is selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. In one embodiment, the unnatural amino acid is selected from hydroxyproline, beta-alanine, citrulline, ornithine, norleucine, 3-nitrotyrosine, nitroarginine, naphthylalanine, aminobutyric acid, 2,4-diaminobutyric acid, methionine sulfoxide, methionine sulfone, and pyroglutamic acid. In one embodiment where the Linker is lysine, glutamic acid, or aspartic acid, the side chain forms an amide bond with the Target binding motif or the LRP1 binding motif.

In certain embodiments, the Linker is a glycine rich peptide. In one embodiment, the Linker is a glycine rich peptide comprising the sequence [Gly-Gly-Gly-Gly-Ser]ₙ (SEQ ID NO:28), where n is 1, 2, 3, 4, 5 or 6.

In certain embodiments, the Linker is a serine rich peptide. In one embodiment, the Linker is a serine rich peptide comprising the sequence [Ser-Ser-Ser-Ser-Gly]_{y} (SEQ ID NO:29) where y is 1. In one embodiment, y is 1, 2, 3, 4, 5, or 6. In one embodiment, the Linker is a serine rich peptide having the sequence Ser-Gly-Ser-Ser-Ser-Ser-Gly-Ser-Ser-Ser-Ser-Gly-Ser (SEQ ID NO:30).

In certain embodiments, the Linker is a polyethylene glycol containing linker having 1-12 ethylene glycol residues.

In certain embodiments, the Linker comprises the structure: —CH₂CH₂(OCH₂CH₂)ₘOCH₂—, —(CH₂)ₘCH₂—, —[N(R^{a})—CH(R^{b})(C═O)]ₘ—, or a polypropylene glycol or polypropylene-co-polyethylene glycol group containing 1-100 alkylene glycol units;
wherein each R^{a} is independently H, C₁-C₃ alkyl, or C₁-C₆ alkanol, or combines with R^{b} to form a pyrrolidine or hydroxypyrroline group;
wherein each R^{b} is independently selected from the group consisting of hydrogen, methyl, isopropyl, —CH(CH₃)CH₂CH₃, —CH₂CH(CH₃)₂, —(CH₂)₃-guanidine, —CH₂C(═O)NH₂, —CH₂C(═O)OH, —CH₂SH, —(CH₂)₂C(═O)NH₂, —(CH₂)₂C(═O)OH, —(CH₂)imidazole, —(CH₂)₄NH₂, —CH₂CH₂SCH₃, benzyl, —CH₂OH, —CH(OH)CH₃, —(CH₂)imidazole, or —(CH₂)phenol; and
wherein m is an integer ranging from 1 to 15.

In certain embodiments, the Linker comprises the structure —[N(R′—(CH₂)₁₋₁₅—C(═O)]ₘ—, wherein R′ is H or a C₁-C₃ alkyl optionally substituted with 1-2 hydroxyl groups, and m is an integer ranging from 1 to 100.

In certain embodiments, the Linker comprises the structure

—Z-D-Z′—,

wherein:
Z and Z′ are each independently a bond, —(CH₂)ᵢ—O—, —(CH₂)ᵢ—S—, —(CH₂)ᵢ—N(R)—,

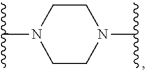 [SMILES: *N1CCN(*)CC1]

—(CH₂)ᵢ—C(R²)═C(R²)— (cis or trans), —(CH₂)ᵢ-≡-, or —Y—C(═O)—Y—;
each R is independently H, C₁-C₃ alkyl, or C₁-C₆ alkanol;
each R² is independently H or C₁-C₃ alkyl;
each Y is independently a bond, O, S, or N(R);
each i is independently 0 to 100; in certain embodiments 0 to 75; in certain
   embodiments 1 to 60; in certain embodiments 1 to 55; in certain
   embodiments 1 to 50; in certain embodiments 1 to 45; in certain
   embodiments 1 to 40; in certain embodiments 2 to 35; in certain
   embodiments 3 to 30; in certain embodiments 1 to 15; in certain
   embodiments 1 to 10; in certain embodiments 1 to 8; in certain
   embodiments 1 to 6; in certain embodiments 0, 1, 2, 3, 4 or 5;
D is a bond, —(CH₂)ᵢ—Y—C(═O)—Y—(CH₂)ᵢ—, —(CH₂)_{m′}—, or —[(CH₂)ₙ—X₁)]ⱼ—, with the proviso that Z, Z′, and D are not each simultaneously bonds;
X₁ is O, S, or N(R);
j is an integer ranging from 1 to 100; in certain embodiments 1 to 75; in certain embodiments 1 to 60; in certain embodiments 1 to 55; in certain
   embodiments 1 to 50; in certain embodiments 1 to 45; in certain
   embodiments 1 to 40; in certain embodiments 2 to 35; in certain
   embodiments 3 to 30; in certain embodiments 1 to 15; in certain
   embodiments 1 to 10; in certain embodiments 1 to 8; in certain
   embodiments 1 to 6; in certain embodiments 1, 2, 3, 4 or 5;
m′ is an integer ranging from 1 to 100; in certain embodiments 1 to 75; in certain embodiments 1 to 60; in certain embodiments 1 to 55; in certain
   embodiments 1 to 50; in certain embodiments 1 to 45; in certain
   embodiments 1 to 40; in certain embodiments 2 to 35; in certain
   embodiments 3 to 30; in certain embodiments 1 to 15; in certain
   embodiments 1 to 10; in certain embodiments 1 to 8; in certain
   embodiments 1 to 6; in certain embodiments 1, 2, 3, 4 or 5;
n is an integer ranging from 1 to 100; in certain embodiments 1 to 75; in certain embodiments 1 to 60; in certain embodiments 1 to 55; in certain
   embodiments 1 to 50; in certain embodiments 1 to 45; in certain
   embodiments 1 to 40; in certain embodiments 2 to 35; in certain
   embodiments 3 to 30; in certain embodiments 1 to 15; in certain
   embodiments 1 to 10; in certain embodiments 1 to 8; in certain
   embodiments 1 to 6; in certain embodiments 1, 2, 3, 4 or 5.

In certain embodiments, the Linker comprises a structure:

CH₂—(OCH₂CH₂)ₙ—CH₂—, —(CH₂CH₂O)_{n′}CH₂CH₂—, or —(CH₂CH₂CH₂O)ₙ—,

wherein each n and n′ is independently an integer ranging from 1 to 25; in certain embodiments 1 to 15; in certain embodiments 1 to 12; in certain embodiments 2 to 11; in certain embodiments 2 to 10; in certain embodiments 2 to 8; in certain embodiments 2 to 6; in certain embodiments 2 to 5; in certain embodiments 2 to 4; in certain embodiments 2 or 3; in certain embodiments 1, 2, 3, 4, 5, 6, 7, or 8.

In certain embodiments, the Linker comprises a structure:

-PEG-CON-PEG-

wherein each PEG is independently a polyethylene glycol group containing from 1-12 ethylene glycol residues and CON is a triazole group

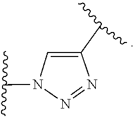 [SMILES: *c1cn(*)nn1]

In certain embodiments, the CON comprises a structure:

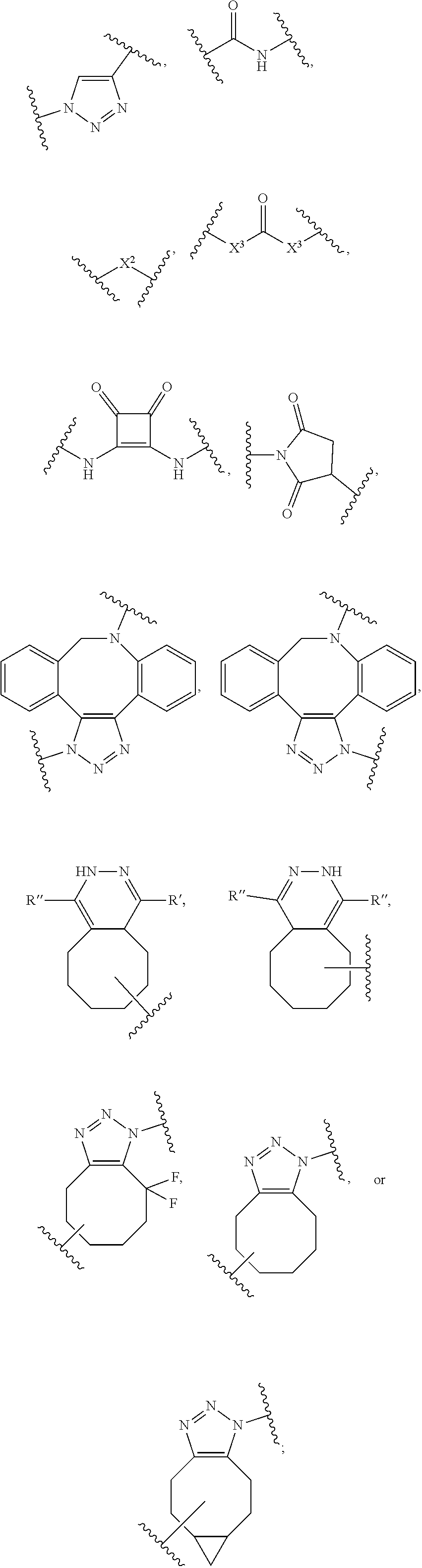

wherein R′ and R″ are each independently H, methyl, or a bond.

In certain embodiments, the CON comprises a diamide structure:

—C(═O)—N(R¹)—(CH₂)_{n″}—N(R¹)C(═O)—,

—N(R¹)—C(═O)(CH₂)_{n″}—C(═O)N(R¹)—, or

—N(R¹)—C(═O)(CH₂)_{n″}—N(R¹)C(═O)—;

wherein each R¹ is independently H or C₁-C₃ alkyl, and n″ is independently an integer from 0 to 8, in certain embodiments 1 to 7, in certain embodiments 1, 2, 3, 4, 5 or 6.

In certain embodiments, the CON comprises a structure:

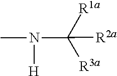

wherein:
R^{1a}, R^{2a} and R^{3a} are each independently H, —(CH₂)_{M1}—, —(CH₂)_{M2}C(═O)_{M3}(NR⁴)_{M3}—(CH₂)_{M2}—, —(CH₂)_{M2}(NR⁴)_{M3}C(O)_{M3}—(CH₂)_{M2}— or —(CH₂)_{M2}O—(CH₂)_{M1}—C(O)NR⁴—, with the proviso that R^{1a}, R^{2a} and R^{3a} are not simultaneously H;
each M1 is independently 1, 2, 3, or 4; in certain embodiments, 1 or 2;
each M2 is independently 0, 1, 2, 3, or 4; in certain embodiments, 0, 1 or 2;
each M3 is independently 0 or 1; and
each R⁴ is independently H, C₁-C₃ alkyl, C₁-C₆ alkanol, or —C(═O)(C₁-C₃ alkyl), with the proviso that M2, and M3 within the same R^{1a}, R^{2a} and R^{3a} cannot all be simultaneously 0.

In certain embodiments, the CON comprises a structure:

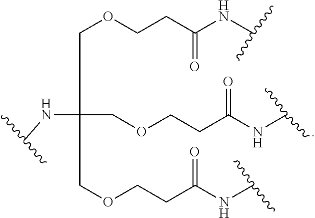 [SMILES: *NC(=O)CCOCC(COCCC(=O)N*)(COCCC(=O)N*)N*]

In other embodiments, the CON comprises a structure:

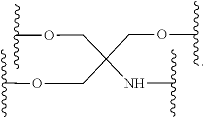 [SMILES: *NC(CO*)(CO*)CO*]

### Additional Galactose- and Talose-Based ASGPR Binding Moieties

In one embodiment, the present invention is directed to compounds which are useful for removing circulating proteins which are associated with a disease state or condition in a patient or subject according to the general chemical structure of Formula II:

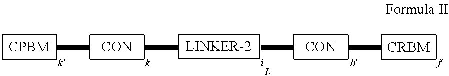

The term “Extracellular Protein Targeting Ligand” as used herein is interchangeably used with the term CPBM (cellular protein binding moiety). The term “ASGPR Ligand” as used herein is interchangeably used with an asiaglycoprotein receptor (ASGPR) binding moiety as defined herein.

In the compound of Formula II, each [CON] is an optional connector chemical moiety which, when present, connects directly to [CPBM] or to [CRBM] or connects the [LINKER-2] to [CPBM] or to [CRBM].

In the compound of Formula II:
[LINKER-2] is a chemical moiety having a valency from 1 to 15 which covalently attaches to one or more [CRBM] and/or [CPBM] group, optionally through a [CON], including a [MULTICON] group, wherein said [LINKER-2] optionally itself contains one or more [CON] or [MULTICON] group(s);
k′ is an integer from 1 to 15;
j′ is an integer from 1 to 15;
h and h′ are each independently an integer from 0 to 15;
i_{L} is an integer from 0 to 15;
with the proviso that at least one of h, h′ and i_{L} is at least 1, or a pharmaceutically acceptable salt, stereoisomer, solvate or polymorph thereof.

A [MULTICON] group can connect one or more of a [CRBM] or [CPBM] to one or more of a [LINKER-2]. In various embodiments, [LINKER-2] has a valency of 1 to 10. In various embodiments, [LINKER-2] has a valency of 1 to 5. In various embodiments, [LINKER-2] has a valency of 1, 2 or 3. In various embodiments, in the compound of Formula II, the [LINKER-2] includes one or more of Linker^{A}, Linker^{B}, Linker^{C}, Linker^{D}, and/or combinations thereof as defined herein.

In the compound of Formula II, xx is independently selected from 0, 1, 2, 3, 4, 5, 6, 7, 8, 9, 10, 11, 12, 13, 14, 15, 16, 17, 18, 19, 20, 21, 22, 23, 24, and 25.

In the compound of Formula II, yy is independently selected from 0, 1, 2, 3, 4, 5, 6, 7, 8, 9, 10, 11, 12, 13, 14, 15, 16, 17, 18, 19, 20, 21, 22, 23, 24, and 25.

In the compound of Formula II, zz is independently selected from 0, 1, 2, 3, 4, 5, 6, 7, 8, 9, 10, 11, 12, 13, 14, 15, 16, 17, 18, 19, 20, 21, 22, 23, 24, and 25.

In the compound of Formula II, X¹ is 1 to 5 contiguous atoms independently selected from O, S, N(R^{b}), and C(R⁴)(R⁴), wherein if X¹ is 1 atom then X¹ is O, S, N(R⁶), or C(R⁴)(R⁴), if X¹ is 2 atoms then no more than 1 atom of X¹ is O, S, or N(R⁶), if X¹ is 3, 4, or 5 atoms then no more than 2 atoms of X¹ are O, S, or N(R⁶)
R³ at each occurrence is independently selected from hydrogen, alkyl, heteroalkyl, haloalkyl (including —CF₃, —CHF₂, —CH₂F, —CH₂CF₃, —CH₂CH₂F, and —CF₂CF₃), arylalkyl, heteroarylalkyl, alkenyl, alkynyl, and, heteroaryl, heterocycle, —OR⁸, and —NR⁸R⁹;
R⁴ is independently selected at each occurrence from hydrogen, heteroalkyl, alkyl, haloalkyl, arylalkyl, heteroarylalkyl, alkenyl, alkynyl, aryl, heteroaryl, heterocycle, —OR⁶, —NR⁶R⁷,
R⁶ and R⁷ are independently selected at each occurrence from hydrogen, heteroalkyl, alkyl, arylalkyl, heteroaryl alkyl, alkenyl, alkynyl, and, haloalkyl, heteroaryl, heterocycle, -alkyl-OR⁸, -alkyl-NR⁸R⁹, C(O)R³, S(O)R³, C(S)R³, and S(O)₂R³;
R⁸ and R⁹ are independently selected at each occurrence from hydrogen, heteroalkyl, alkyl, arylalkyl, heteroarylalkyl, alkenyl, alkynyl, aryl, heteroaryl, and heterocycle.

A. Galactose-Based ASGPR-Binding Cellular Receptor Binding Moieties of Formula II

In certain embodiments, the compound of Formula II is selected from.

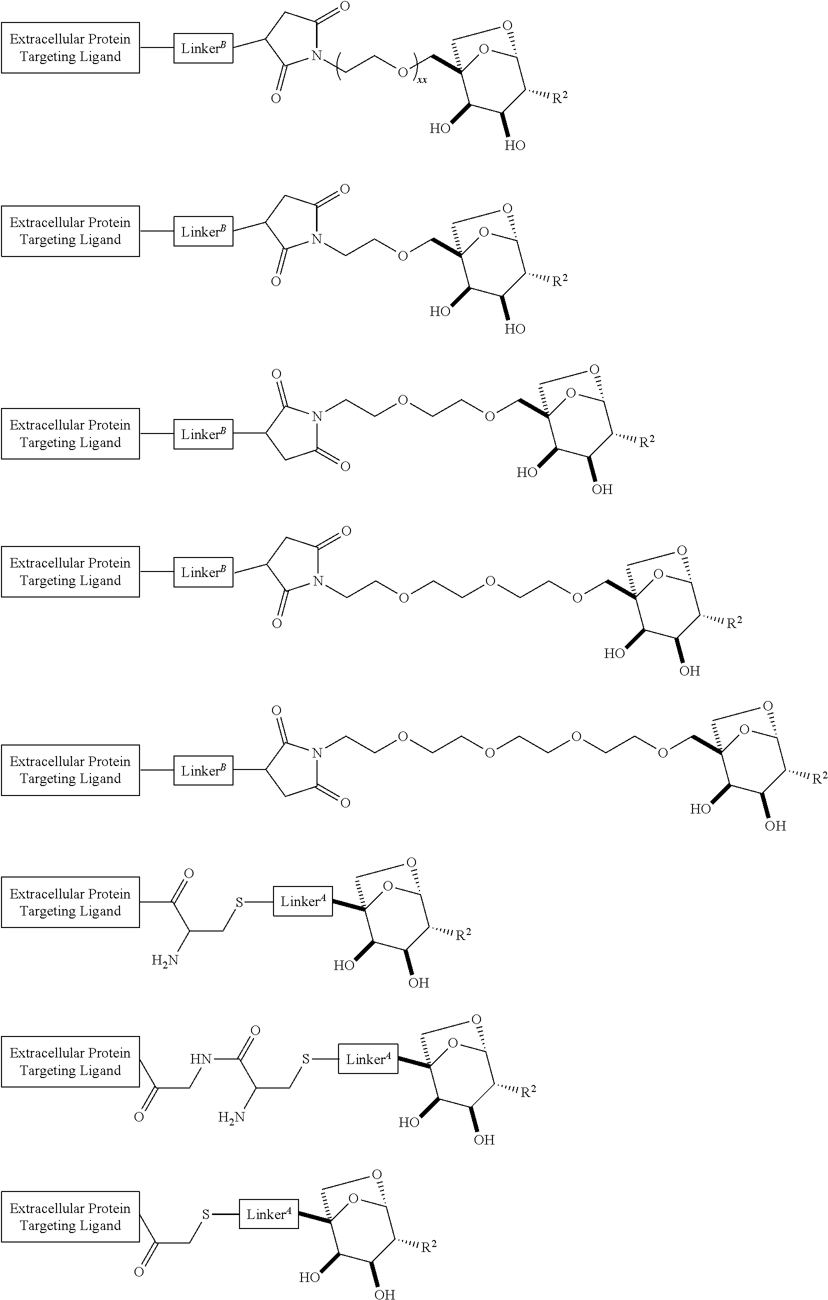

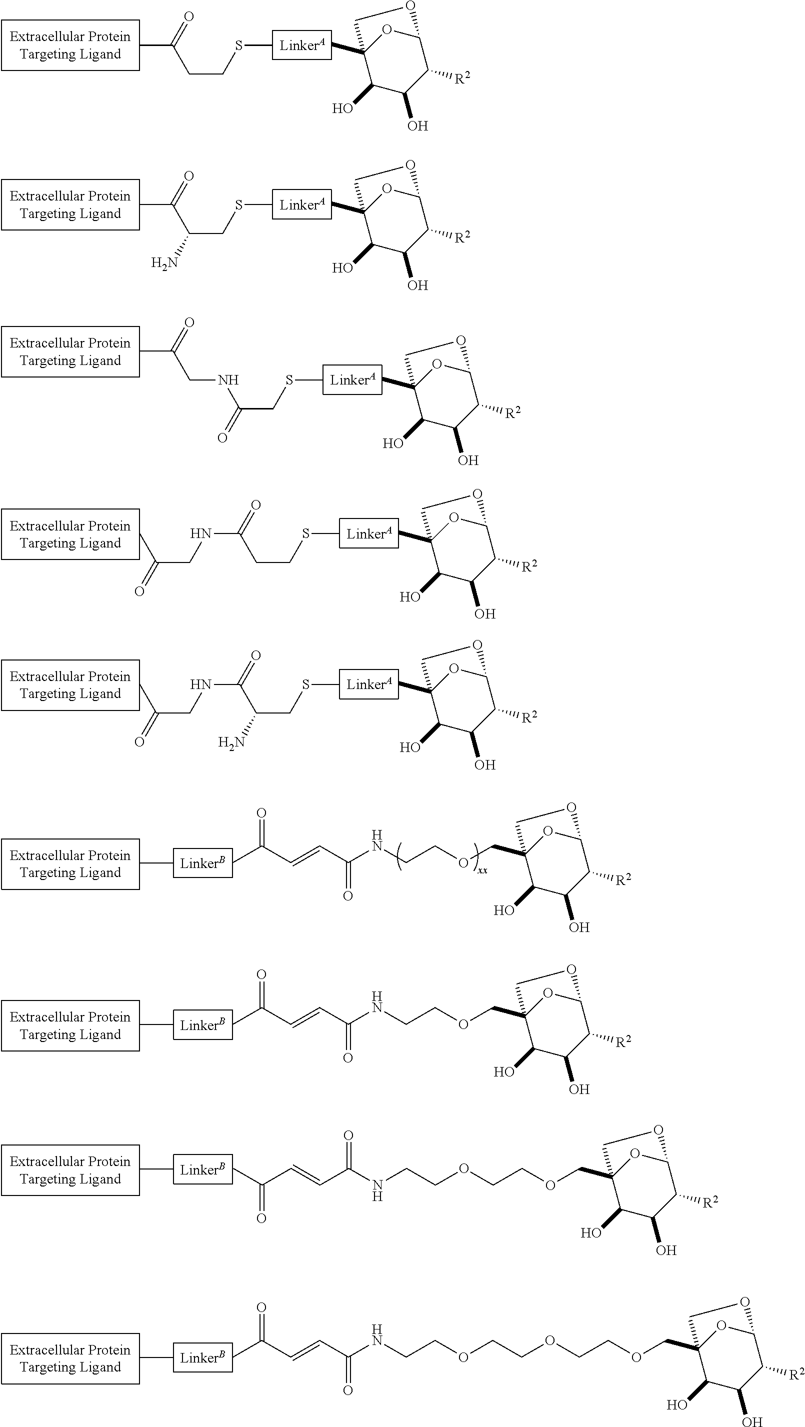

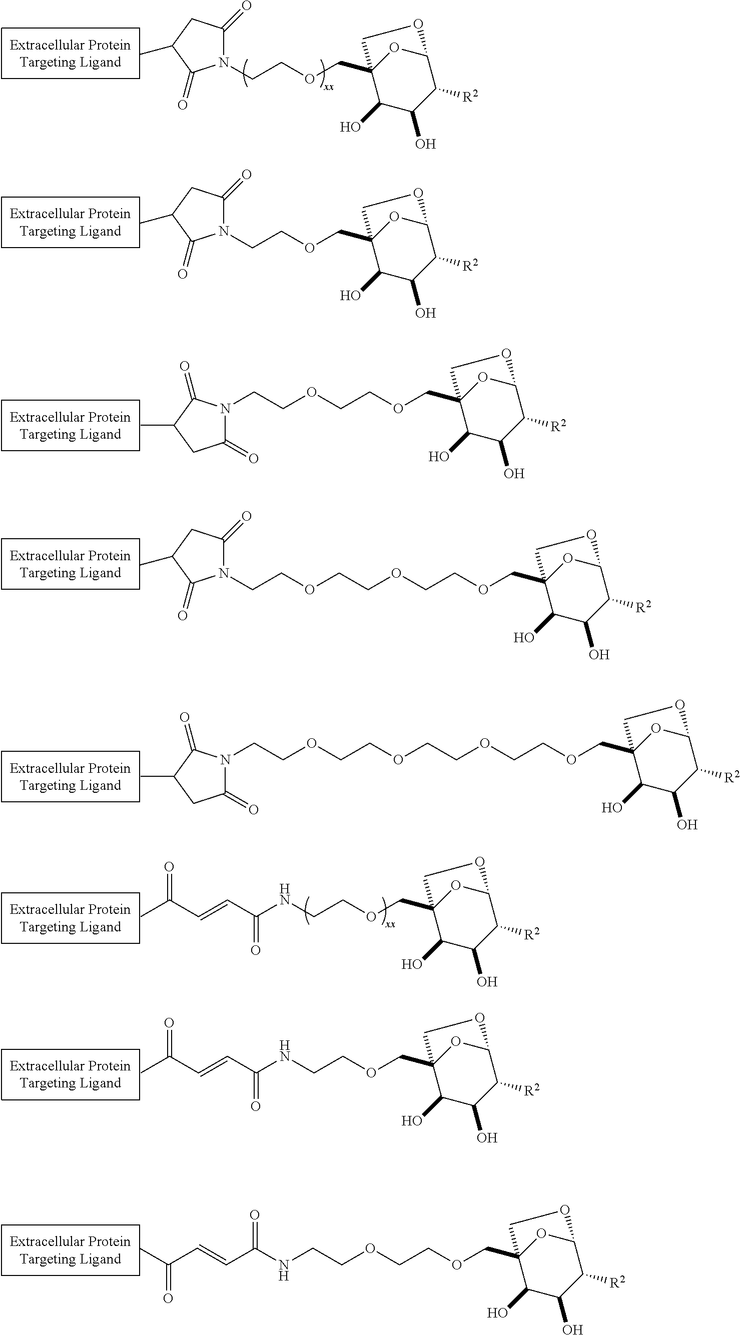

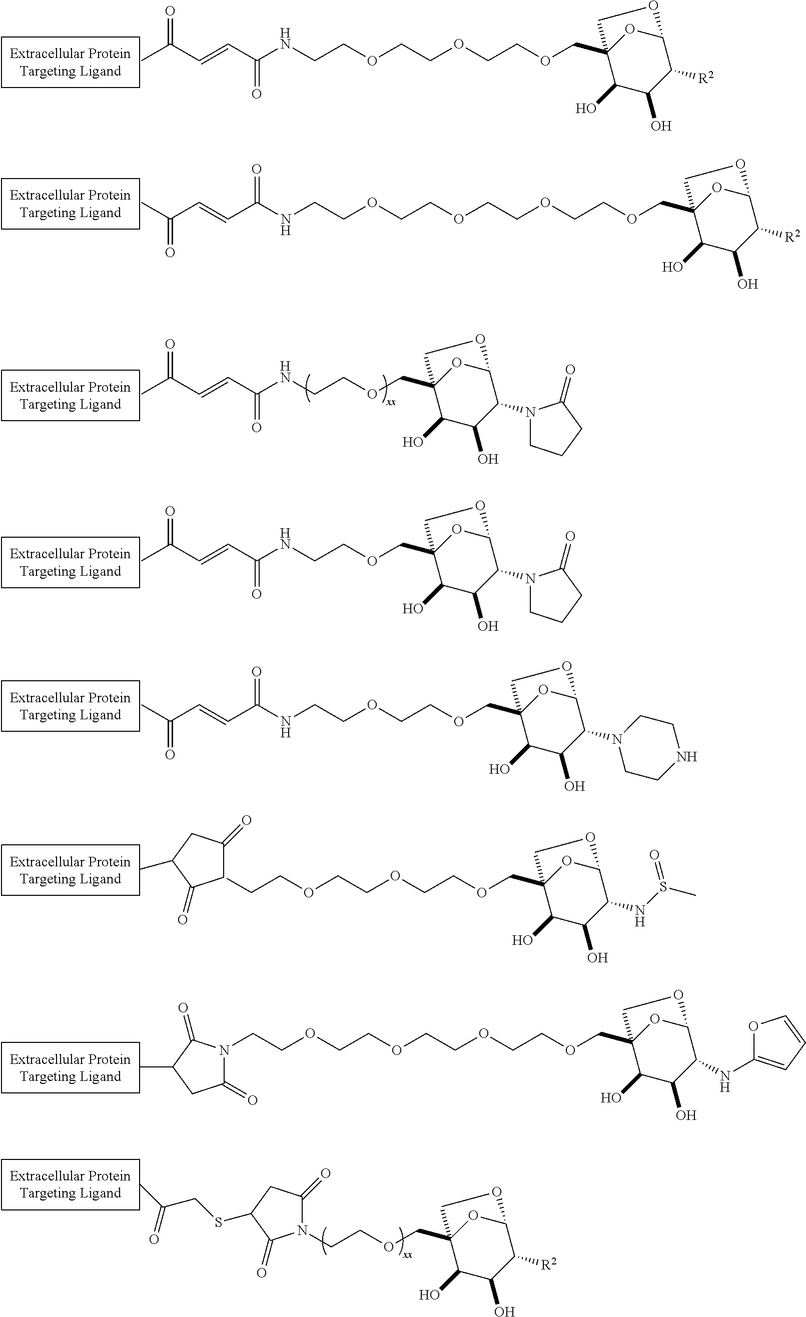

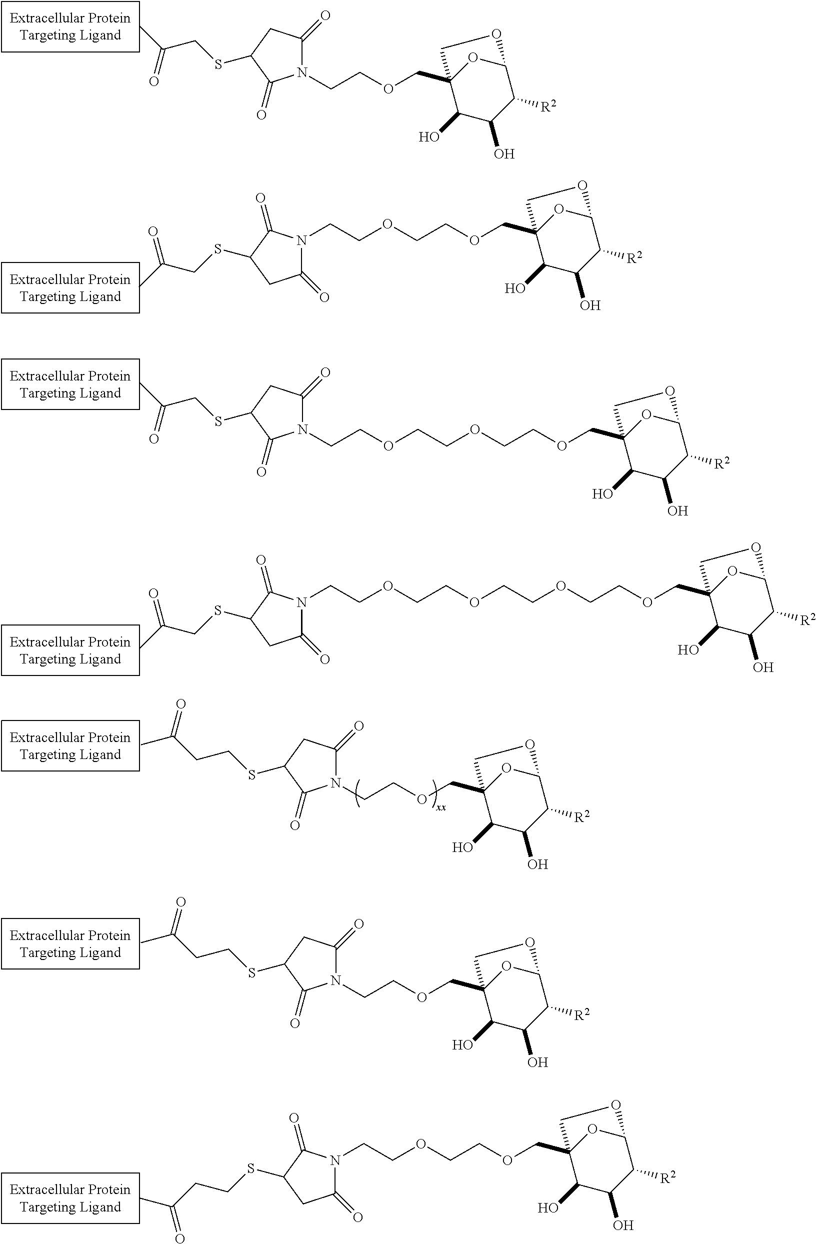

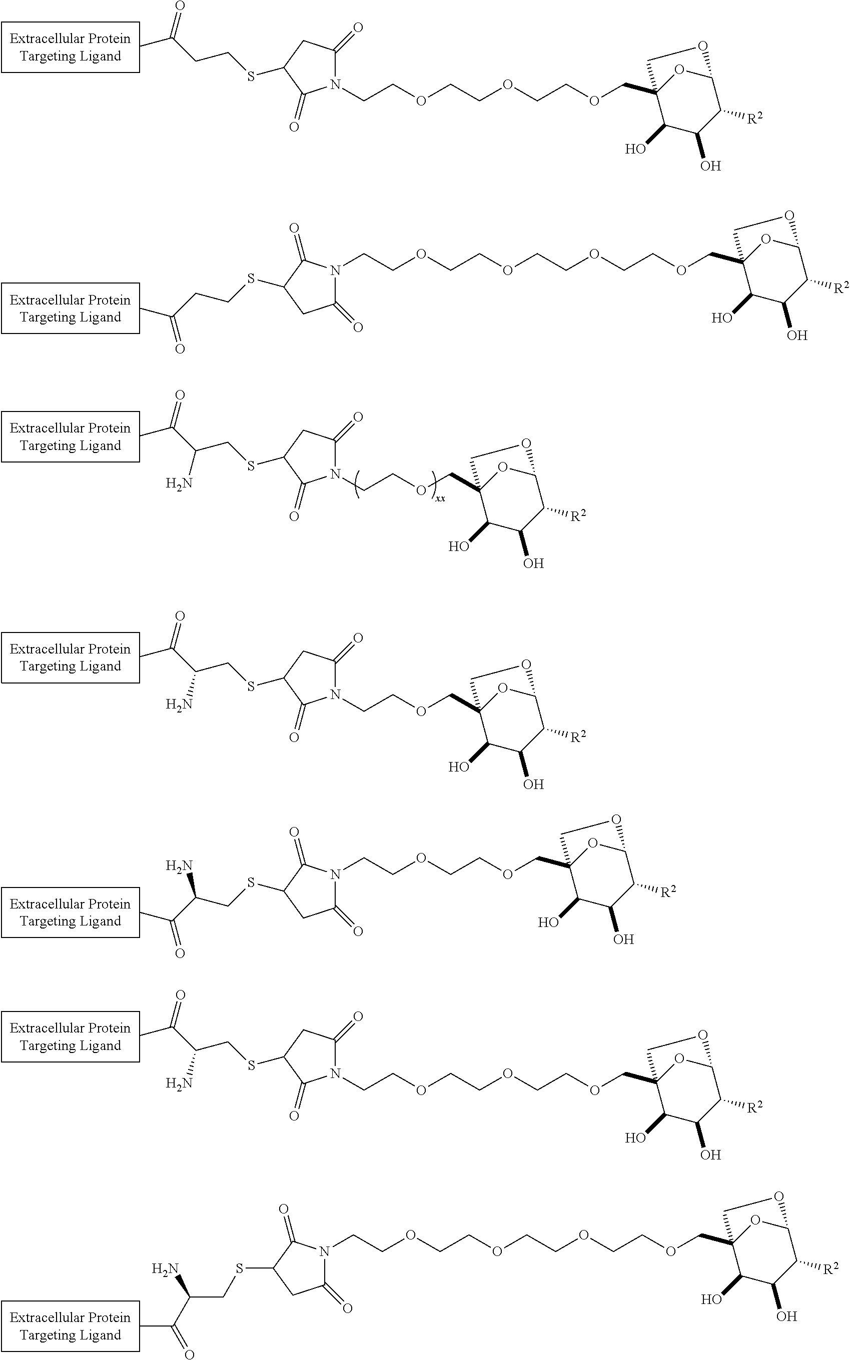

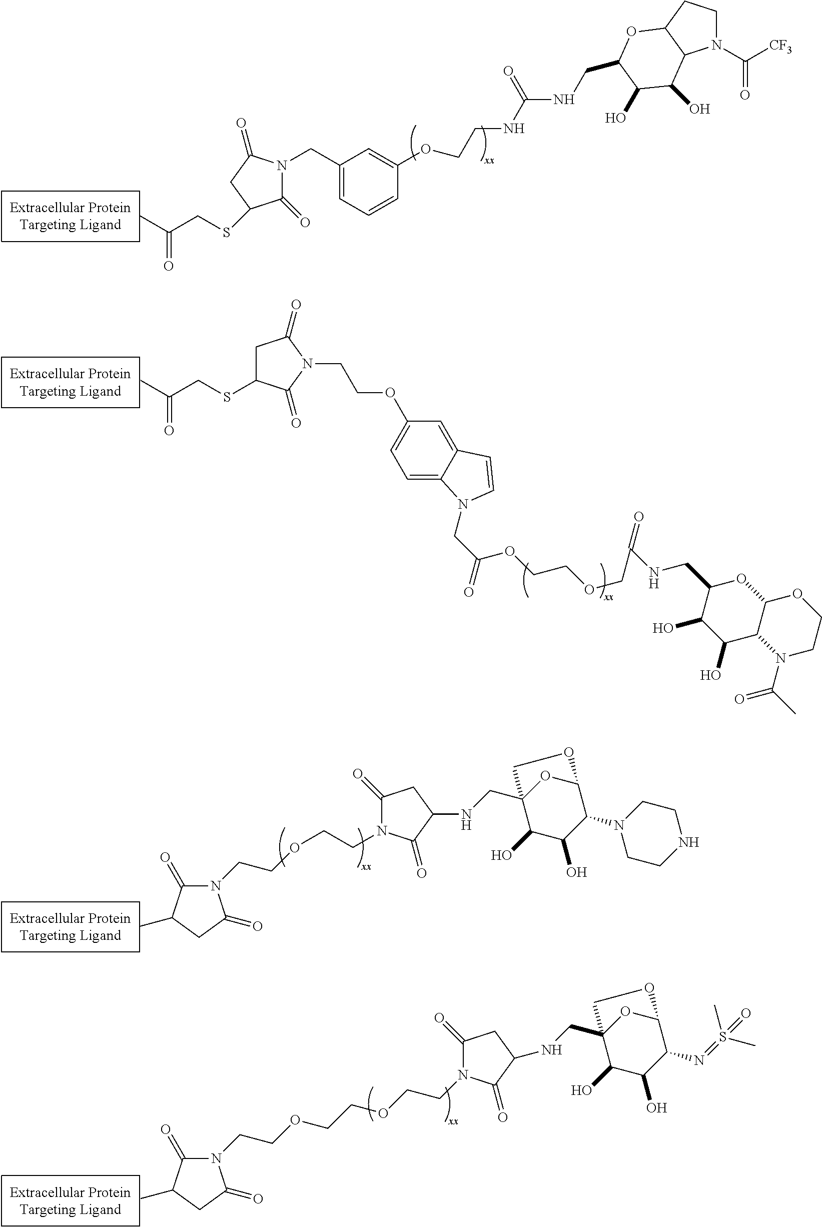

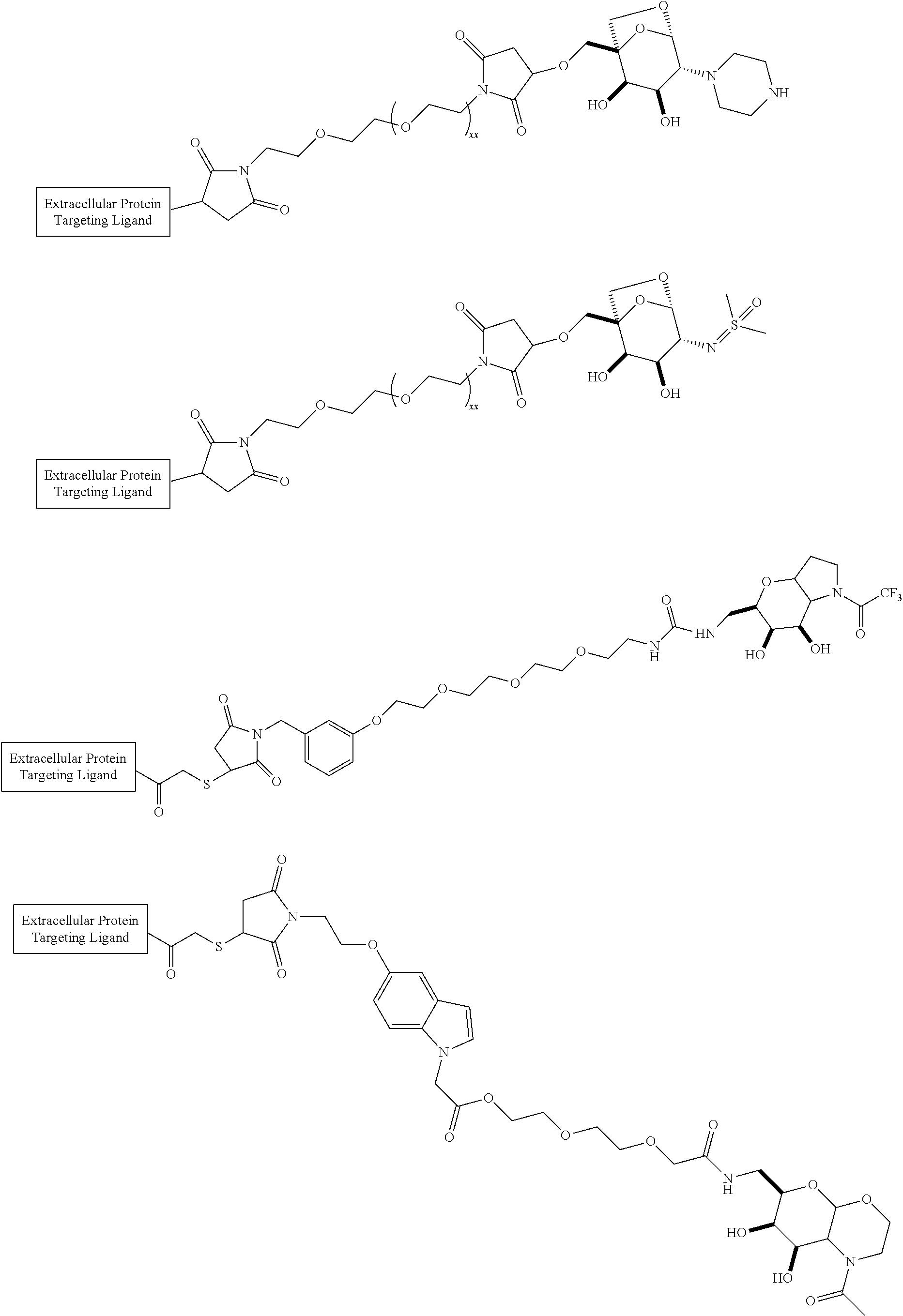

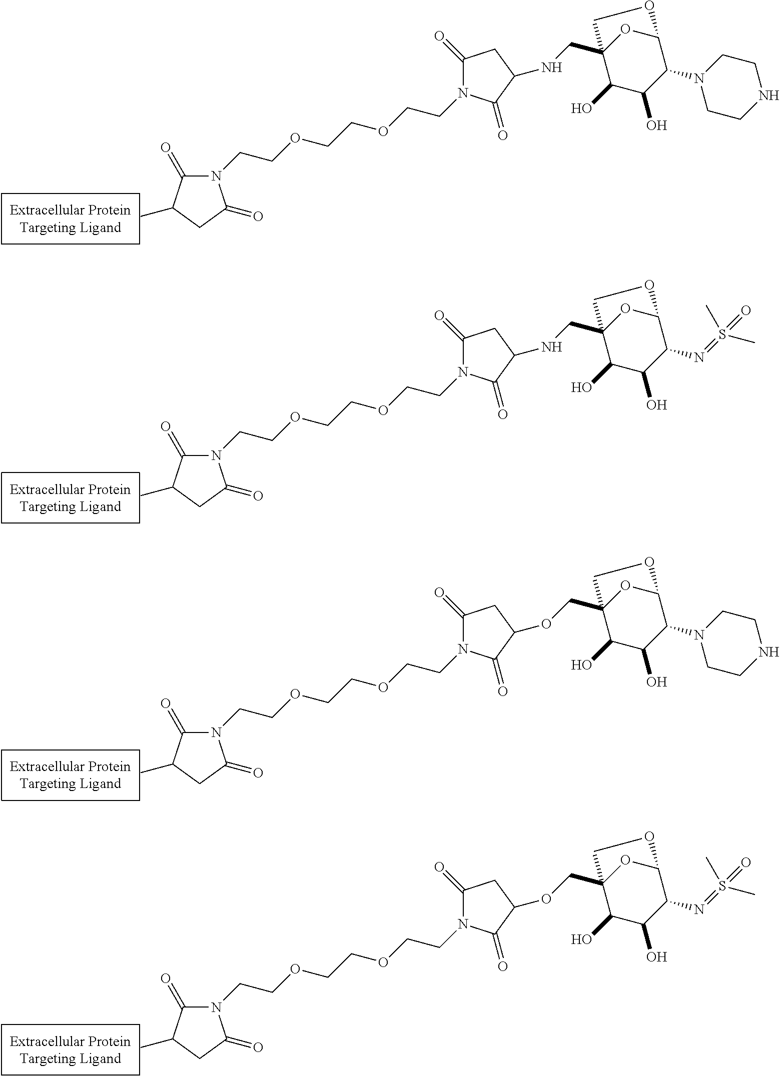

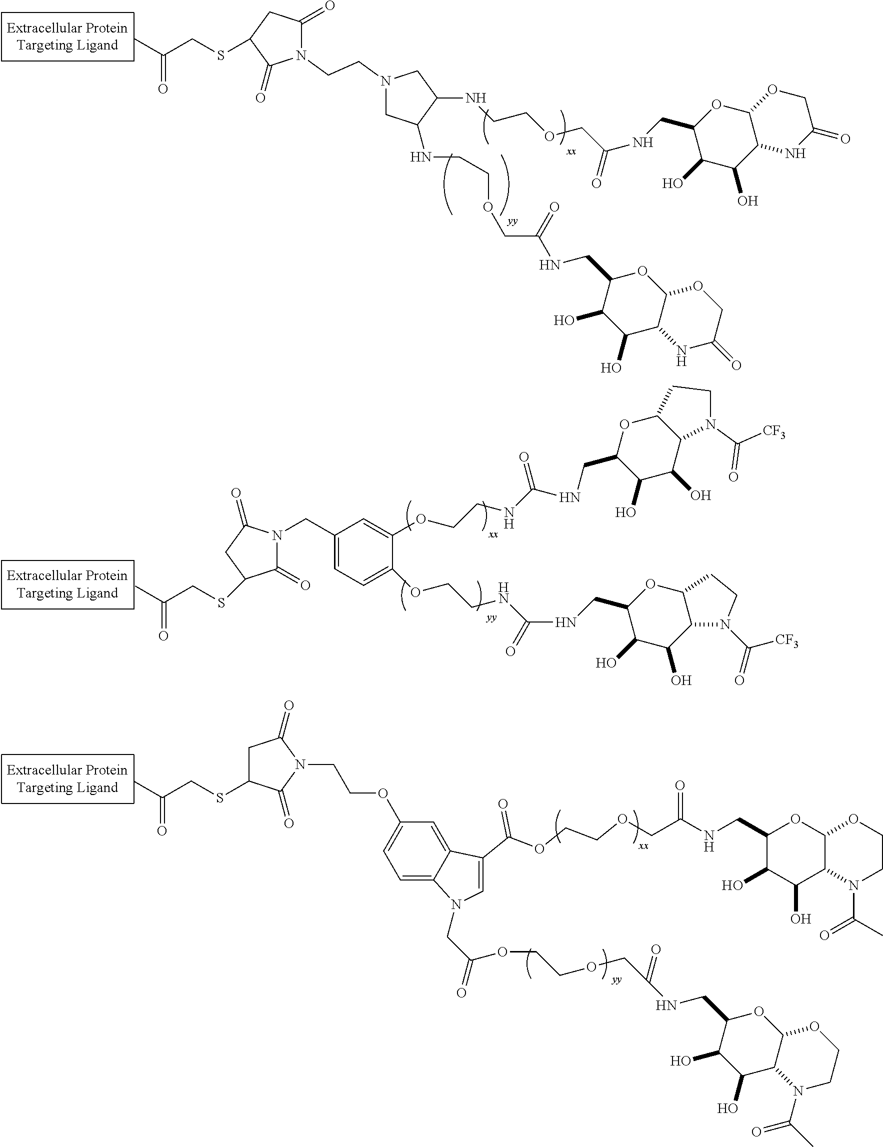

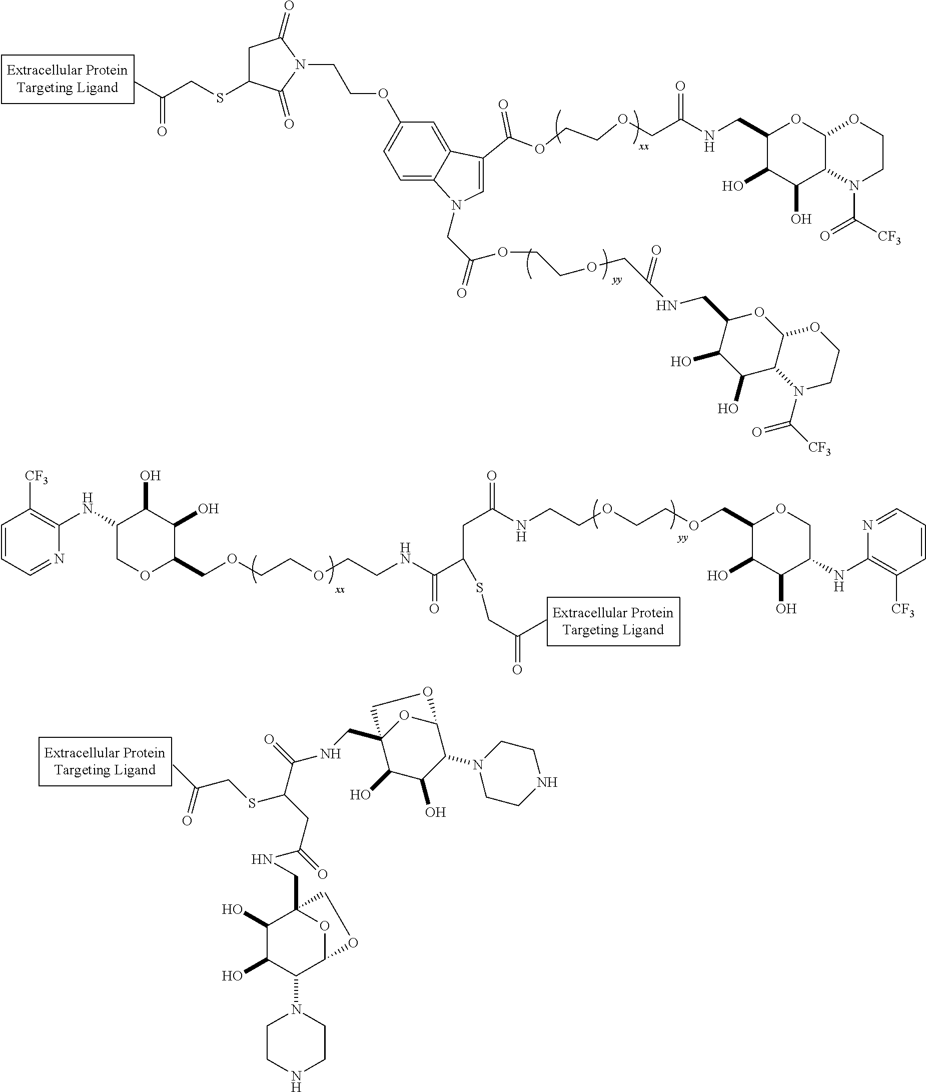

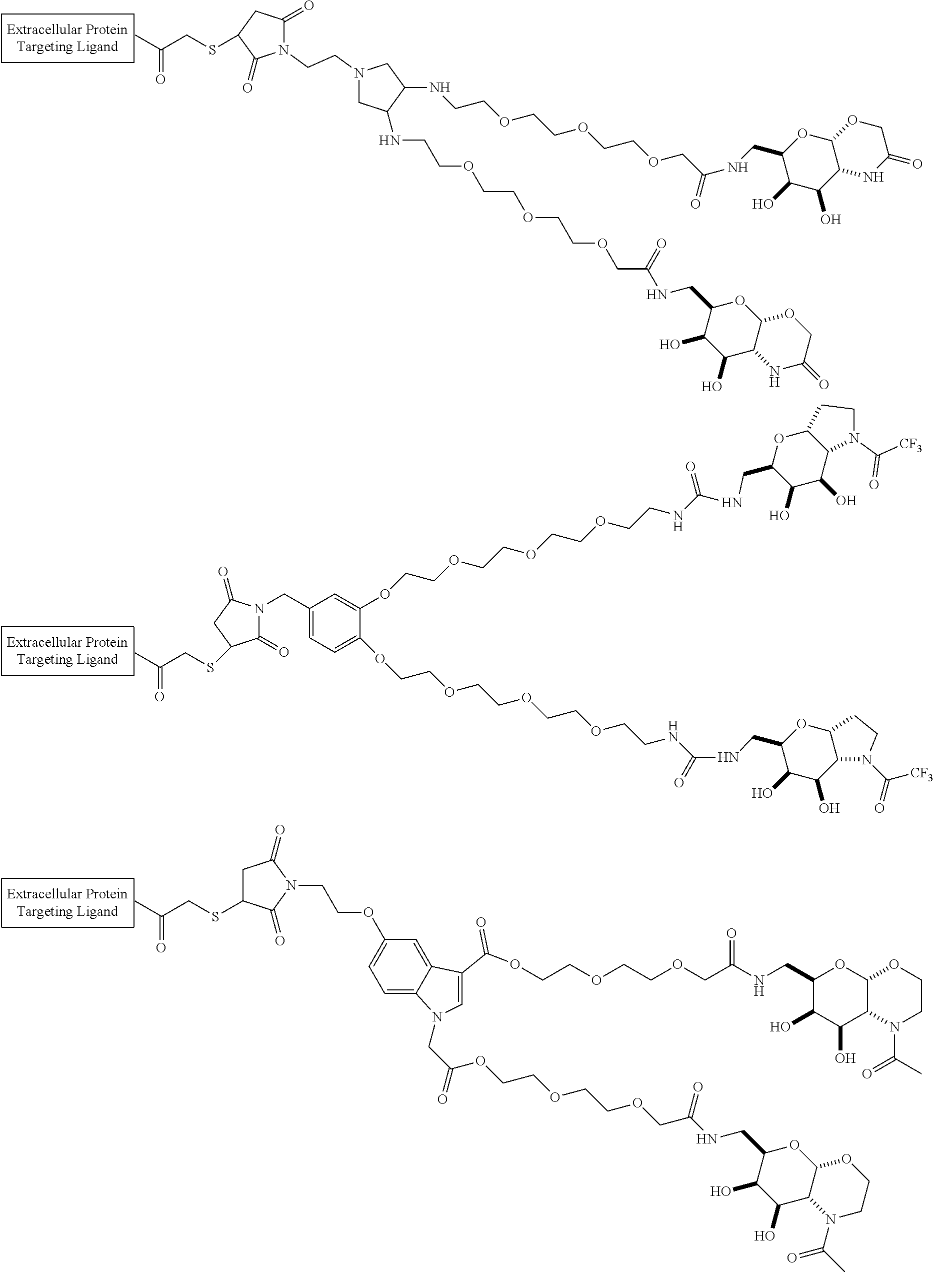

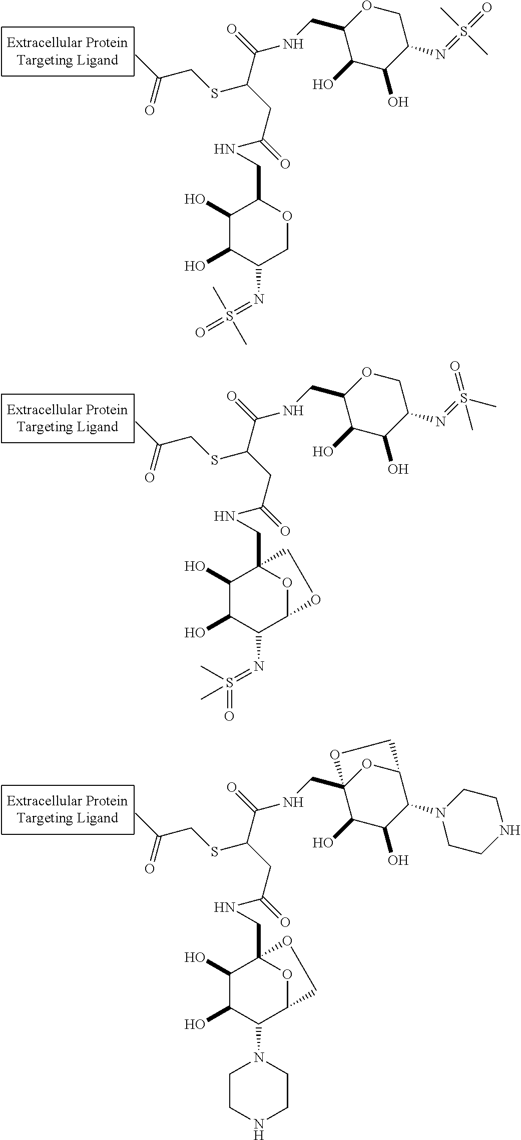

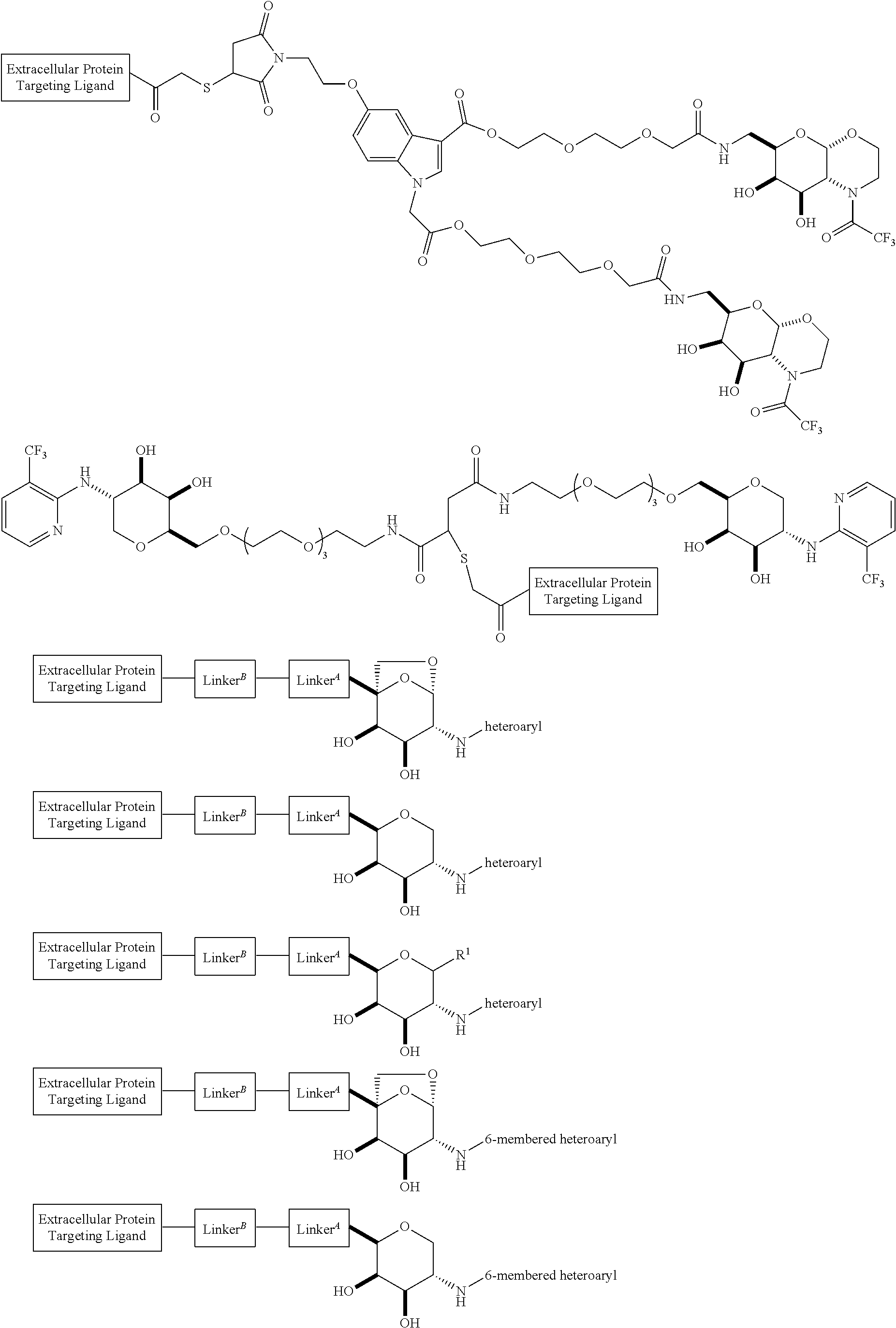

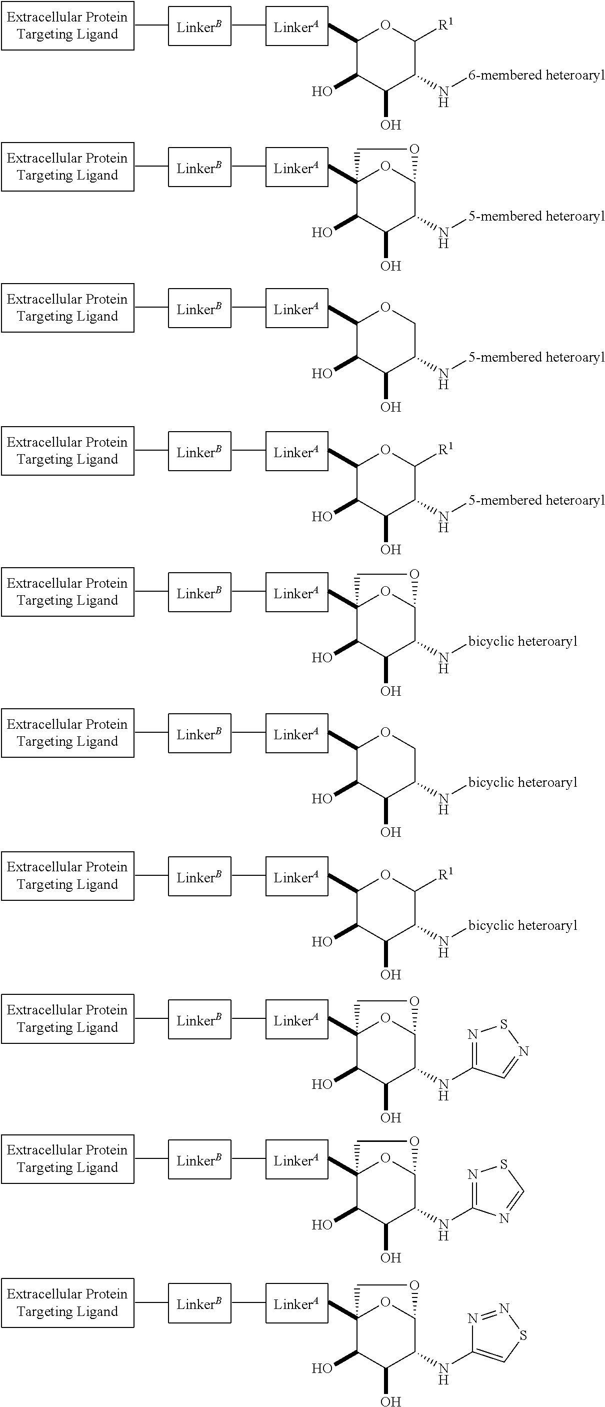

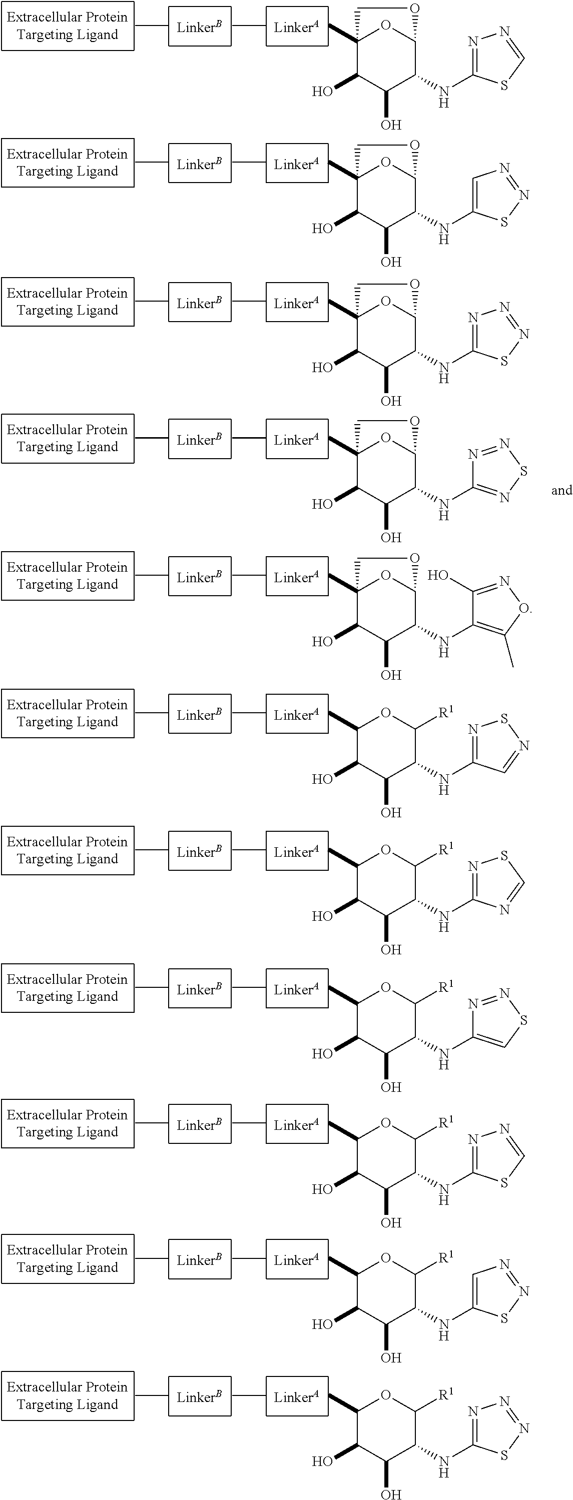

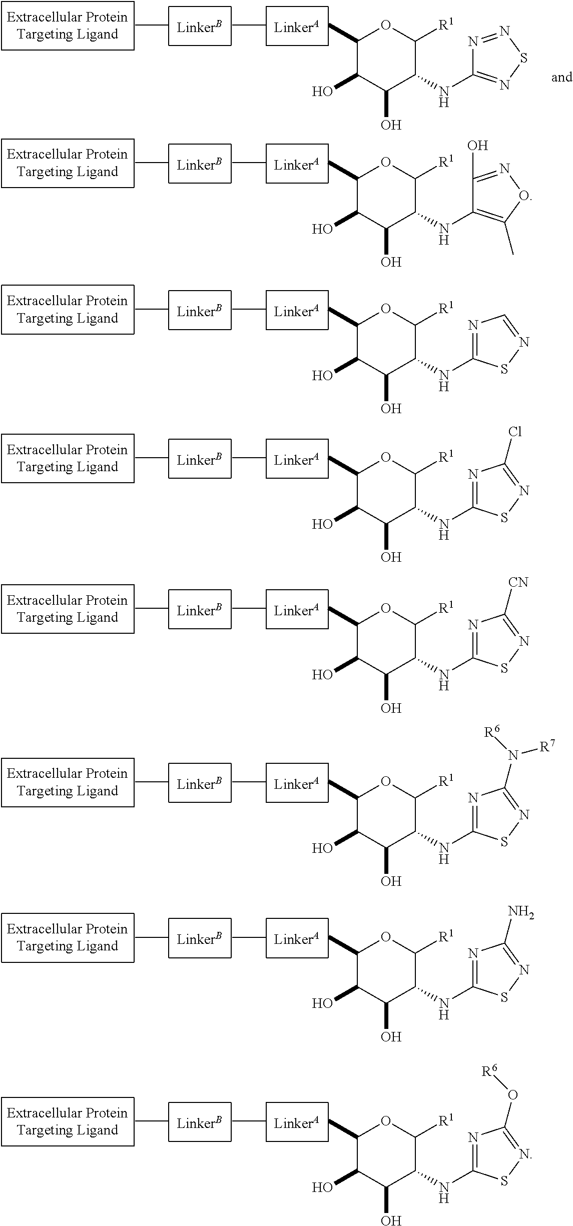

In one embodiment, the compound of Formula II has one of the following structures.

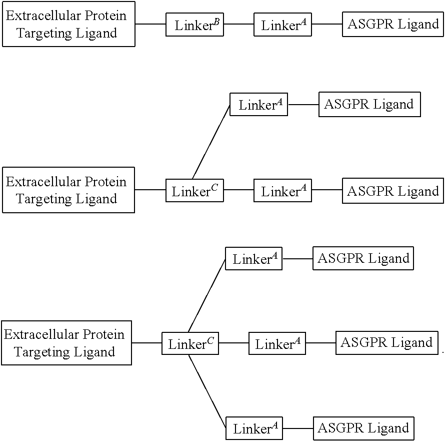

In various embodiments, the ASGPR ligand is linked at either the C¹ or C⁵ (R¹ or R⁵) position to form a degrading compound. In various embodiments, the ASGPR ligand is linked at C⁶ position to form a degrading compound. For example, when the ASGPR ligand is

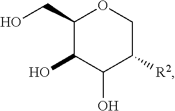

then non-limiting examples of ASGPR binding compounds of Formula II include:

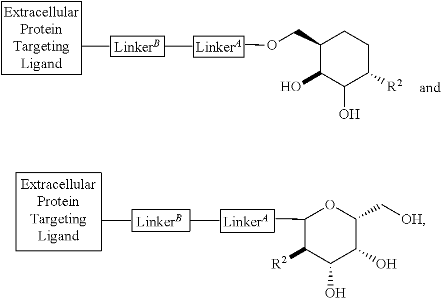

or the bi- or tri-substituted versions thereof or pharmaceutically acceptable salts thereof, where the bi- or tri-substitution refers to the number additional galactose derivatives attached to a linker moiety.

In any of the embodiments herein where an ASGPR ligand is drawn for use in a degrader the ASGPR ligand is typically linked through to the Extracellular Protein Targeting Ligand in the C⁵ position (e.g., which can refer to the adjacent C⁶ carbon hydroxyl or other functional moiety that can be used for linking purposes). When the linker and Extracellular Protein Targeting Ligand is connected through the C¹ position, then that carbon is appropriately functionalized for linking, for example with a hydroxyl, amino, allyl, alkyne or hydroxyl-allyl group.

In various embodiments, the ASGPR ligand is not linked in the C³ or C⁴ position, because these positions chelate with the calcium for ASGPR binding in the liver. In certain embodiments, an ASGPR ligand useful for incorporation into a compound of Formula II is selected from.

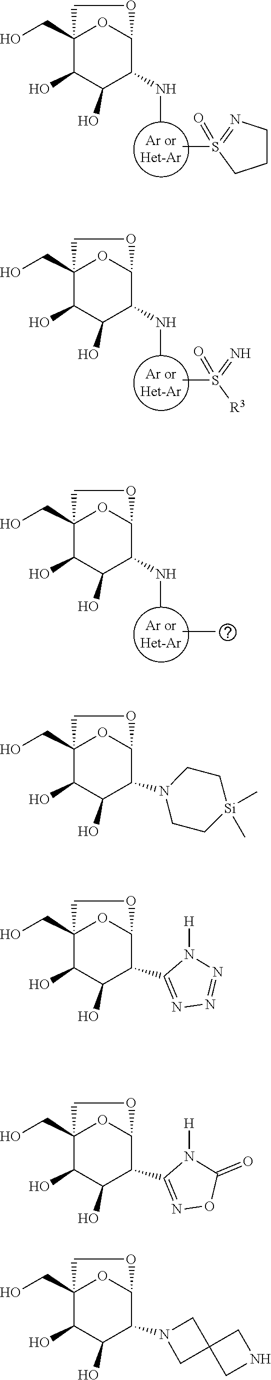

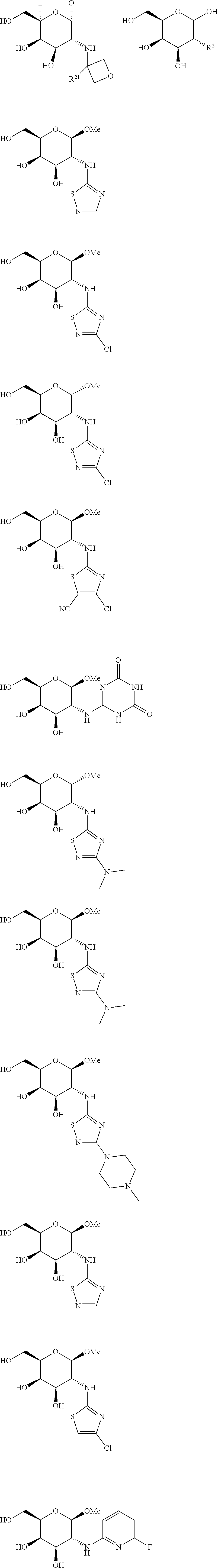

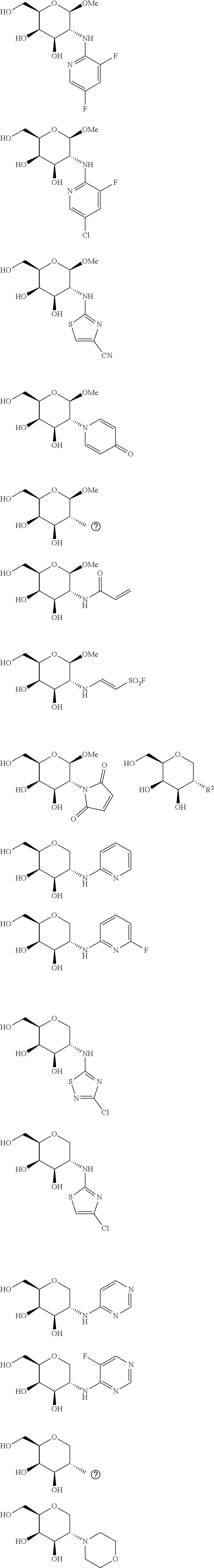

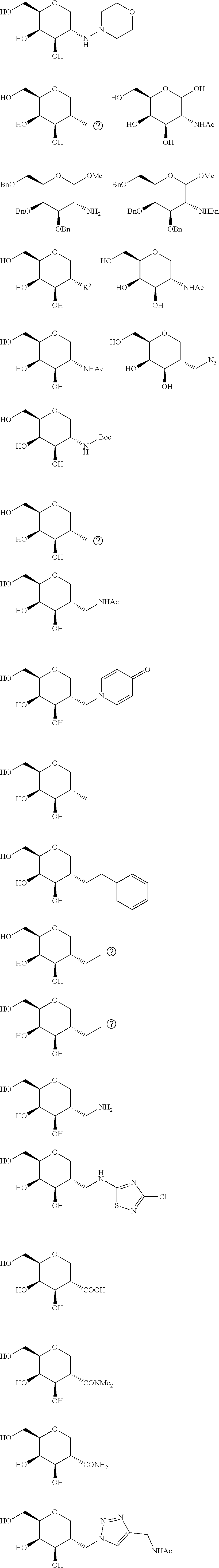

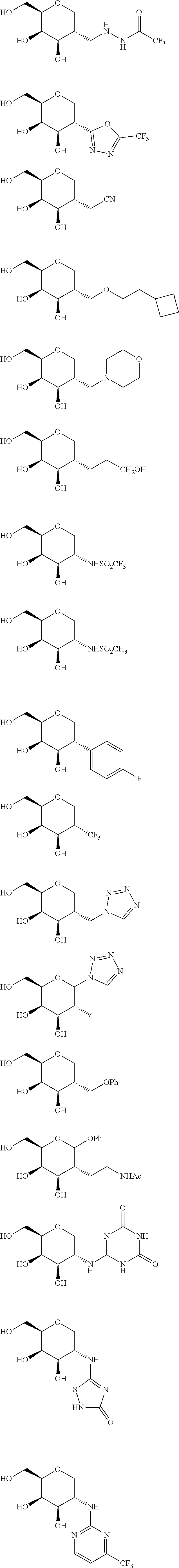

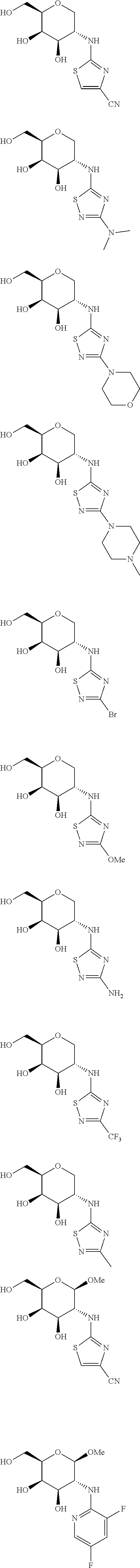

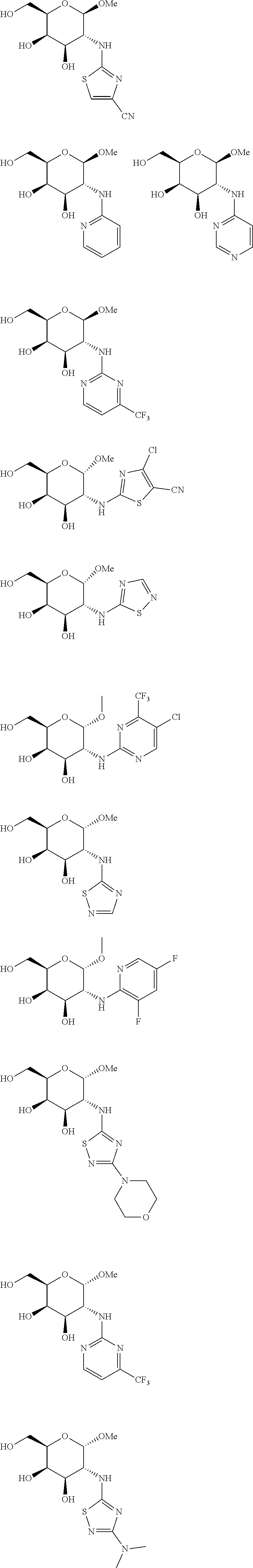

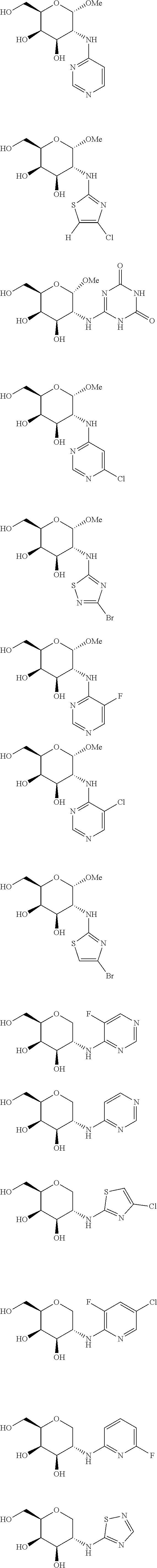

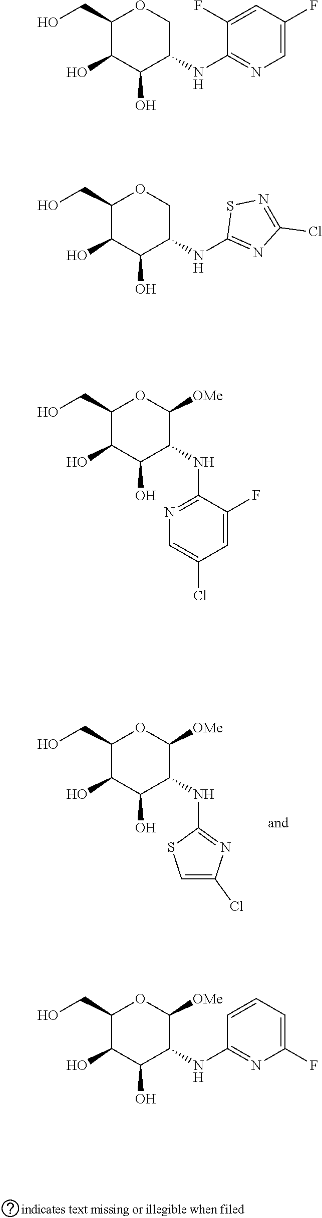 [SMILES: OC[C@H]1OC[C@H](Nc2ncc(F)cc2F)[C@@H](O)[C@H]1O ; OC[C@H]1OC[C@H](Nc2nc(Cl)ns2)[C@@H](O)[C@H]1O ; CO[C@@H]1O[C@H](CO)[C@H](O)[C@H](O)[C@H]1Nc1ncc(Cl)cc1F ; CO[C@@H]1O[C@H](CO)[C@H](O)[C@H](O)[C@H]1Nc1nc(Cl)cs1 ; CO[C@@H]1O[C@H](CO)[C@H](O)[C@H](O)[C@H]1Nc1cccc(F)n1]

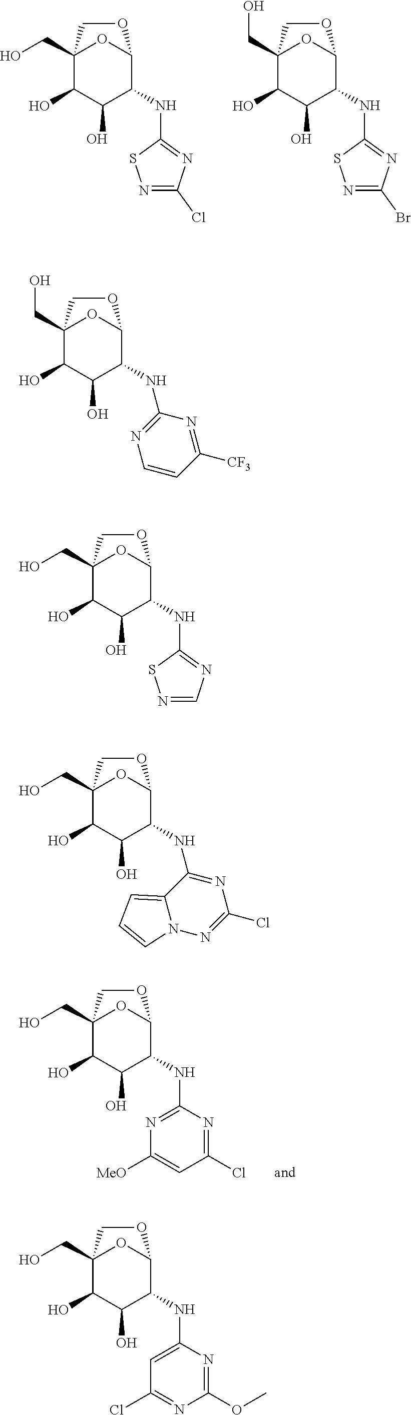

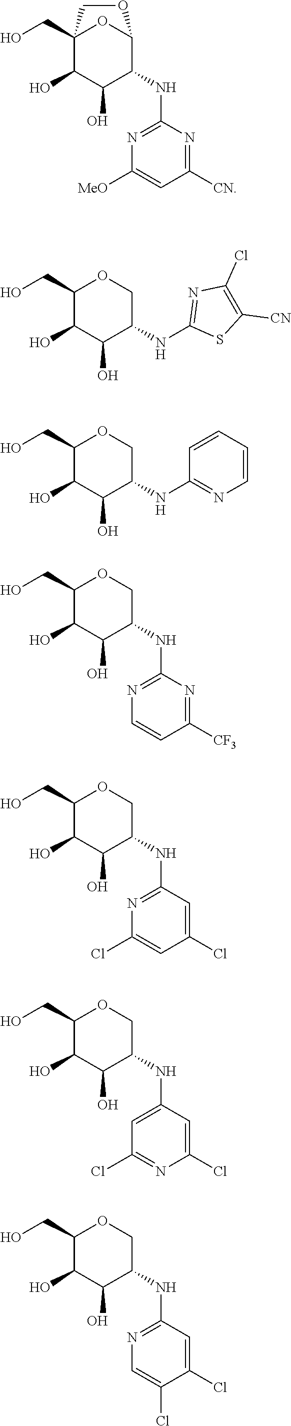

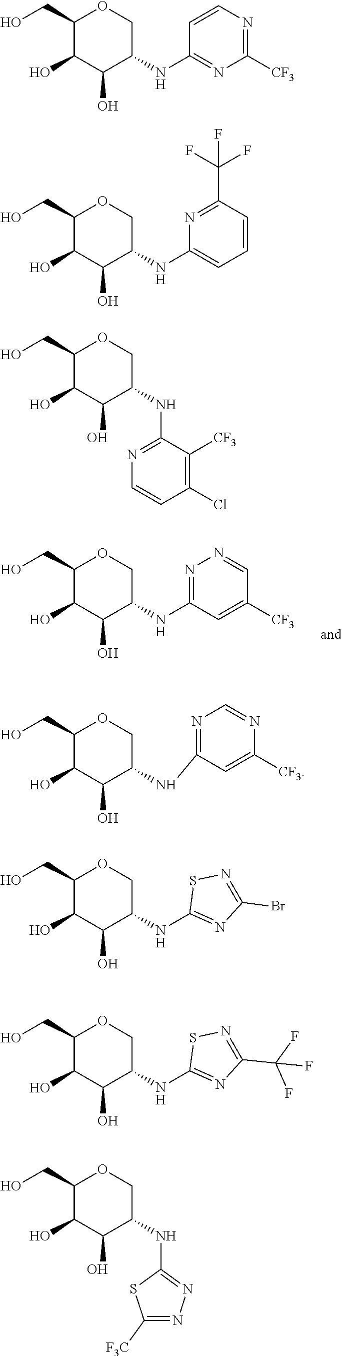

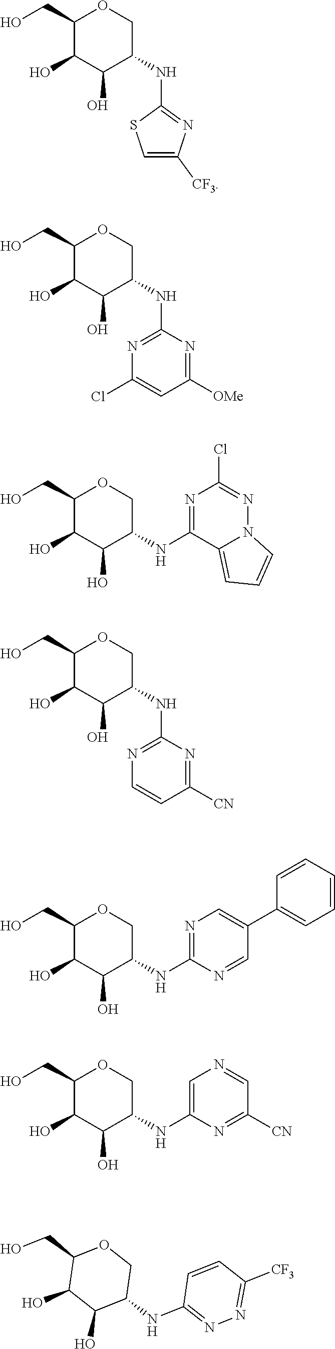

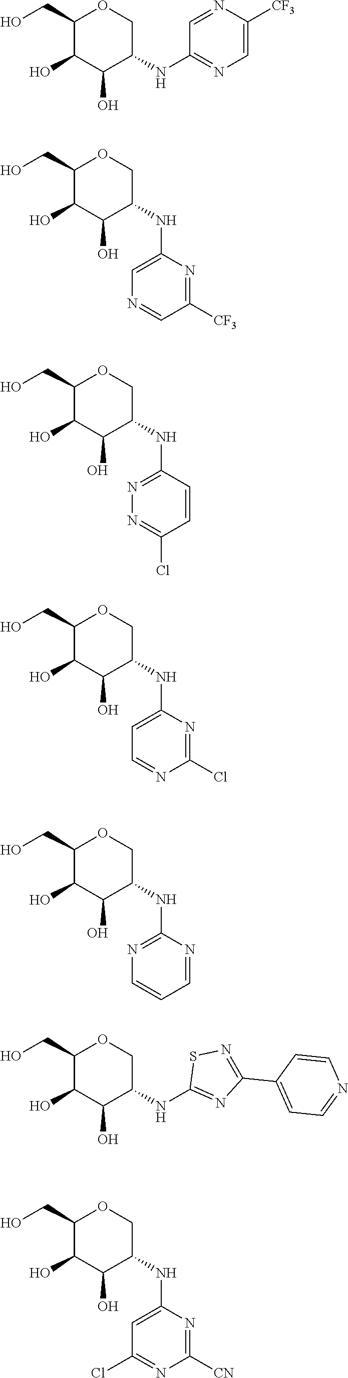

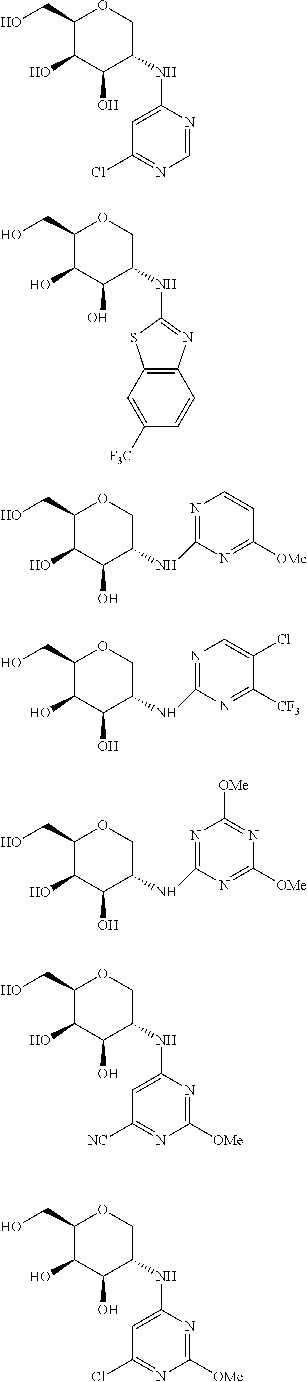

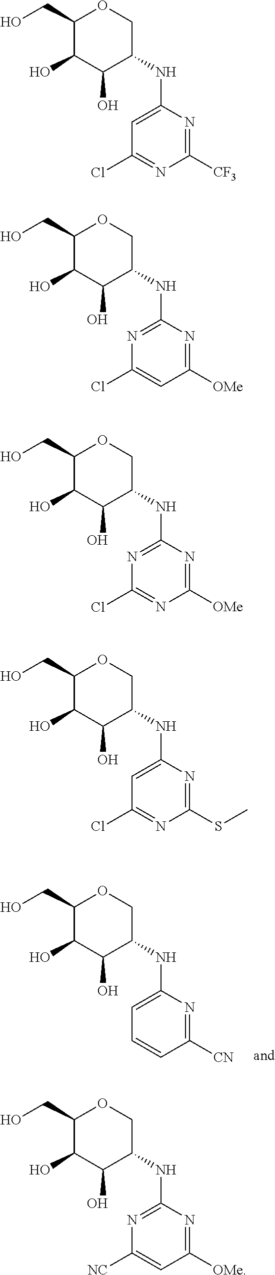 [SMILES: OC[C@H]1OC[C@H](Nc2cc(Cl)nc(C(F)(F)F)n2)[C@@H](O)[C@H]1O ; COc1cc(Cl)nc(N[C@H]2CO[C@H](CO)[C@H](O)[C@@H]2O)n1 ; COc1nc(Cl)nc(N[C@H]2CO[C@H](CO)[C@H](O)[C@@H]2O)n1 ; CSc1nc(Cl)cc(N[C@H]2CO[C@H](CO)[C@H](O)[C@@H]2O)n1 ; N#Cc1cccc(N[C@H]2CO[C@H](CO)[C@H](O)[C@@H]2O)n1 ; COc1cc(C#N)nc(N[C@H]2CO[C@H](CO)[C@H](O)[C@@H]2O)n1]

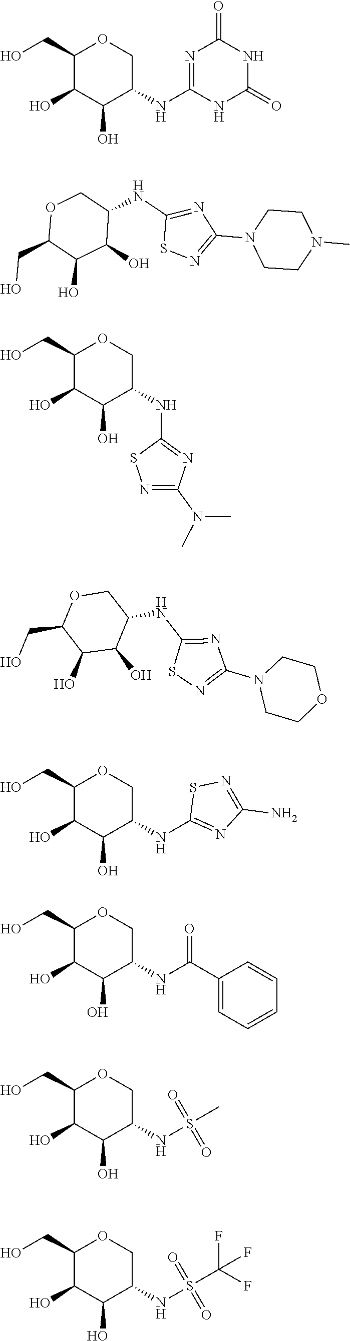

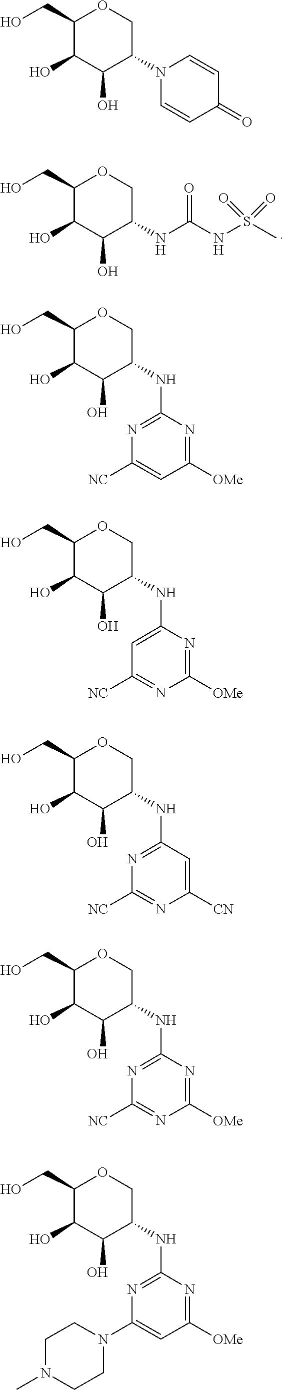

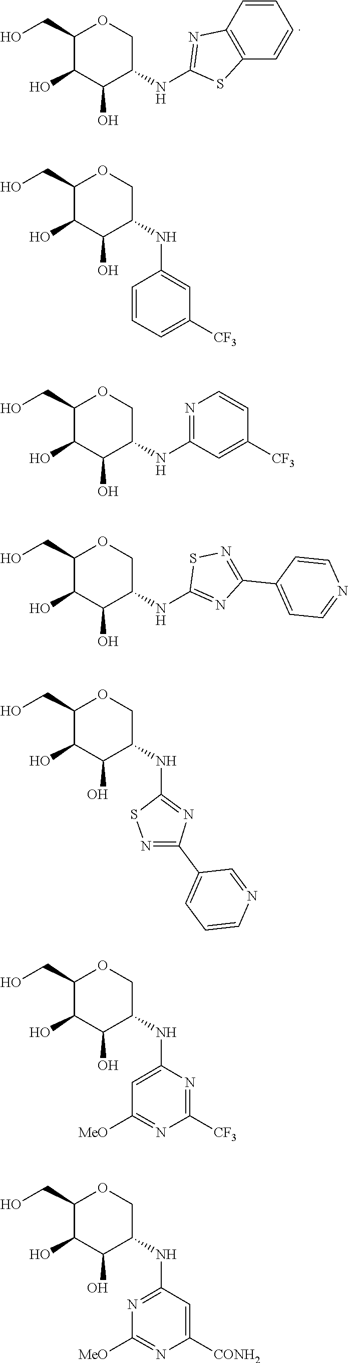

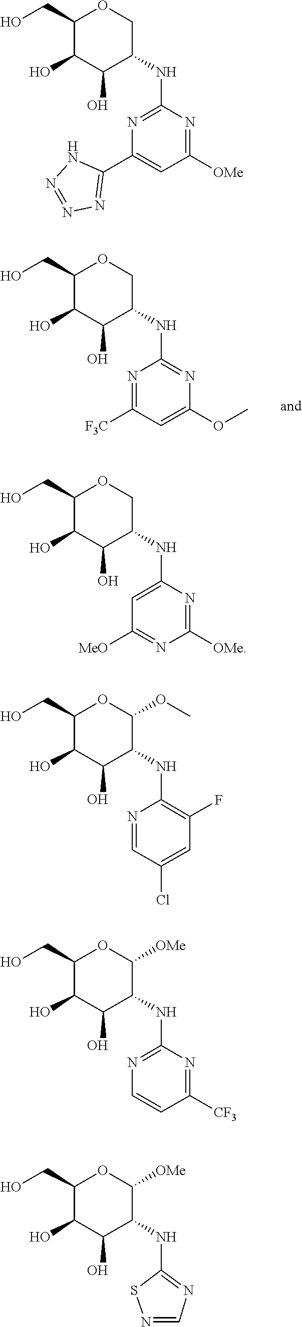

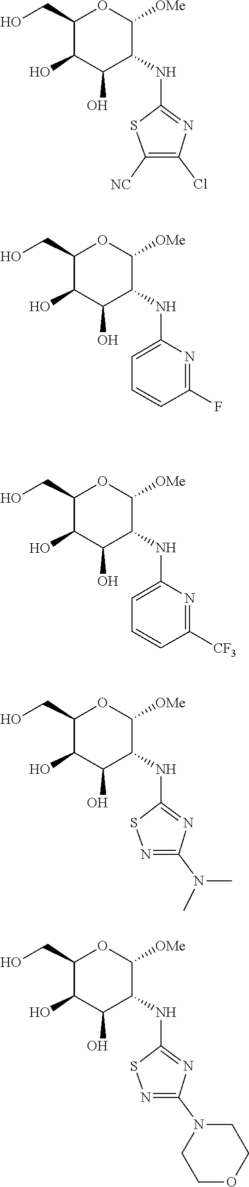

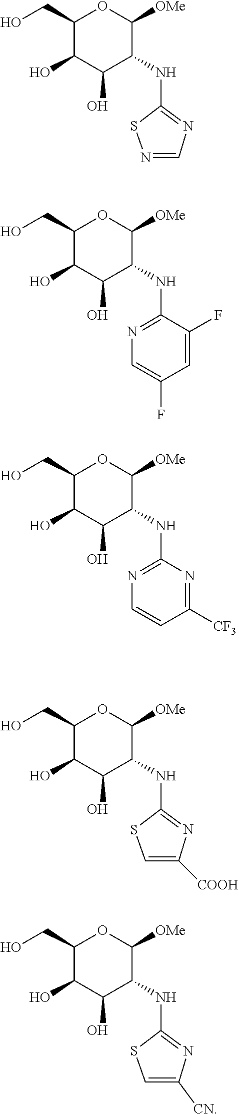

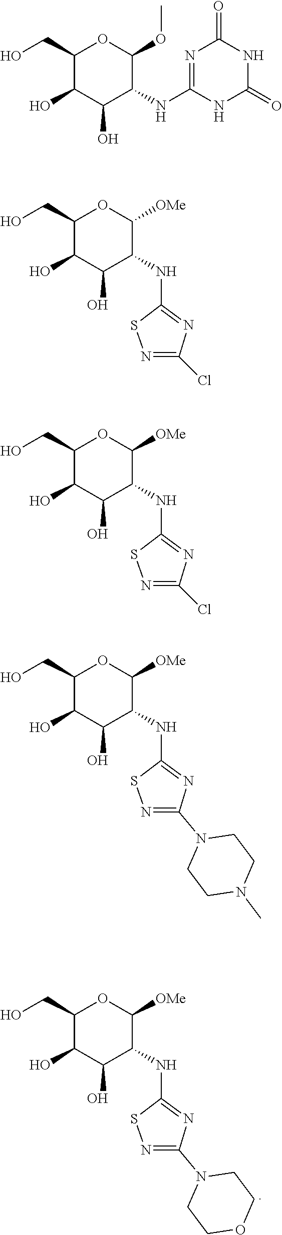 [SMILES: CO[C@@H]1O[C@H](CO)[C@H](O)[C@H](O)[C@H]1Nc1nc(=O)[nH]c(=O)[nH]1 ; CO[C@H]1O[C@H](CO)[C@H](O)[C@H](O)[C@H]1Nc1nc(Cl)ns1 ; CO[C@@H]1O[C@H](CO)[C@H](O)[C@H](O)[C@H]1Nc1nc(Cl)ns1 ; CO[C@@H]1O[C@H](CO)[C@H](O)[C@H](O)[C@H]1Nc1nc(N2CCN(C)CC2)ns1 ; CO[C@@H]1O[C@H](CO)[C@H](O)[C@H](O)[C@H]1Nc1nc(N2CCOCC2)ns1]

In certain embodiments, the compound of Formula II is selected from:

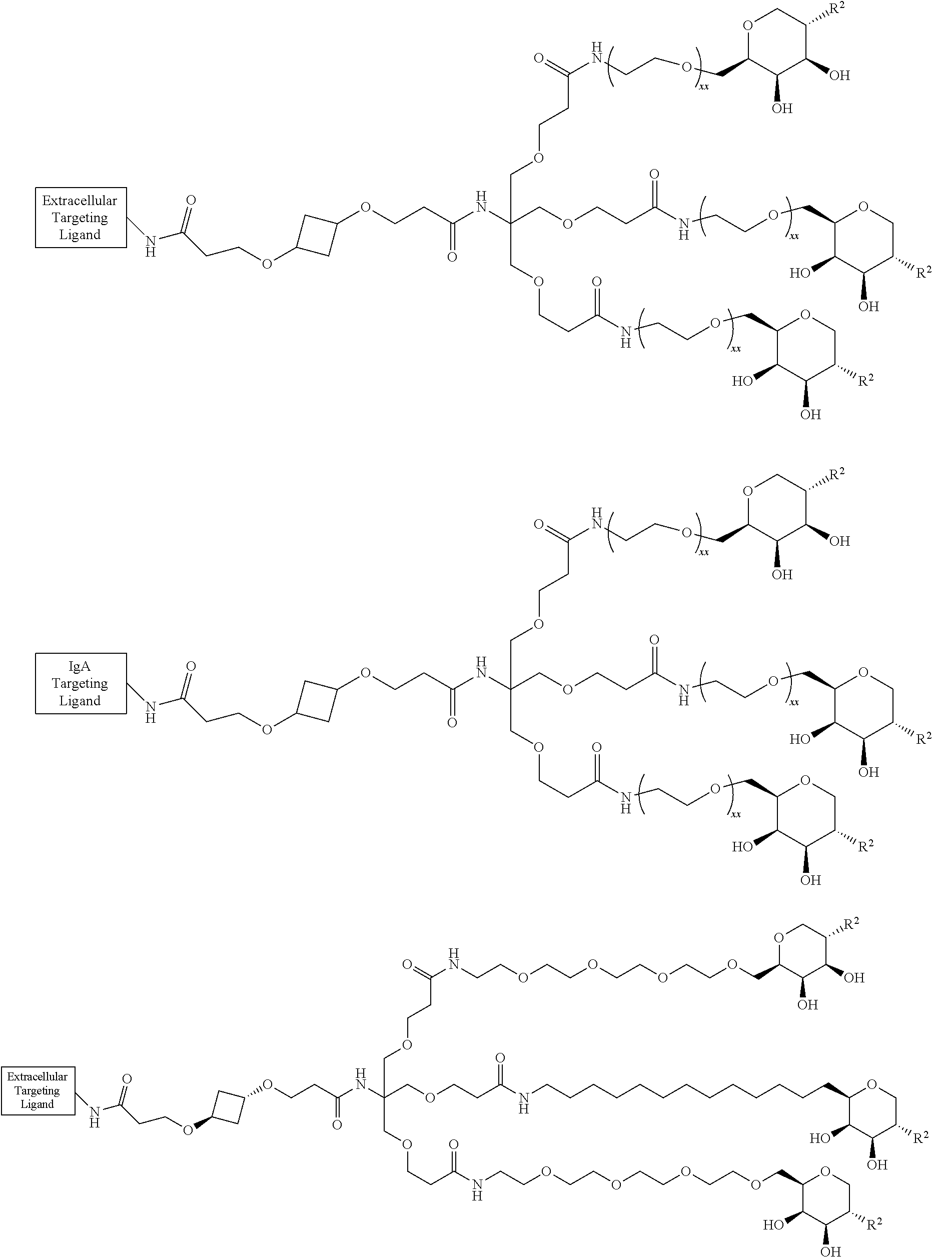

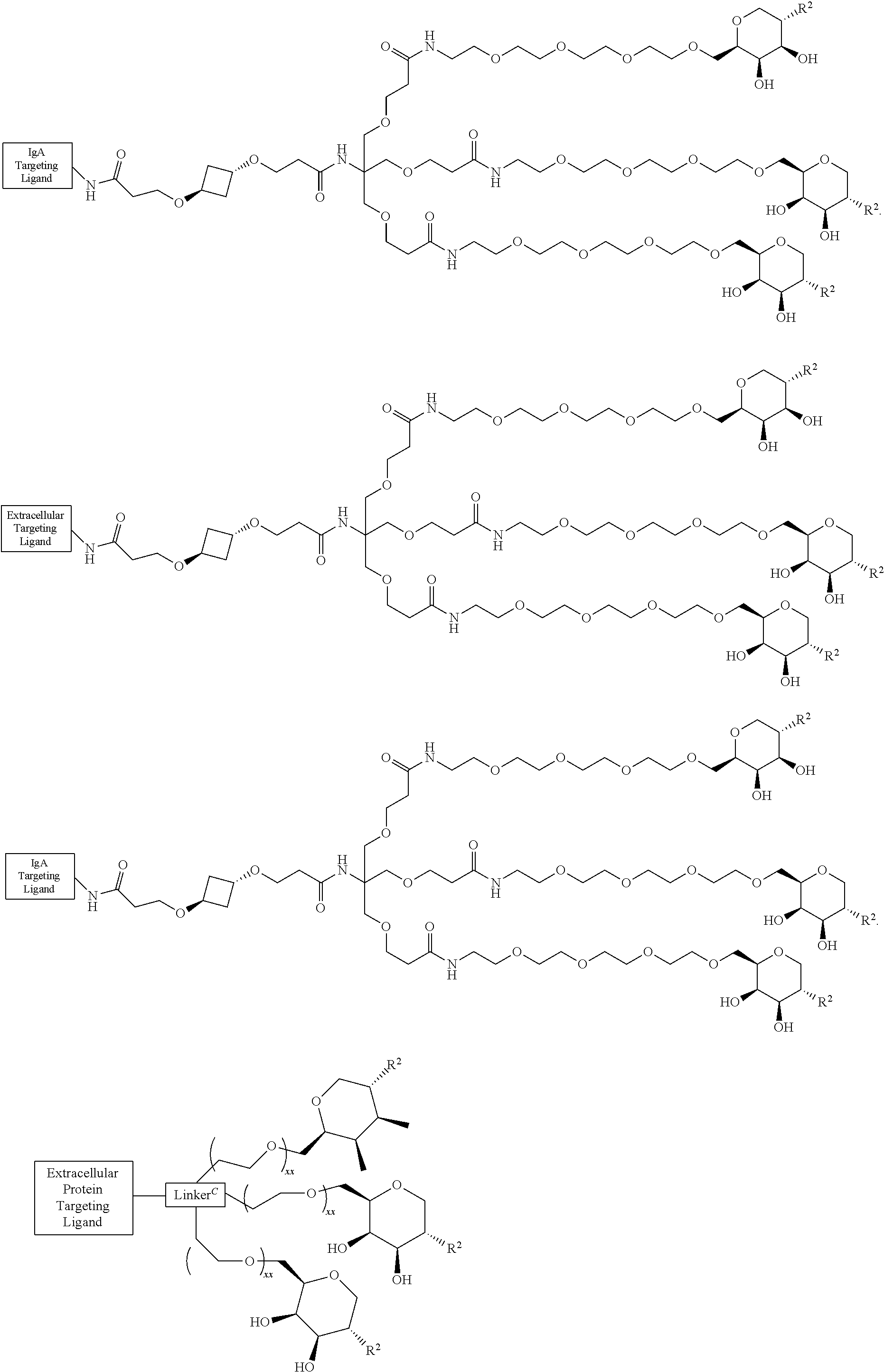

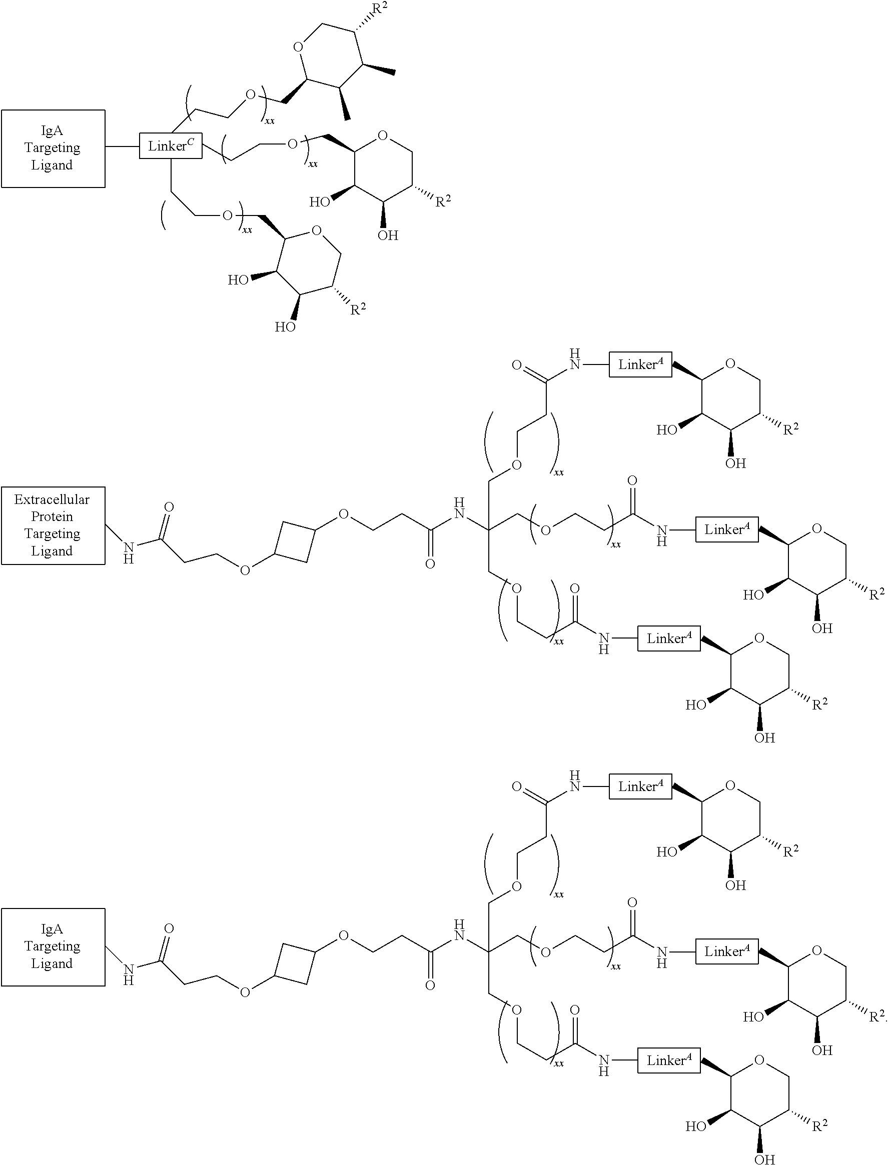

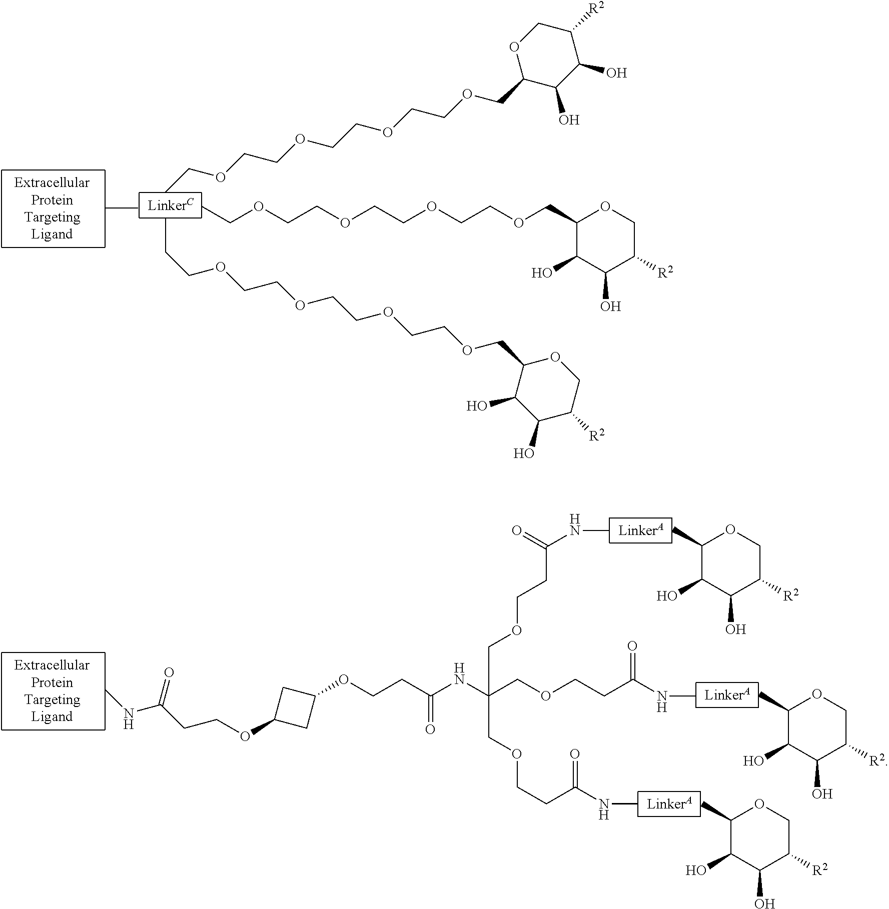

B. Talose-Based ASGPR-Binding Cellular Receptor Binding Moieties of Formula II

In certain embodiments, the compound of Formula II is selected from:

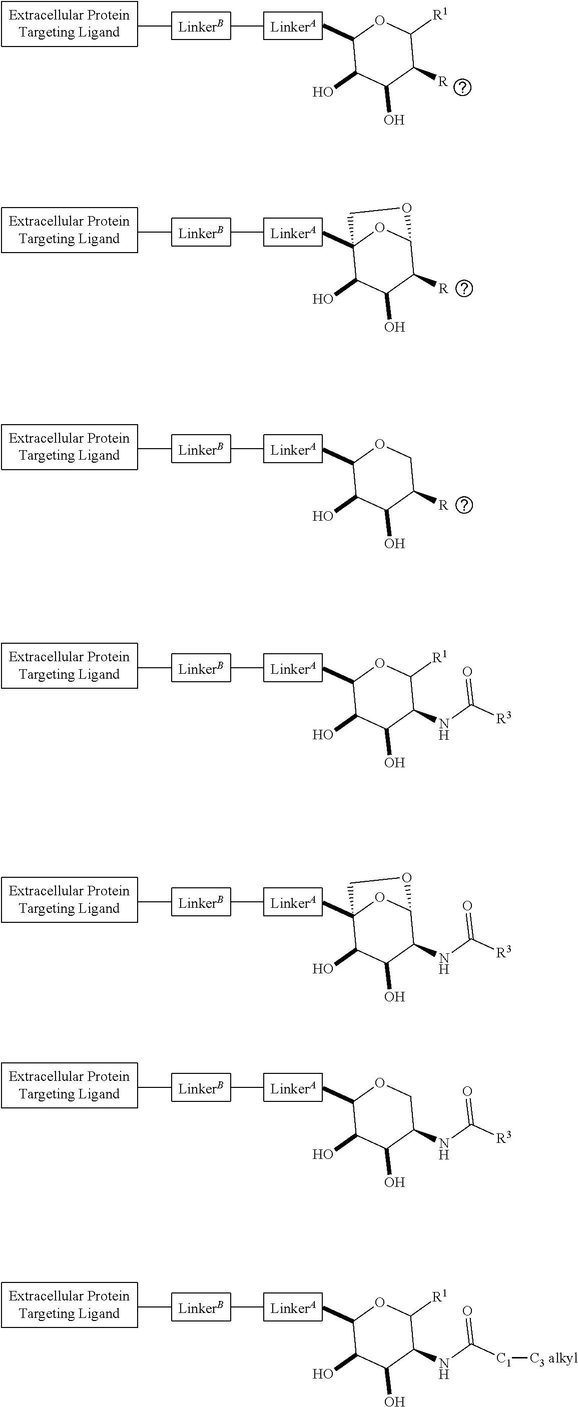

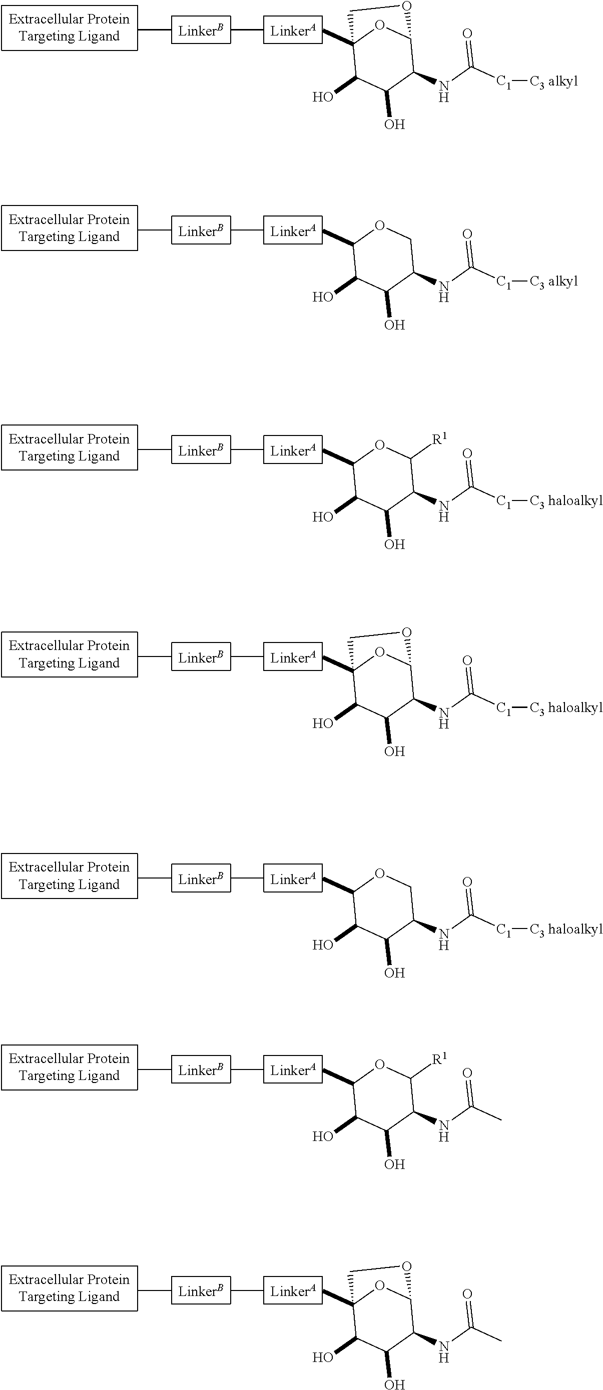

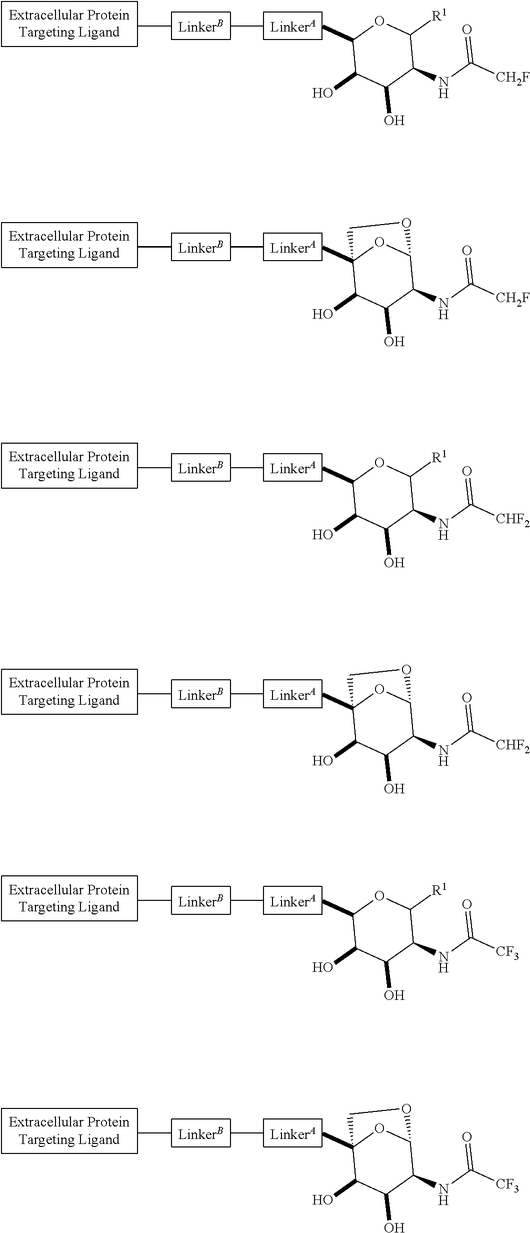

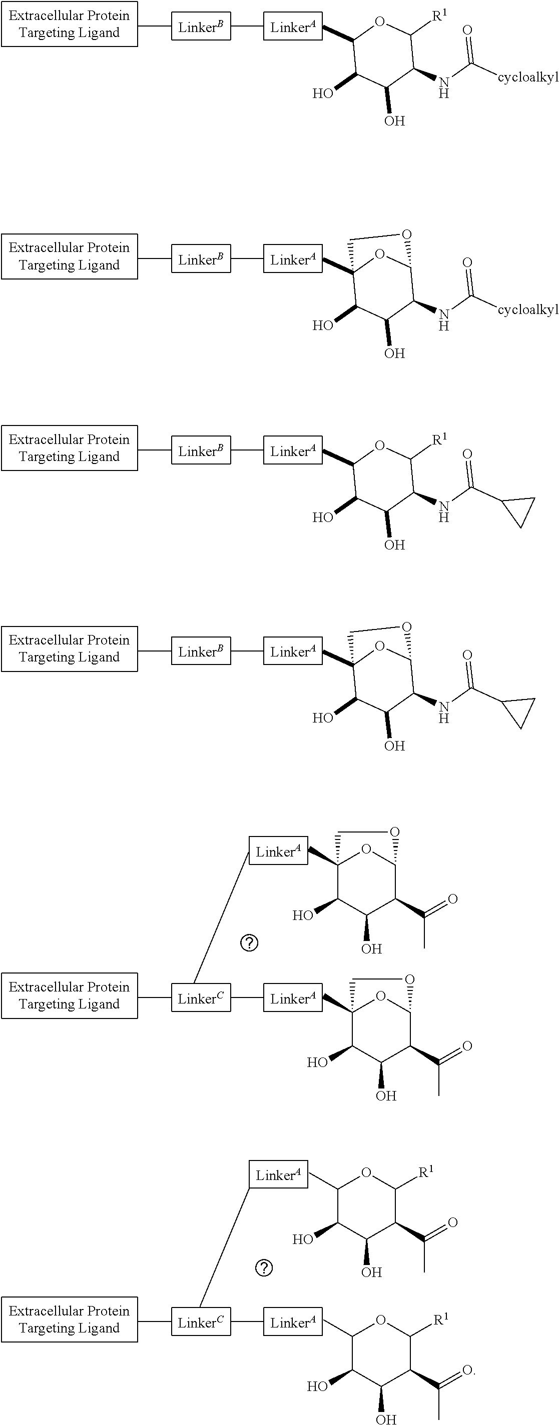

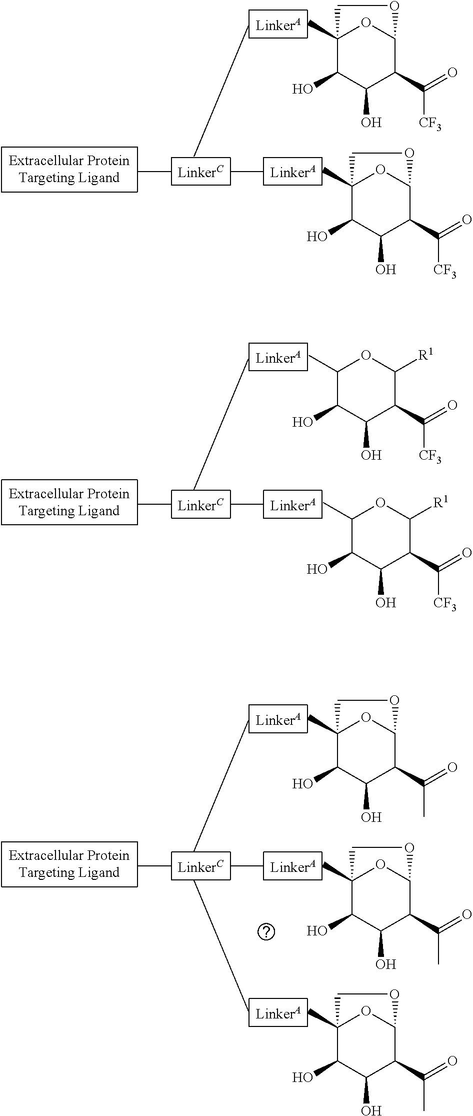

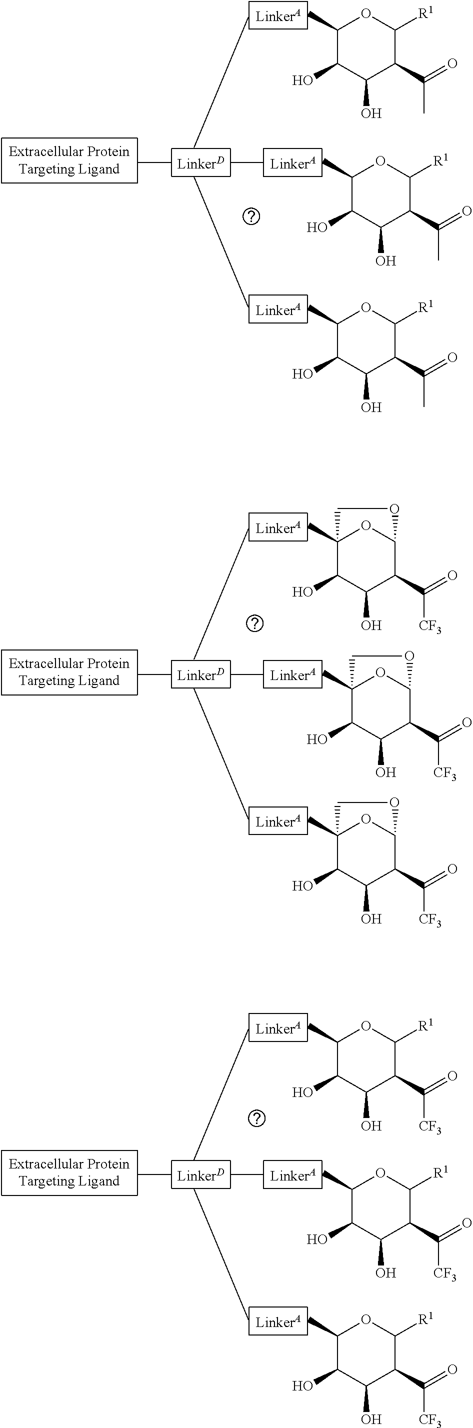

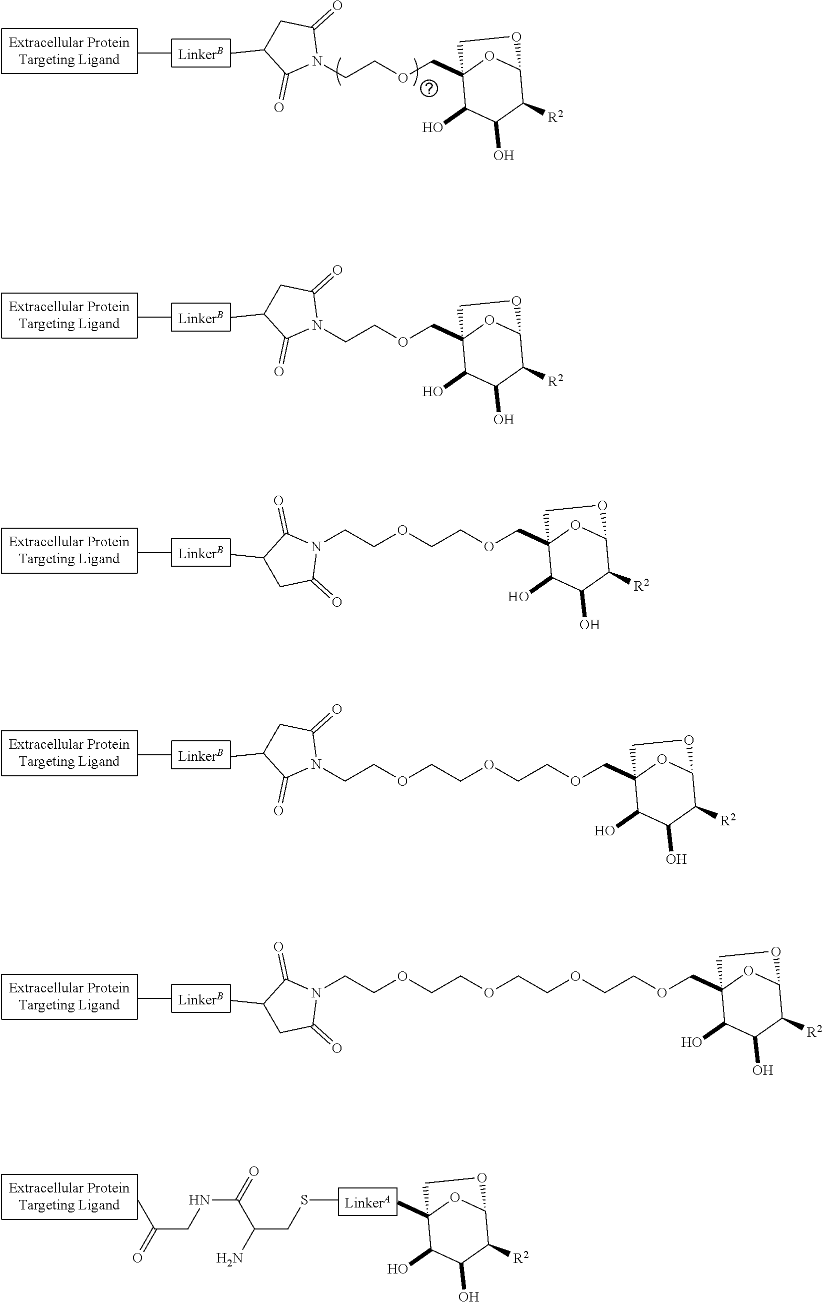

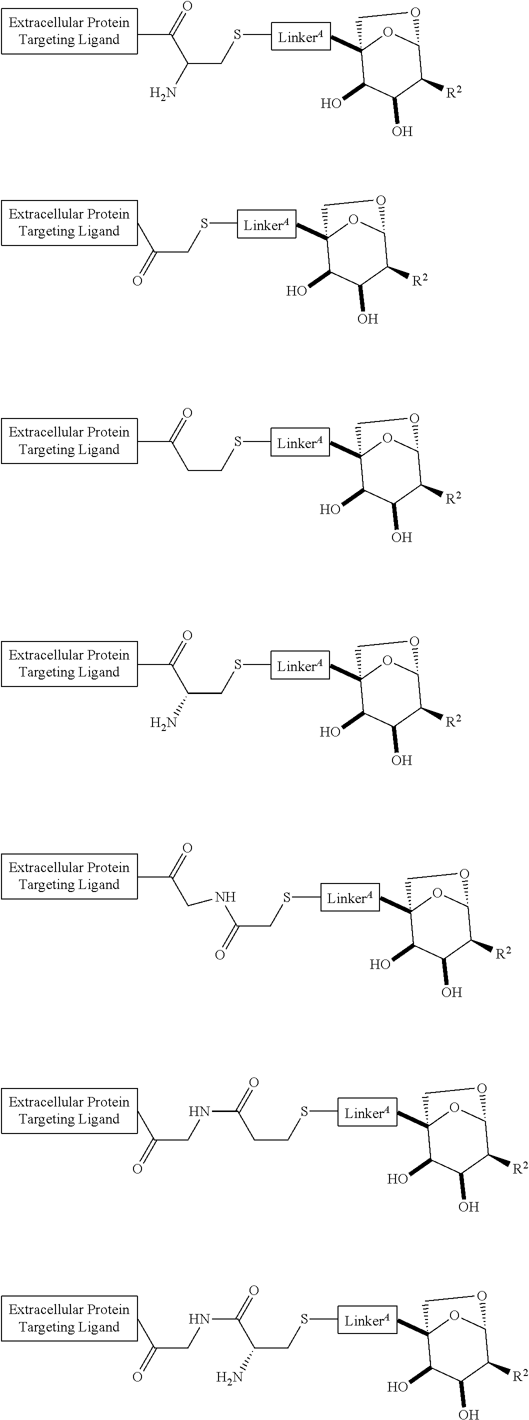

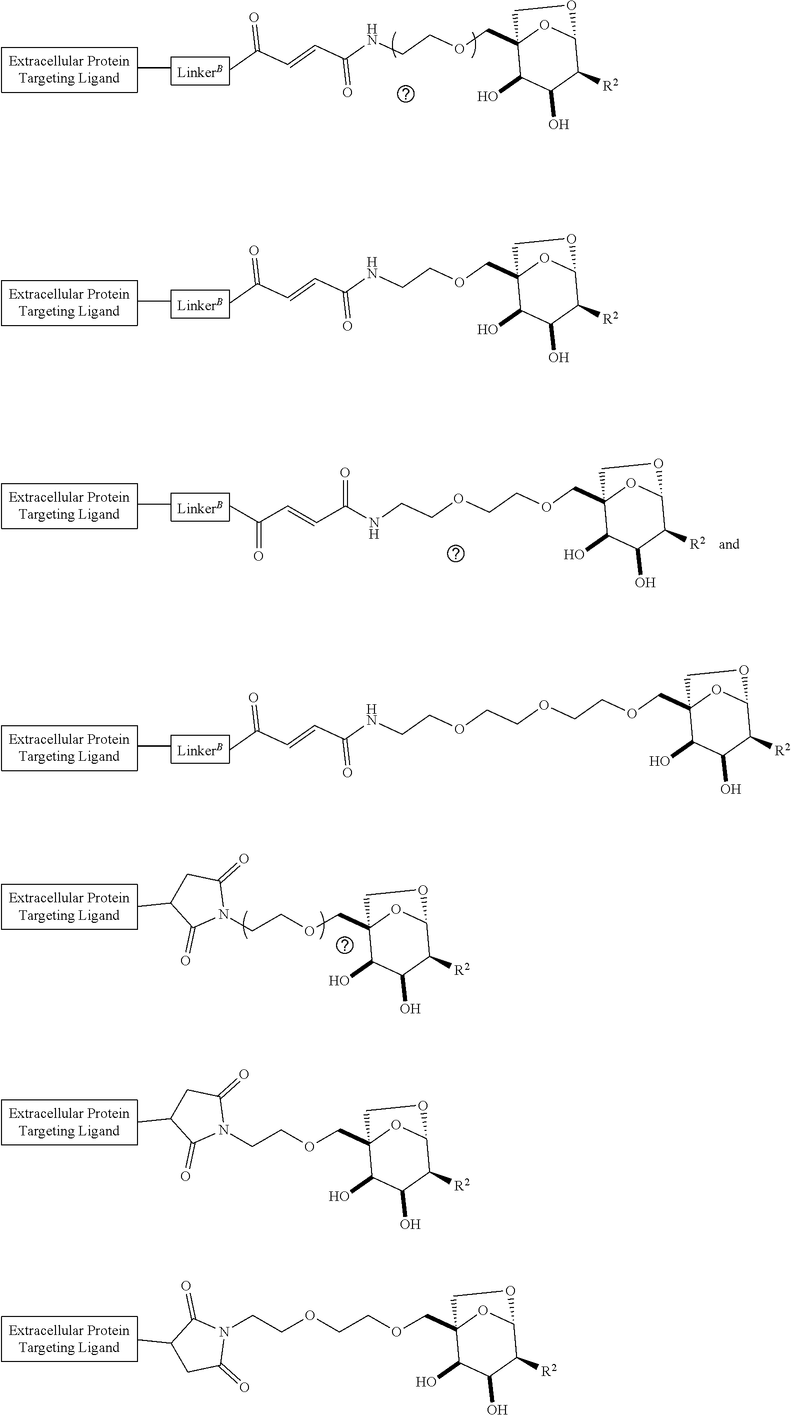

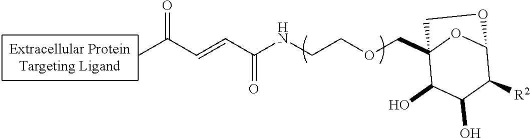

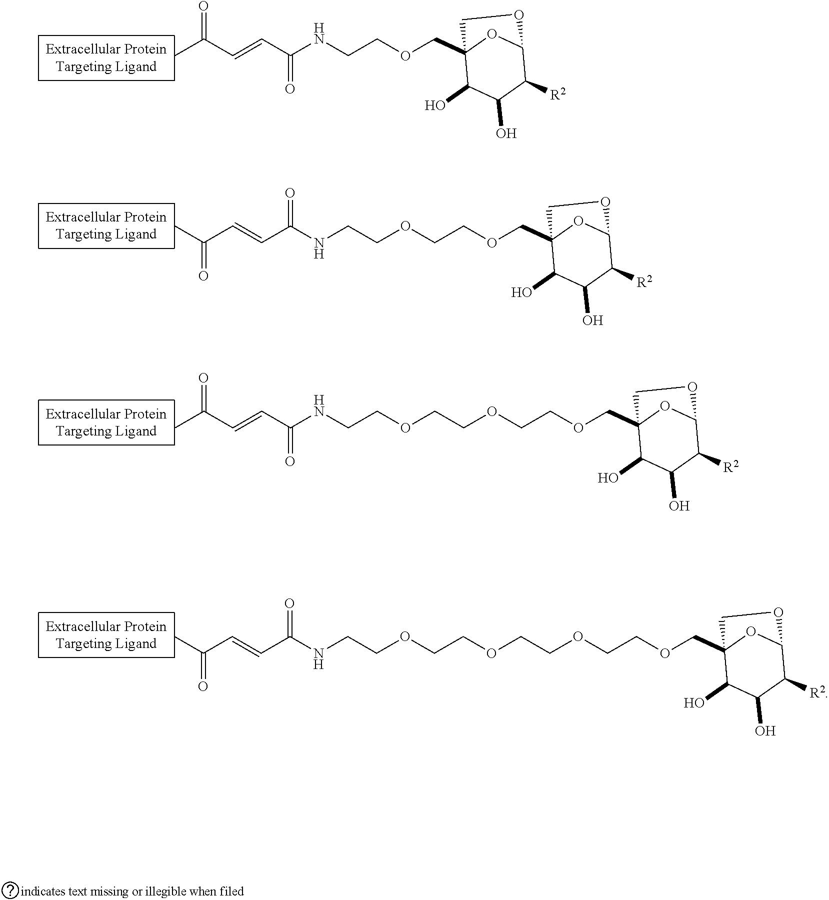

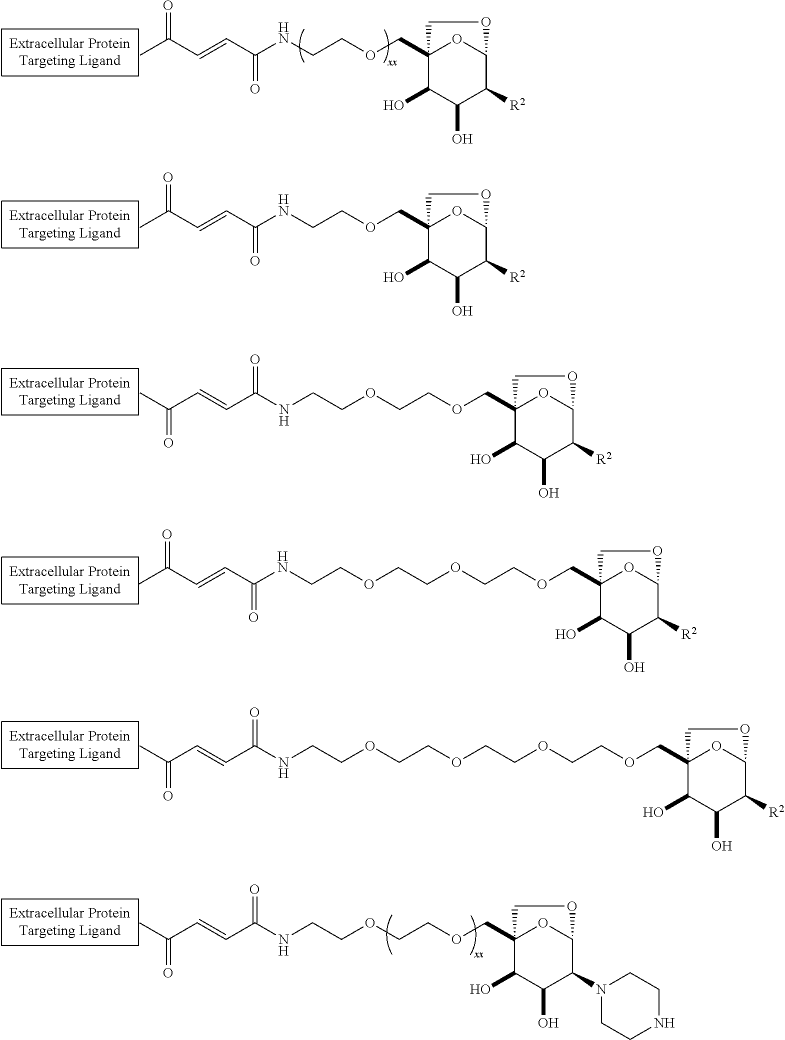

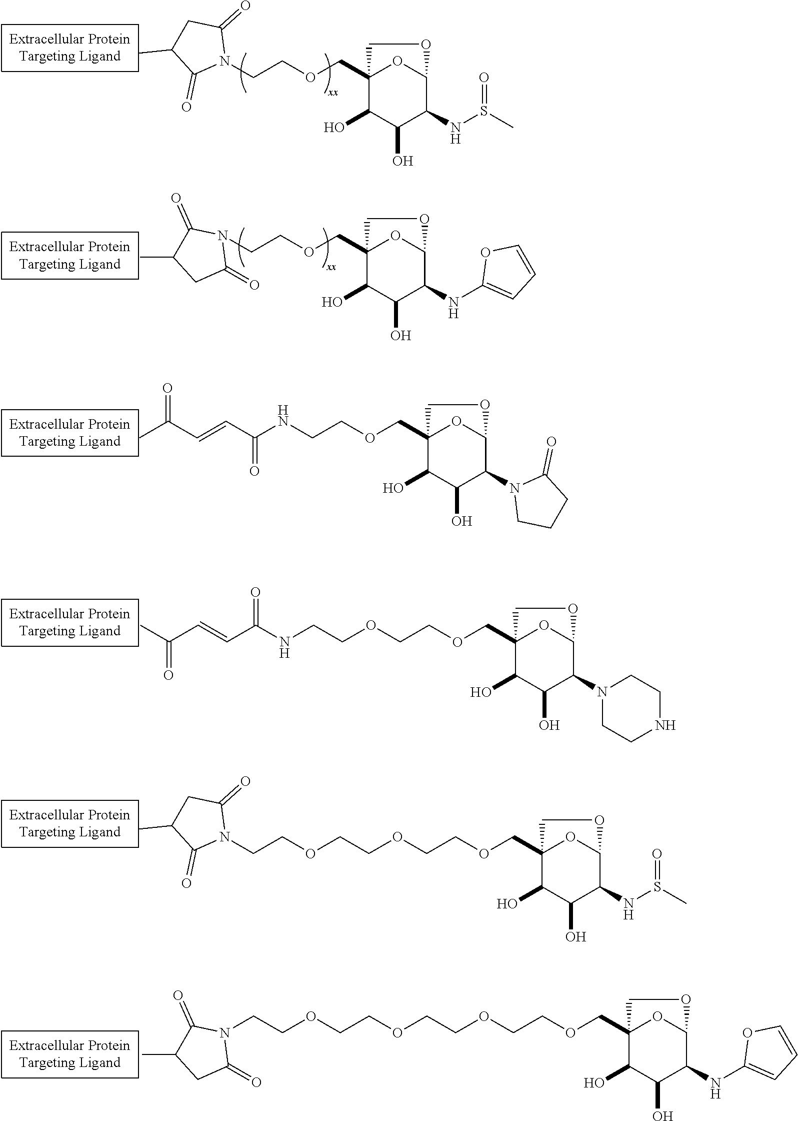

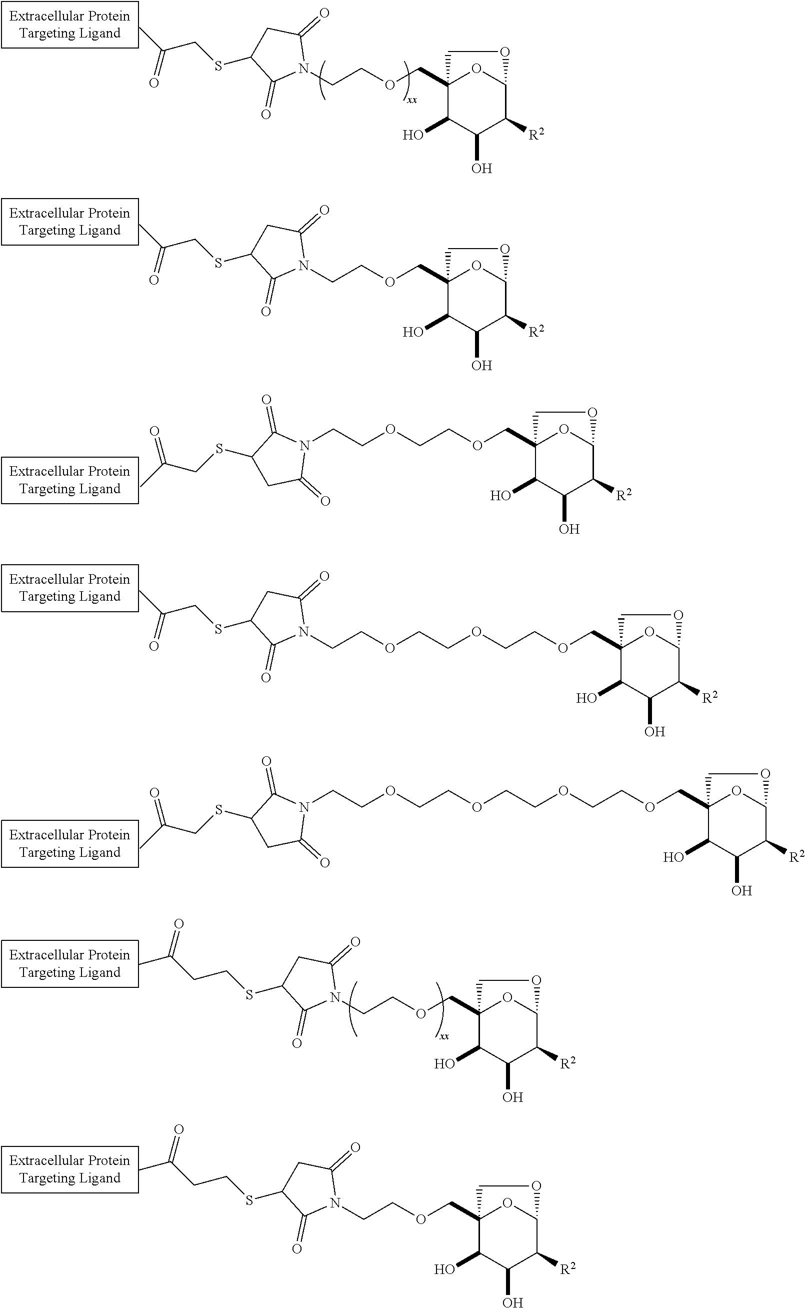

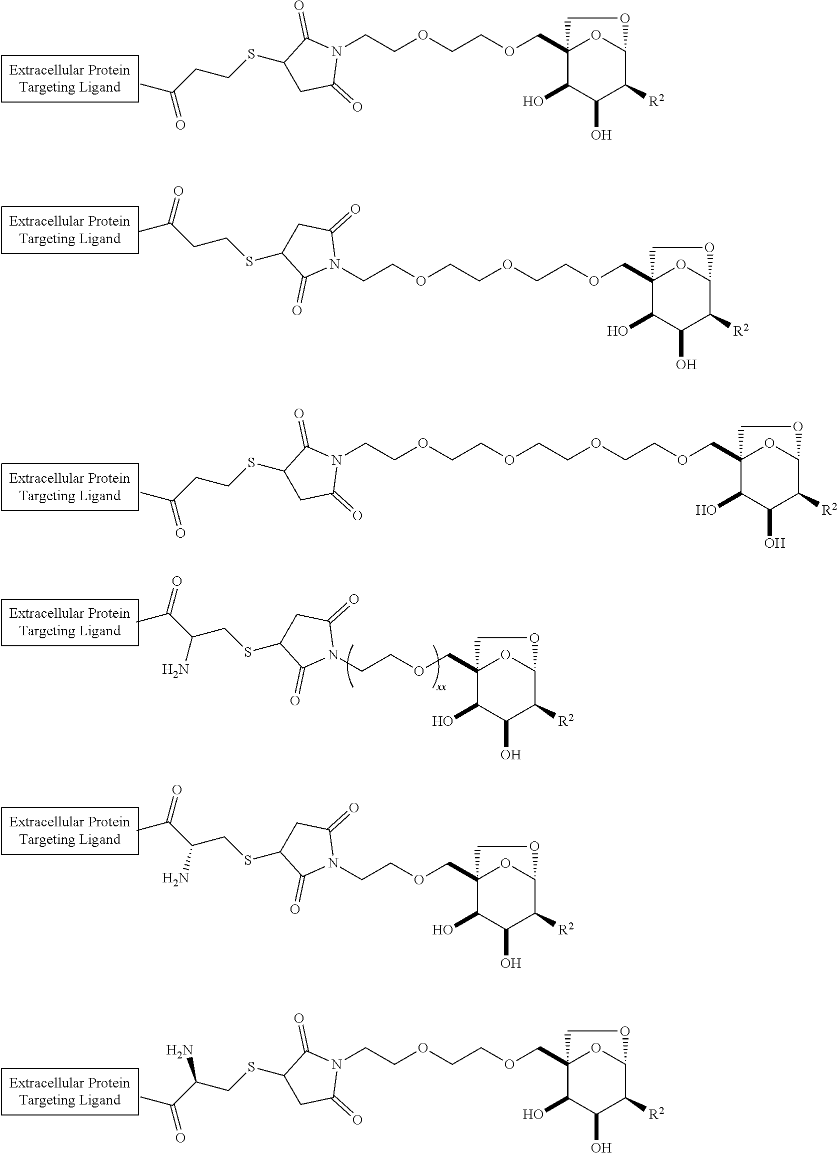

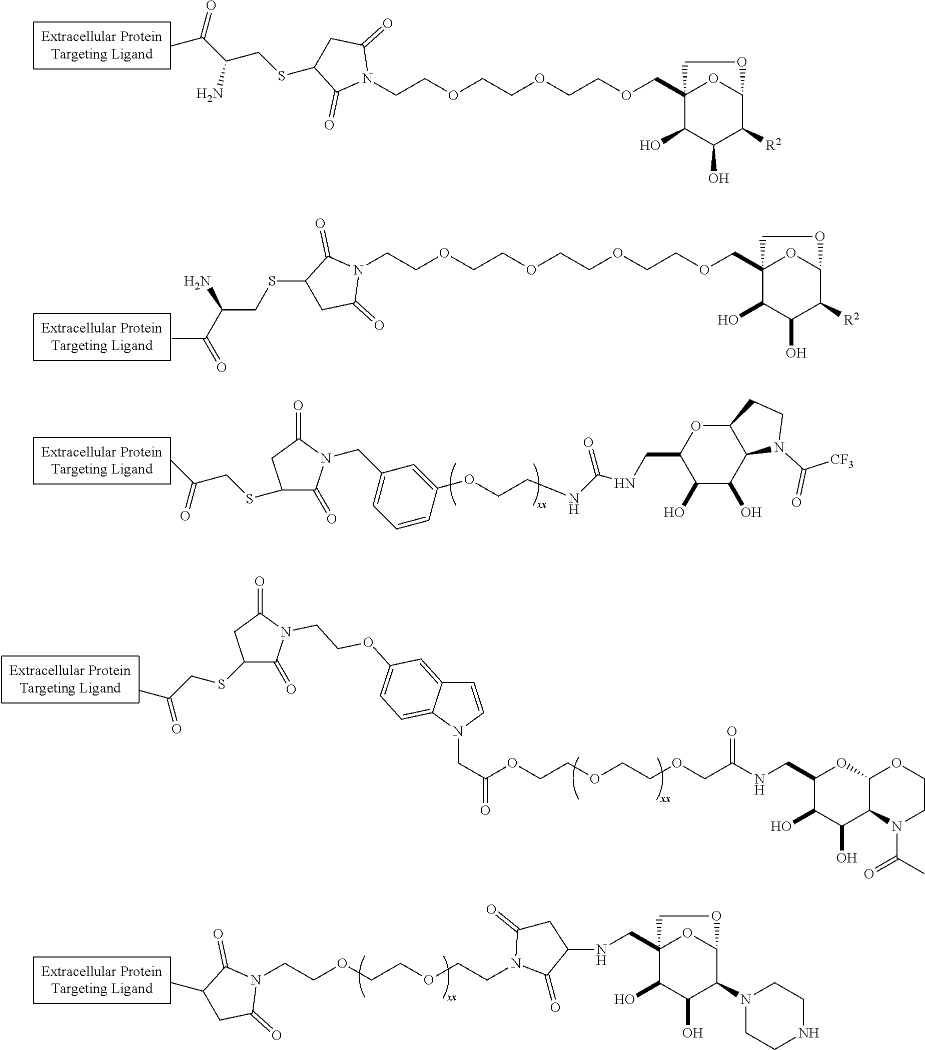

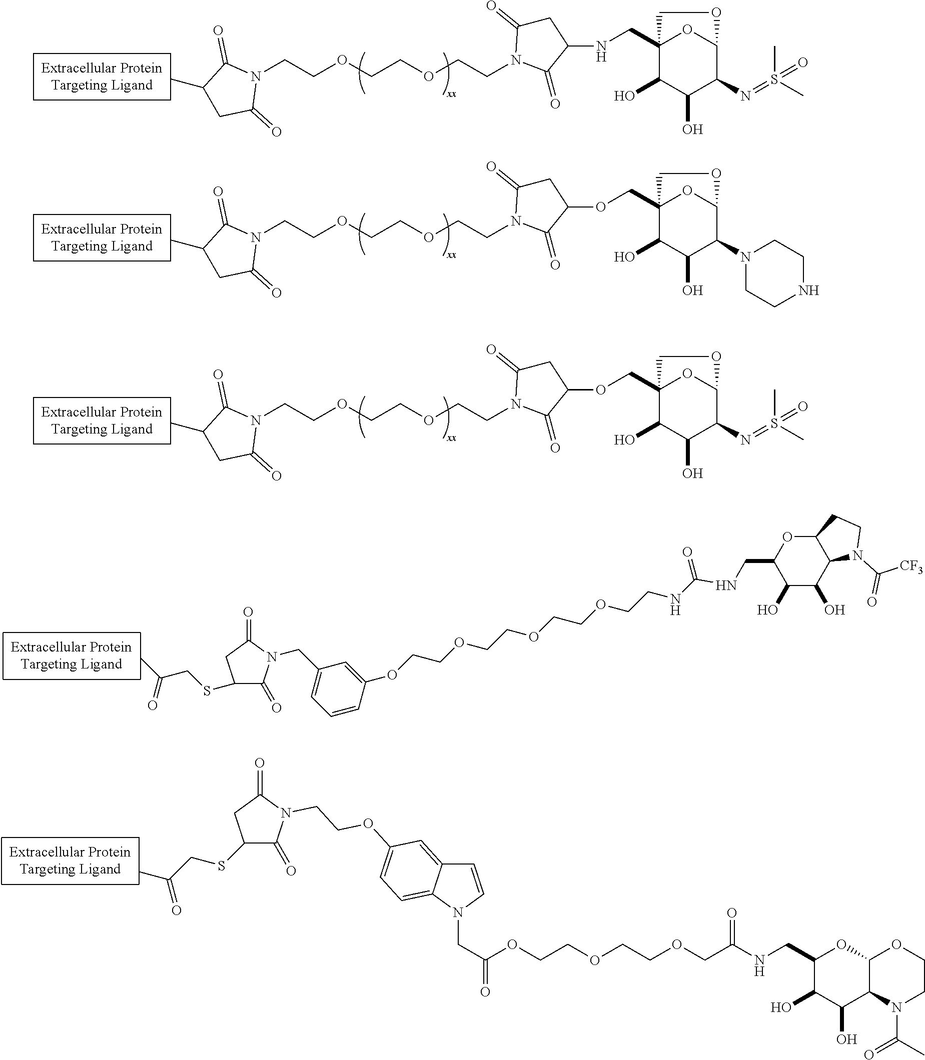

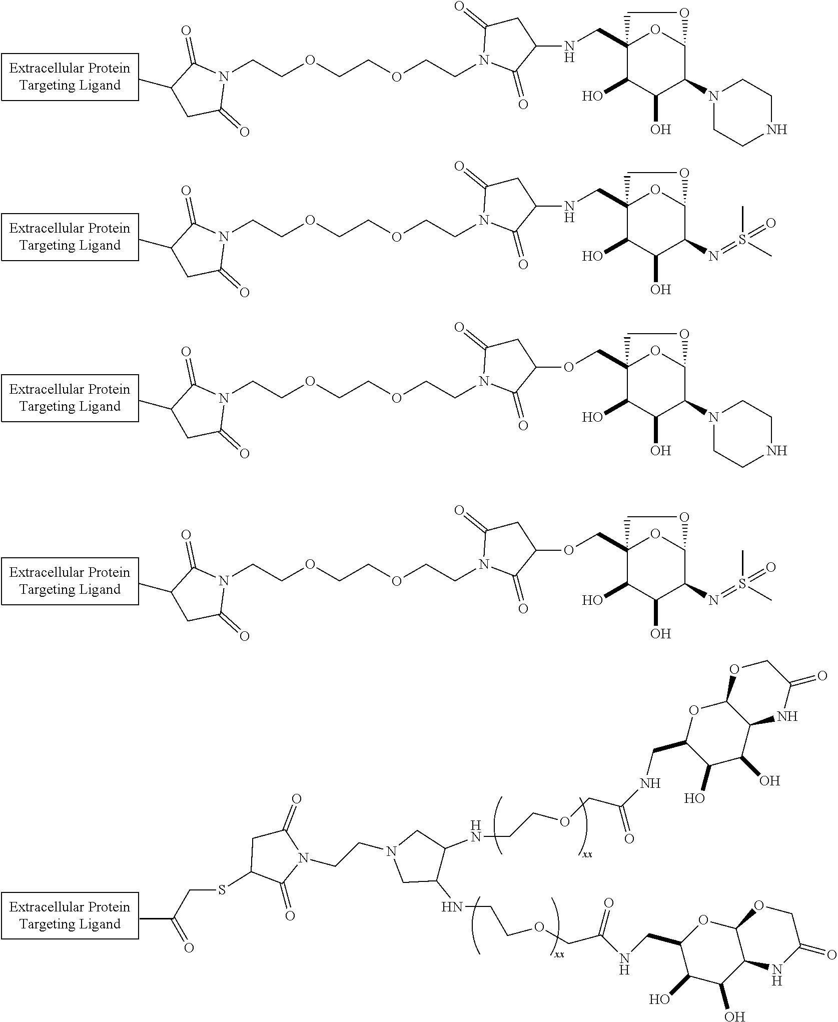

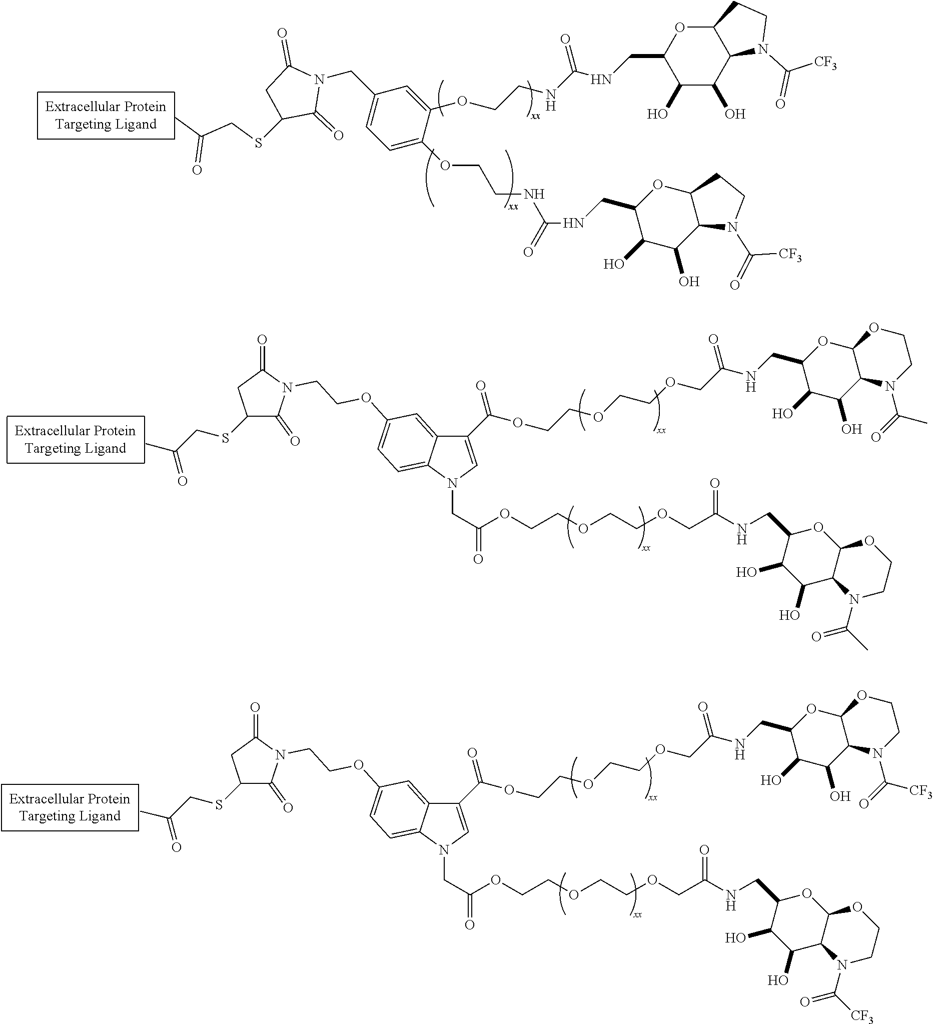

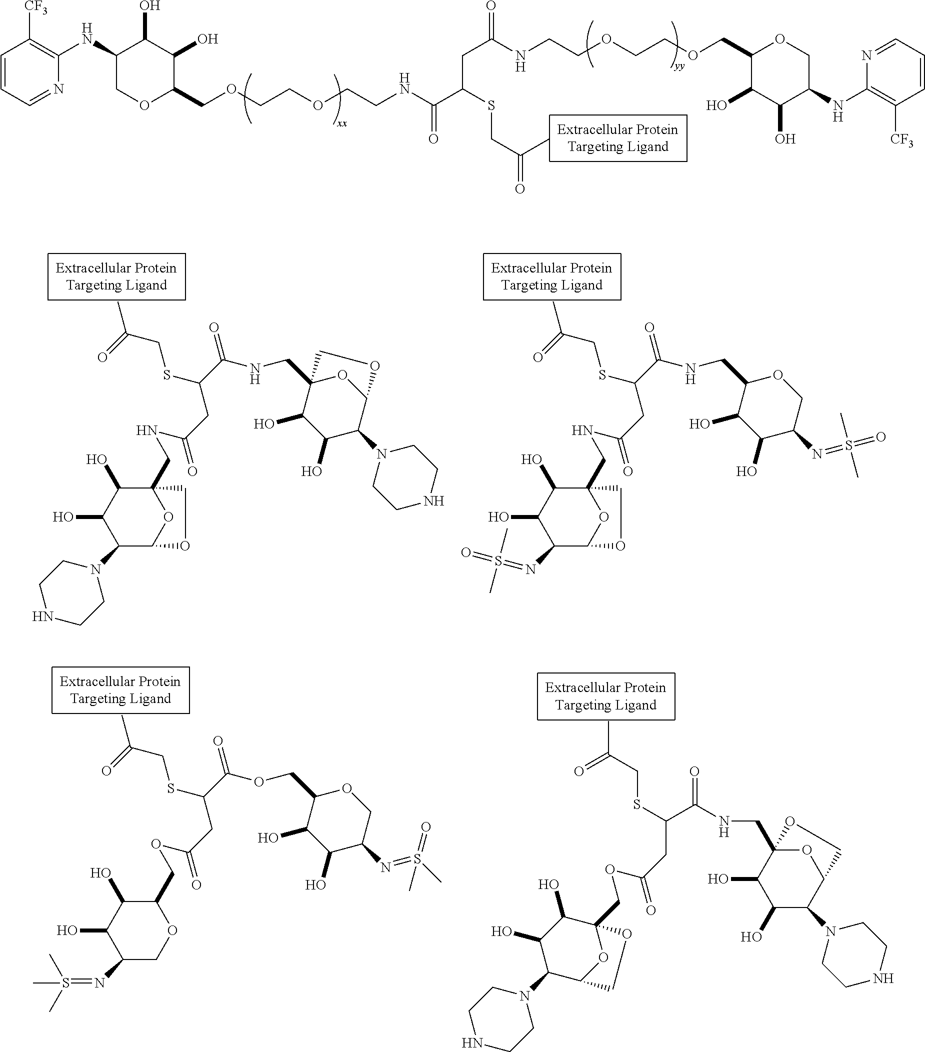

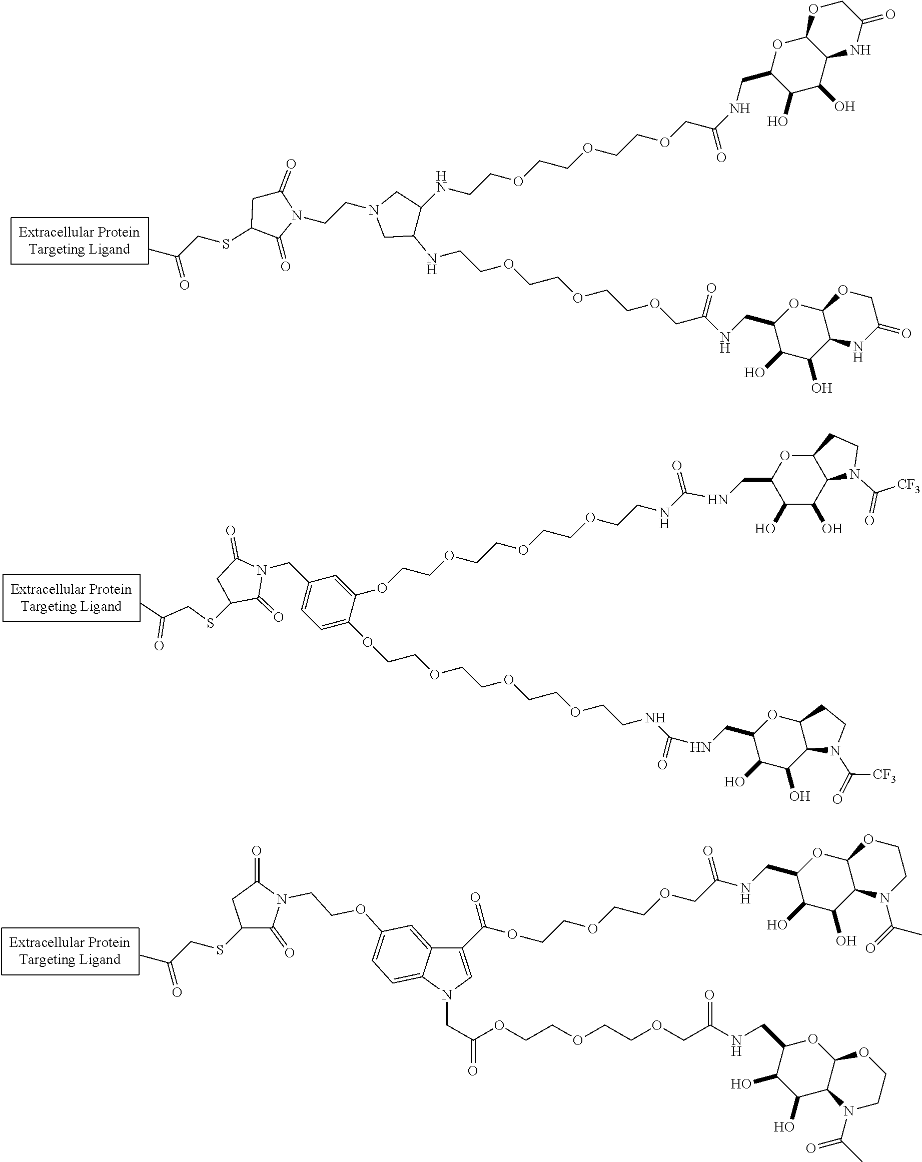

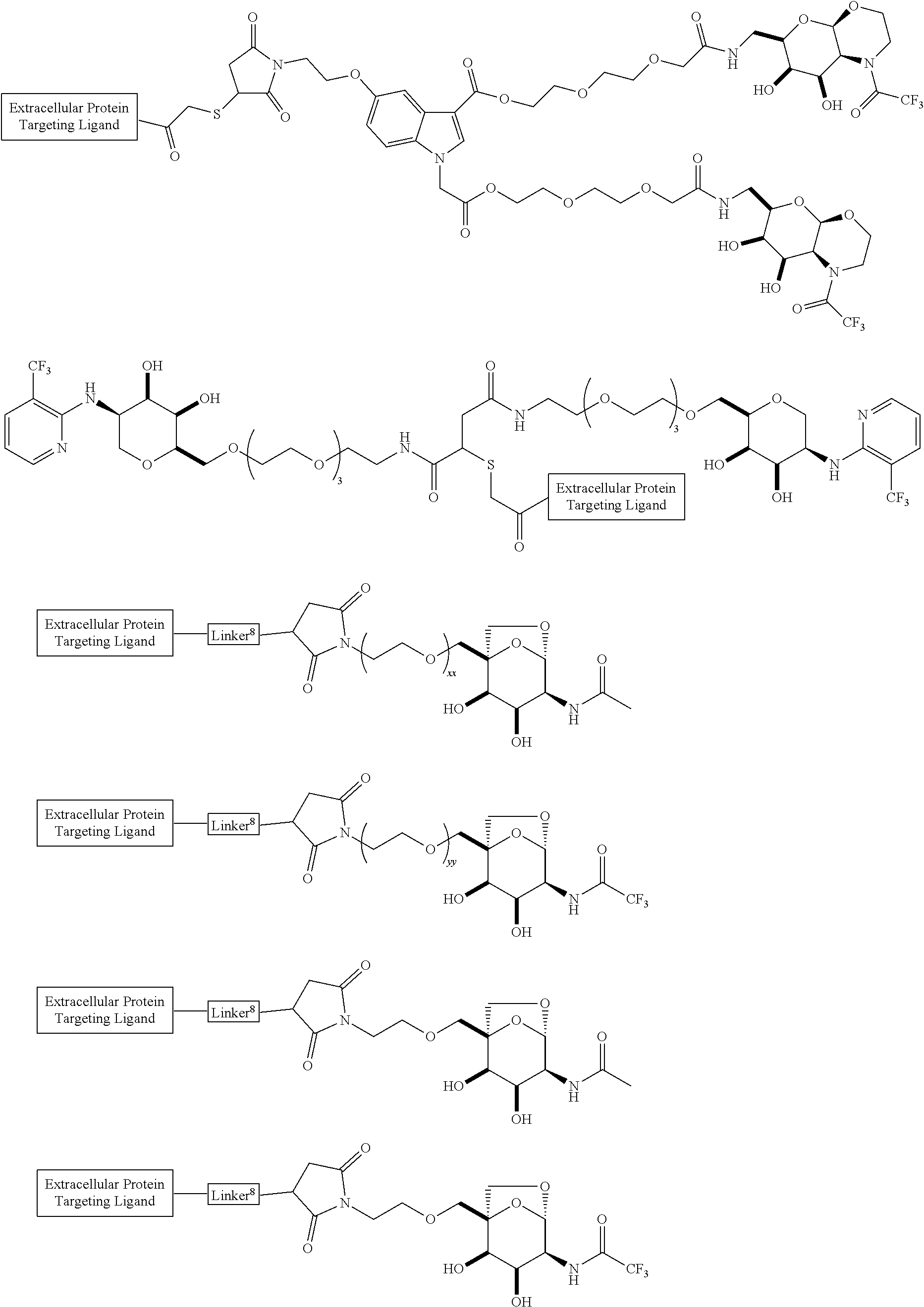

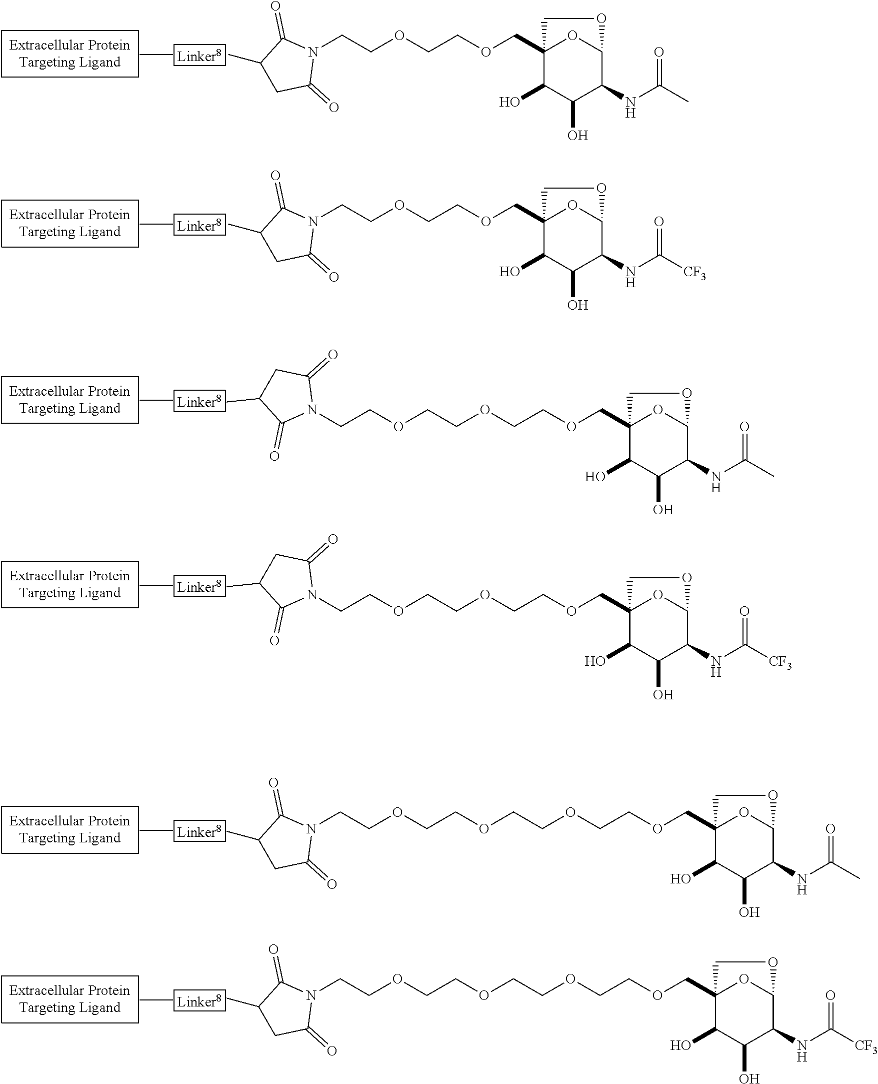

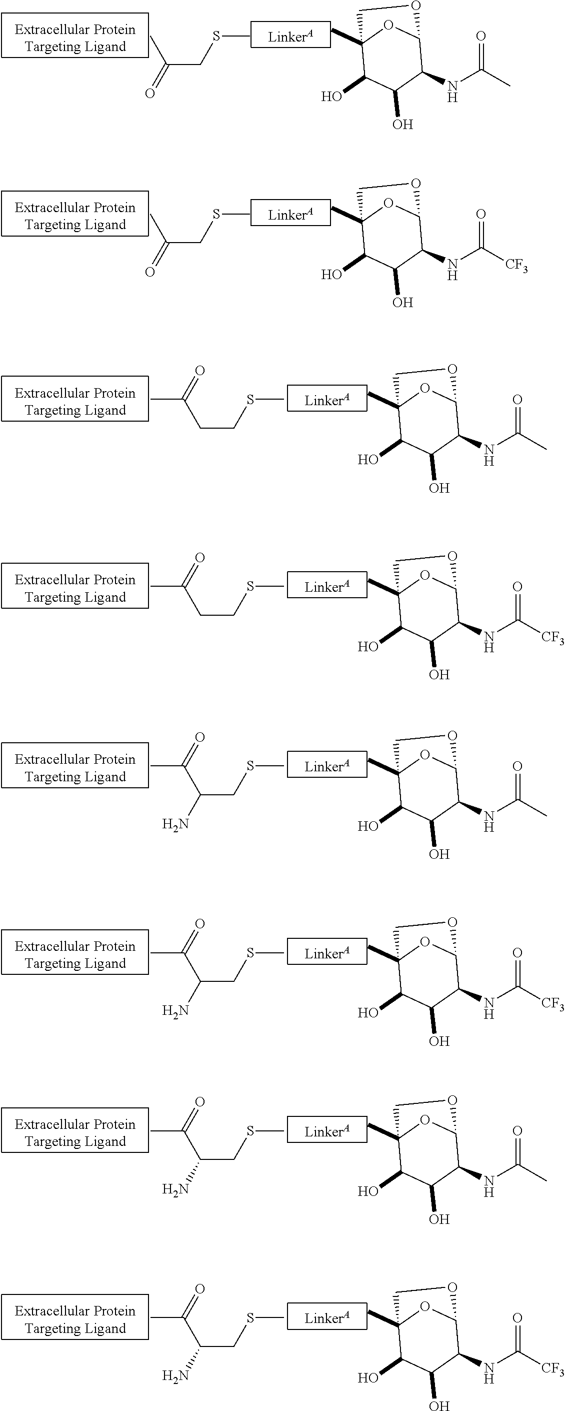

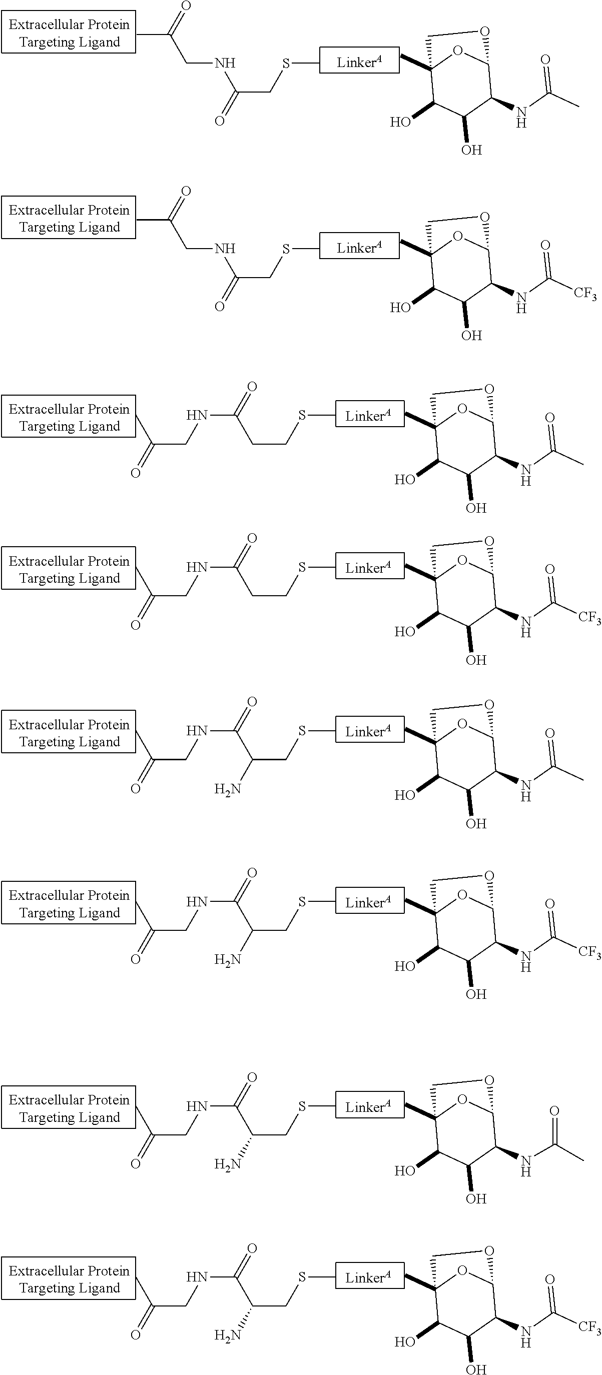

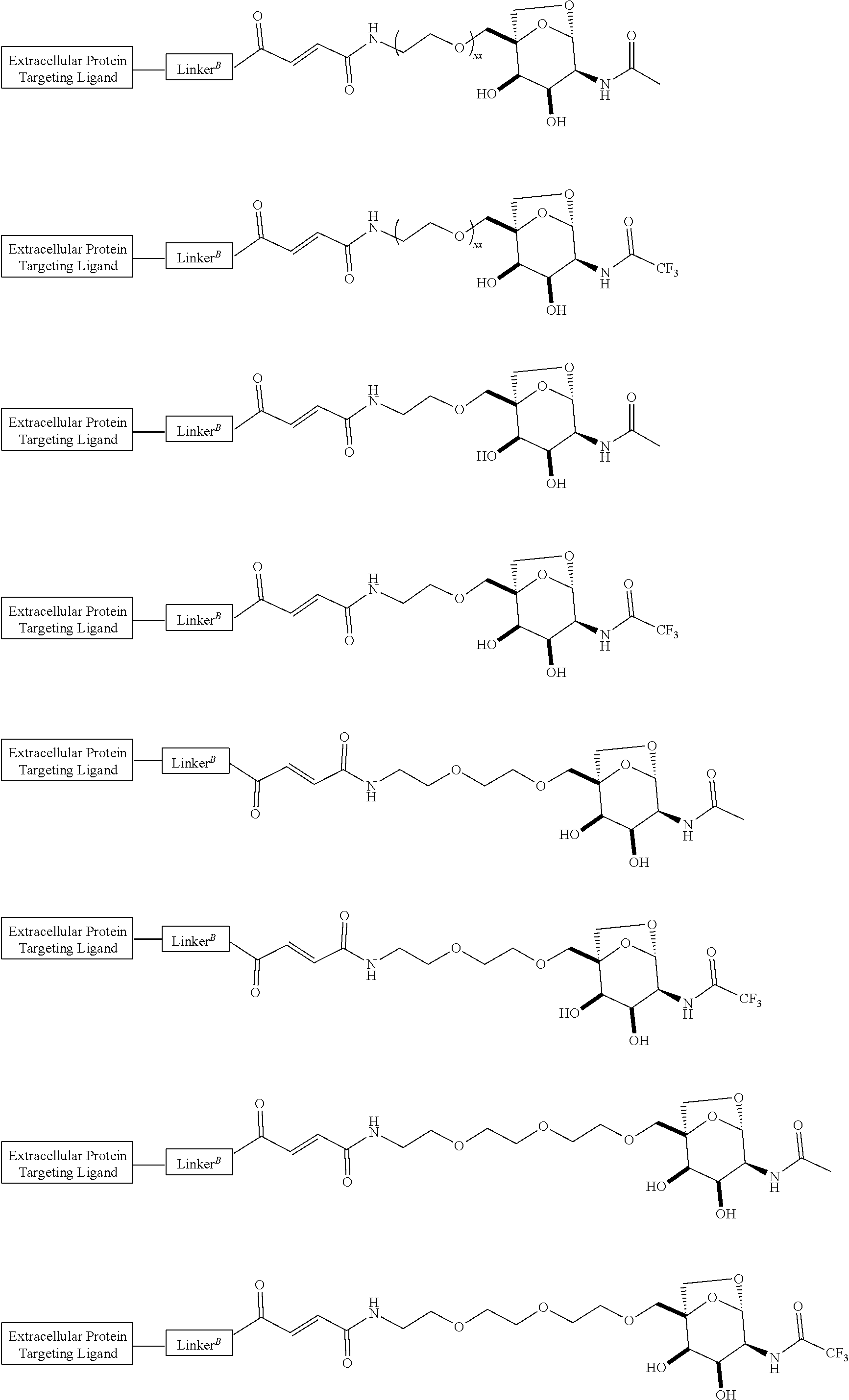

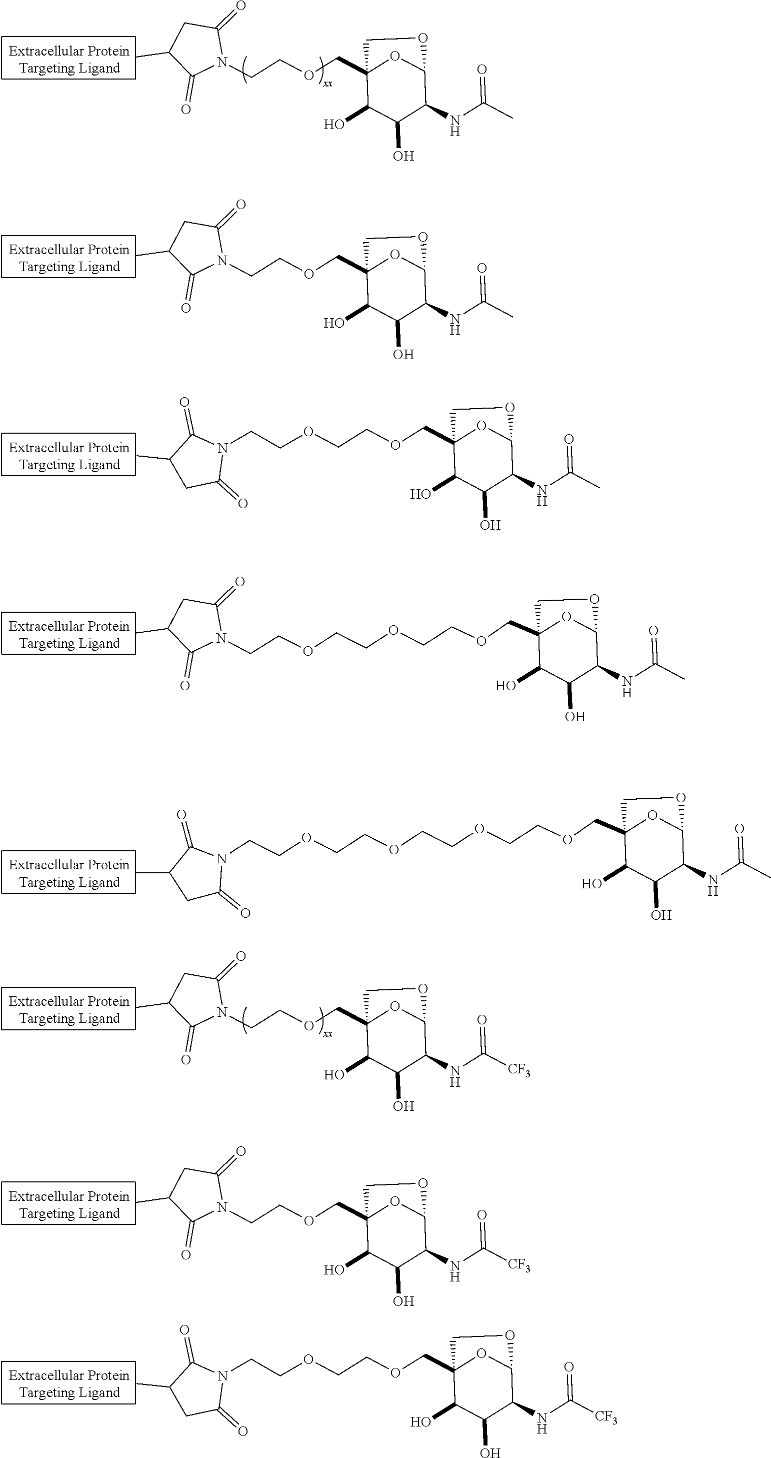

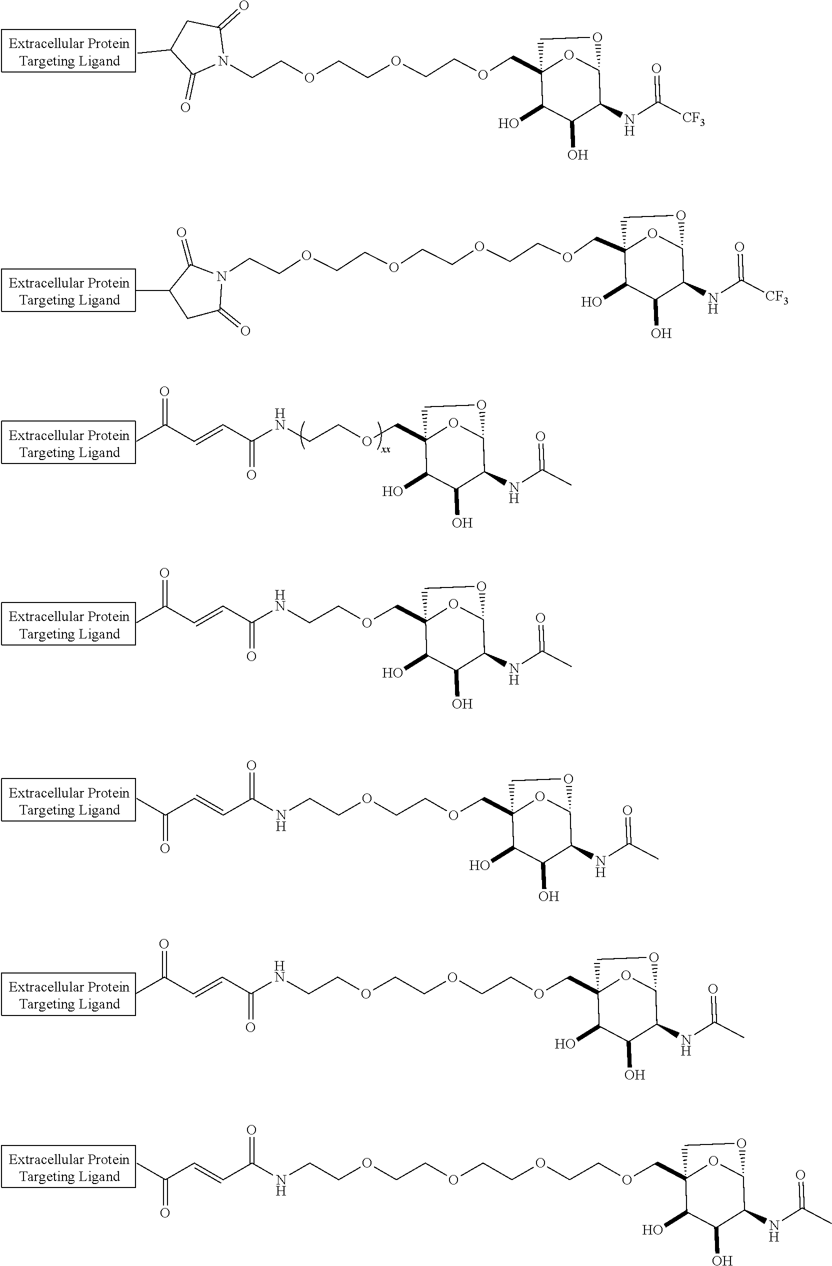

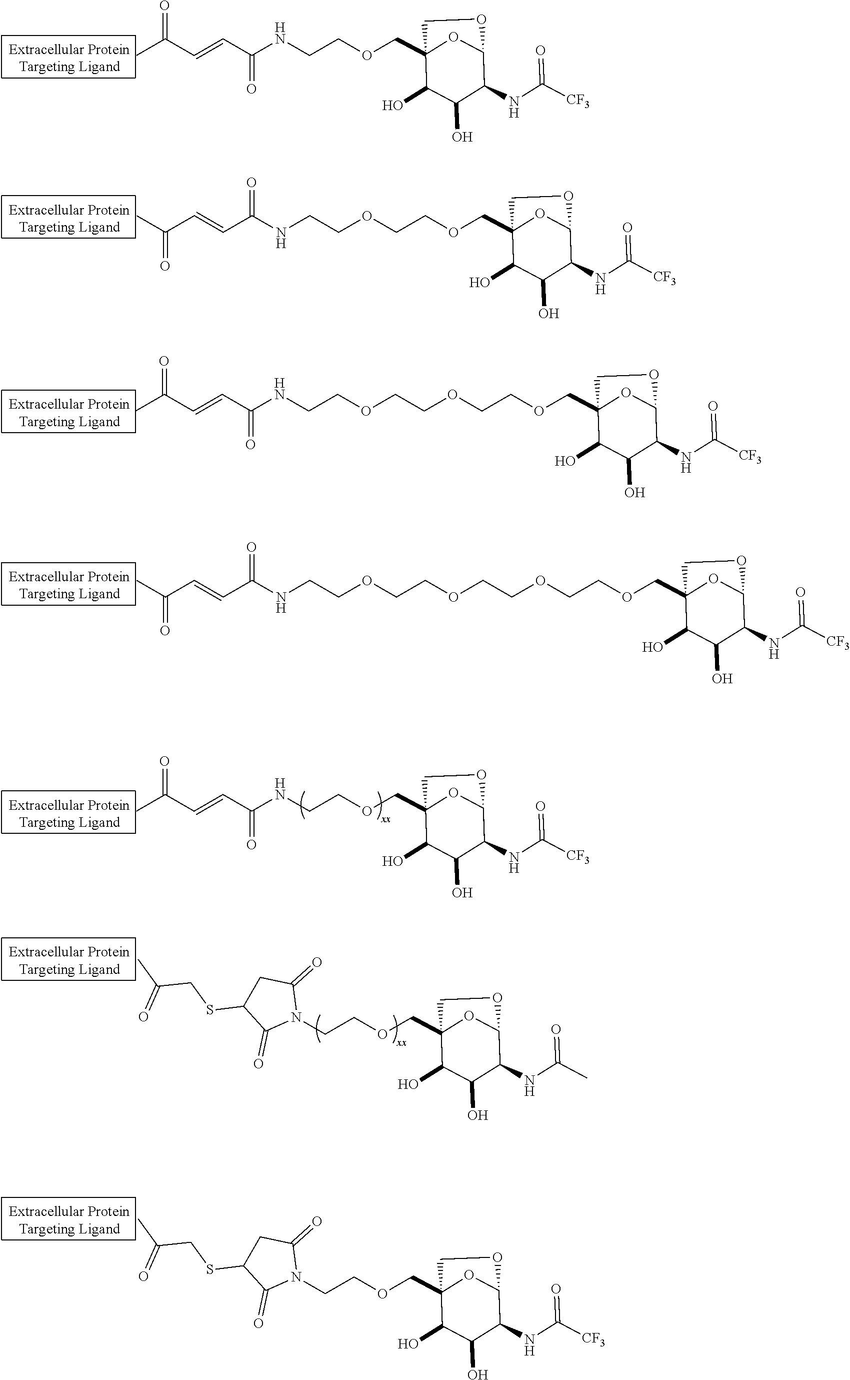

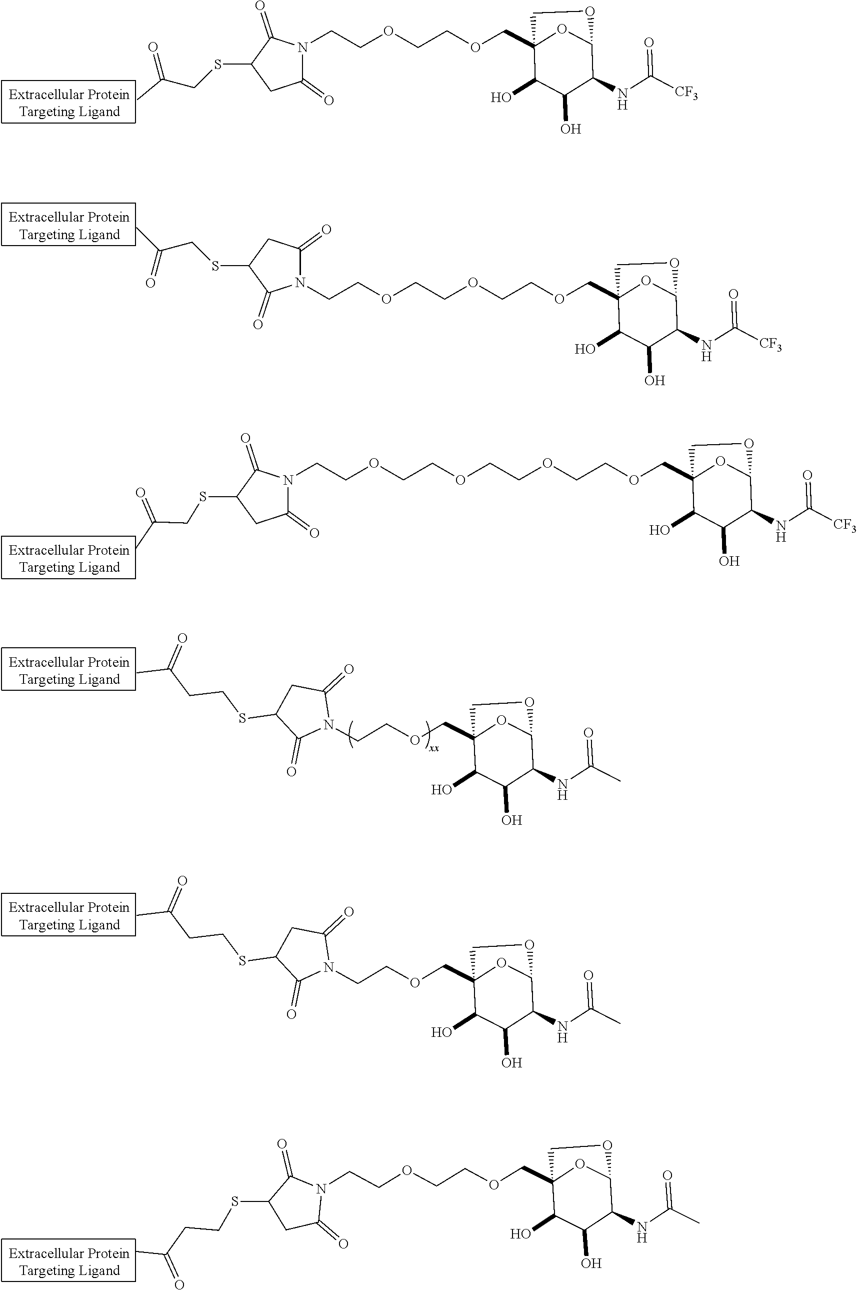

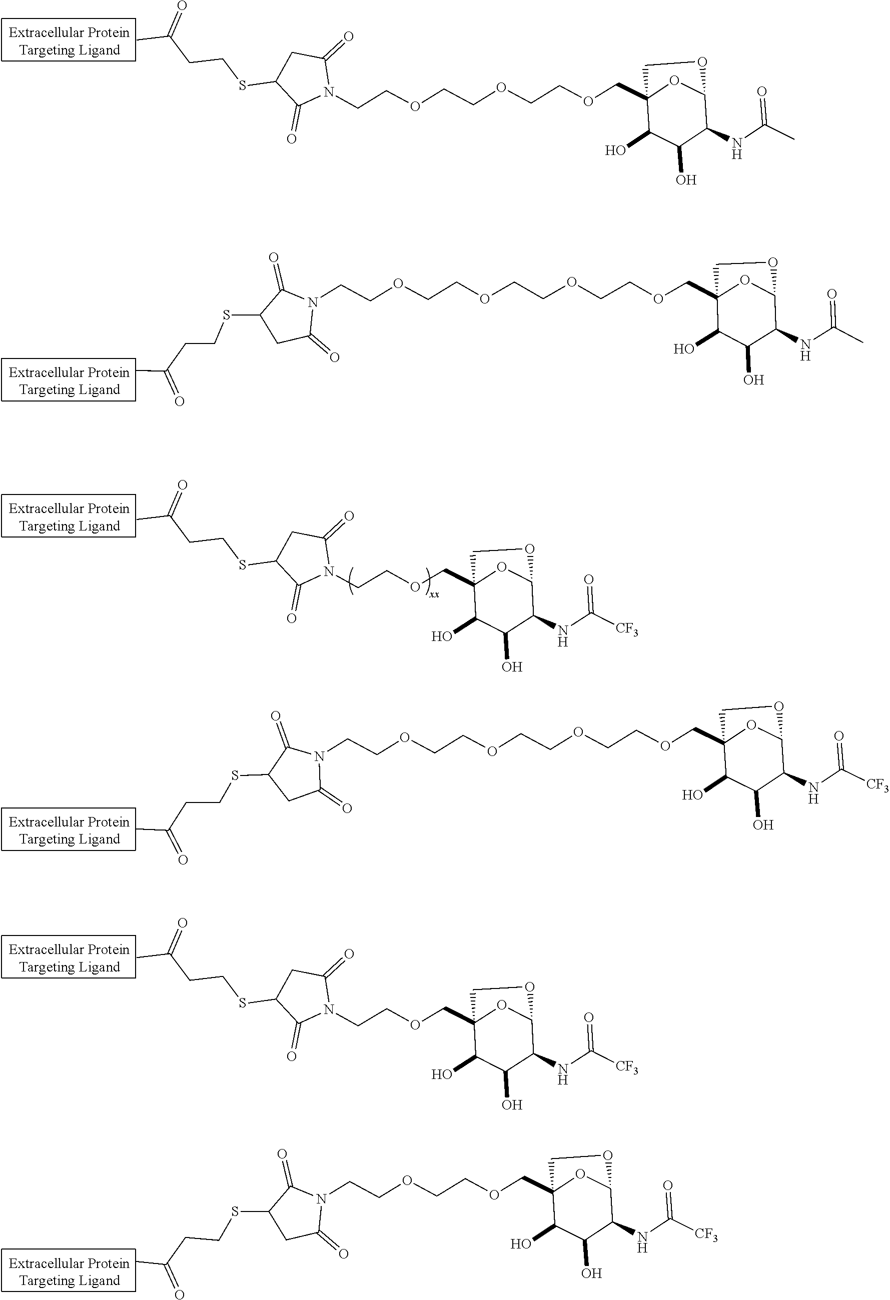

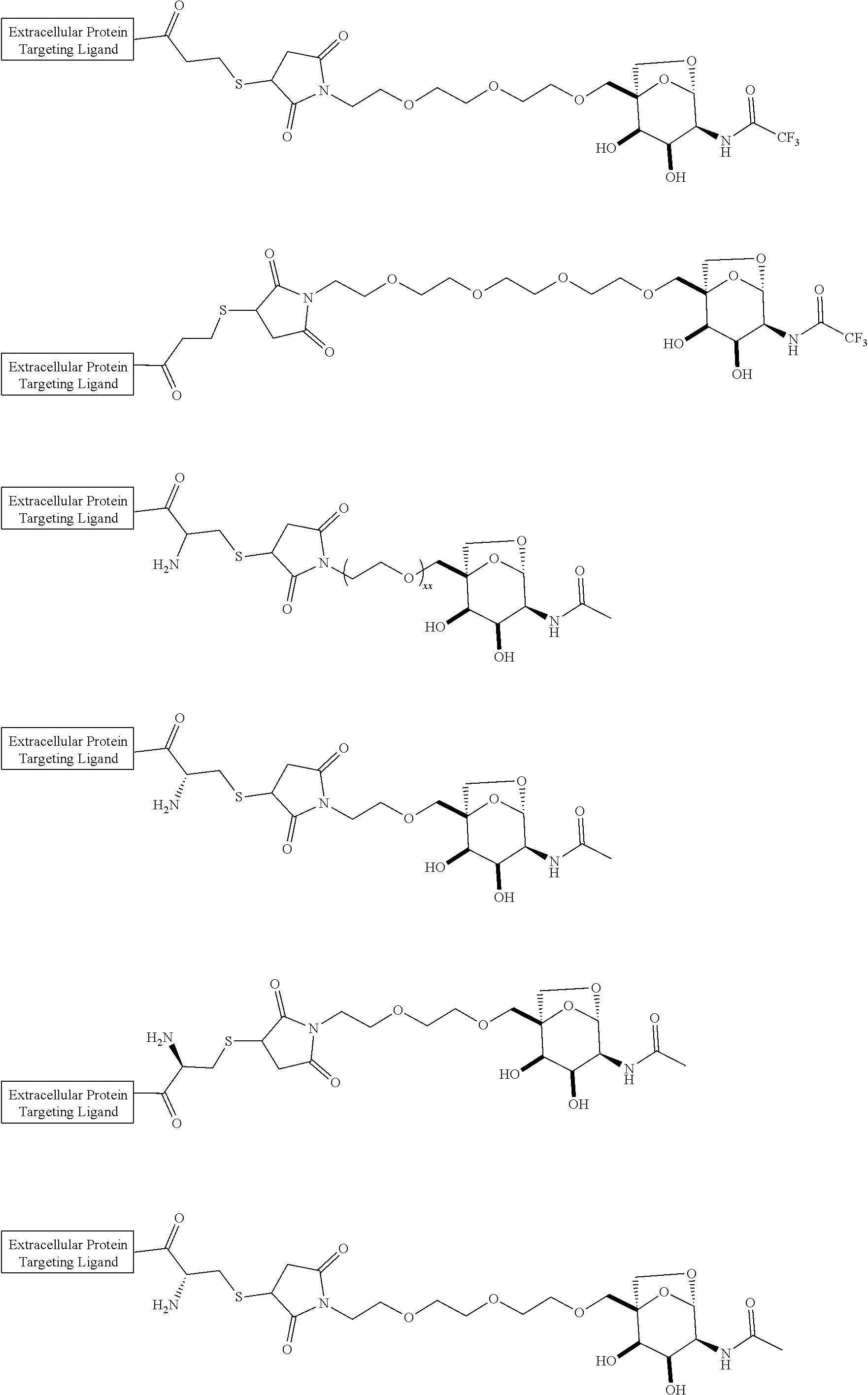

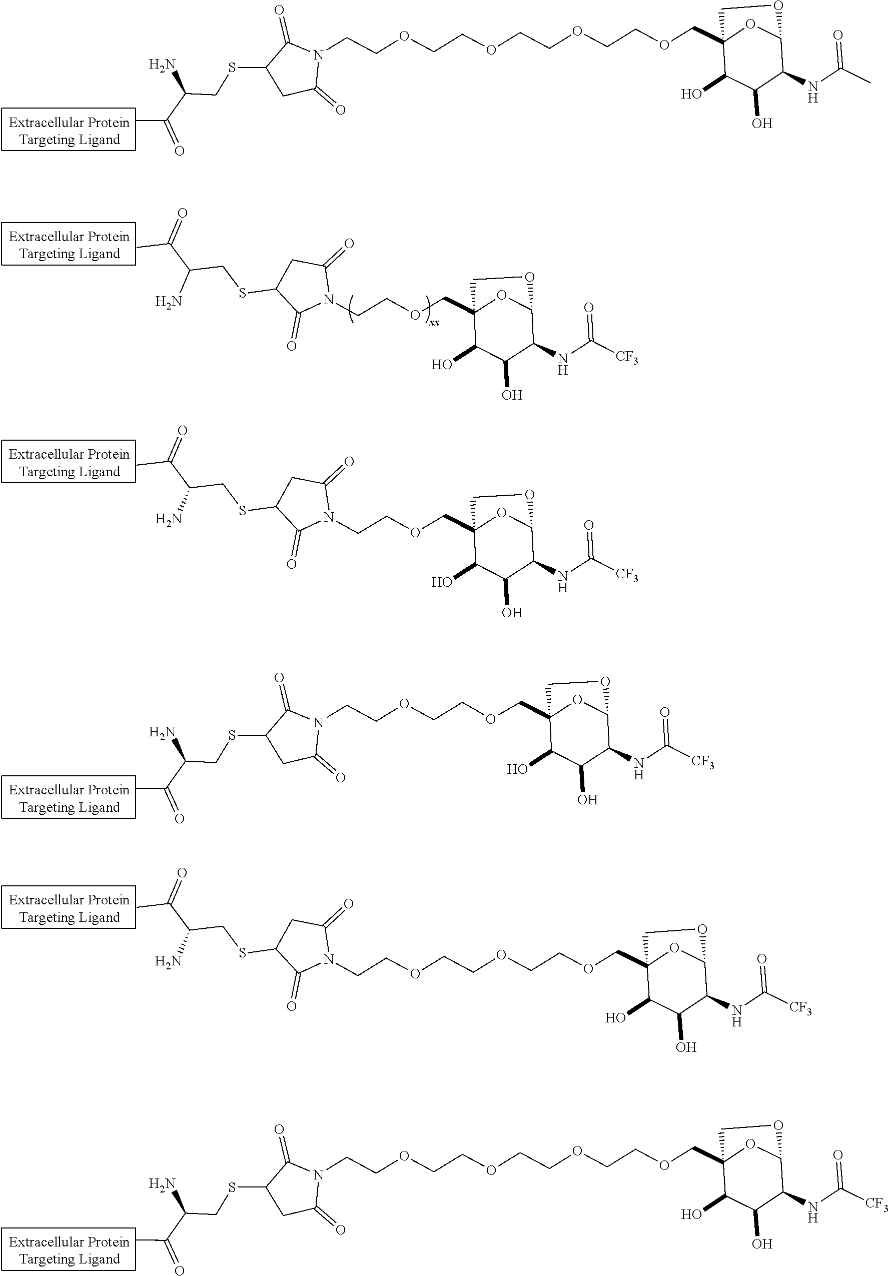

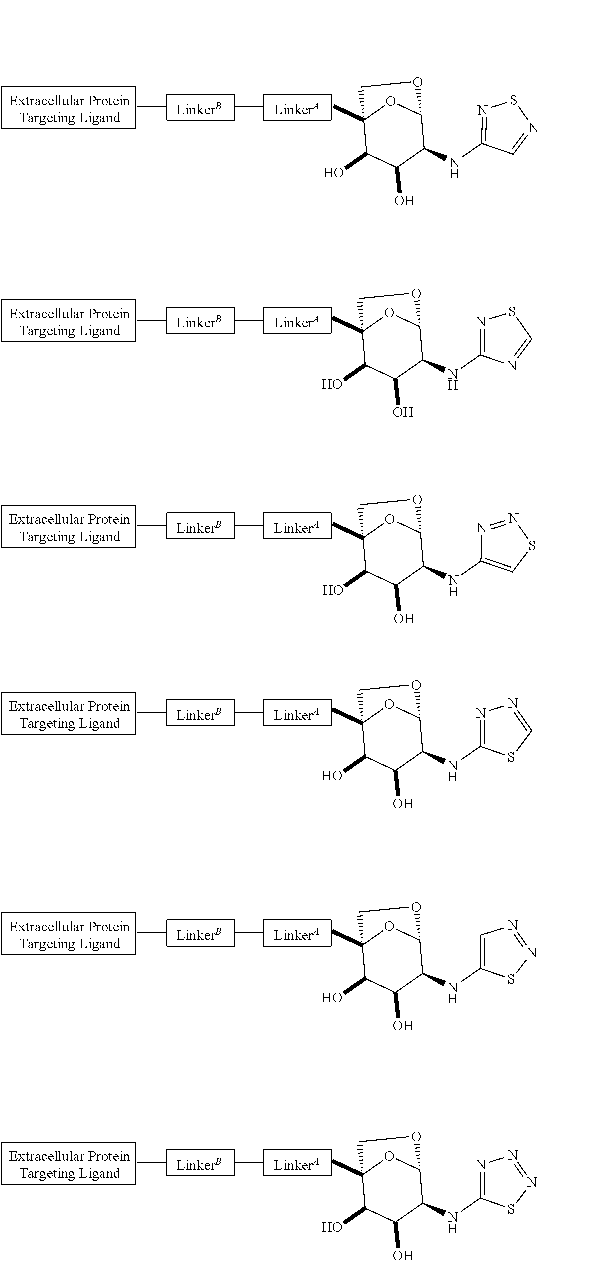

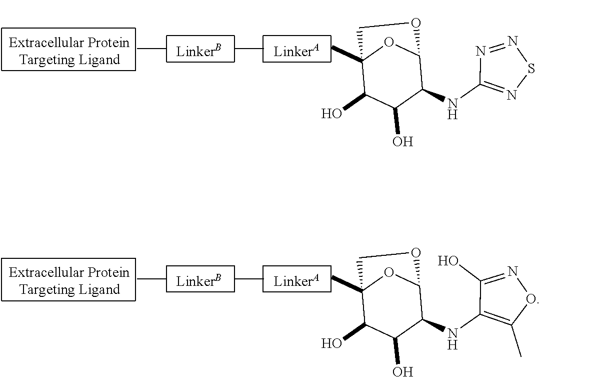

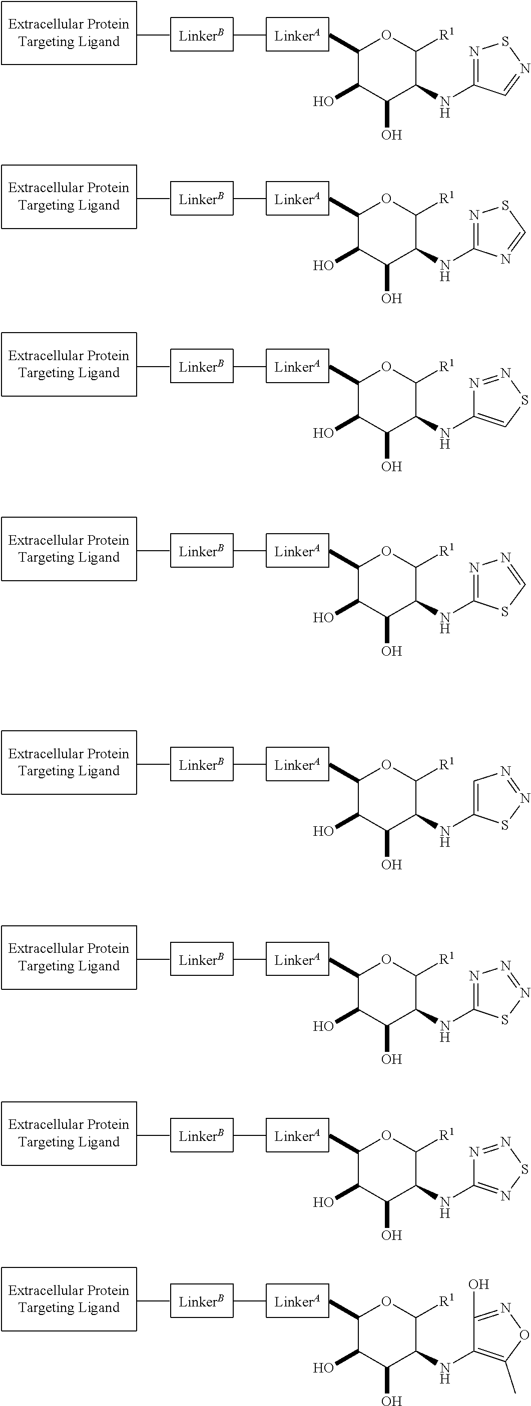

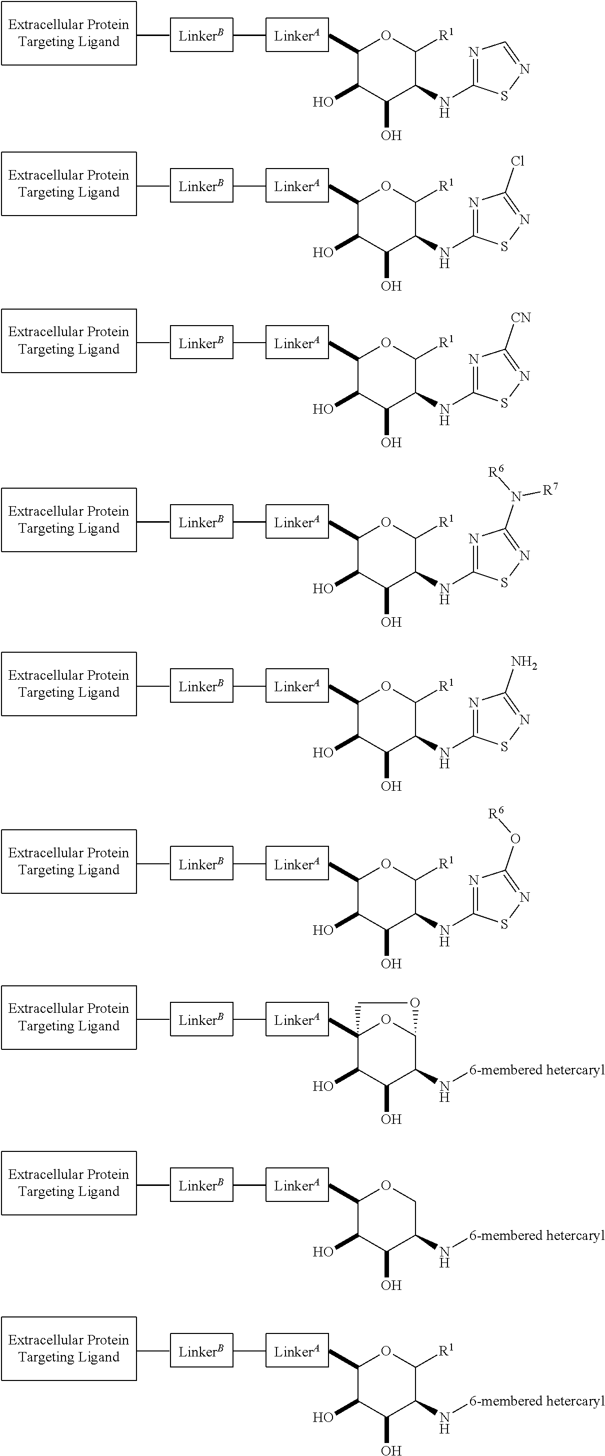

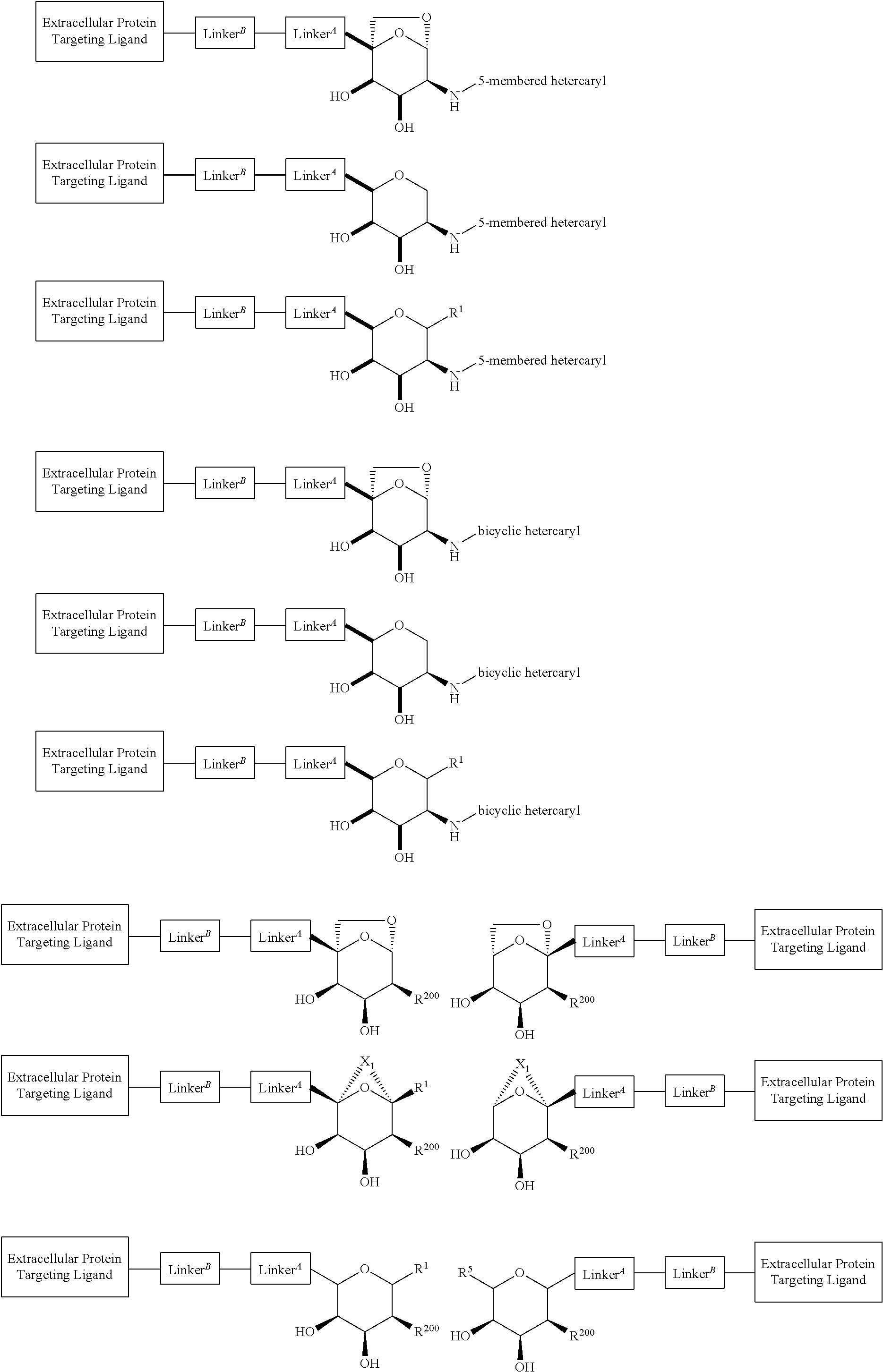

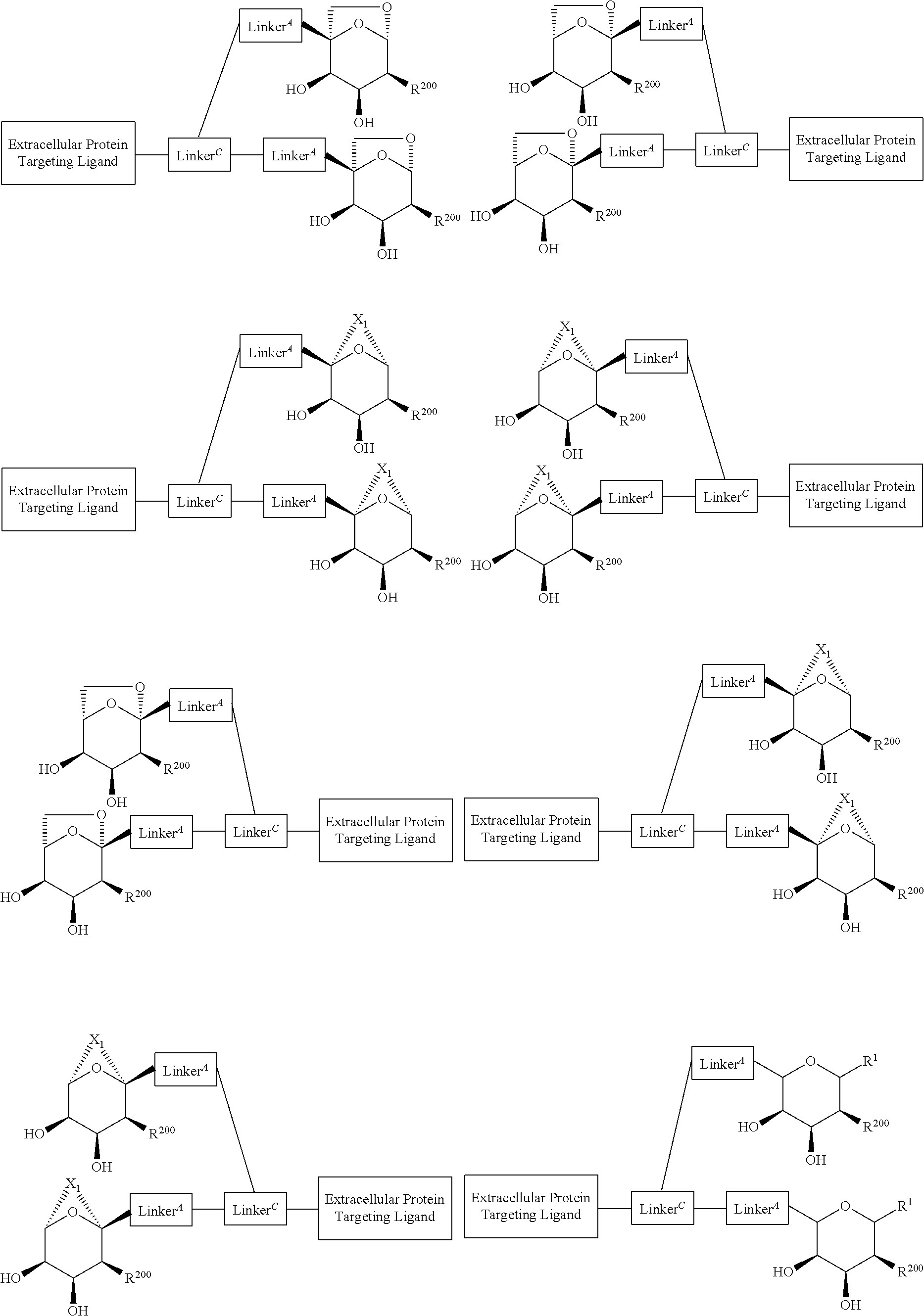

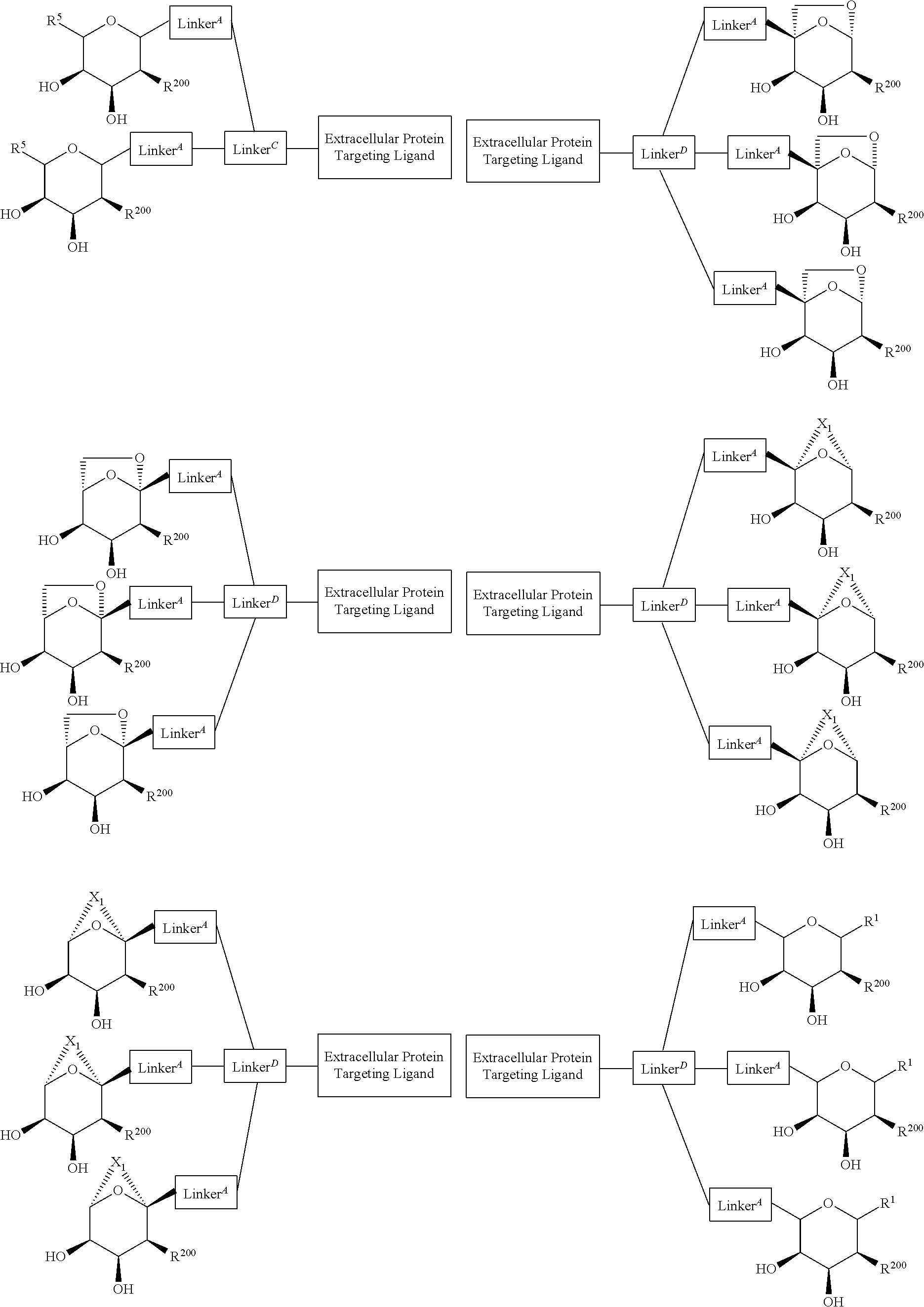

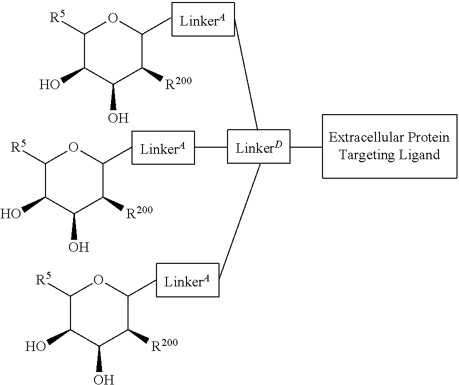

In one embodiment, the compound of Formula II is an Extracellular Protein degrading compound in which the ASGPR ligand is a ligand as described herein

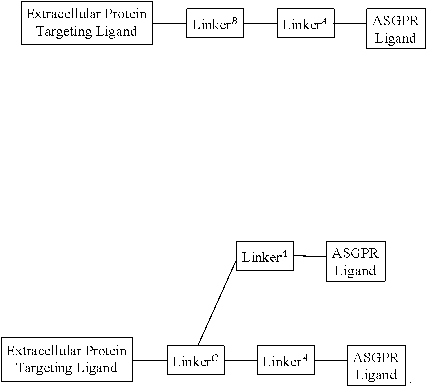

In one embodiment, in the compound of Formula II, the ASGPR ligand is linked at either the C1 or C5 (R¹ or R₅) position to form a degrading compound. In one embodiment, in the compound of Formula II, the ASGPR ligand is linked at C6. In various embodiments, when the ASGPR ligand is

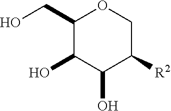

then non-limiting examples of ASGPR binding compounds of Formula II include:

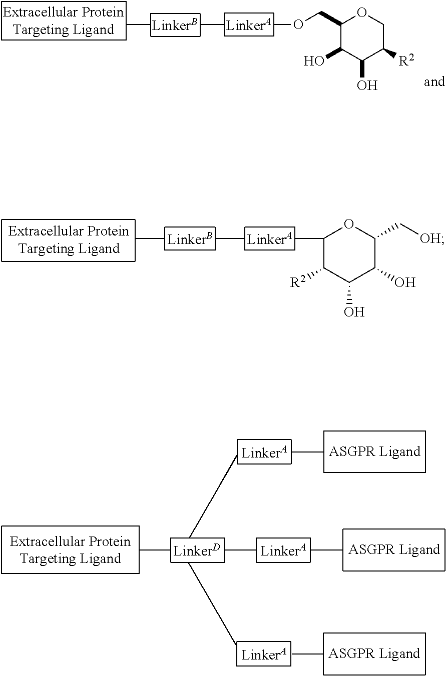

or the bi- or tri-substituted versions thereof or pharmaceutically acceptable salts thereof, where the bi- or tri-substitution refers to the number additional galactose derivatives attached to a linker moiety. In certain embodiments the compound of Formula II is selected from:

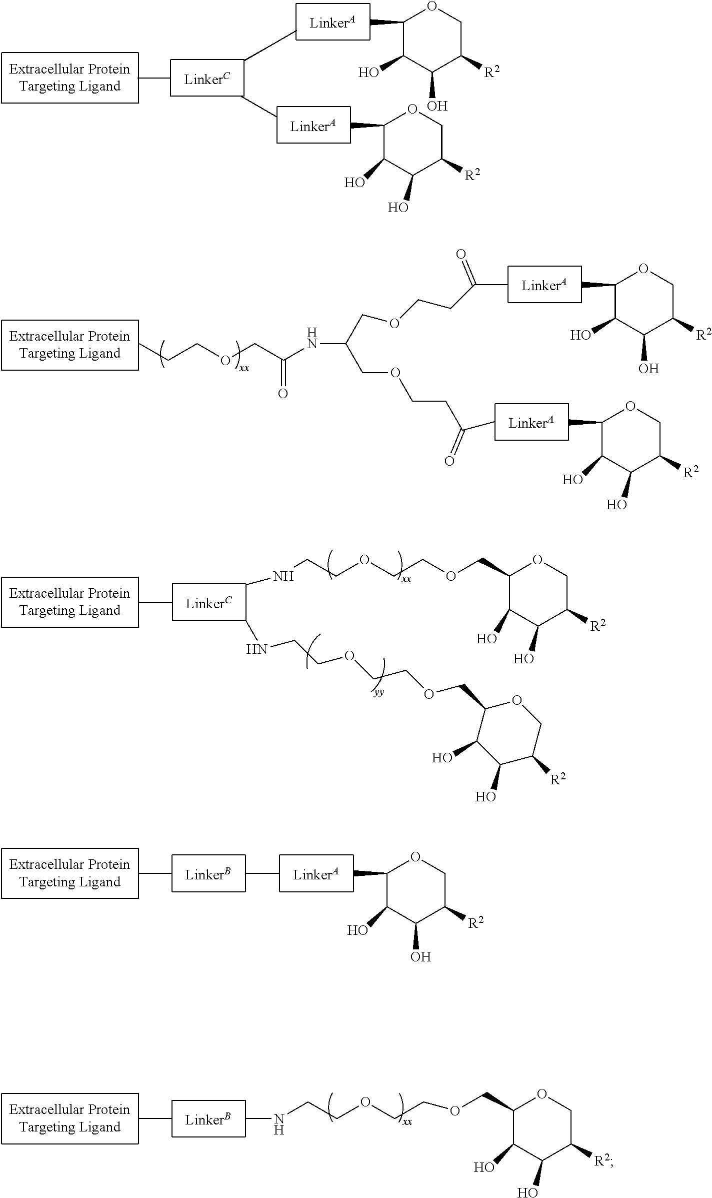

wherein in certain embodiments R² is selected from —NR⁶COR³, —NR⁶-(5-membered heteroaryl), and —NR⁶-(6-membered heteroaryl), each of which R² groups is optionally substituted with 1, 2, 3, or 4 independent, substituents as described herein, for example 1, 2, 3 or 4 substituents independently selected from F, Cl, Br, haloalkyl, or alkyl.

In certain embodiments, the compound of Formula II is selected from:

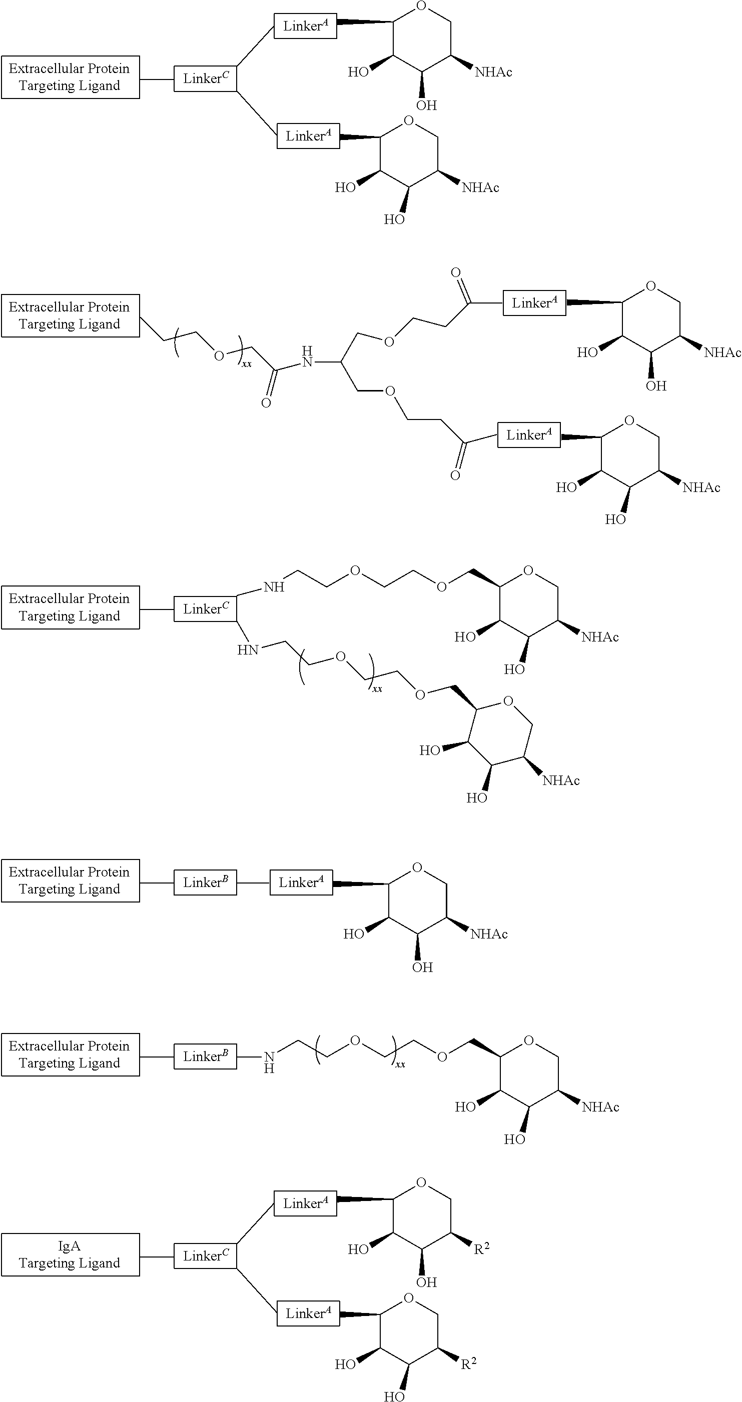

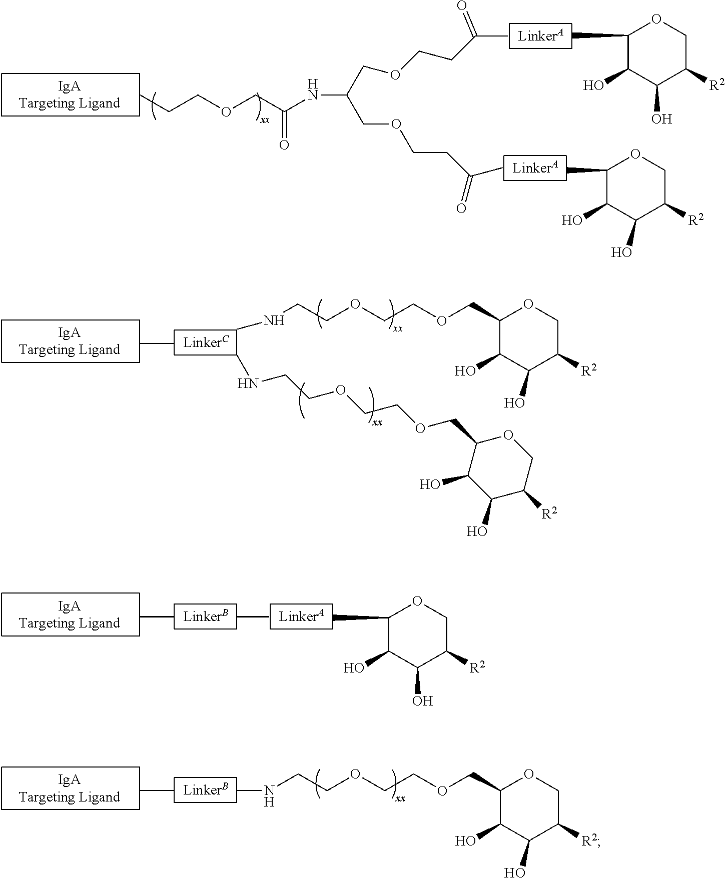

wherein in certain embodiments R² is selected from —NR⁶COR³, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

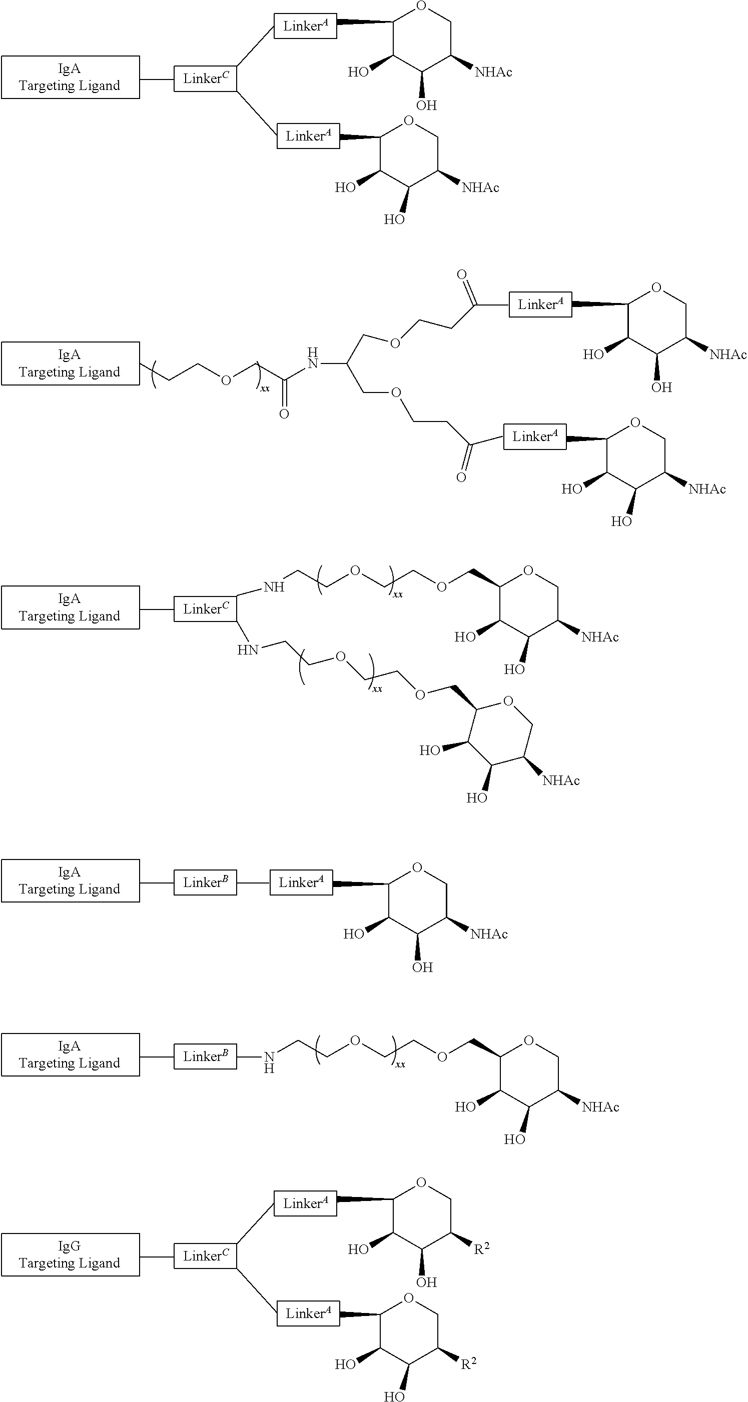

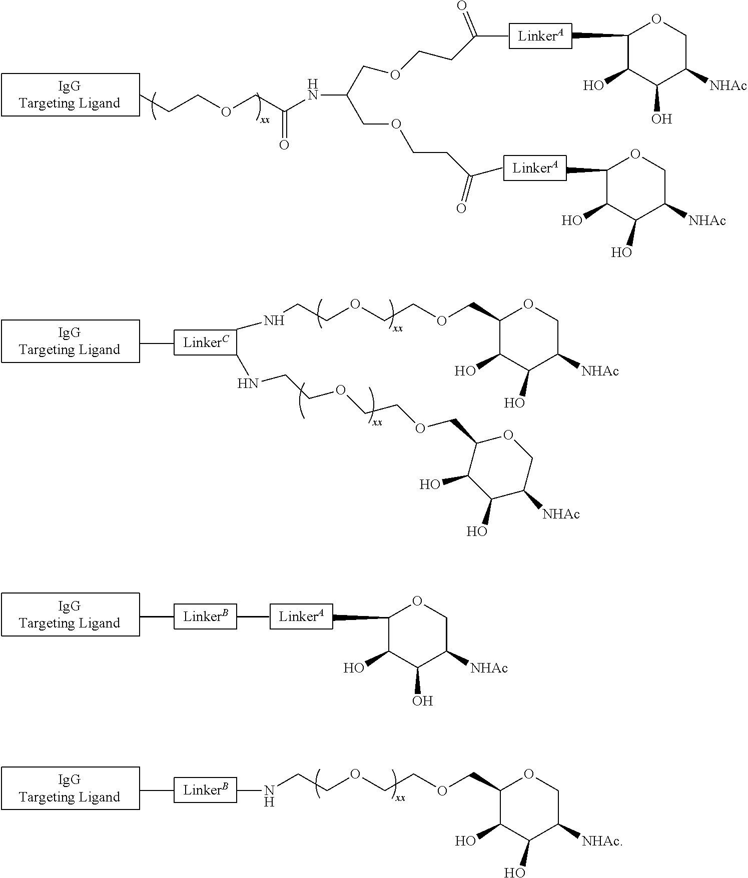

wherein in certain embodiments R² is selected from —NR⁶COR³, —NR⁶-(5-membered heteroaryl), and —NR⁶-(6-membered heteroaryl), each of which R² groups is optionally substituted with 1, 2, 3, or 4 independent, substituents as described herein, for example 1, 2, 3 or 4 substituents independently selected from F, Cl, Br, haloalkyl, or alkyl.

In certain embodiments, the compound of Formula II is selected from:

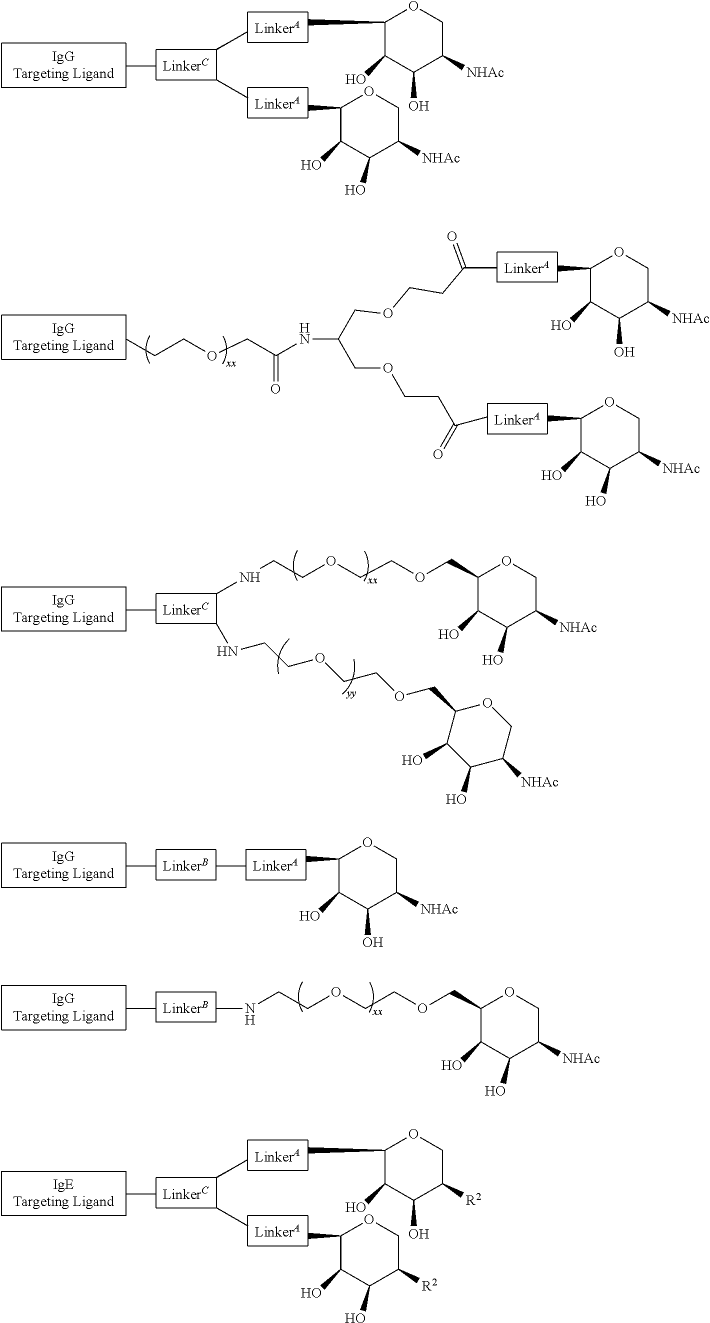

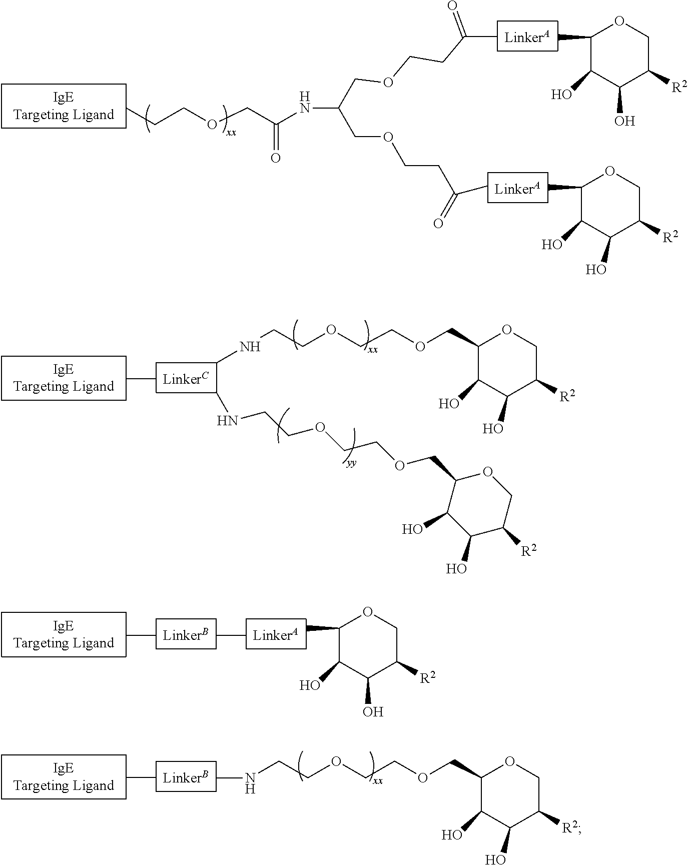

wherein in certain embodiments R² is selected from —NR⁶COR³, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

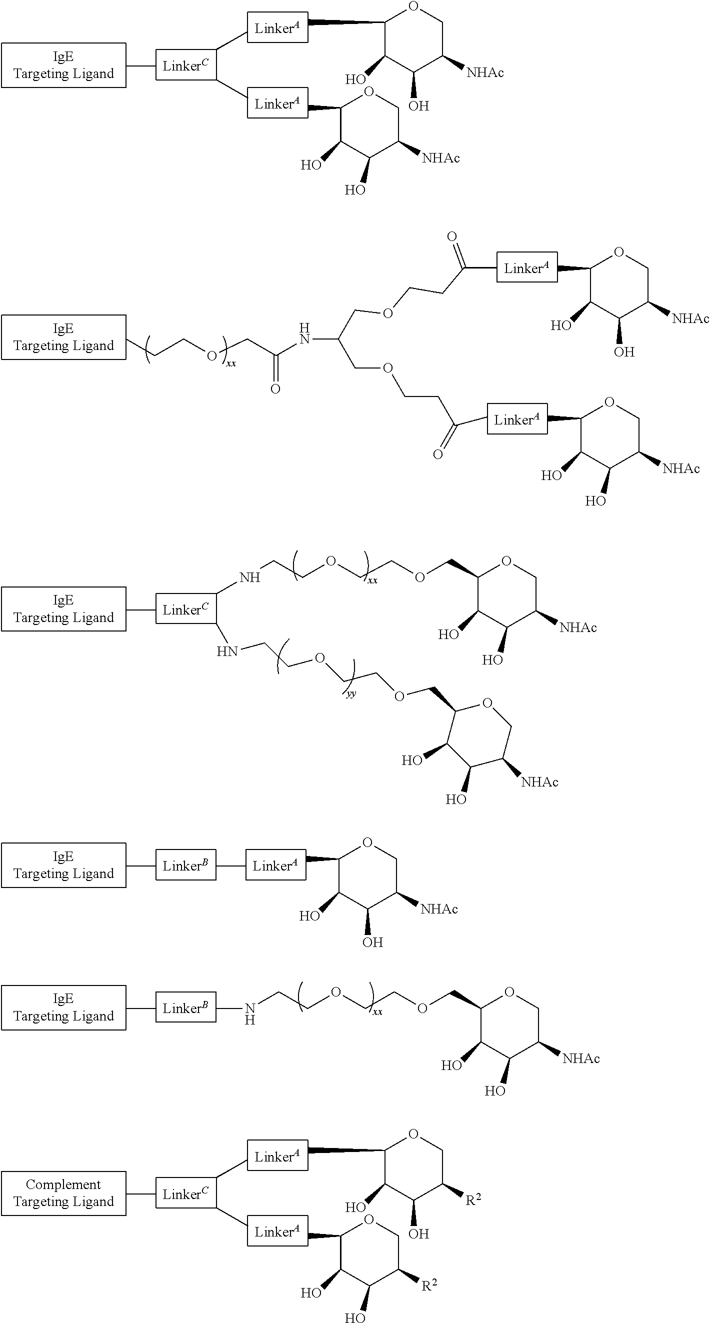

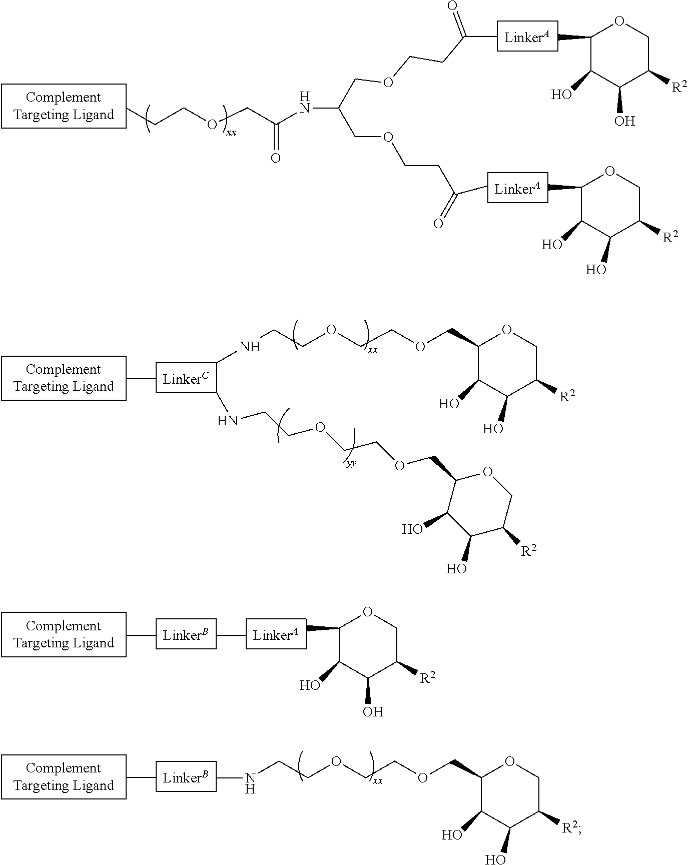

wherein in certain embodiments R² is selected from —NR⁶COR³, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

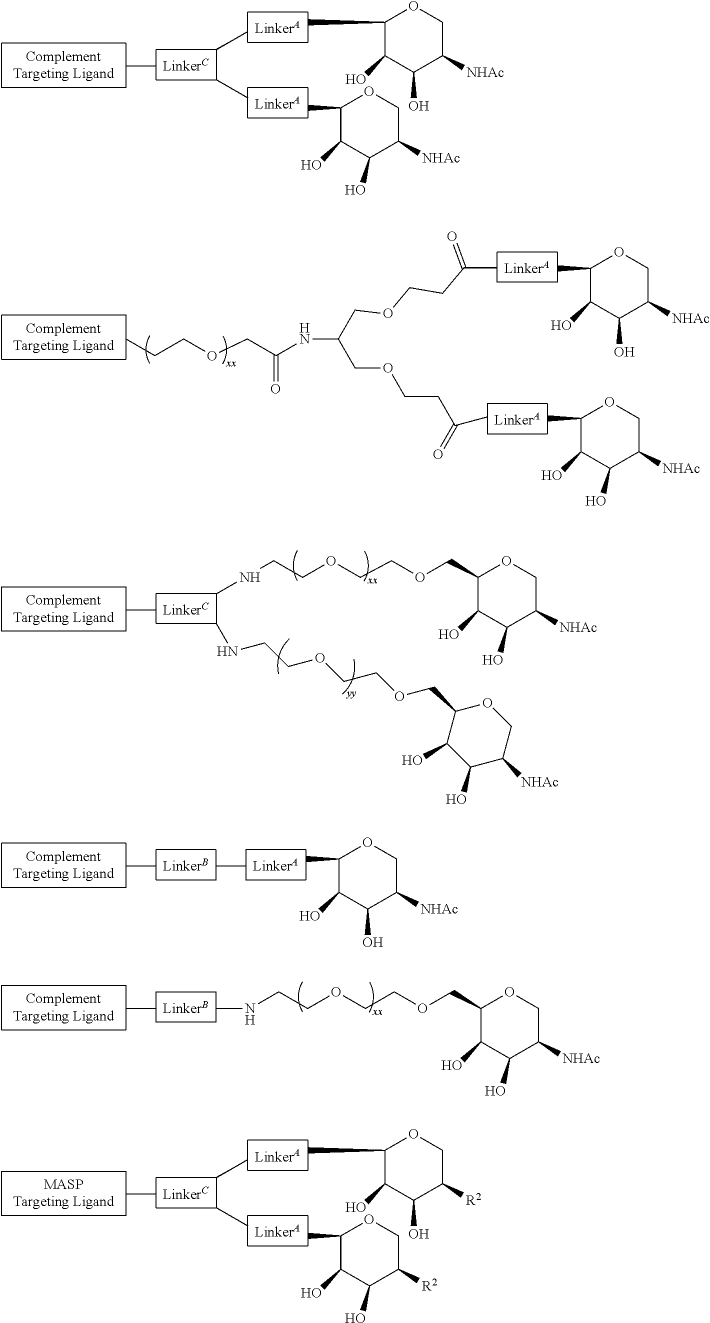

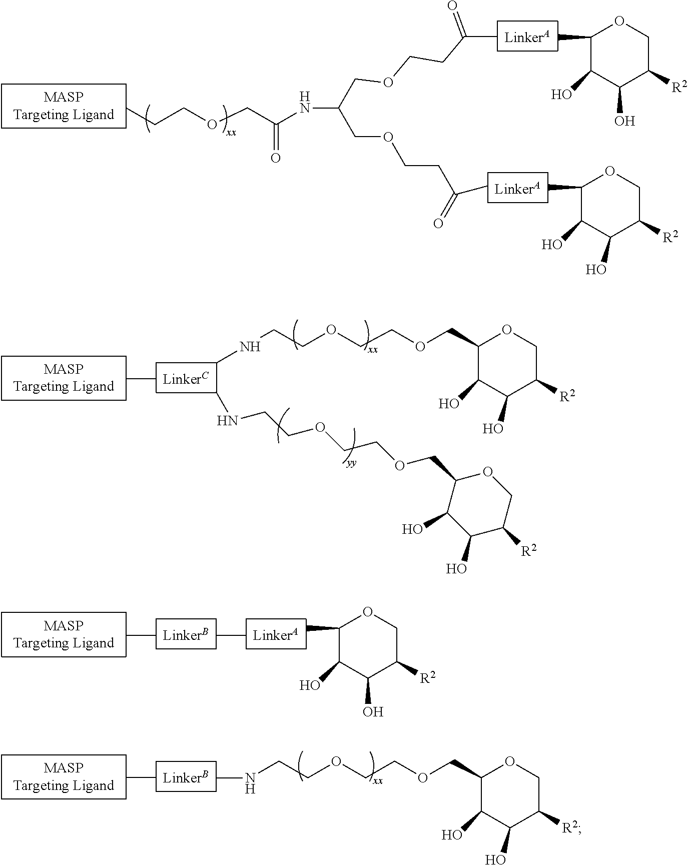

wherein in certain embodiments R² is selected from —NR⁶COR³, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

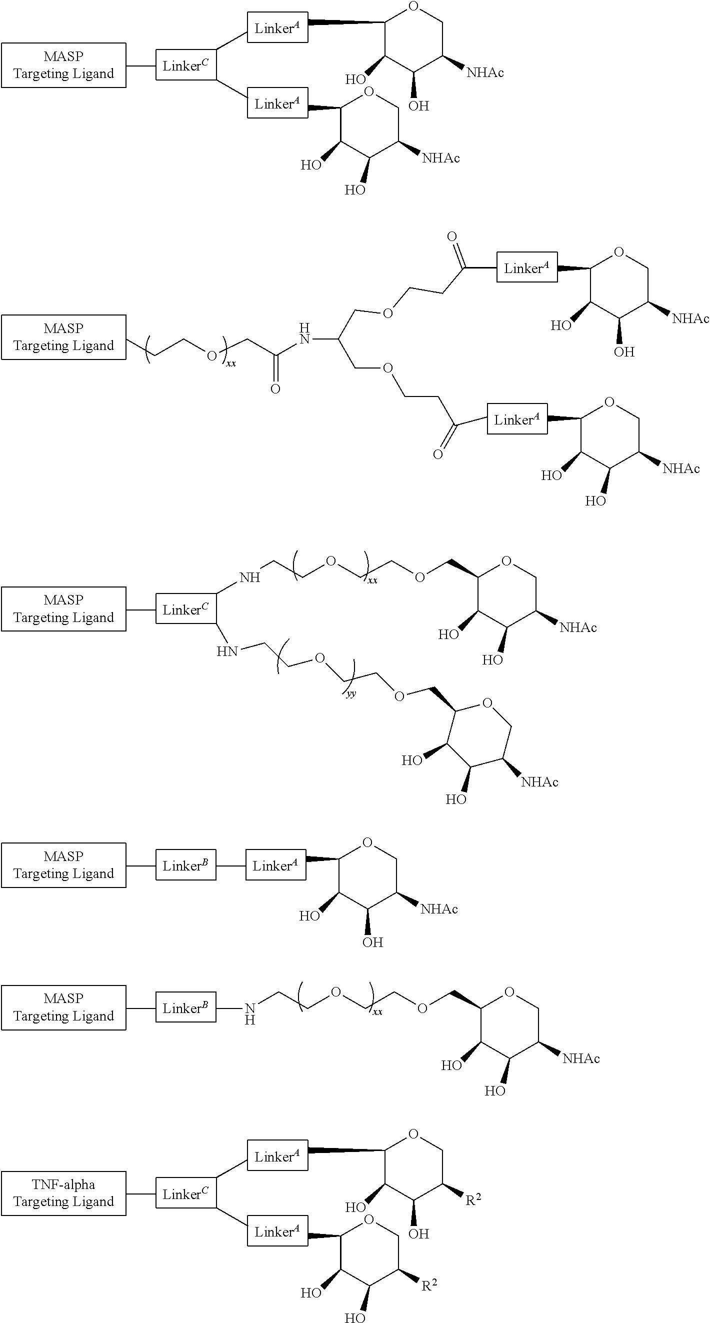

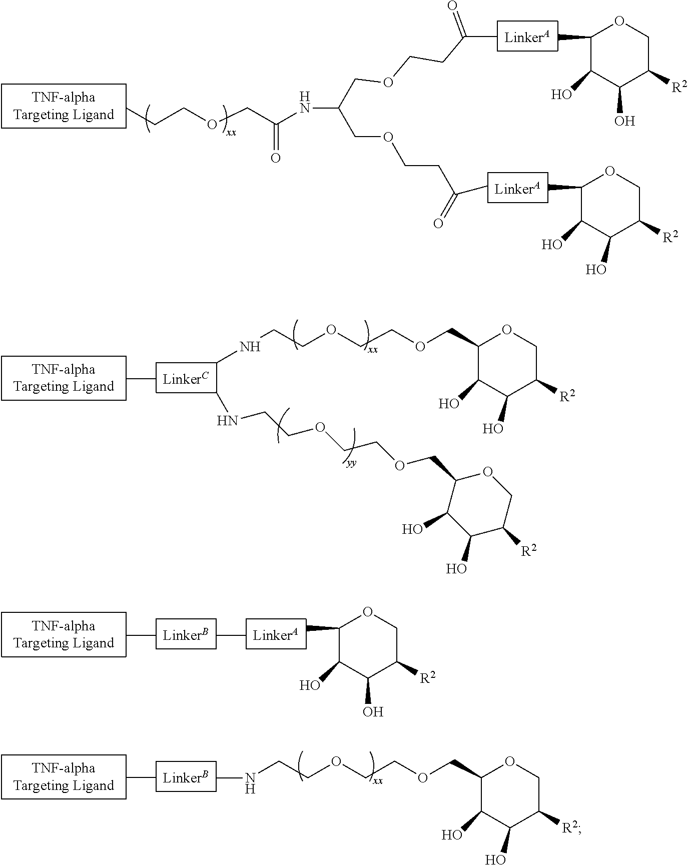

wherein in certain embodiments R² is selected from —NR⁶COR³, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

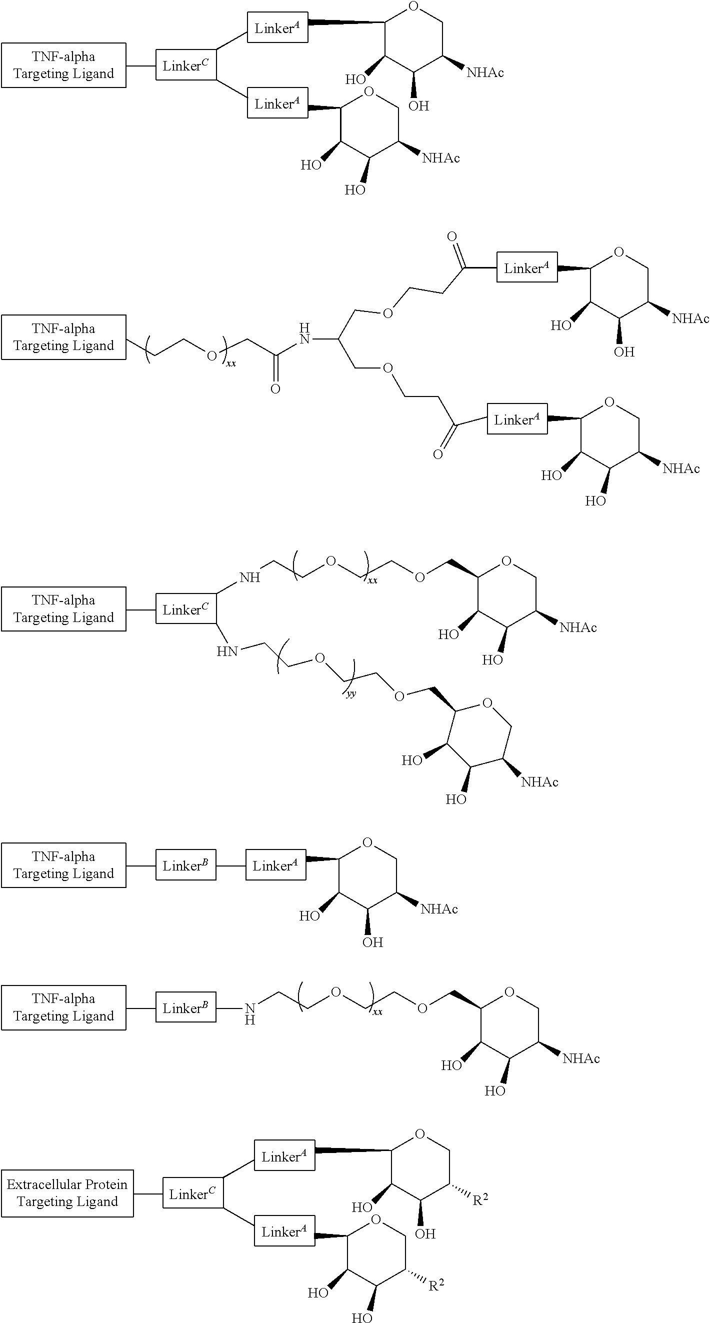

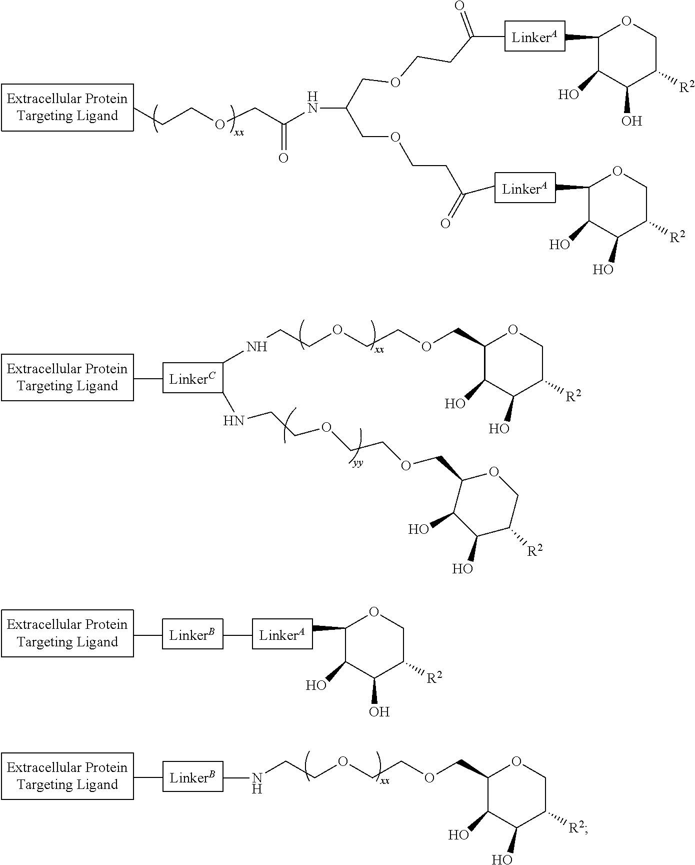

wherein in certain embodiments R² is selected from —NR⁶COR¹, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

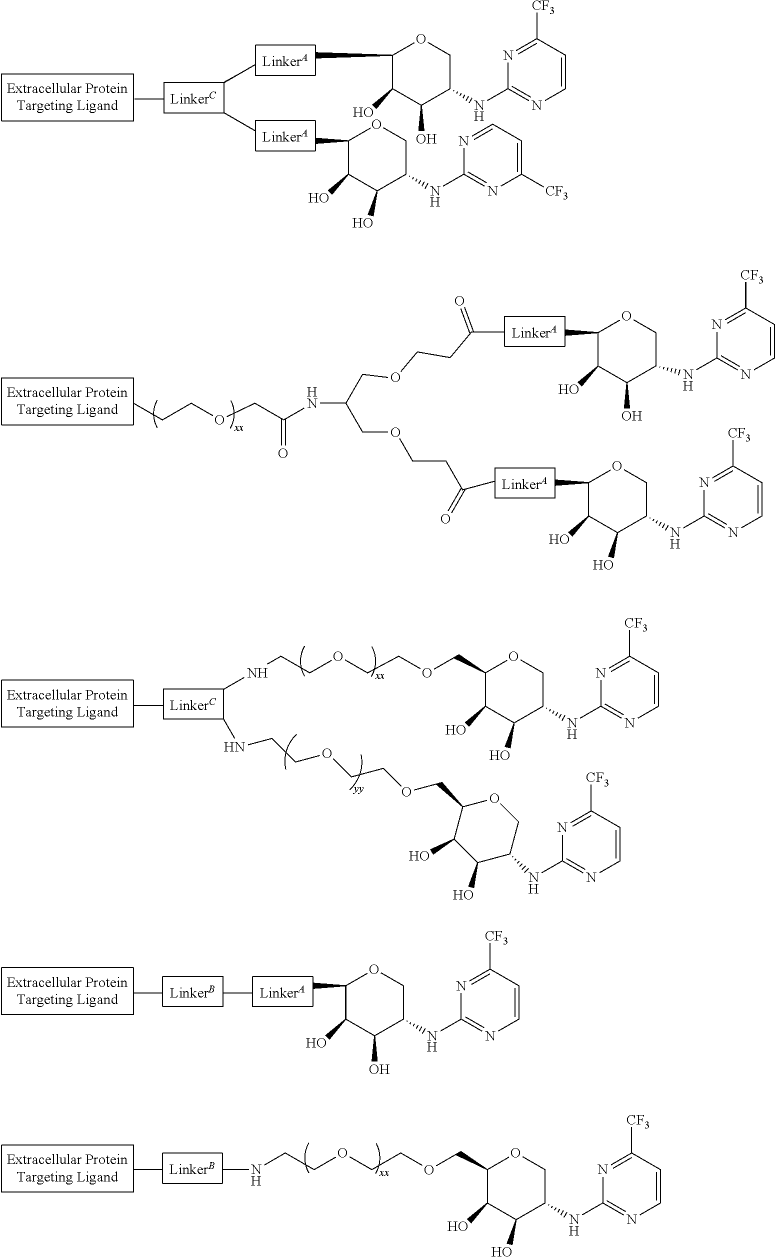

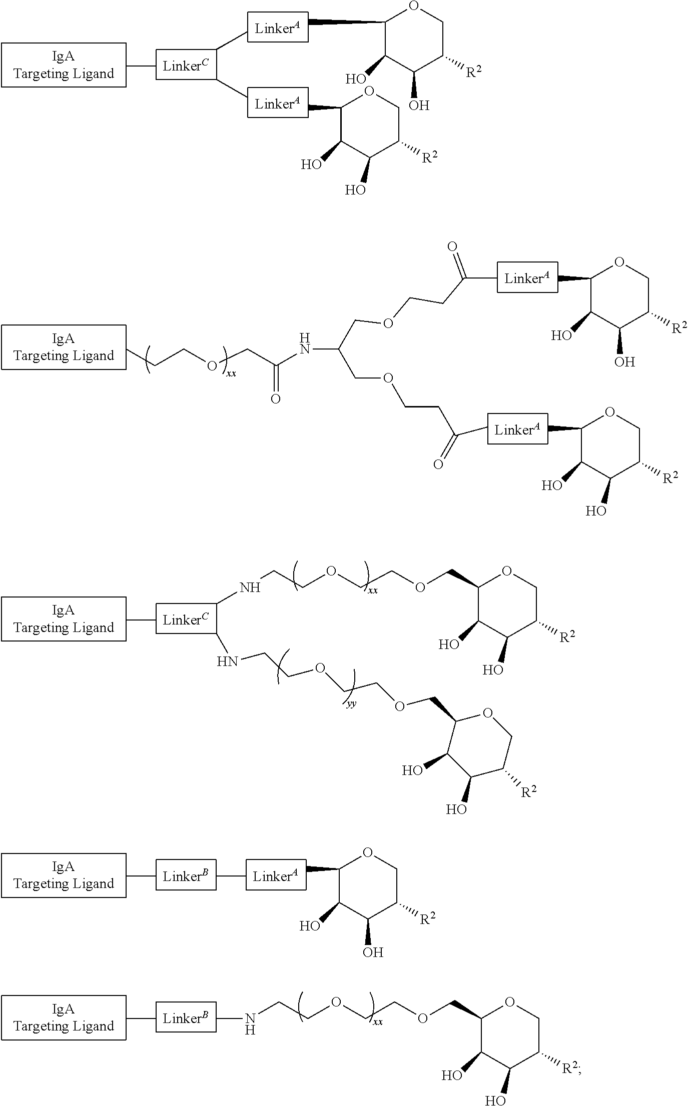

wherein in certain embodiments R² is selected from —NR⁶COR¹, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

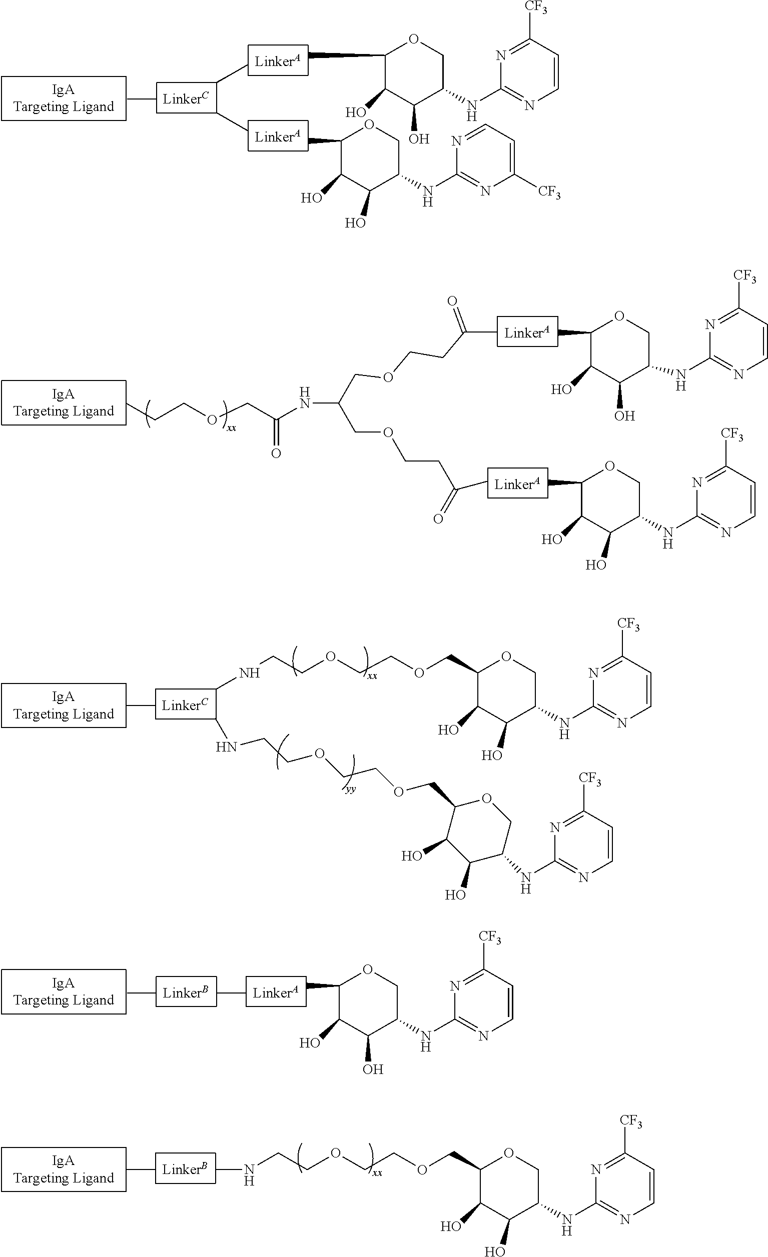

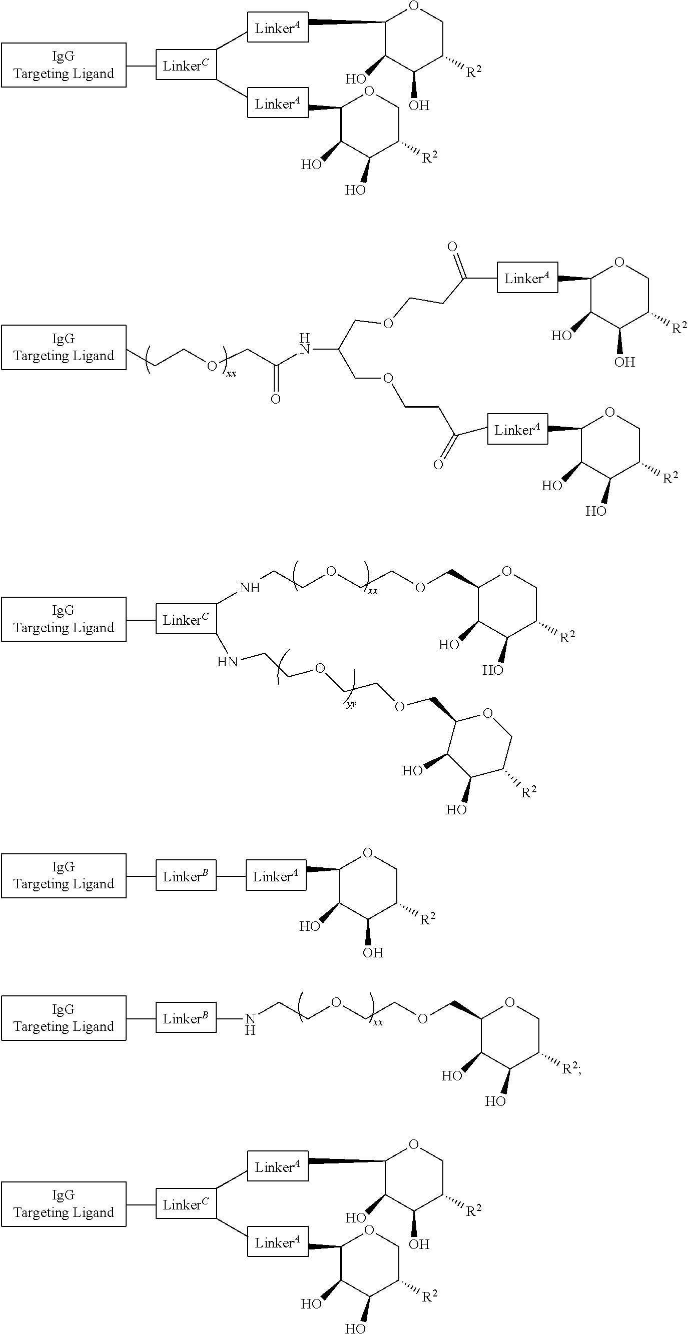

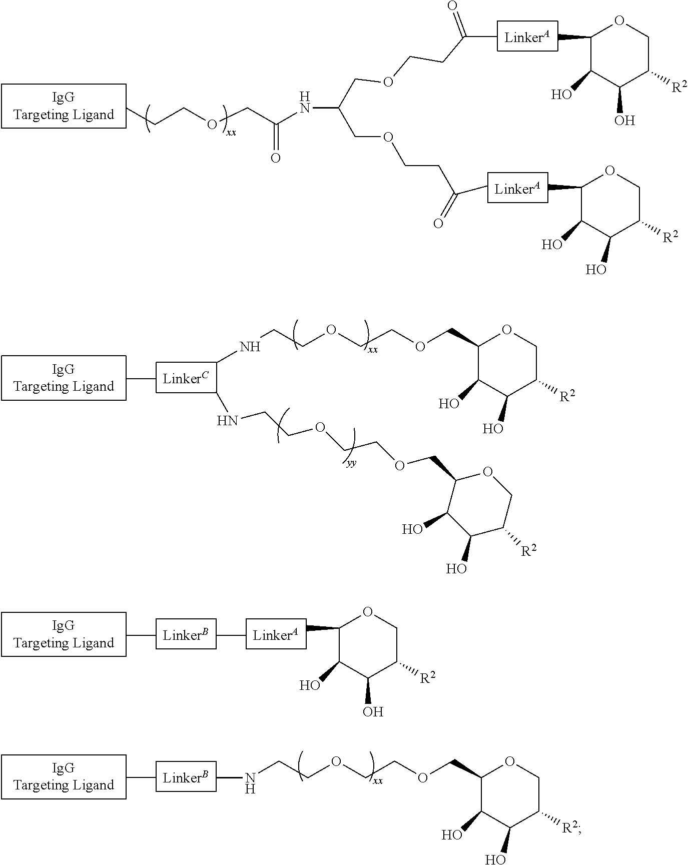

wherein in certain embodiments R² is selected from —NR⁶COR¹, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

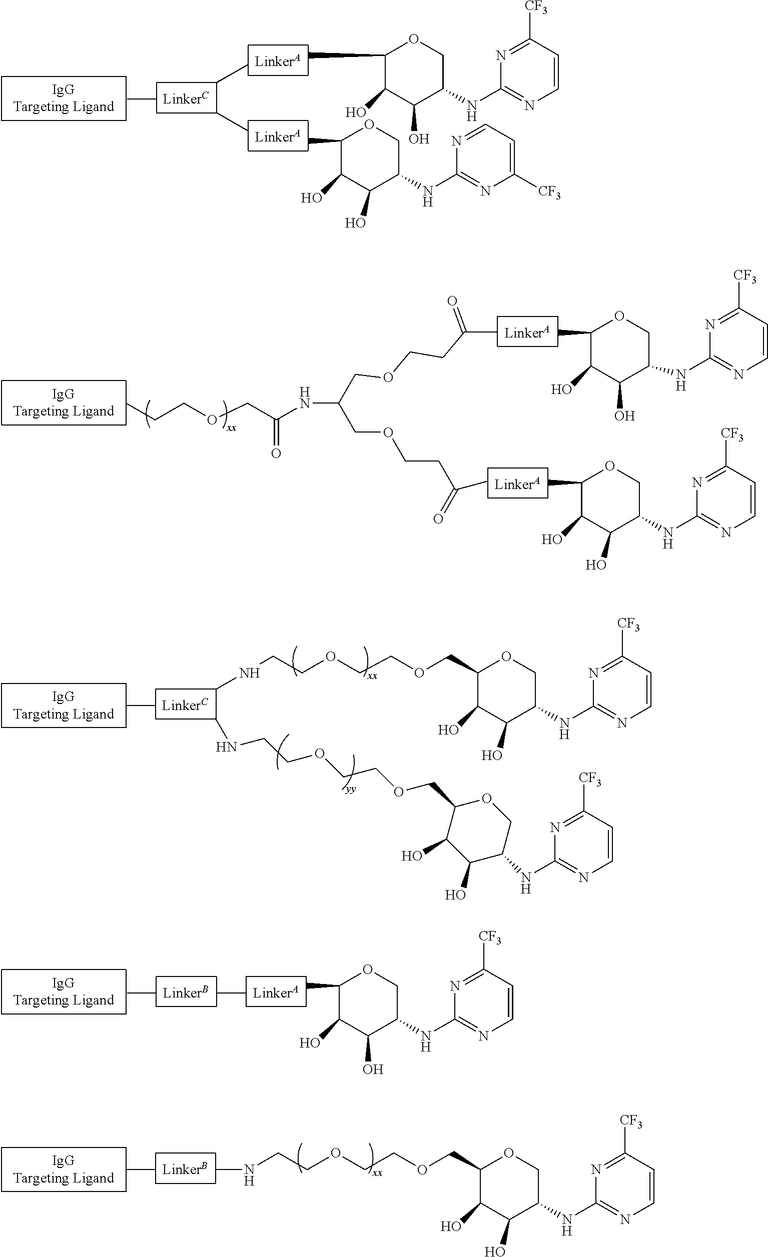

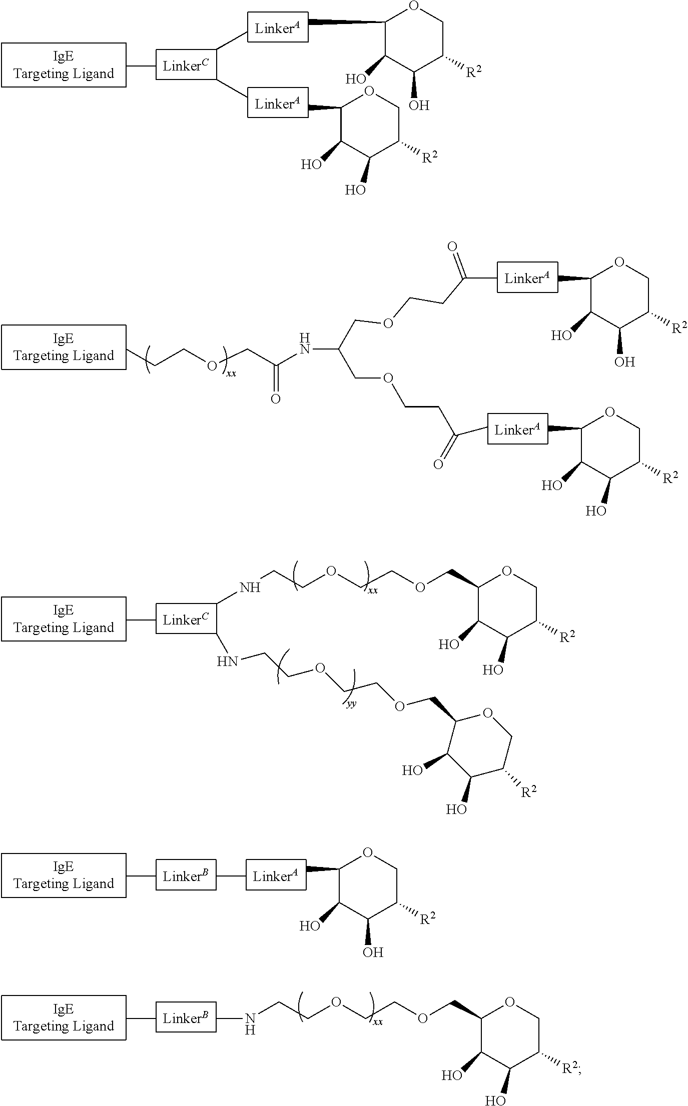

wherein in certain embodiments R² is selected from —NR⁶COR¹, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

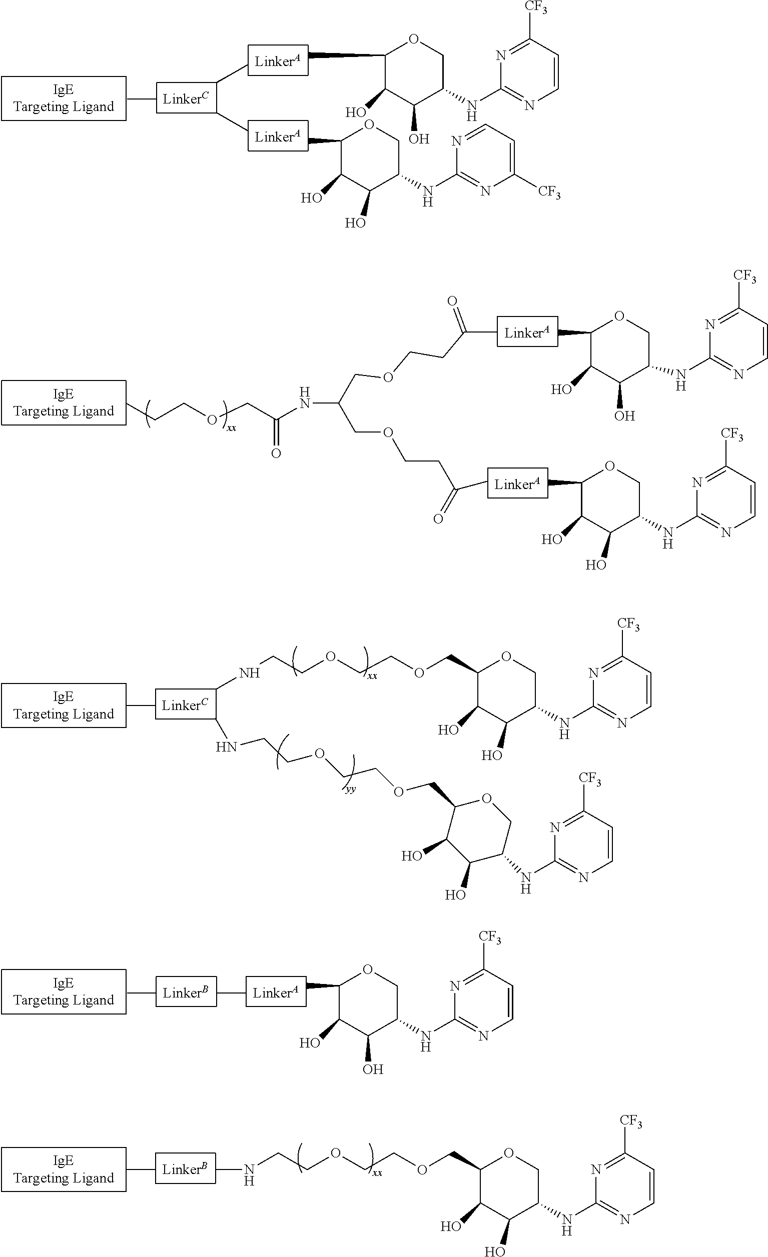

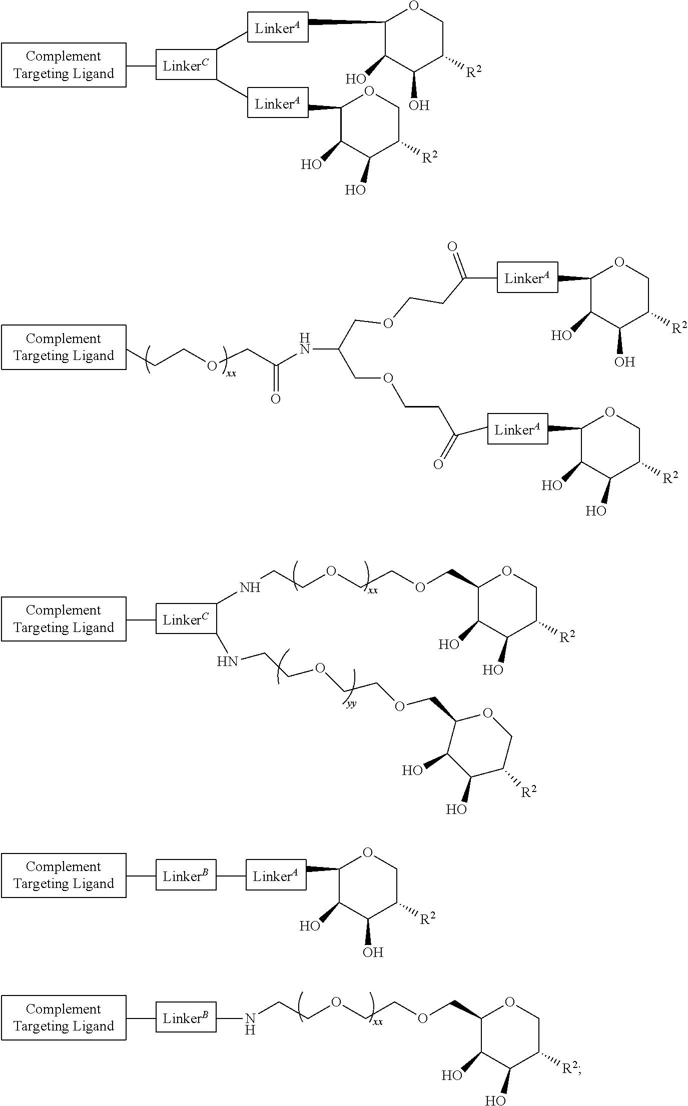

wherein in certain embodiments R² is selected from —NR⁶COR¹, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

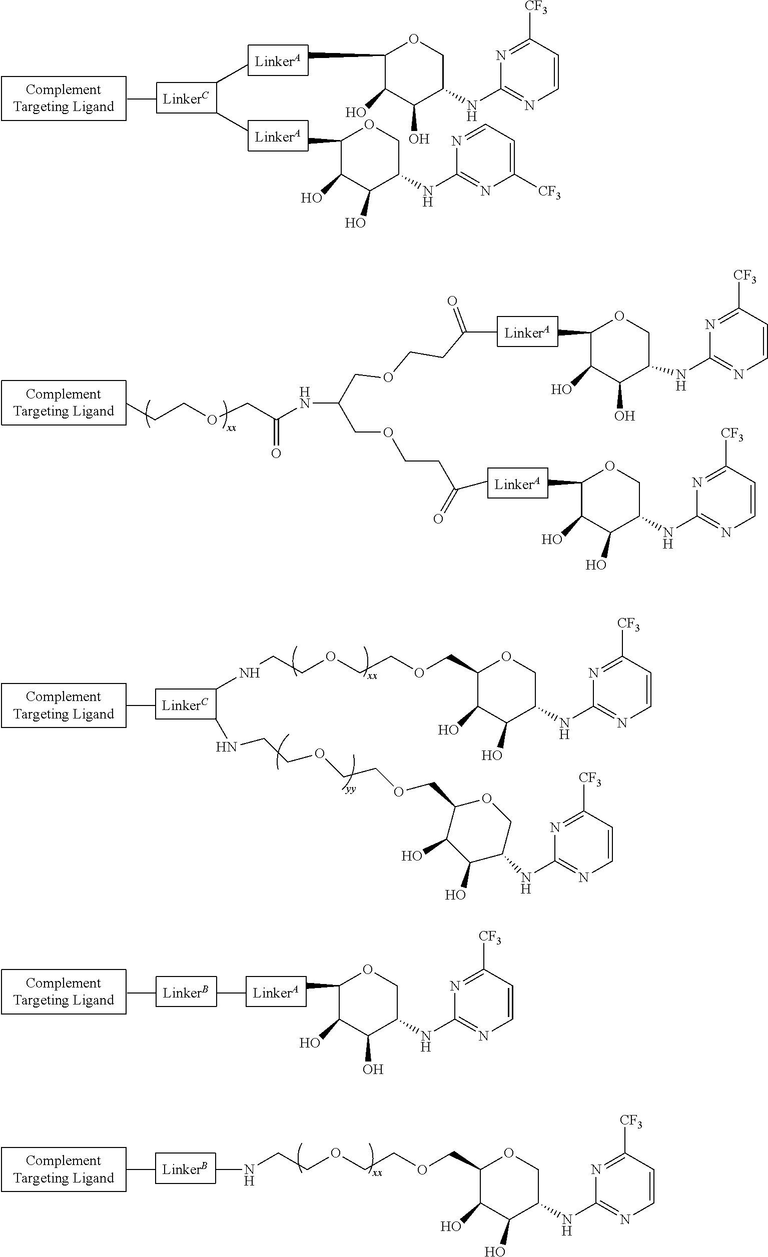

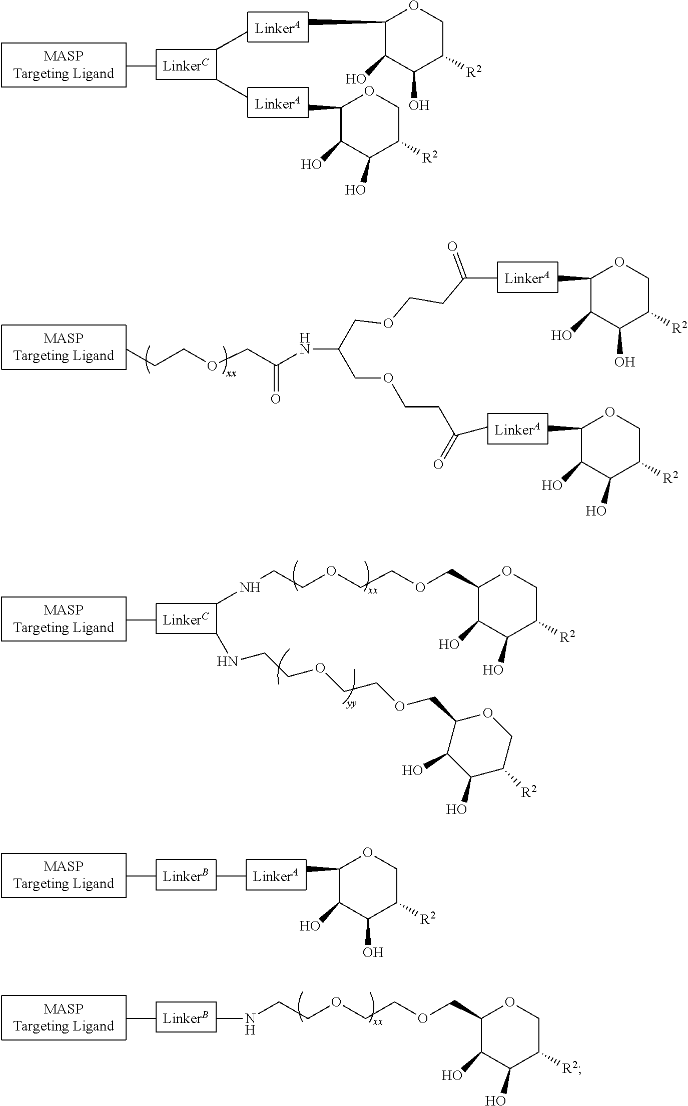

wherein in certain embodiments R² is selected from —NR⁶COR¹, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

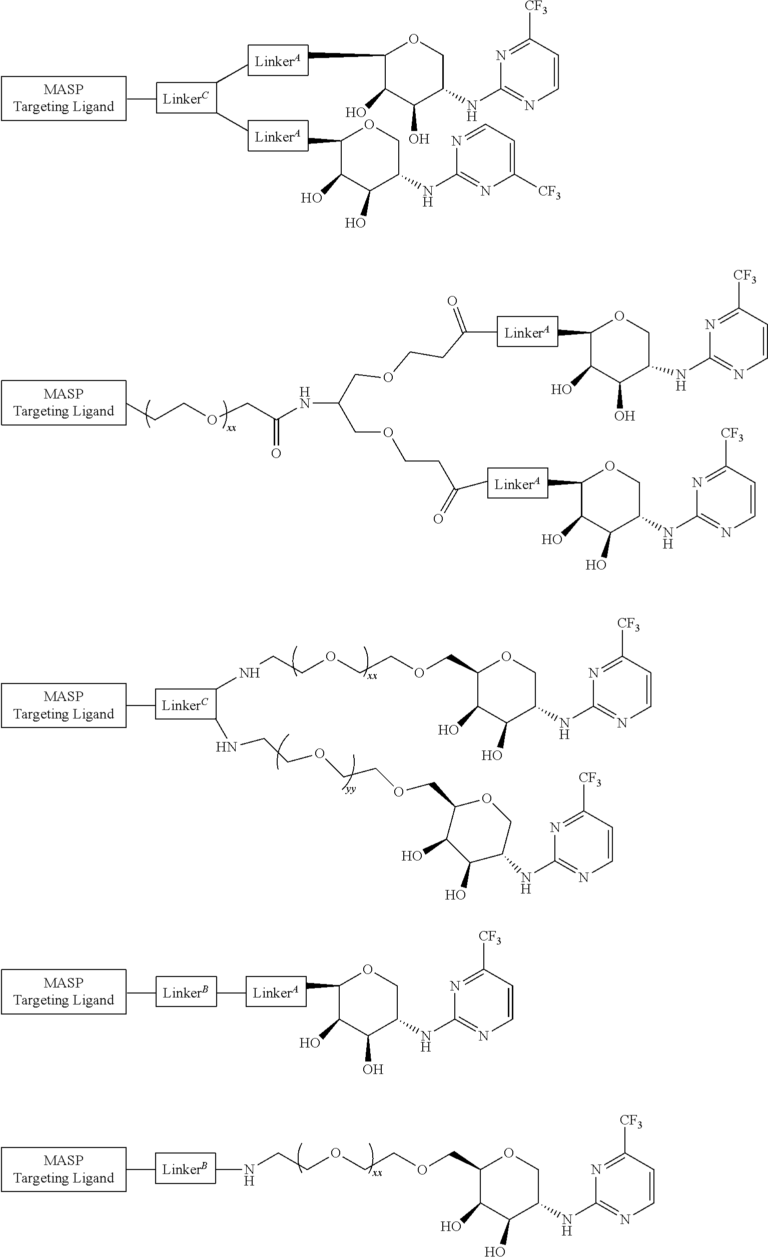

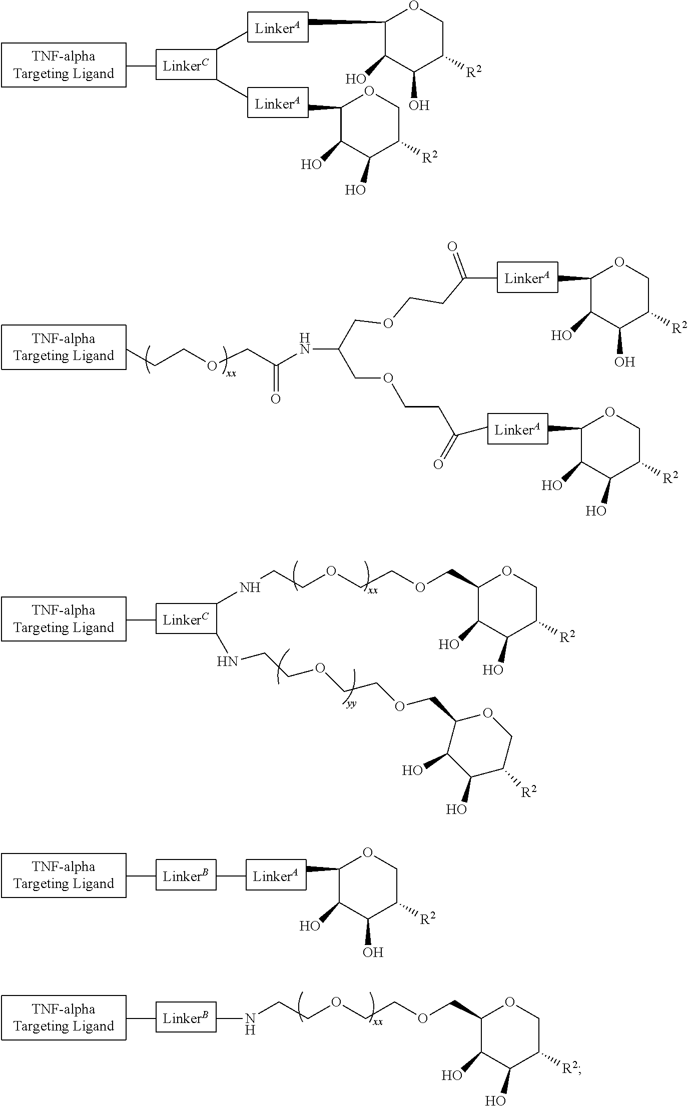

wherein in certain embodiments R² is selected from —NR⁶COR¹, —NR⁶-(5-membered heteroaryl), and —NR⁶-(6-membered heteroaryl), each of which R² groups is optionally substituted with 1, 2, 3, or 4 independent, substituents as described herein, for example 1, 2, 3, or 4 substituents independently selected from F, Cl, Br, haloalkyl, or alkyl.

In certain embodiments, the compound of Formula II is selected from:

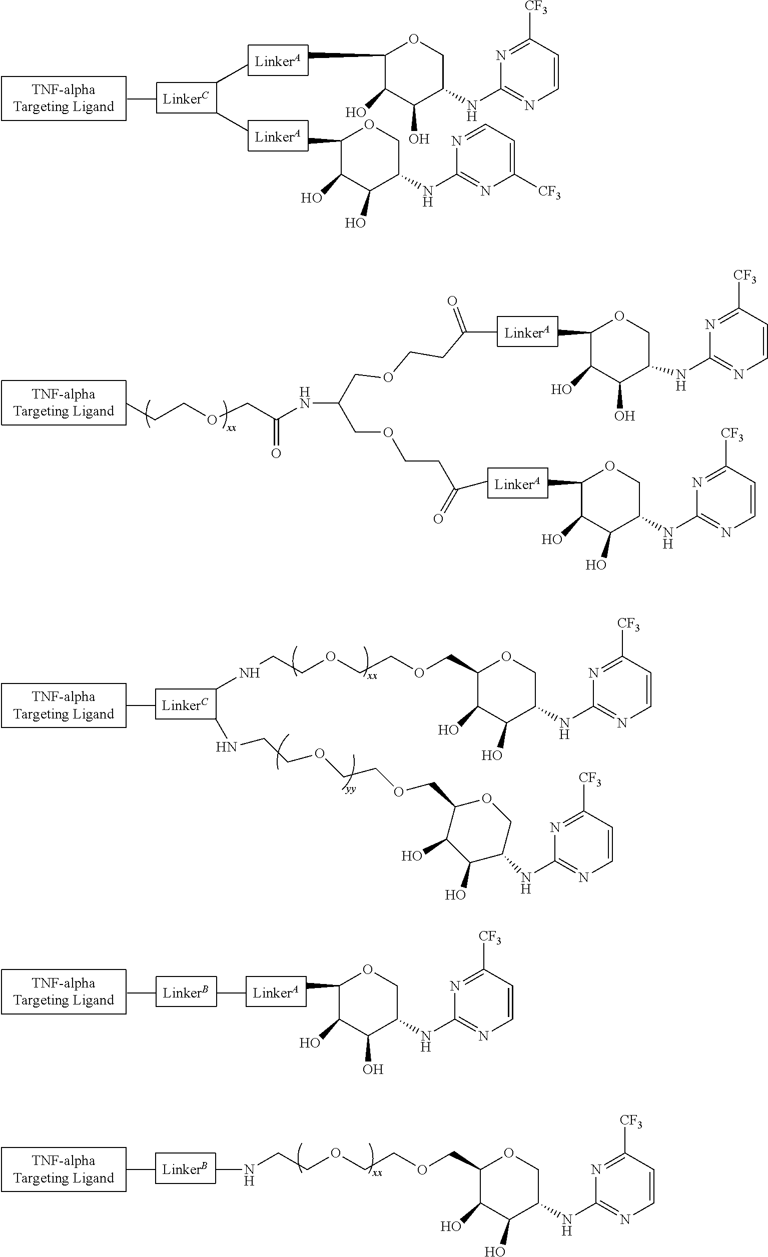

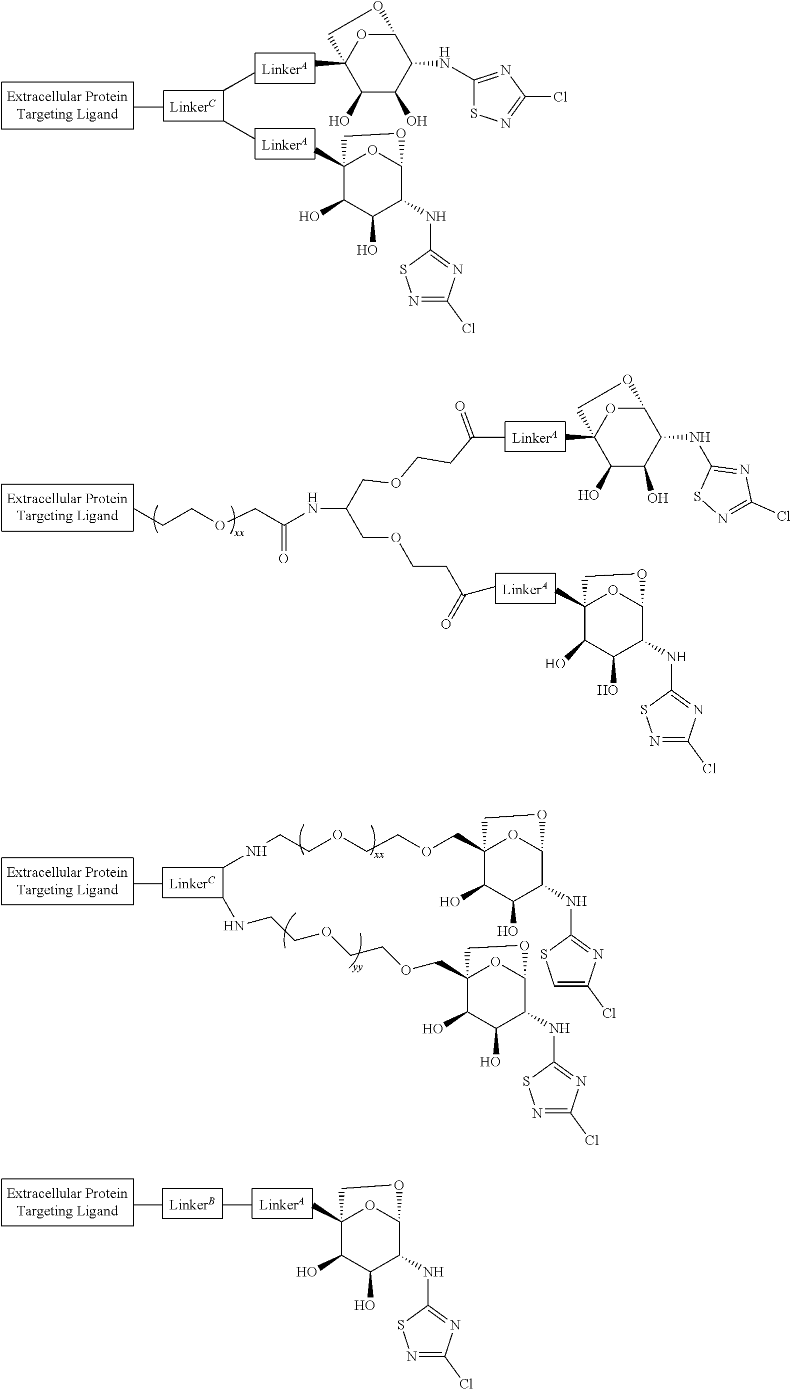

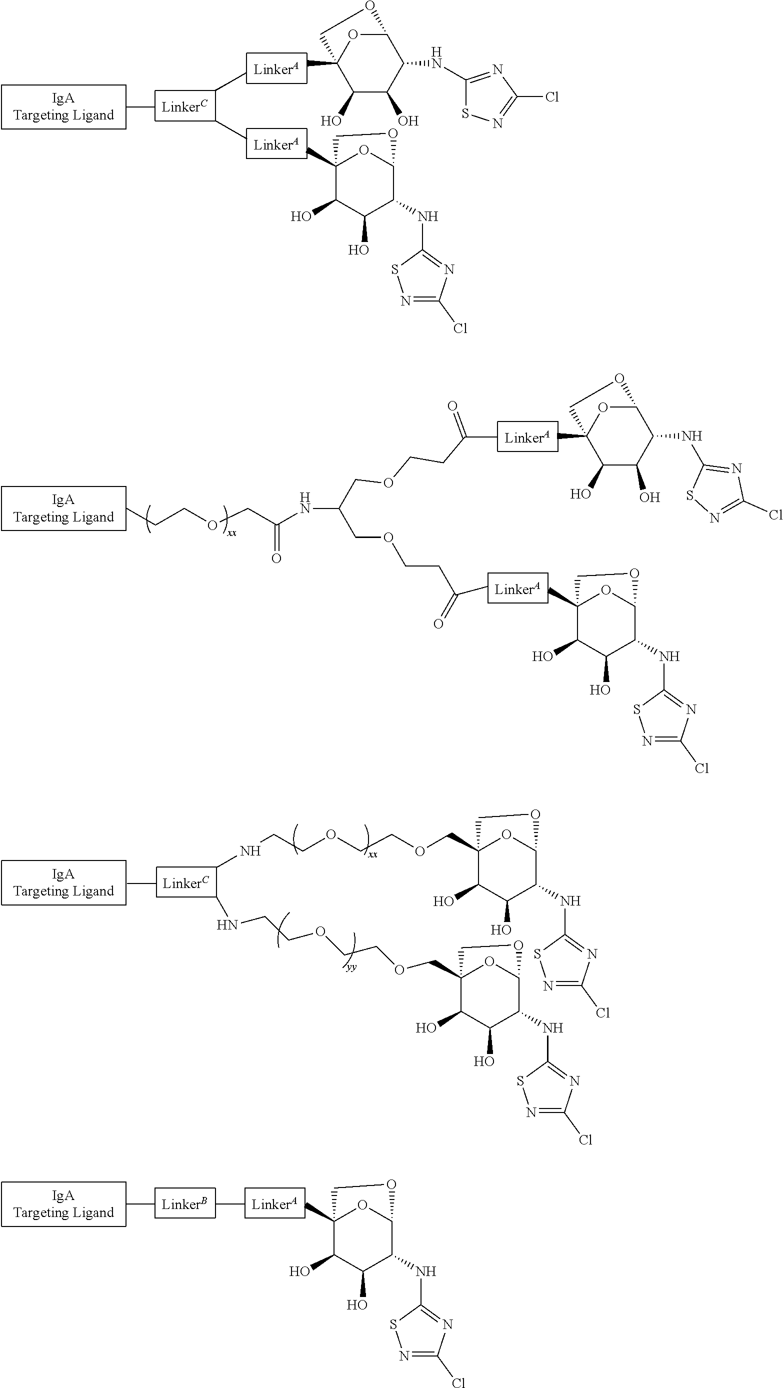

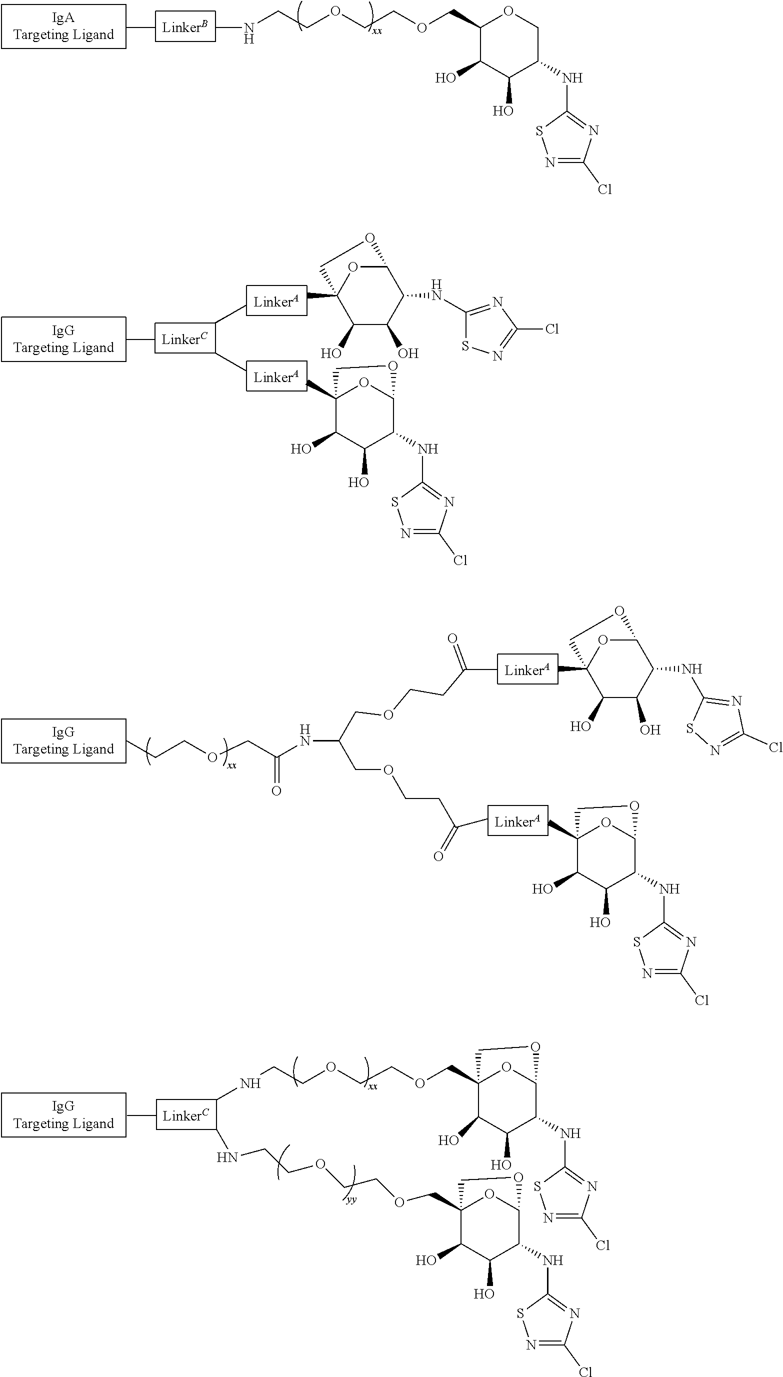

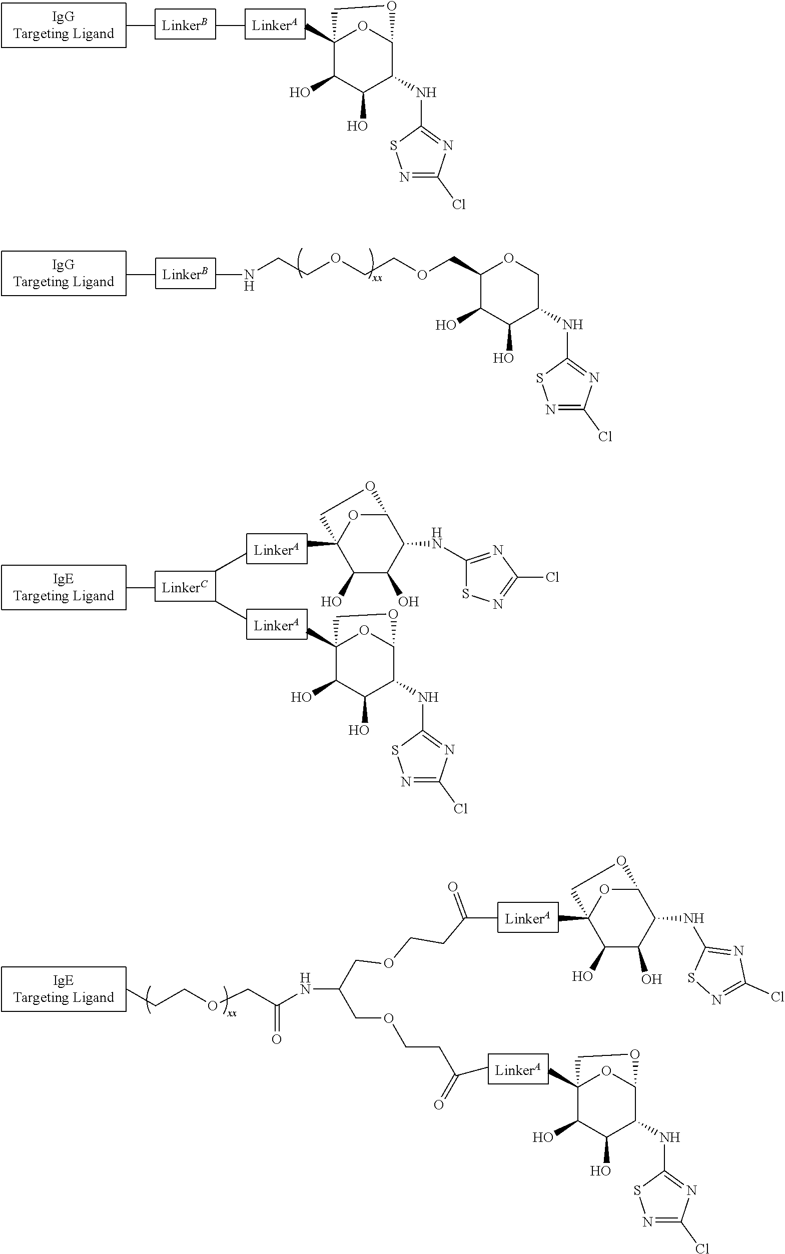

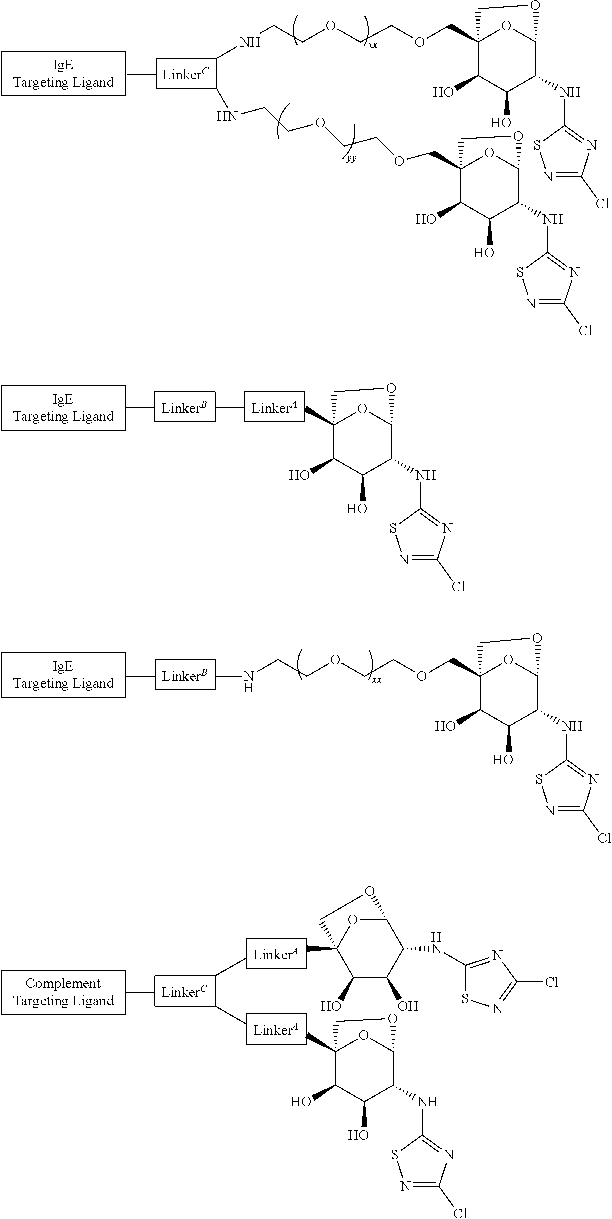

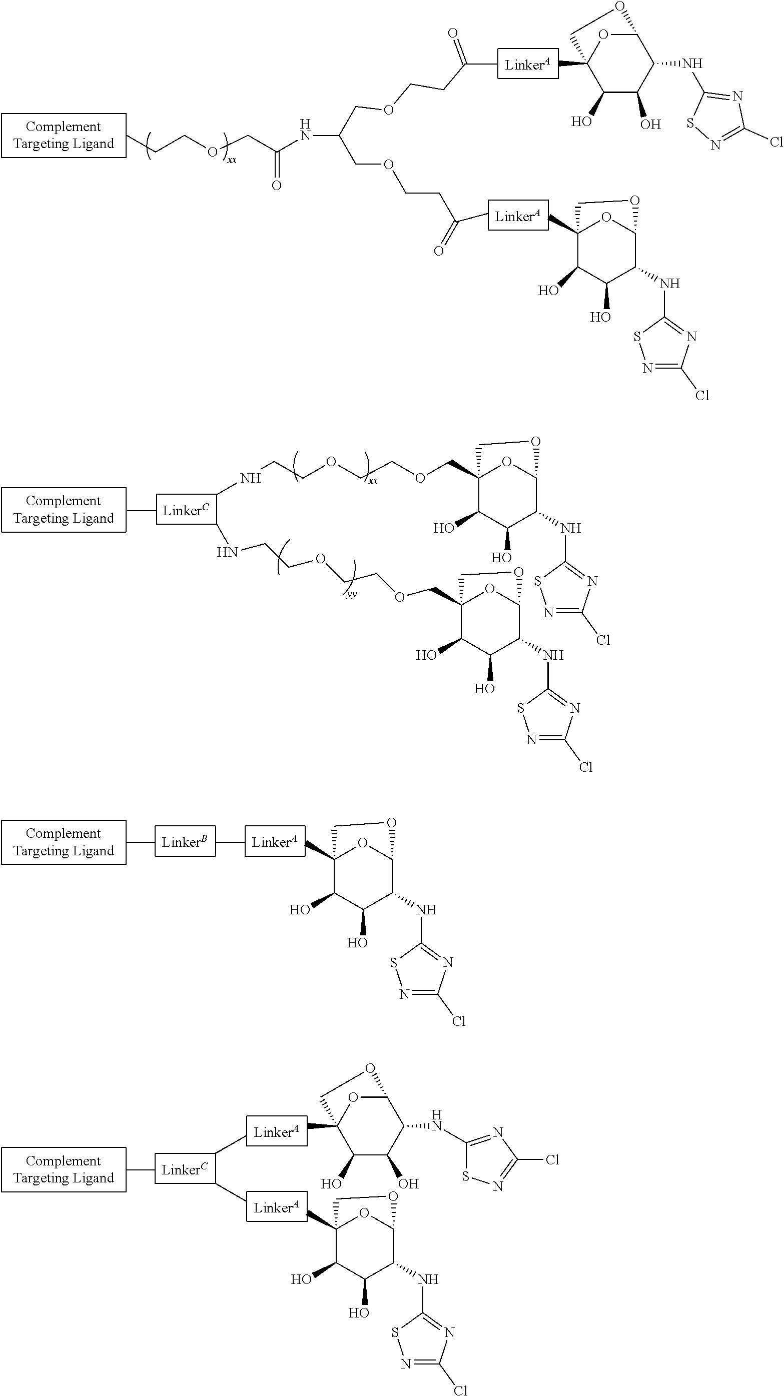

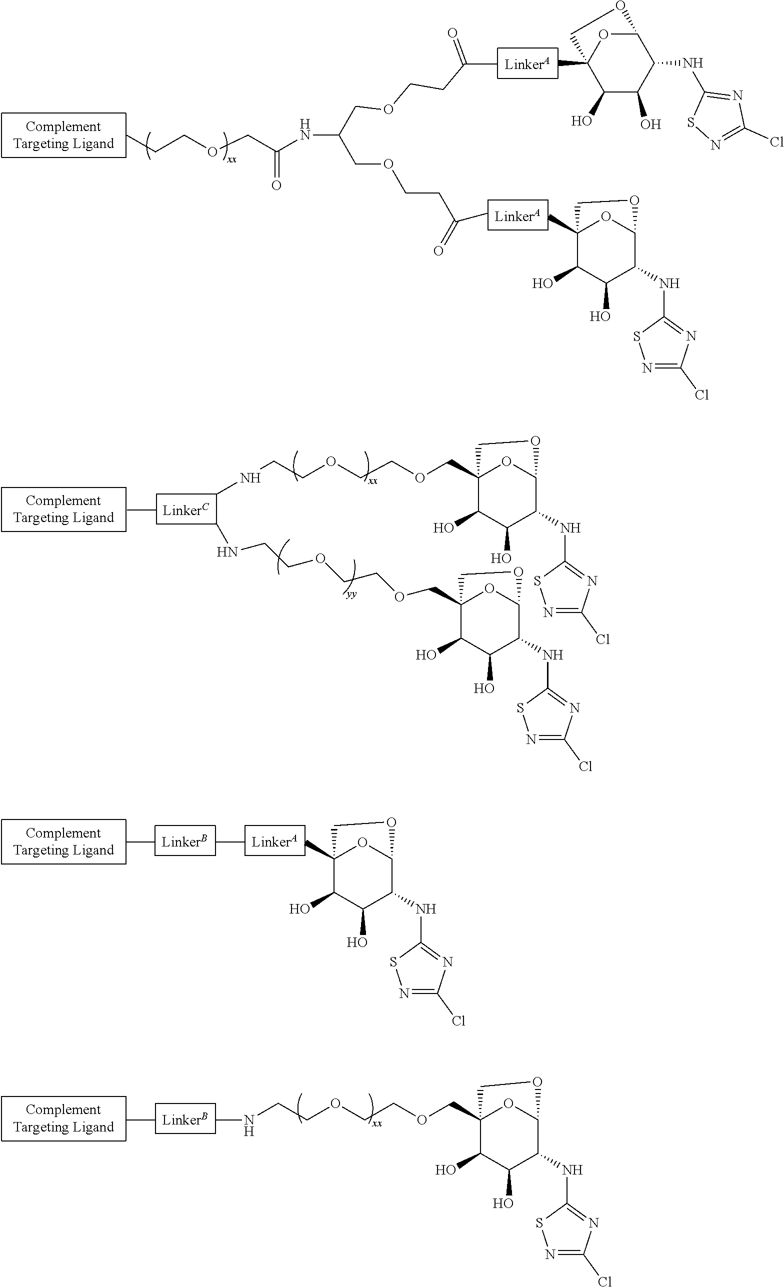

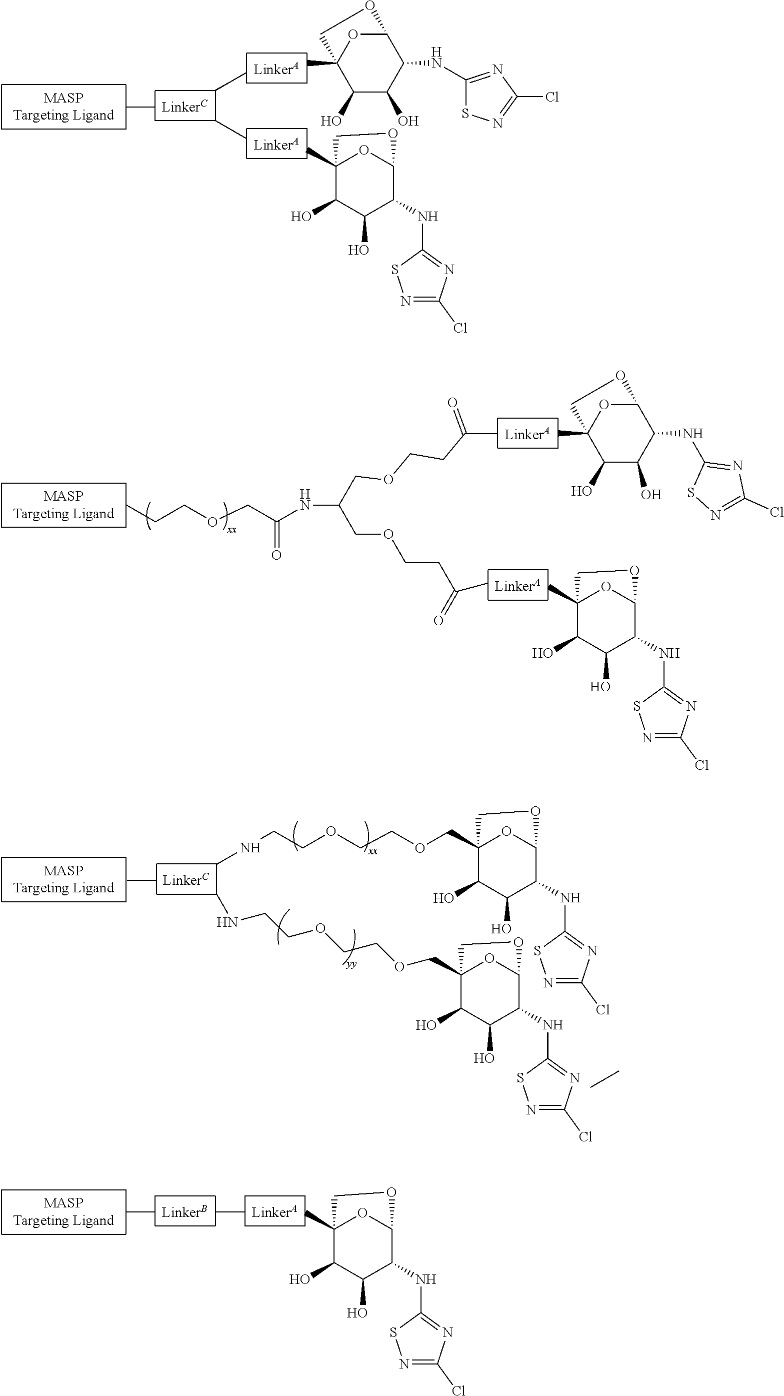

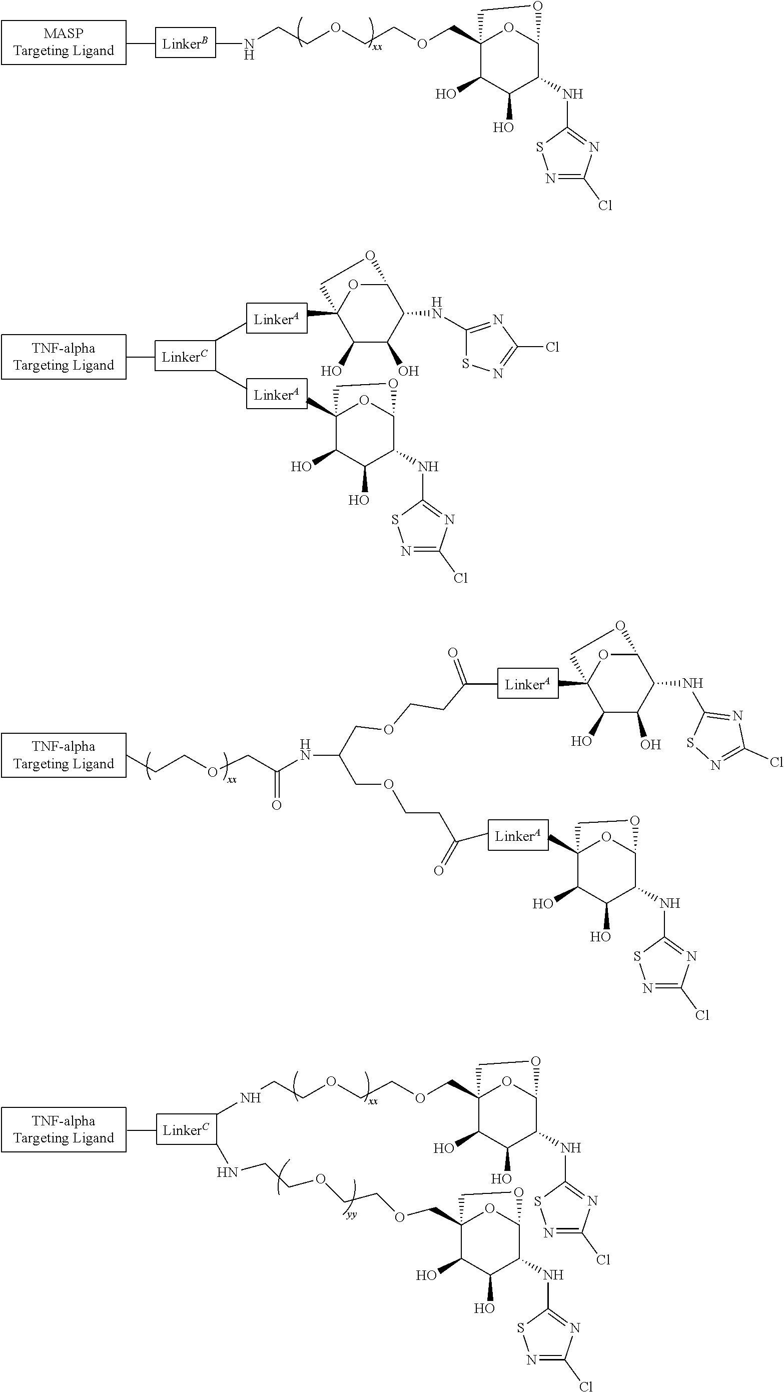

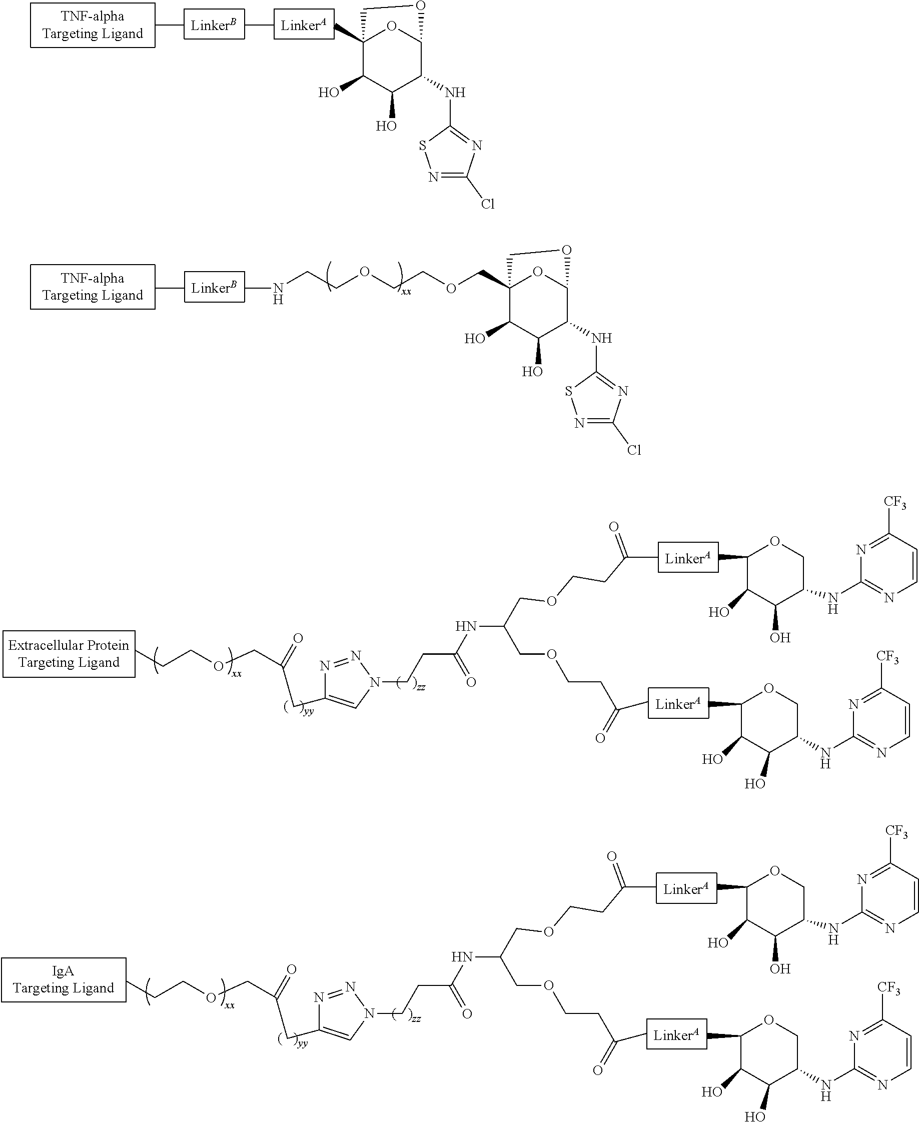

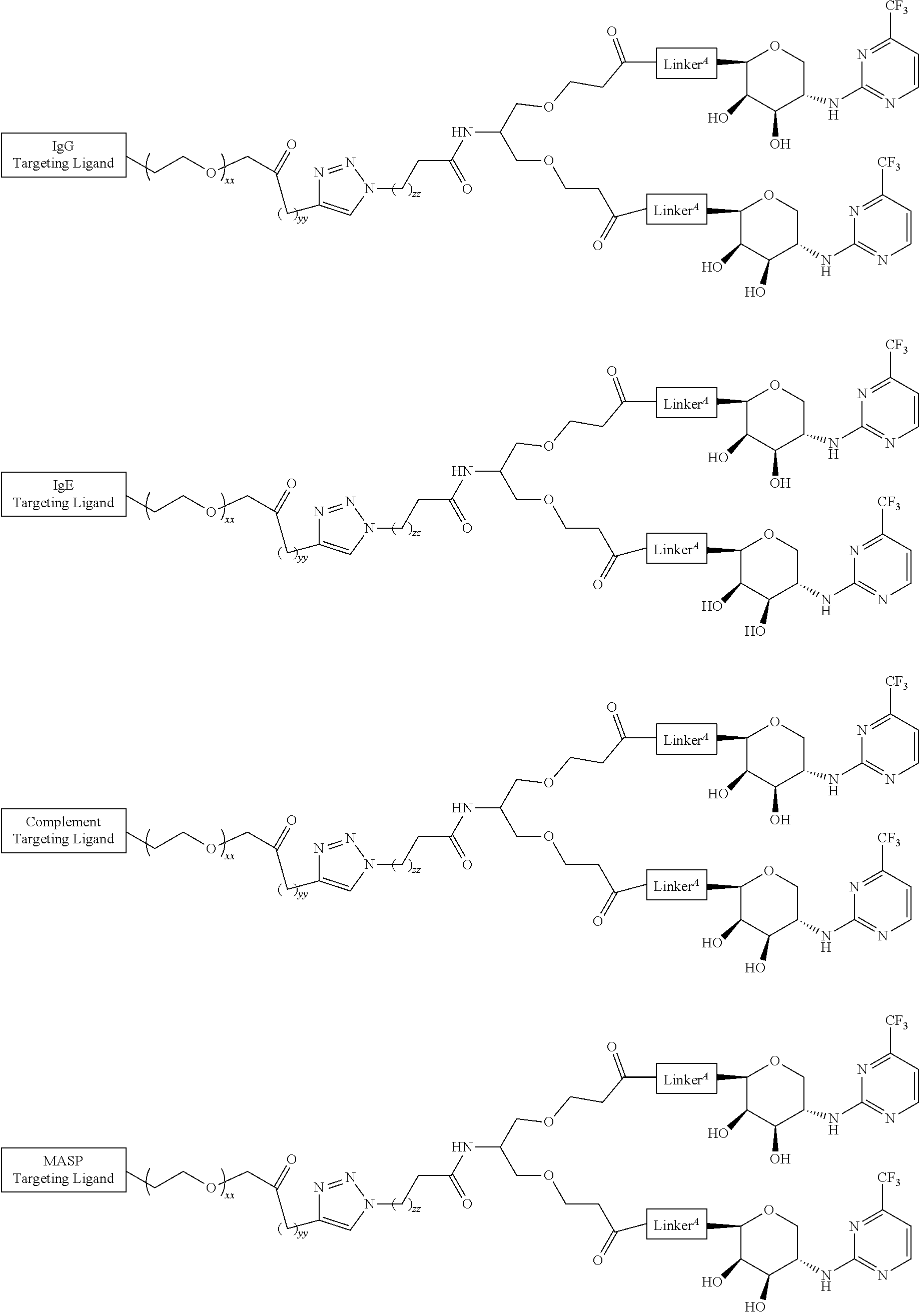

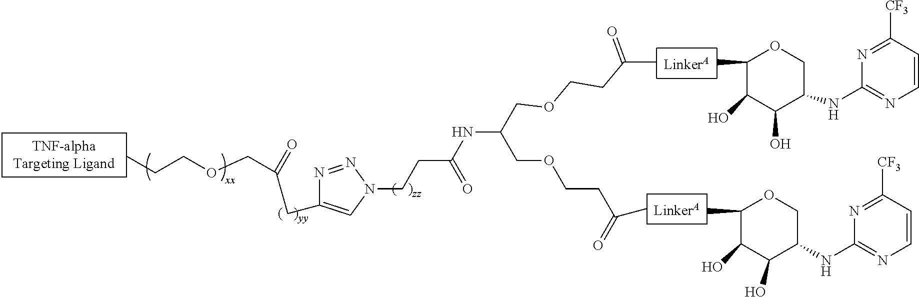

In certain embodiments, an ASGPR ligand useful for incorporation into a compound of Formula II is selected from:

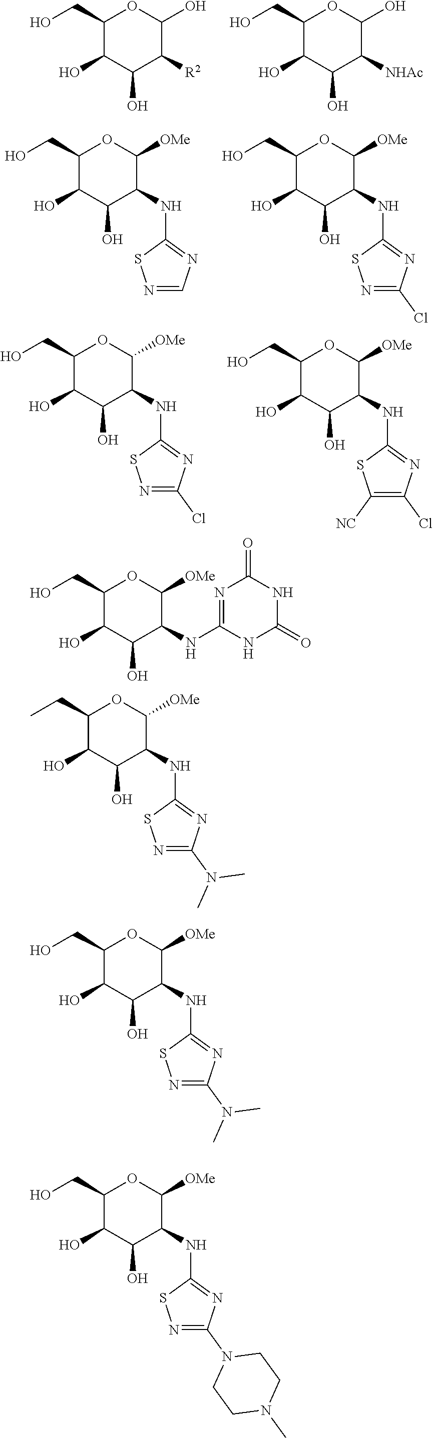

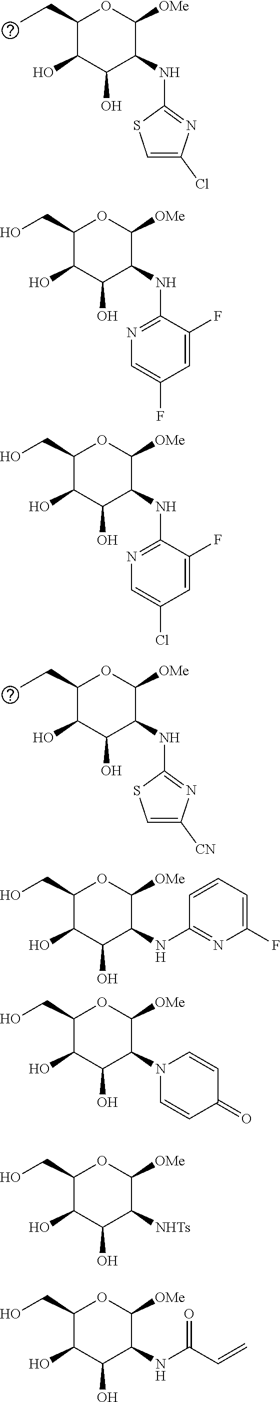

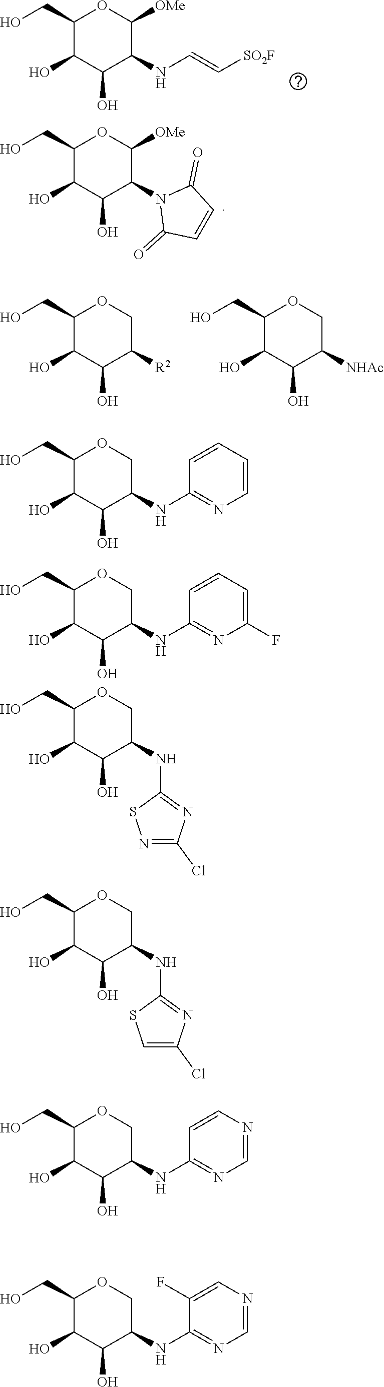

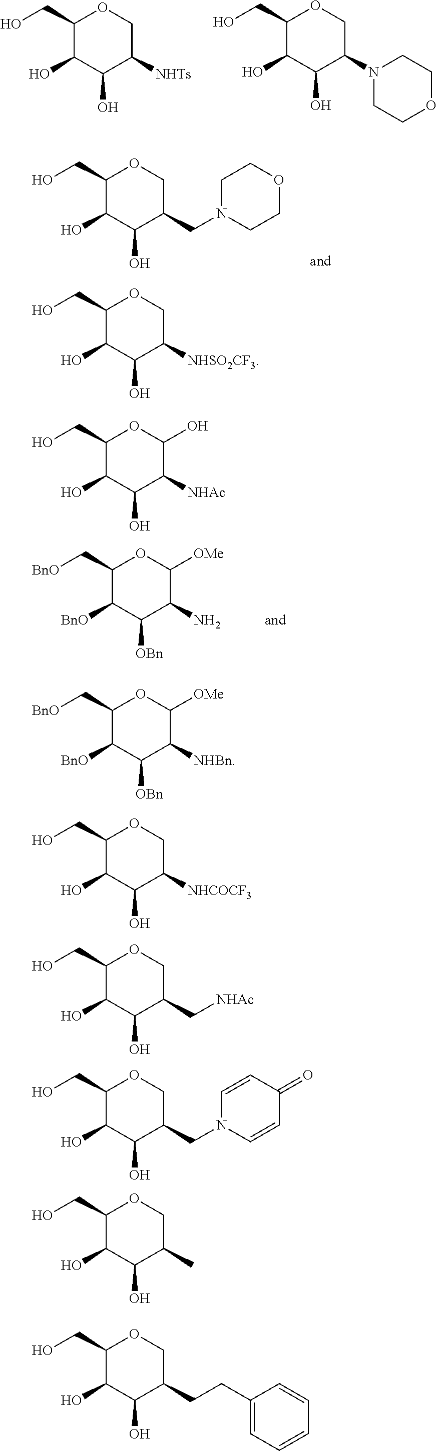

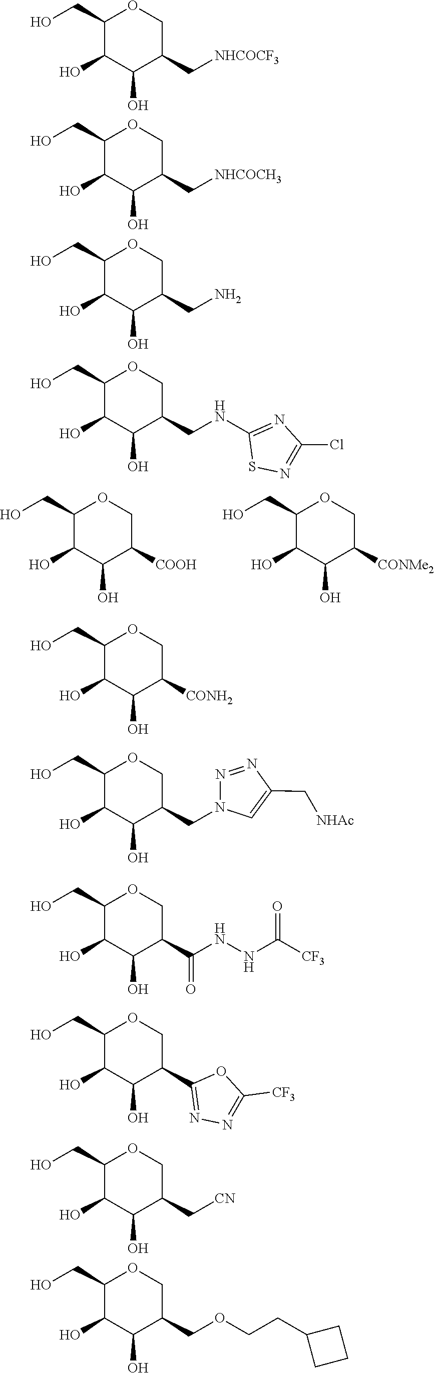

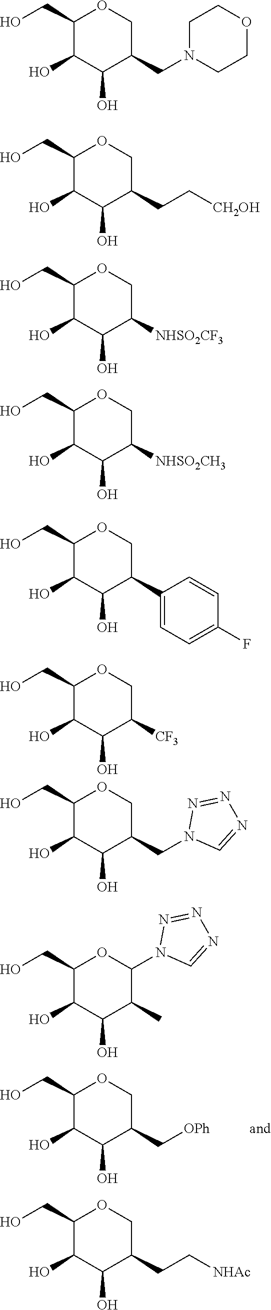

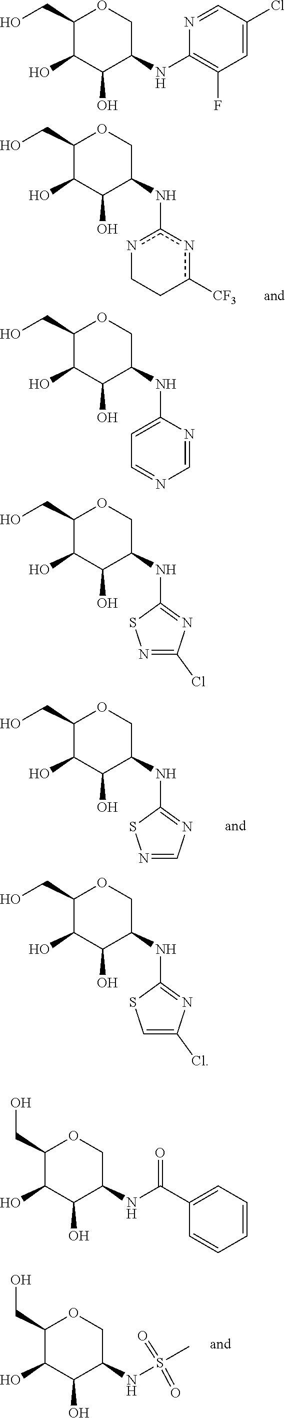

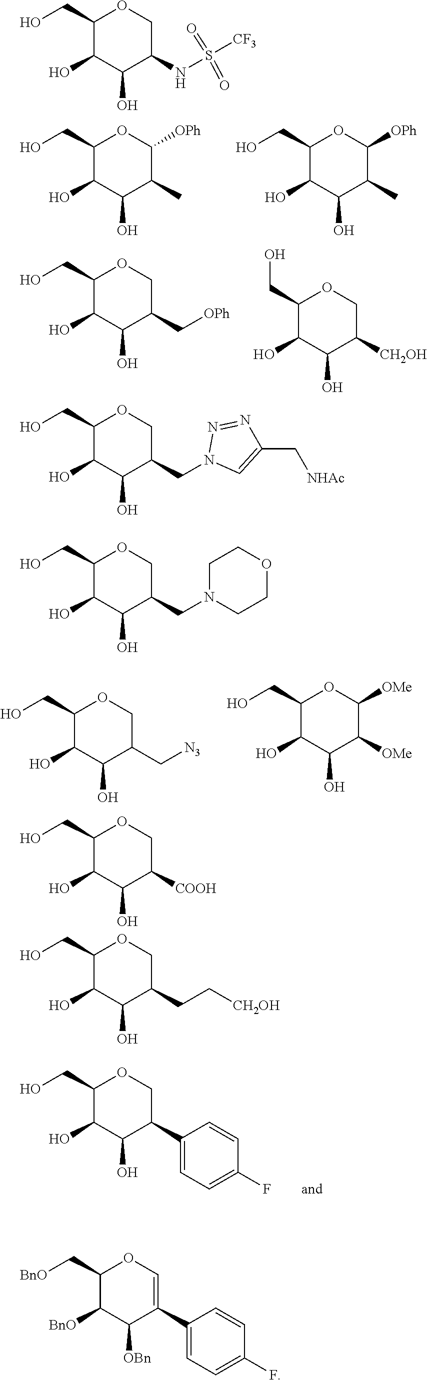

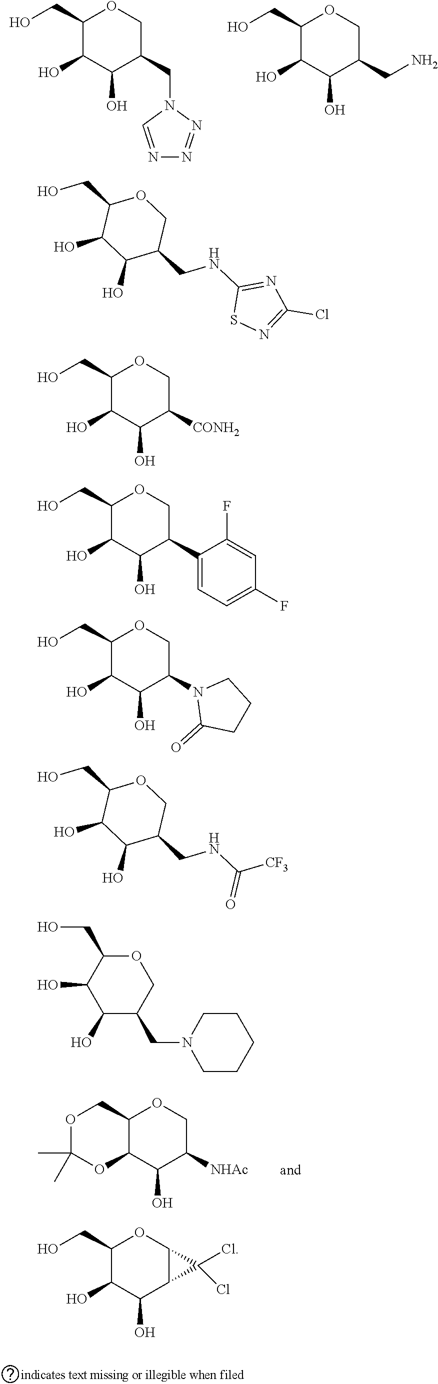

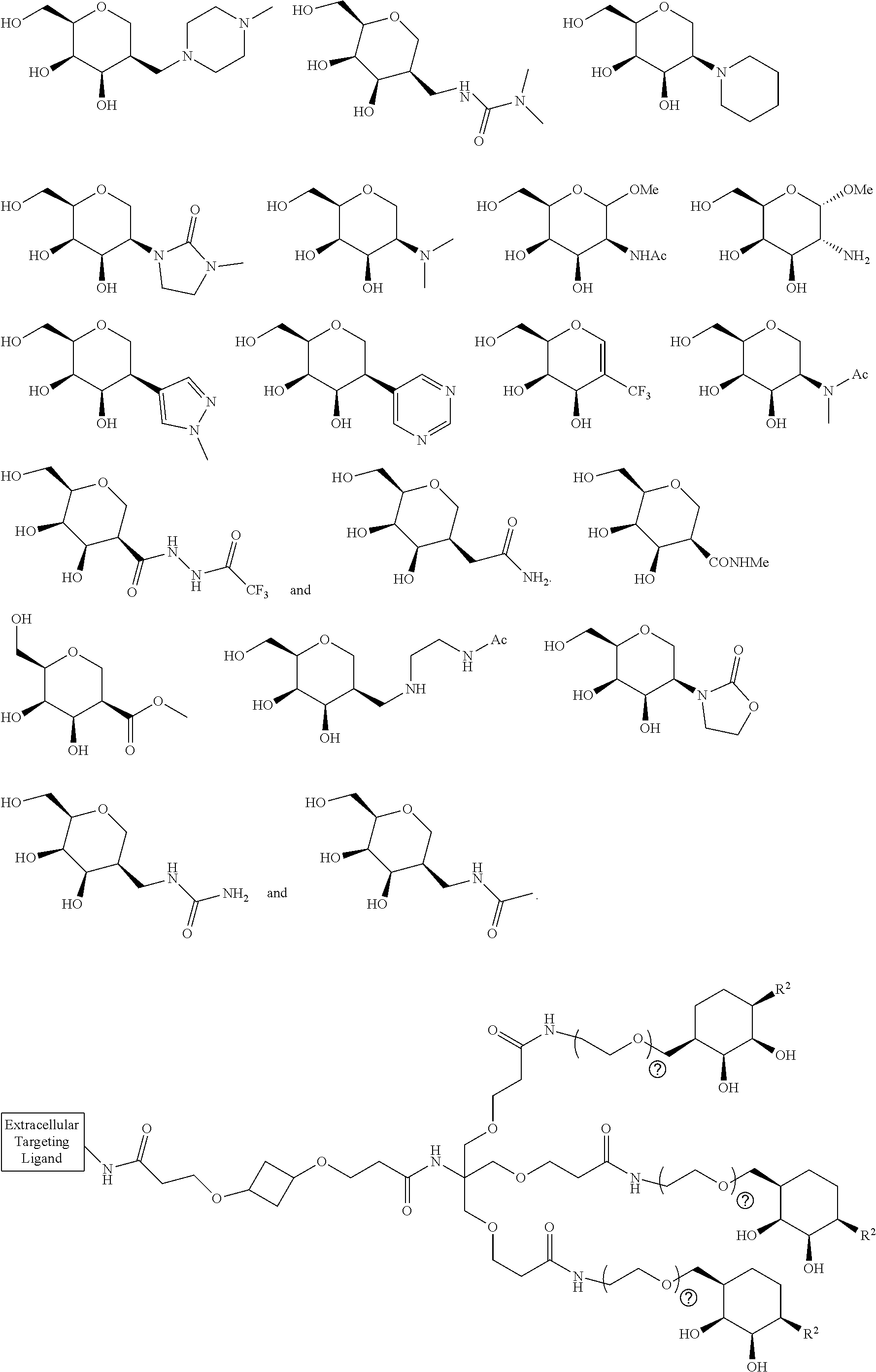

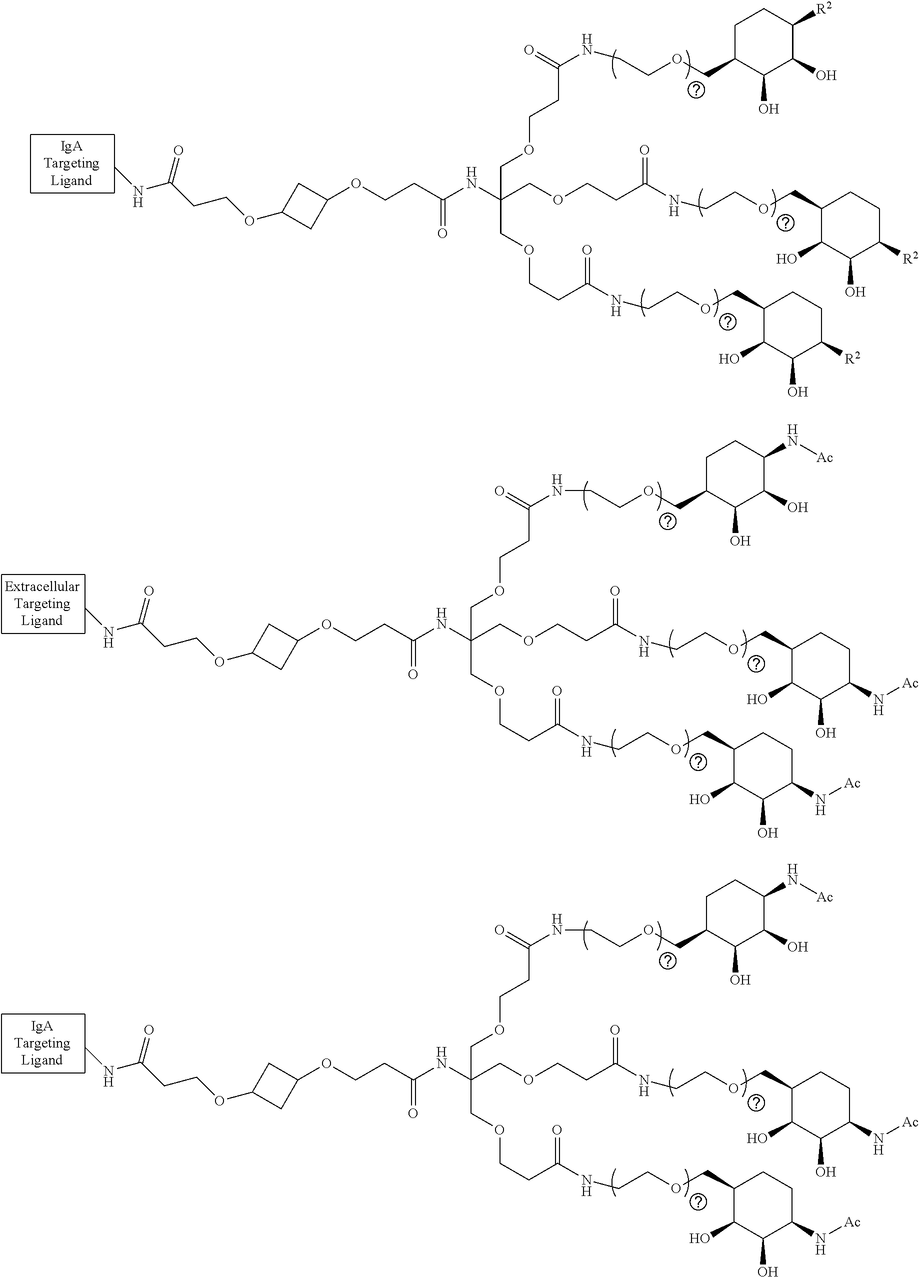

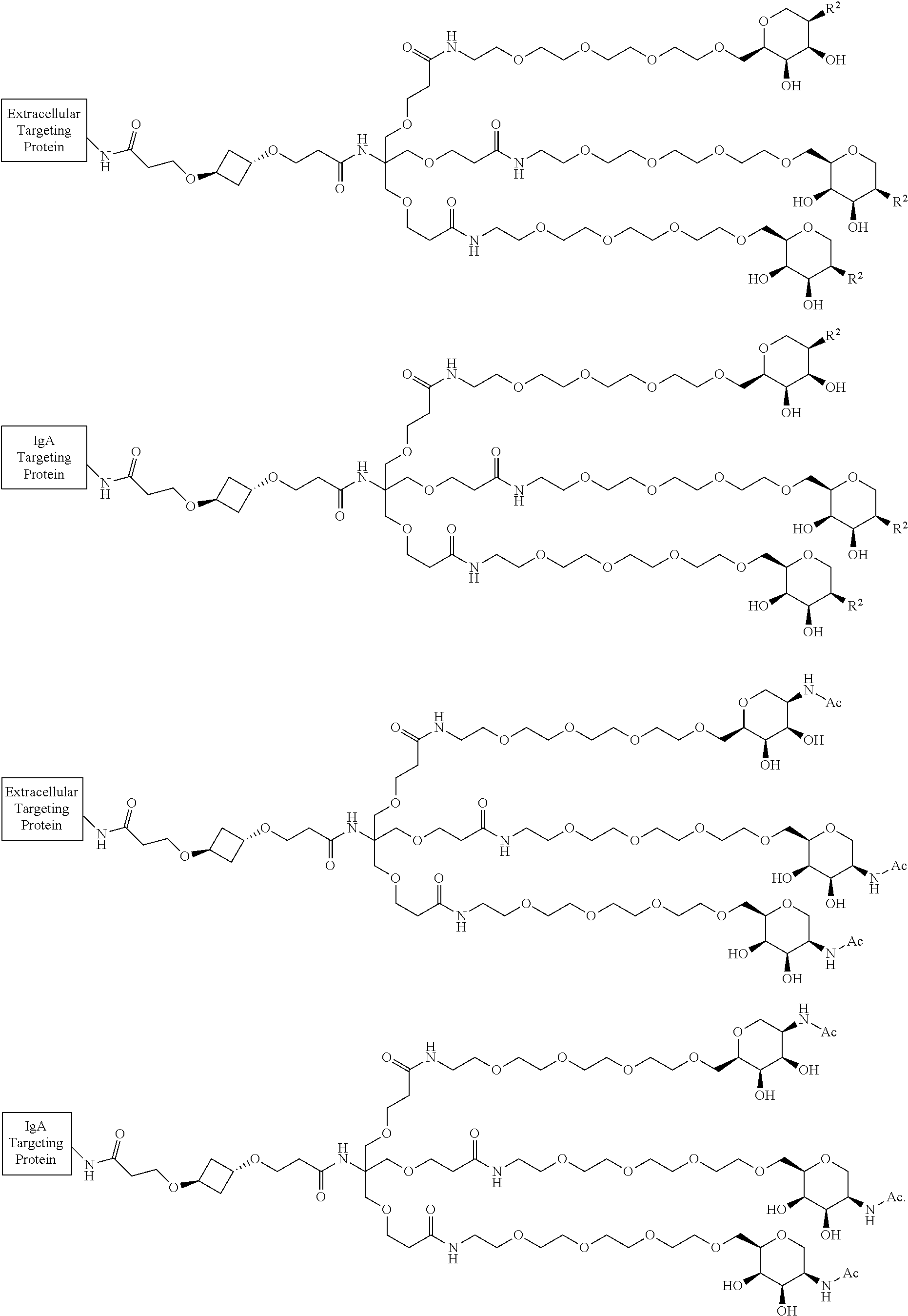

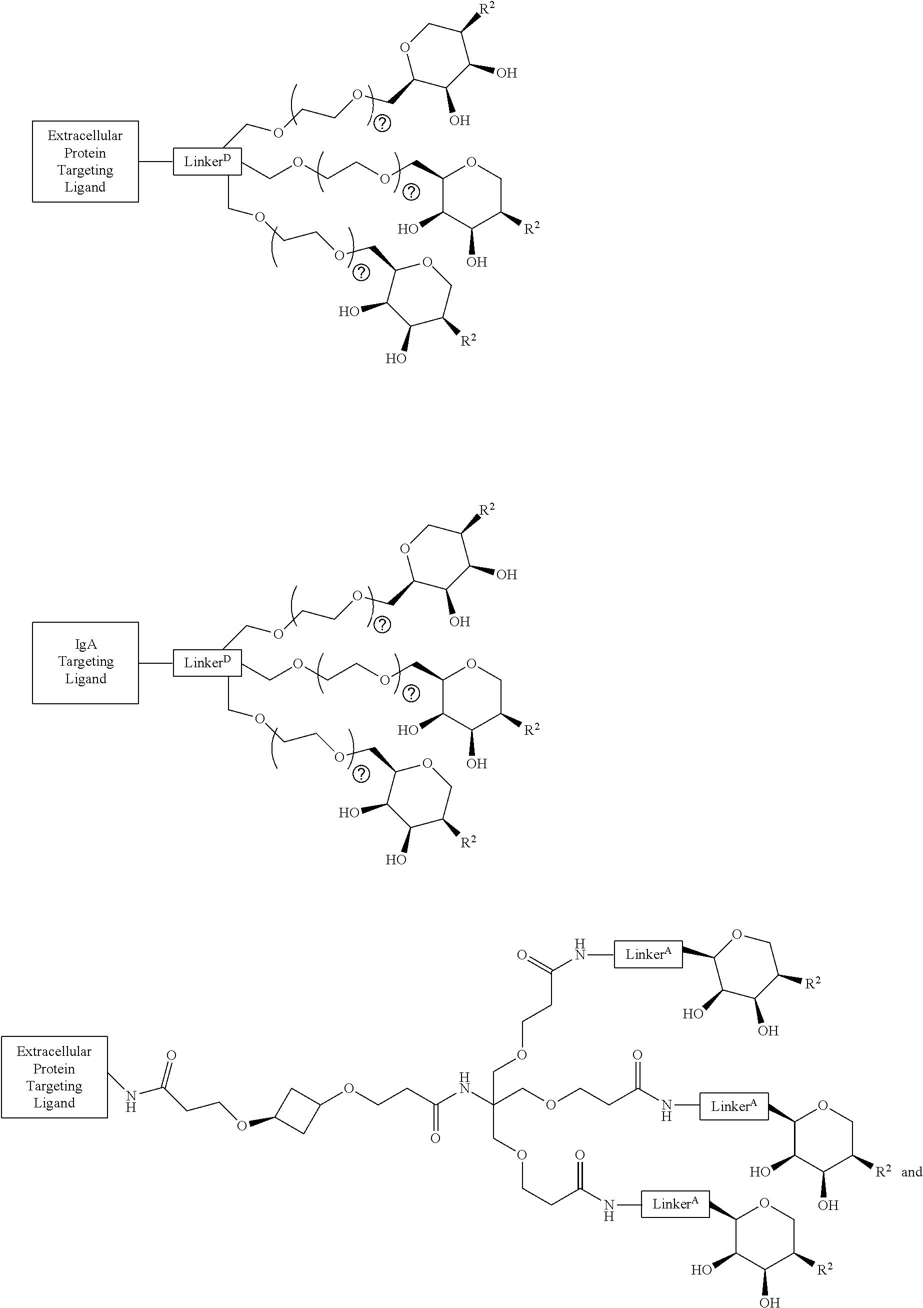

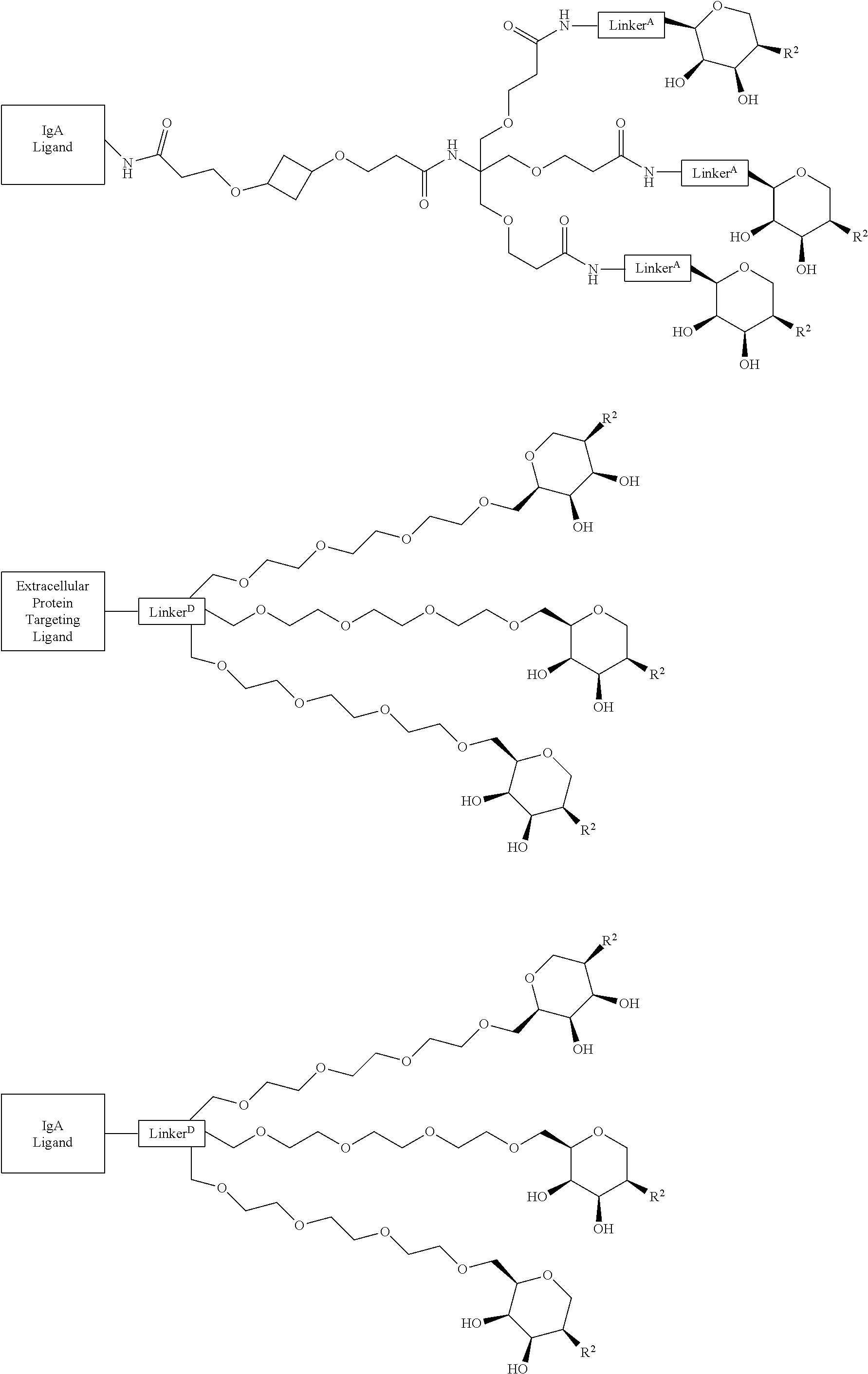

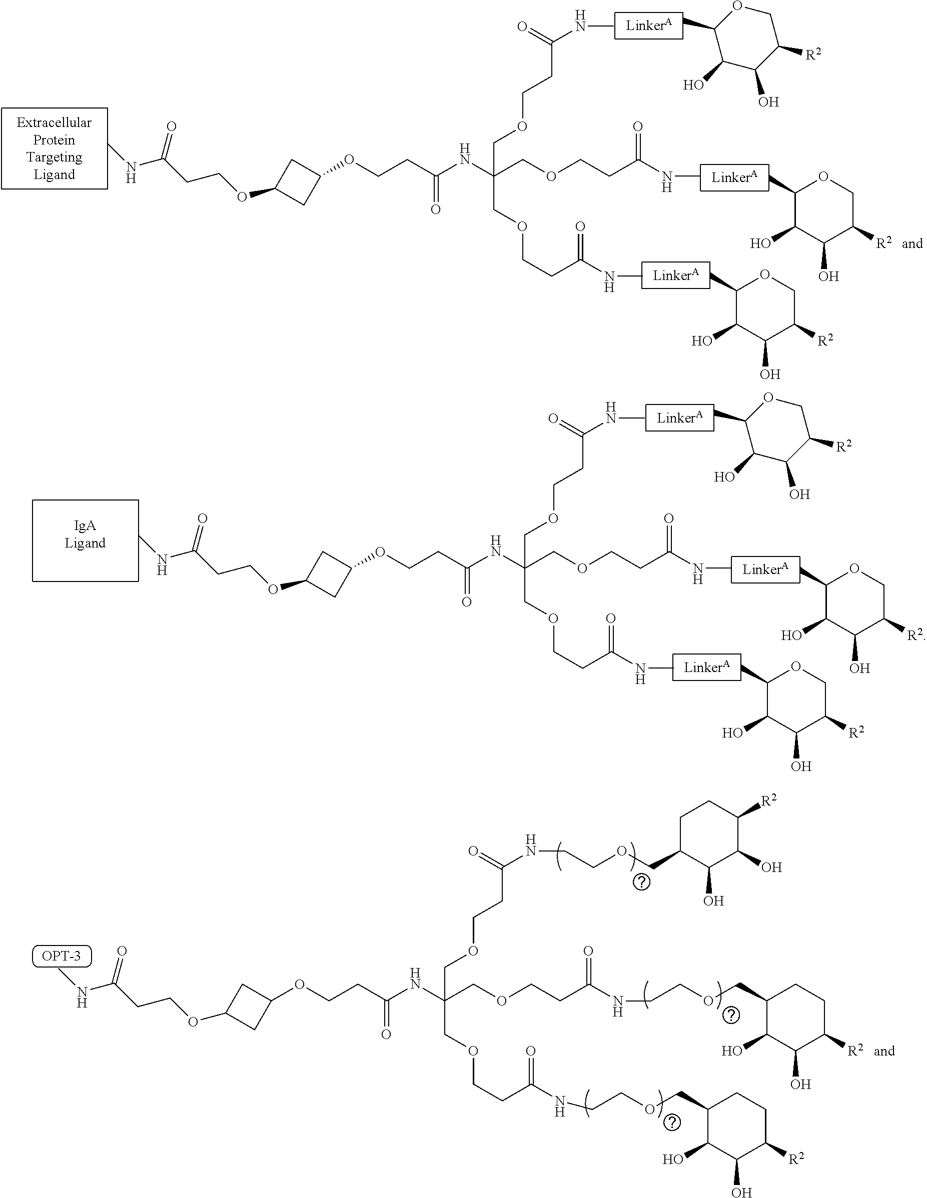

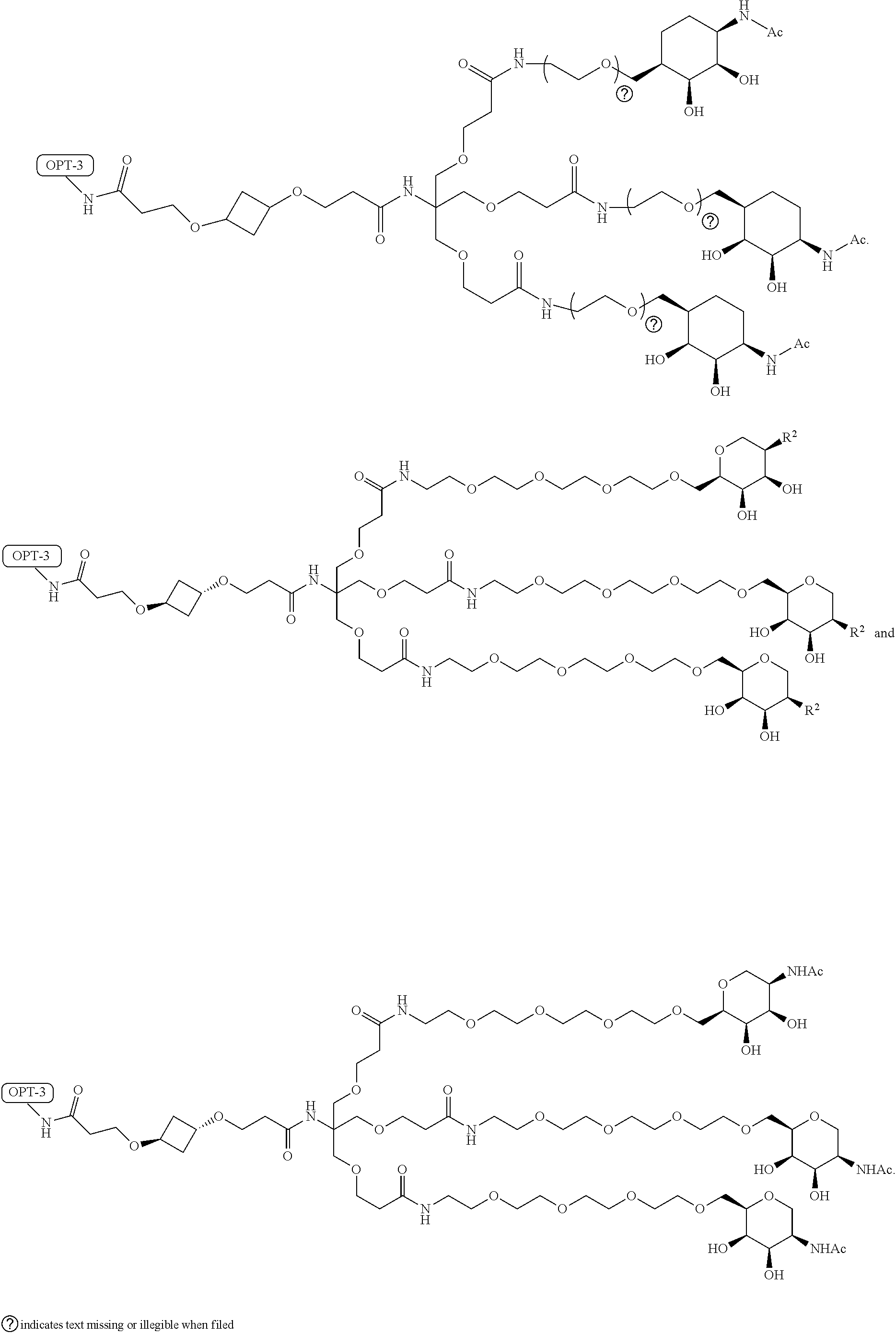

C. The ASGPR Ligand/Binding Moiety in Compounds of Formula II

In certain embodiments, in the compound of Formula II, R¹ is hydrogen.

In certain embodiments, in the compound of Formula II, R¹ is

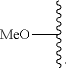 [SMILES: *OC]

In certain embodiments, in the compound of Formula II, R¹ is

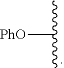 [SMILES: *Oc1ccccc1]

In certain embodiments, in the compound of Formula II, R¹ is

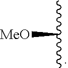

In certain embodiments, in the compound of Formula II, R¹ is

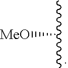

In certain embodiments, in the compound of Formula II, R¹ is

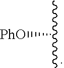

In certain embodiments, in the compound of Formula II, R¹ is

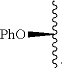

In certain embodiments, in the compound of Formula II, R¹ is C₀-C₆alkyl-cyano optionally substituted with 1, 2, 3, or 4 substituents.

In certain embodiments, in the compound of Formula II, R¹ is alkyl optionally substituted with 1, 2, 3, or 4 substituents.

In certain embodiments, in the compound of Formula II, R¹ is alkenyl optionally substituted with 1, 2, 3, or 4 substituents. In certain embodiments, in the compound of Formula II, R¹ is alkynyl optionally substituted with 1, 2, 3, or 4 substituents. In certain embodiments, in the compound of Formula II, R¹ is haloalkyl optionally substituted with 1, 2, 3, or 4 substituents. In certain embodiments, in the compound of Formula II, R¹ is F.

In certain embodiments, in the compound of Formula II, R¹ is Cl.

In certain embodiments, in the compound of Formula II, R¹ is Br.

In certain embodiments, in the compound of Formula II, R¹ is aryl optionally substituted with 1, 2, 3, or 4 substituents.

In certain embodiments, in the compound of Formula II, R¹ is arylalkyl optionally substituted with 1, 2, 3, or 4 substituents.

In certain embodiments, in the compound of Formula II, R¹ is heteroaryl optionally substituted with 1, 2, 3, or 4 substituents.

In certain embodiments, in the compound of Formula II, R¹ is heteroaryl alkyl optionally substituted with 1, 2, 3, or 4 substituents.

In certain embodiments, in the compound of Formula II, R¹ is heterocycle optionally substituted with 1, 2, 3, or 4 substituents.

In certain embodiments, in the compound of Formula II, R¹ is heterocycloalkyl optionally substituted with 1, 2, 3, or 4 substituents.

In certain embodiments, in the compound of Formula II, R¹ is haloalkoxy optionally substituted with 1, 2, 3, or 4 substituents.

In certain embodiments, in the compound of Formula II, R¹ is —O-alkenyl, —O-alkynyl, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R⁸)—C(S)R³, C₀-C₆alkyl-N(R⁸)—S(O)₂R³ C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, or C₀-C₆alkyl-O—S(O)₂R³, each of which is optionally substituted with 1, 2, 3, or 4 substituents.

In certain embodiments, in the compound of Formula II, R² is aryl optionally substituted with 1, 2, 3, or 4 substituents.

In certain embodiments, in the compound of Formula II, R² is heterocycle optionally substituted with 1, 2, 3, or 4 substituents.

In certain embodiments, in the compound of Formula II, R² is heteroaryl containing 1 or 2 heteroatoms independently selected from N, O, and S optionally substituted with 1, 2, 3, or 4 substituents.

In certain embodiments, in the compound of Formula II, R² is selected from

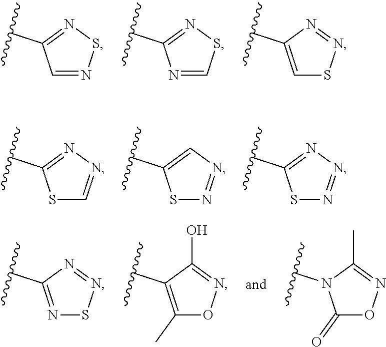

In certain embodiments, in the compound of Formula II, R² is heterocycle optionally substituted with 1, 2, 3, or 4 substituents.

In certain embodiments, in the compound of Formula II, R² is —NR⁸—S(O)—R³ optionally substituted with 1, 2, 3, or 4 substituents.

In certain embodiments, in the compound of Formula II, R² is —NR⁸—C(S)—R³ optionally substituted with 1, 2, 3, or 4 substituents.

In certain embodiments, in the compound of Formula II, R² is —NR⁸—S(O)(NR⁶)—R³ optionally substituted with 1, 2, 3, or 4 substituents.

In certain embodiments, in the compound of Formula II, R² is —N═S(O)(R³)₂ optionally substituted with 1, 2, 3, or 4 substituents.

In certain embodiments, in the compound of Formula II, R² is —NR⁸C(O)NR⁹S(O)₂R³ optionally substituted with 1, 2, 3, or 4 substituents.

In certain embodiments, in the compound of Formula II, R² is —NR⁸—S(O)₂—R¹⁰ optionally substituted with 1, 2, 3, or 4 substituents.

In certain embodiments, in the compound of Formula II, R² is —NR⁸—C(NR⁶)—R³ optionally substituted with 1, 2, 3, or 4 substituents.

In certain embodiments, in the compound of Formula II, R² is hydrogen.

In certain embodiments, in the compound of Formula II, R² is R¹⁰, In certain embodiments, in the compound of Formula II, R² is alkyl-C(O)—R³.

In certain embodiments, in the compound of Formula II, R² is —C(O)—R³.

In certain embodiments, in the compound of Formula II, R² is alkyl.

In certain embodiments, in the compound of Formula II, R² is haloalkyl.

In certain embodiments, in the compound of Formula II, R² is —OC(O)R³.

In certain embodiments, in the compound of Formula II, R² is —NR⁸—C(O)R¹⁰.

In certain embodiments, in the compound of Formula II, R² is alkenyl optionally substituted with 1, 2, 3, or 4 substituents.

In certain embodiments, in the compound of Formula II, R² is allyl optionally substituted with 1, 2, 3, or 4 substituents.

In certain embodiments, in the compound of Formula II, R² is alkynyl optionally substituted with 1, 2, 3, or 4 substituents.

In certain embodiments, in the compound of Formula II, R² is —NR⁶-alkenyl optionally substituted with 1, 2, 3, or 4 substituents.

In certain embodiments, in the compound of Formula II, R² is —O-alkenyl optionally substituted with 1, 2, 3, or 4 substituents.

In certain embodiments, in the compound of Formula II, R² is —NR⁶-alkynyl optionally substituted with 1, 2, 3, or 4 substituents.

In certain embodiments, in the compound of Formula II, R² is —NR⁶-heteroaryl optionally substituted with 1, 2, 3, or 4 substituents.

In certain embodiments, in the compound of Formula II, R² is —NR⁶-aryl optionally substituted with 1, 2, 3, or 4 substituents.

In certain embodiments, in the compound of Formula II, R² is —O-heteroaryl optionally substituted with 1, 2, 3, or 4 substituents.

In certain embodiments, in the compound of Formula II, R² is —O-aryl optionally substituted with 1, 2, 3, or 4 substituents.

In certain embodiments, in the compound of Formula II, R² is —O-alkynyl optionally substituted with 1, 2, 3, or 4 substituents.

In certain embodiments, in the compound of Formula II, R² is selected from and

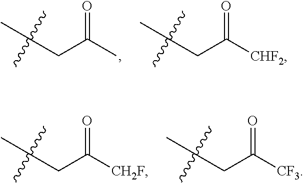

In certain embodiments, in the compound of Formula II, R² is selected from

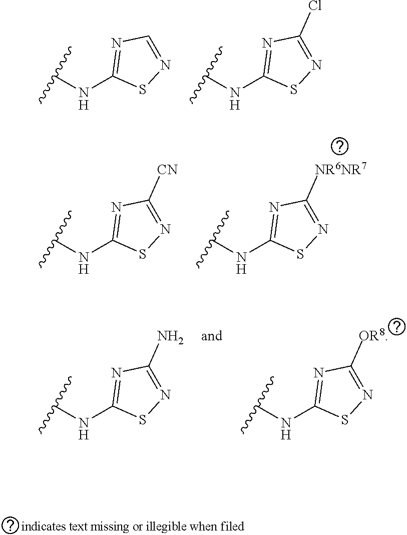

In certain embodiments, in the compound of Formula II, R² is selected from

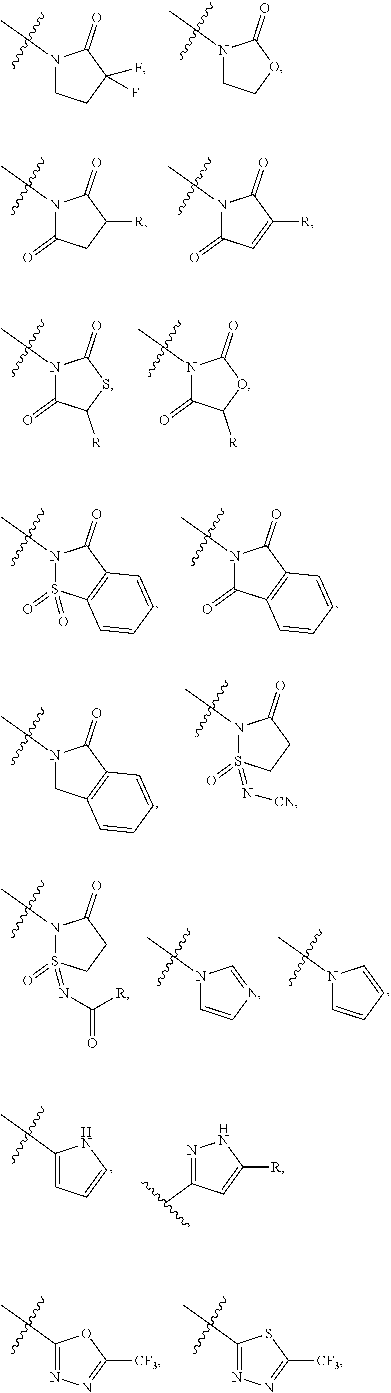

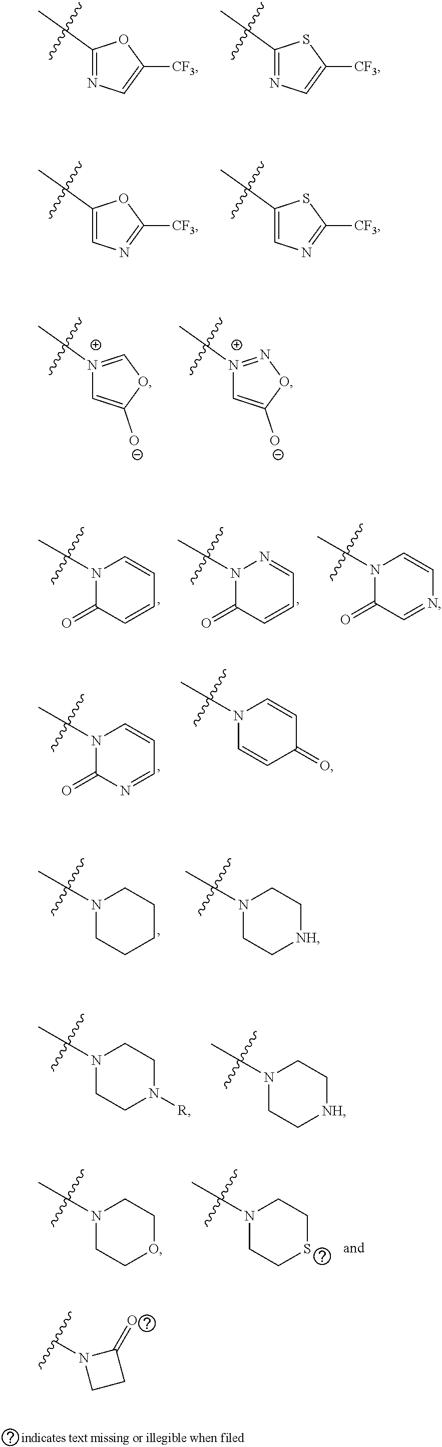

wherein R is an optional substituent as defined herein.

In certain embodiments, in the compound of Formula II, R² is selected from

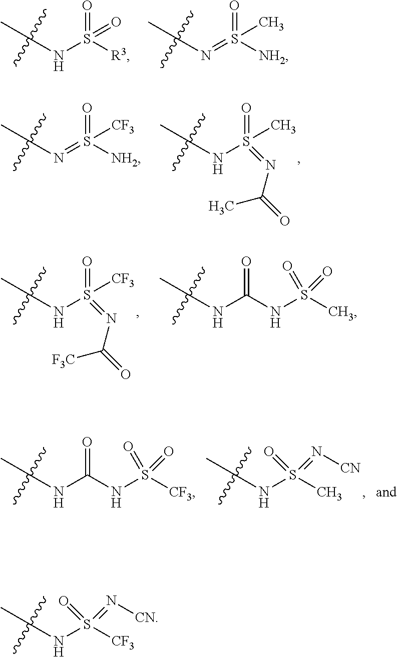

In certain embodiments, in the compound of Formula II, R^{2A} is selected from

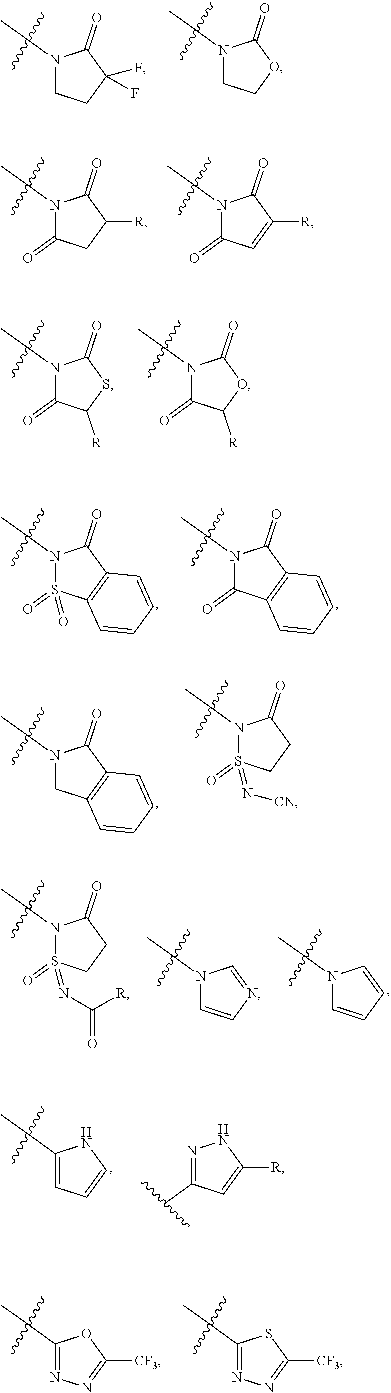

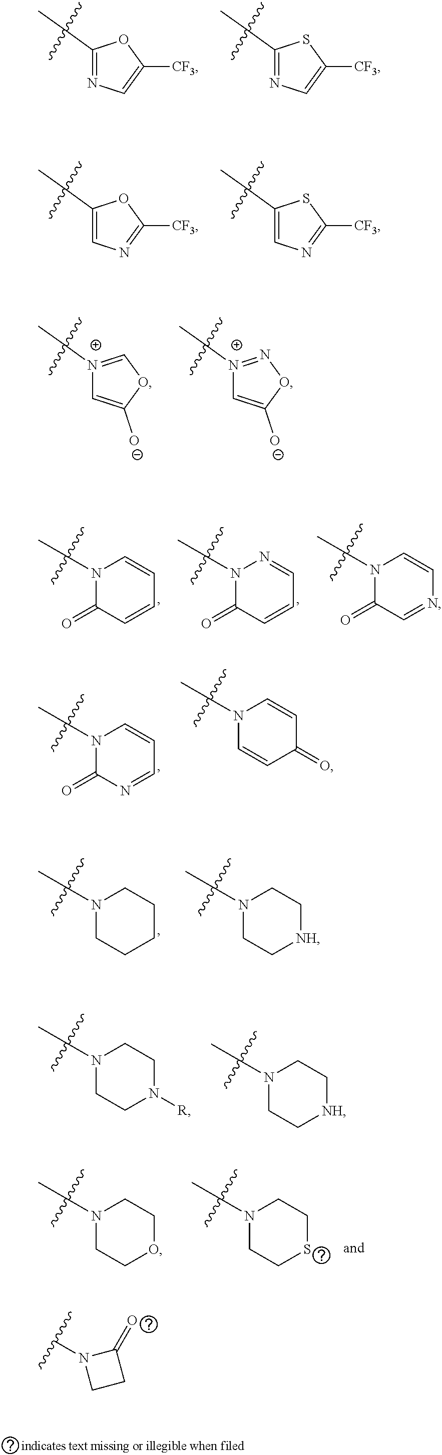

wherein R is an optional substituent as defined herein.

In certain embodiments, in the compound of Formula II, R^{2A} is selected from

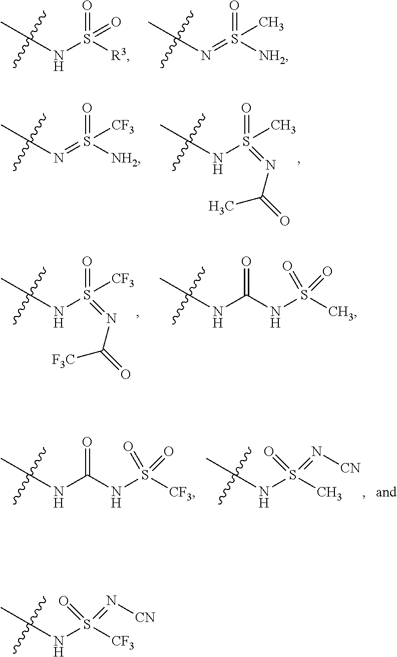

In certain embodiments in the compound of Formula II R² is selected from

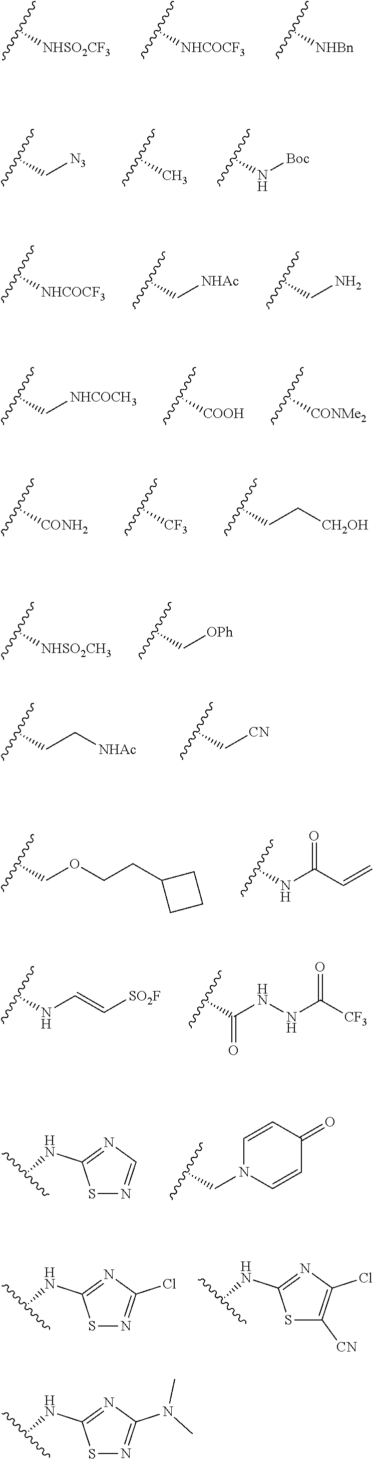

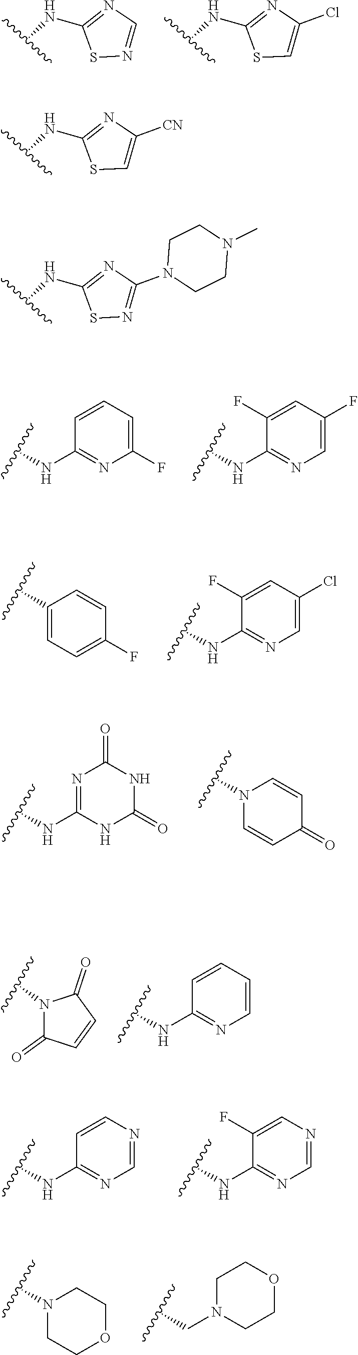

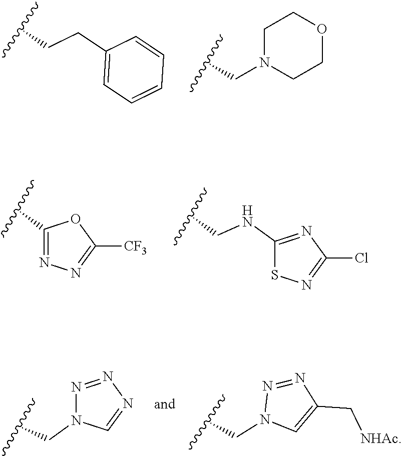

In certain embodiments, in the compound of Formula II, R² is selected from

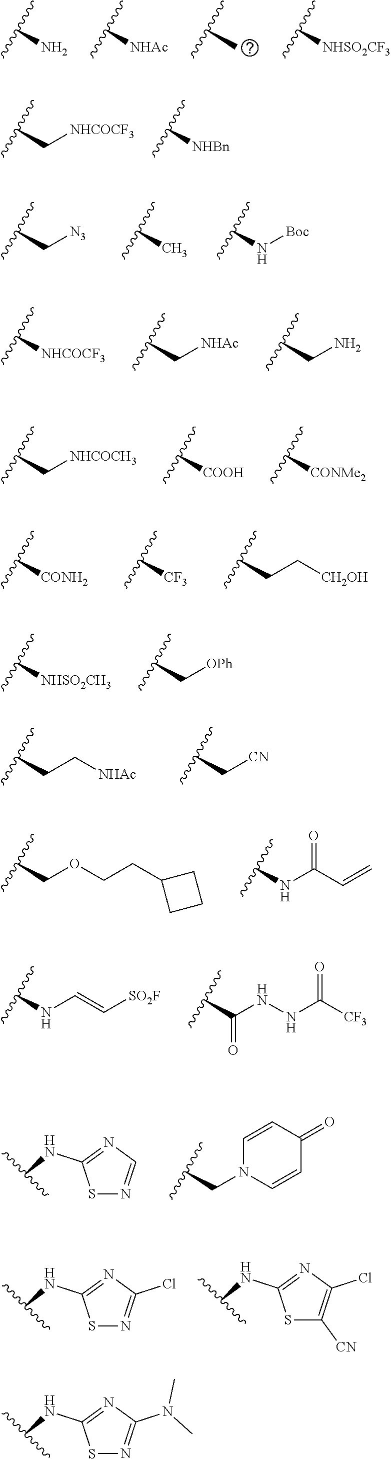

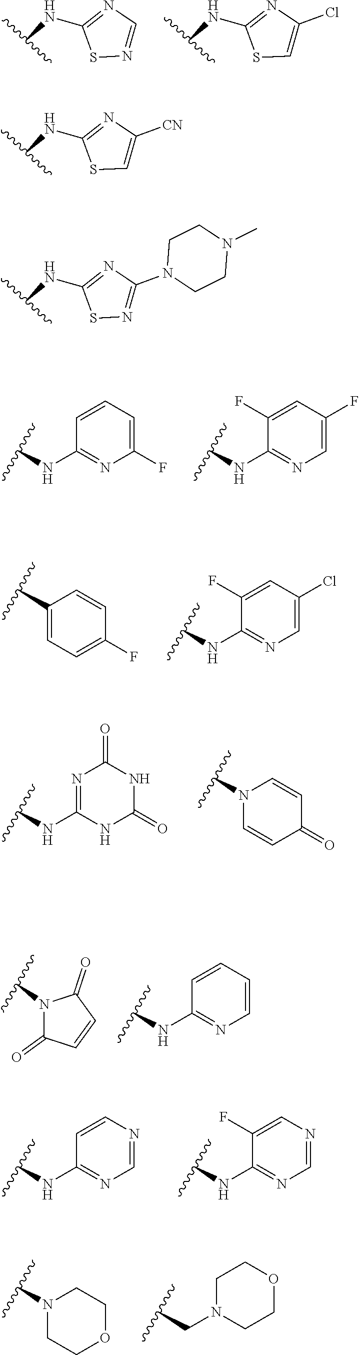

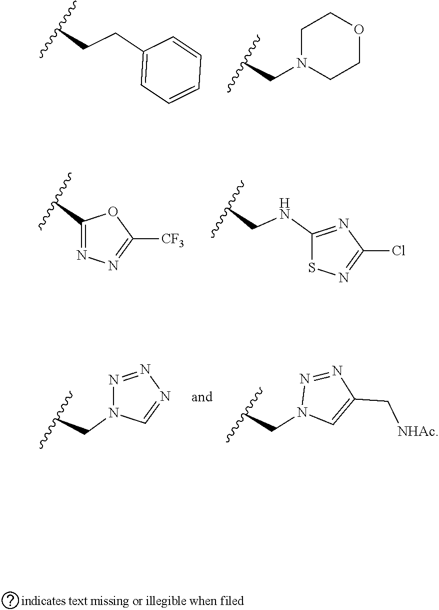

In certain embodiments, in the compound of Formula II, R² is selected from

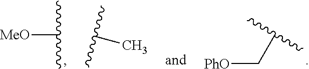

In certain embodiments in the compound of Formula II, R² is selected from

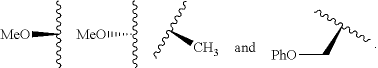

In certain embodiments, in the compound of Formula II, R² is selected from

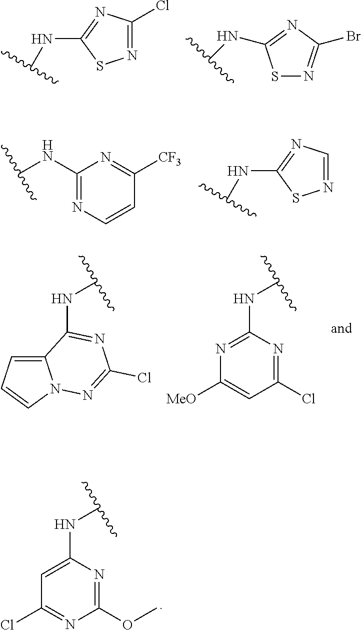

In certain embodiments, in the compound of Formula II, R² is selected from

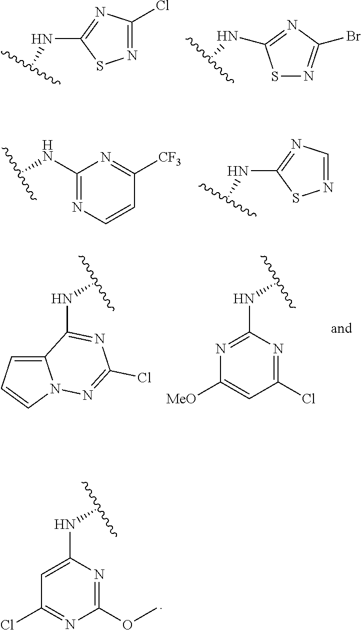

In certain embodiments in the compound of Formula II, R² is selected from

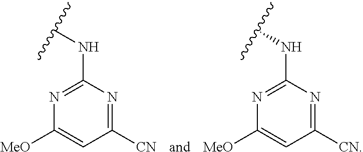

In certain embodiments in the compound of Formula II, R² is selected from

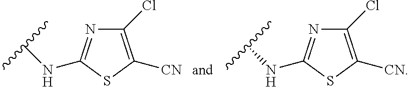

In certain embodiments, in the compound of Formula II, R² is selected from

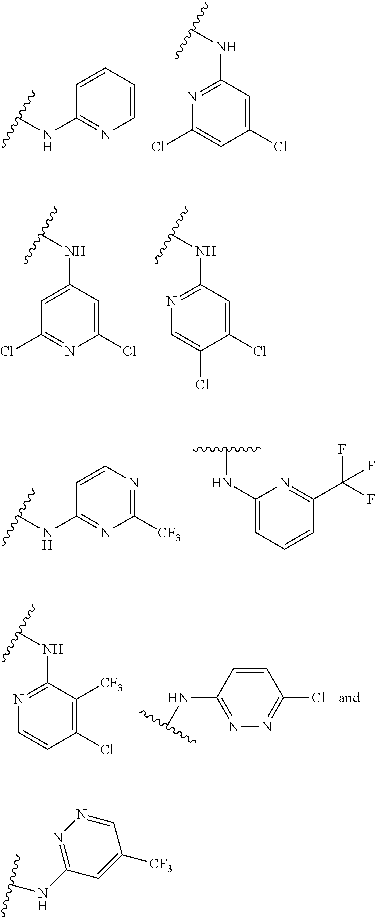

In certain embodiments, in the compound of Formula II, R² is selected from

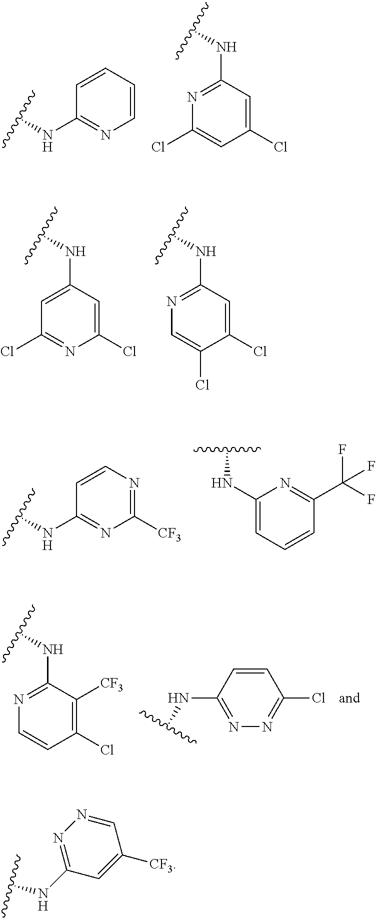

In certain embodiments in the compound of Formula II, R² is selected from

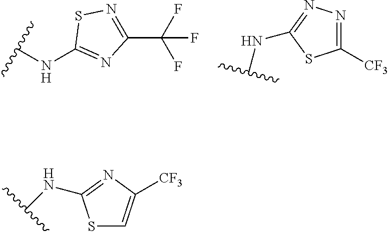 [SMILES: *Nc1nc(C(F)(F)F)ns1 ; *Nc1nnc(C(F)(F)F)s1 ; *Nc1nc(C(F)(F)F)cs1]

In certain embodiments, in the compound of Formula II, R² is selected from

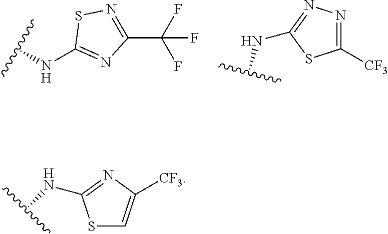

In certain embodiments, in the compound of Formula II, R² is selected from

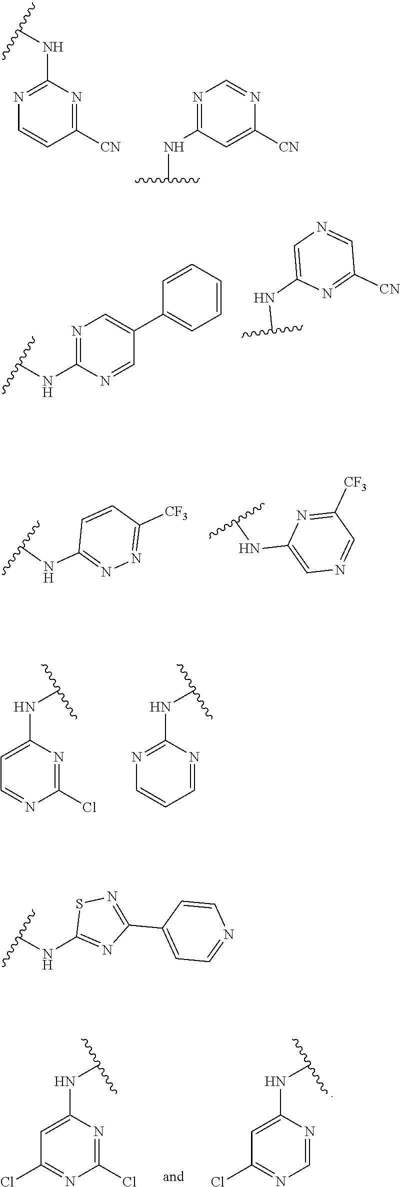

In certain embodiments in the compound of Formula II R² is selected from

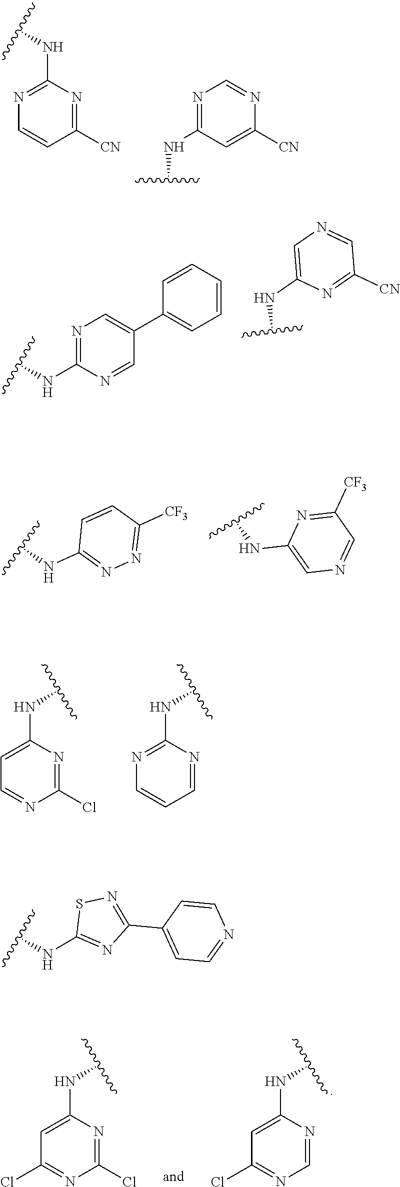

In certain embodiments, in the compound of Formula II, R² is selected from

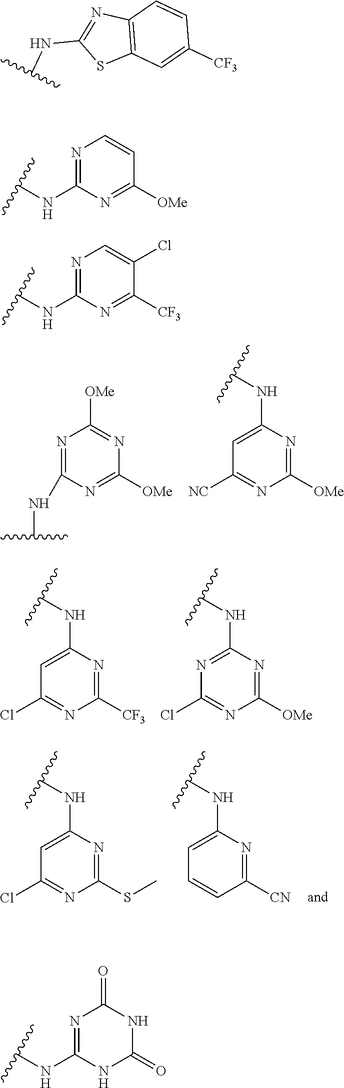

In certain embodiments, in the compound of Formula II, R² is selected from

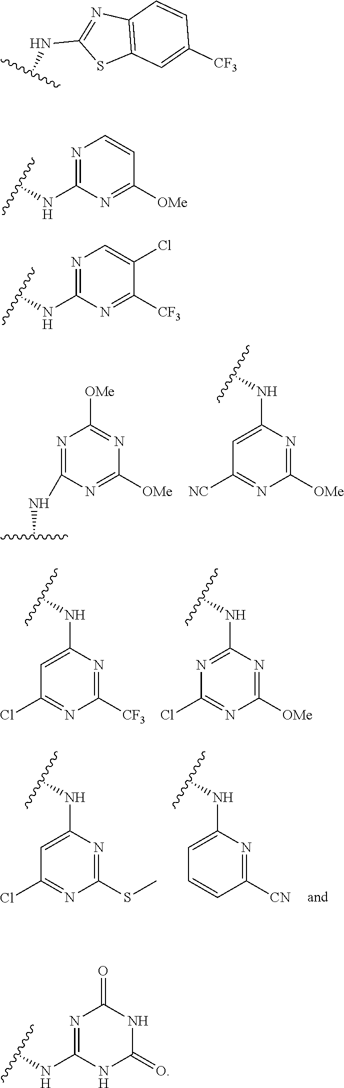

In certain embodiments, in the compound of Formula II, R² is selected from

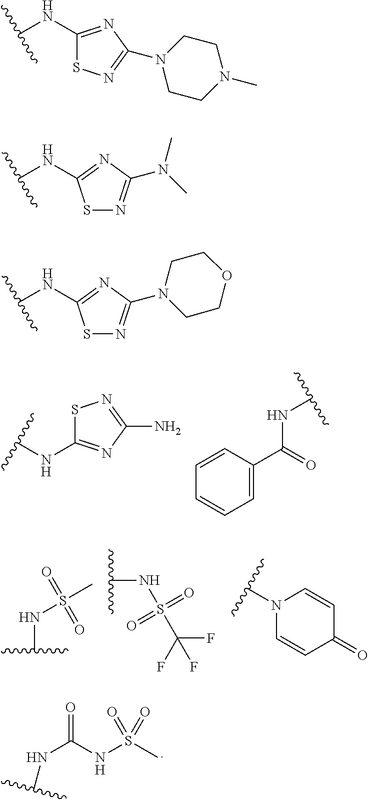

In certain embodiments, in the compound of Formula II, R² is selected from

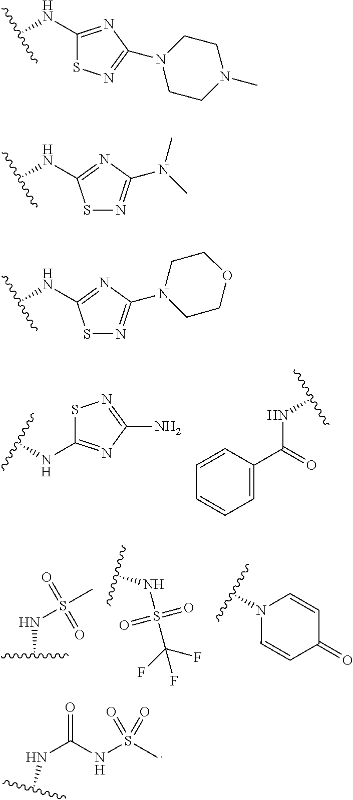

In certain embodiments, in the compound of Formula II, R² is selected from

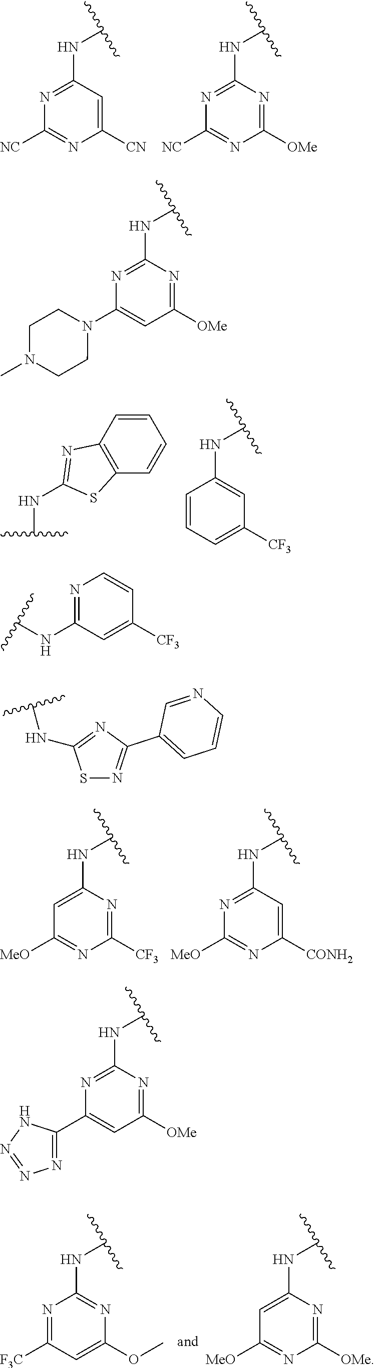

In certain embodiments, in the compound of Formula II, R² is selected from

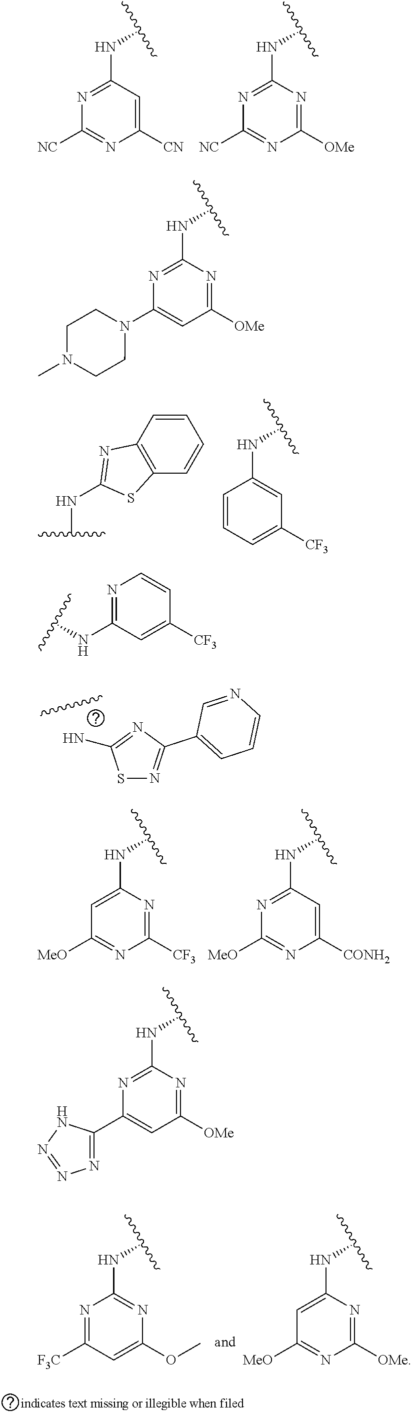

In certain embodiments, in the compound of Formula II, R² is selected from

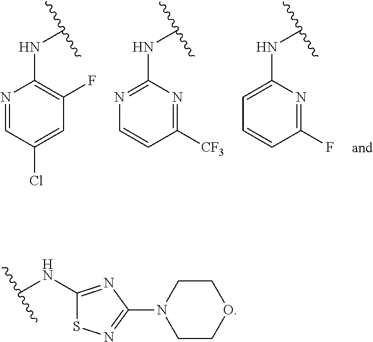

In certain embodiments in the compound of Formula II, R² is selected from

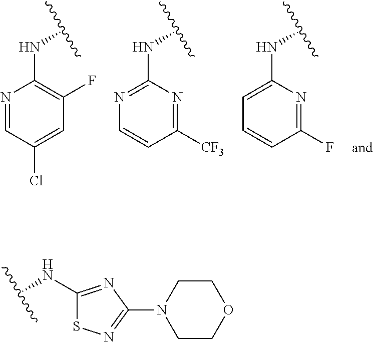

In certain embodiments, in the compound of Formula II, R² is selected from

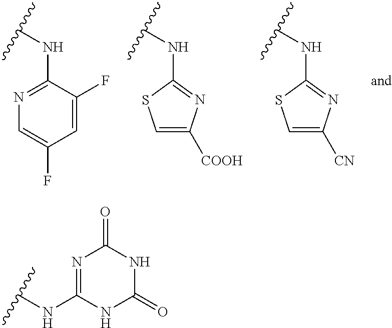

In certain embodiments, in the compound of Formula II, R² is selected from

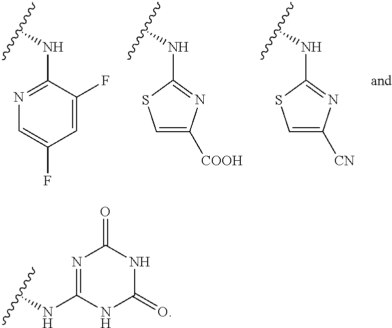

In certain embodiments, in the compound of Formula II, R² is selected from

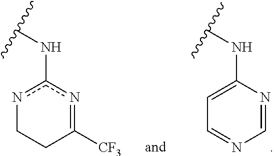

In certain embodiments, in the compound of Formula II, R² is selected from

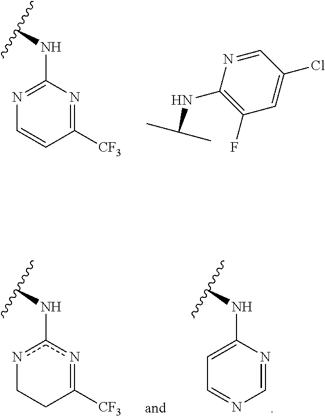

In certain embodiments, in the compound of Formula II, R² is selected from

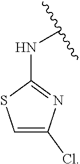 [SMILES: *Nc1nc(Cl)cs1]

In certain embodiments, in the compound of Formula II, R² is selected from

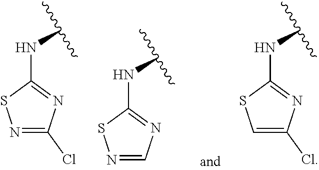

In certain embodiments, in the compound of Formula II, R² is selected from

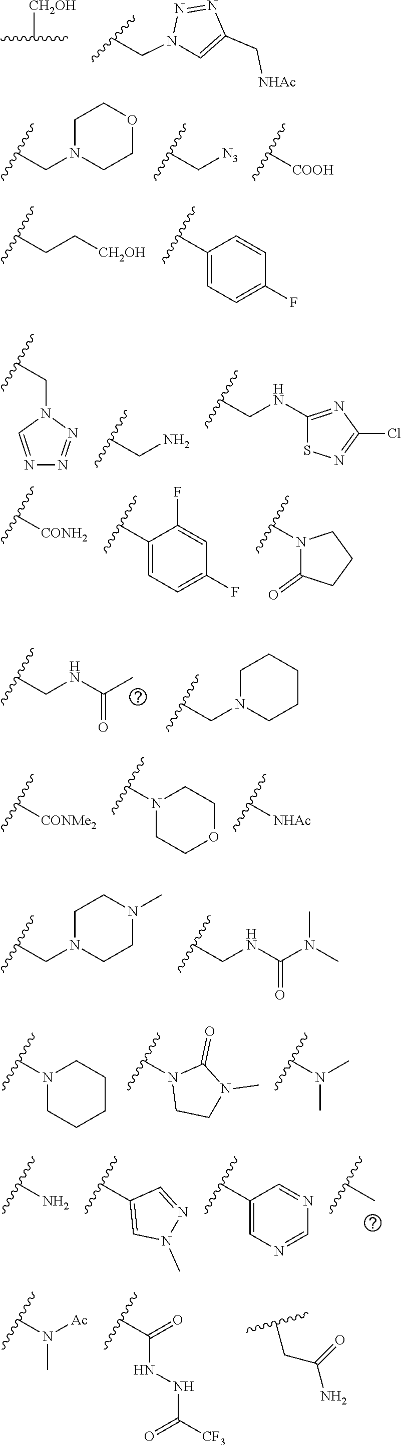

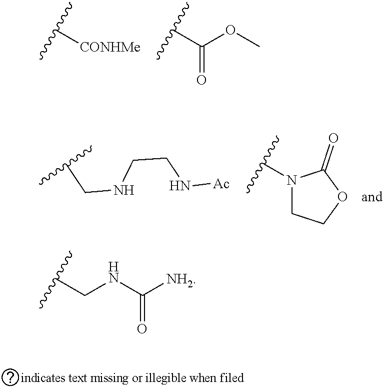

In certain embodiments in the compound of Formula II R² or R^{2A} is selected from

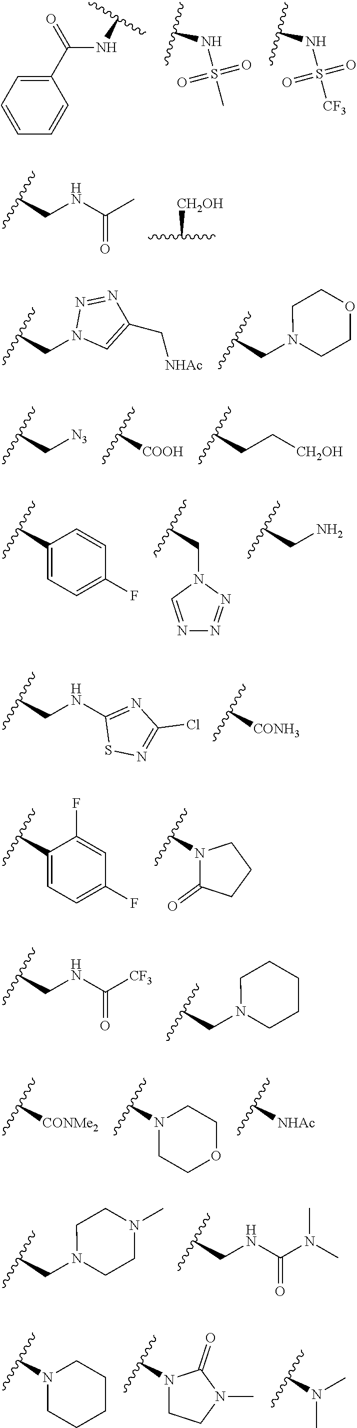

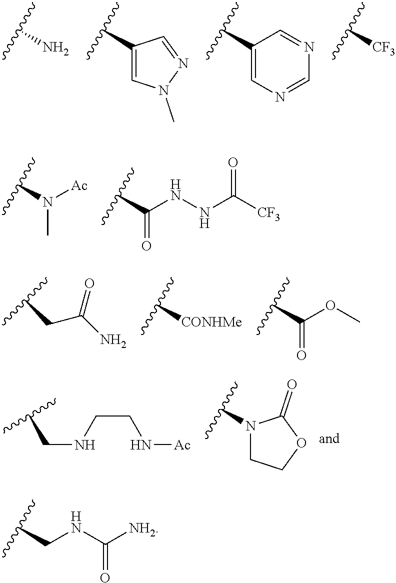

In certain embodiments, in the compound of Formula II, R² is selected from

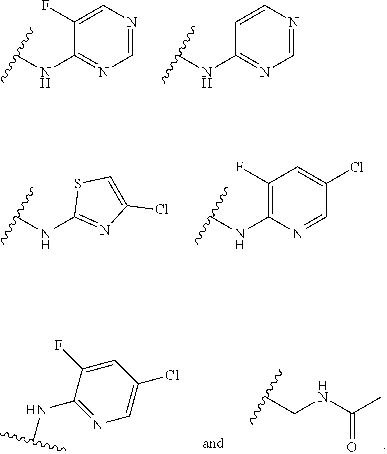

In certain embodiments, in the compound of Formula II, R² is selected from

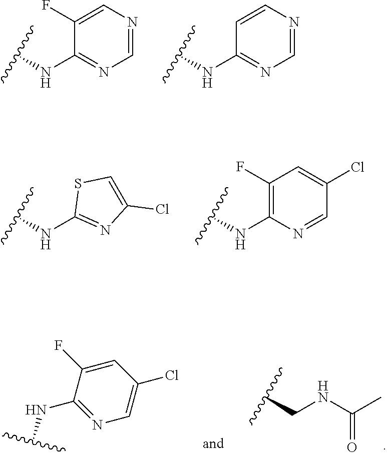

In certain embodiments, in the compound of Formula II, R² is selected from

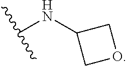 [SMILES: *NC1COC1]

In certain embodiments, in the compound of Formula II, R² is selected from

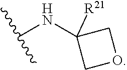

In certain embodiments, in the compound of Formula II, R² is selected from
In certain embodiments, in the compound of Formula II, R² is a spirocyclic heterocycle, for example, and without limitation,

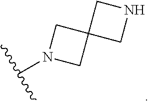 [SMILES: *N1CC2(CNC2)C1]

In certain embodiments, in the compound of Formula II, R² is a silicon containing heterocycle, for example, and without limitation,

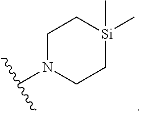 [SMILES: *N1CC[Si](C)(C)CC1]

In certain embodiments, in the compound of Formula II, R² is substituted with SF₅, for example, and without limitation,

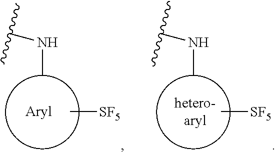

In certain embodiments, in the compound of Formula II R² is substituted with a sulfoxine, for example, and without limitation,

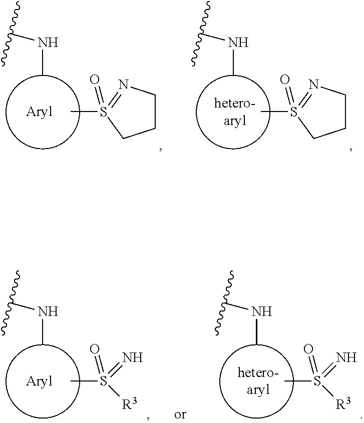

In certain embodiments, in the compound of Formula II, R¹⁰ is selected from bicyclic heterocycle.

In certain embodiments, in the compound of Formula II, R¹⁰ is selected from spirocyclic heterocycle.

In certain embodiments, in the compound of Formula II, R¹⁰ is selected from —NR⁶-heterocycle.

In certain embodiments, in the compound of Formula II, R¹⁰ is selected from

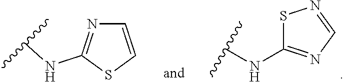 [SMILES: *Nc1nccs1 ; *Nc1ncns1]

In certain embodiments, in the compound of Formula II, R¹⁰ is selected from

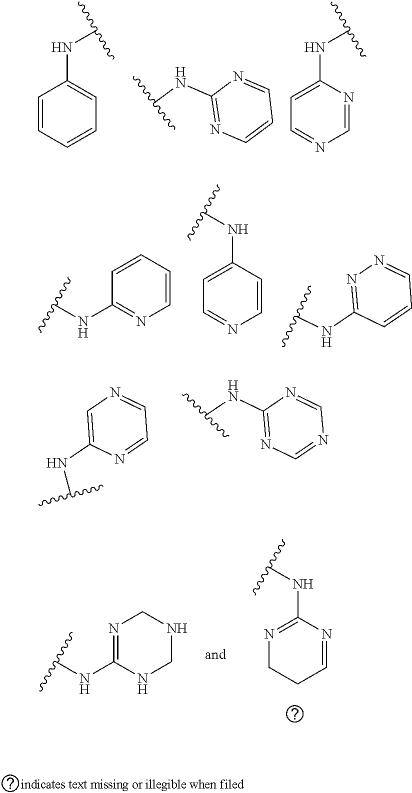

In certain embodiments in the compound of Formula II, R¹⁰ is selected from

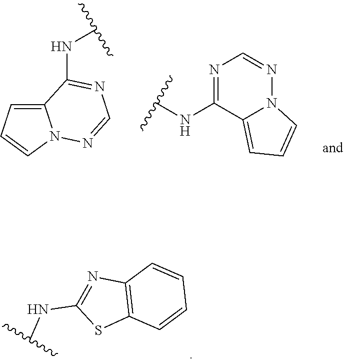 [SMILES: *Nc1ncnn2cccc12 ; *Nc1ncnn2cccc12 ; *Nc1nc2ccccc2s1]

In certain embodiments in the compound of Formula II, R¹⁰ is selected from

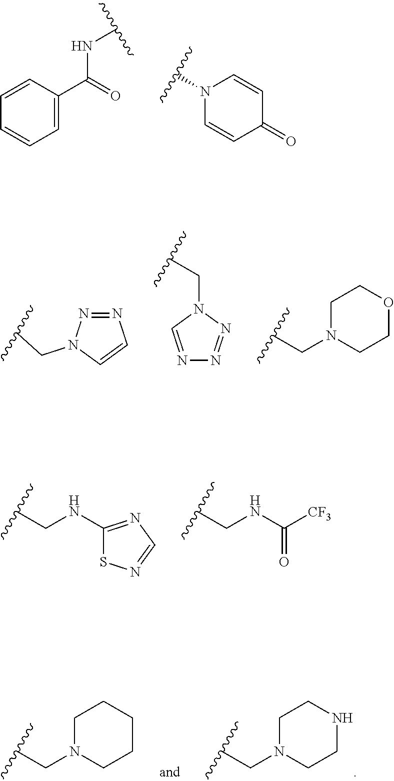

In certain embodiments in the compound of Formula II, Cycle is selected from

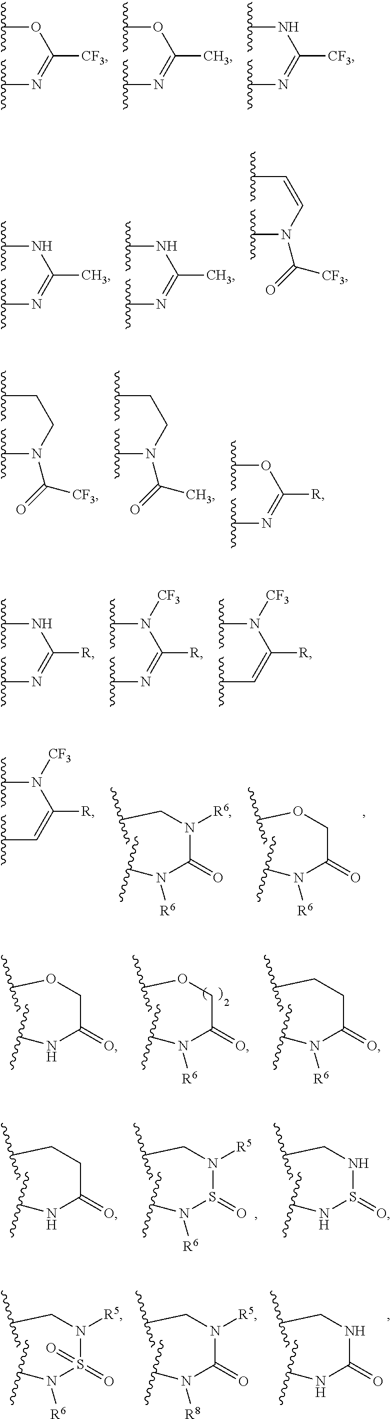

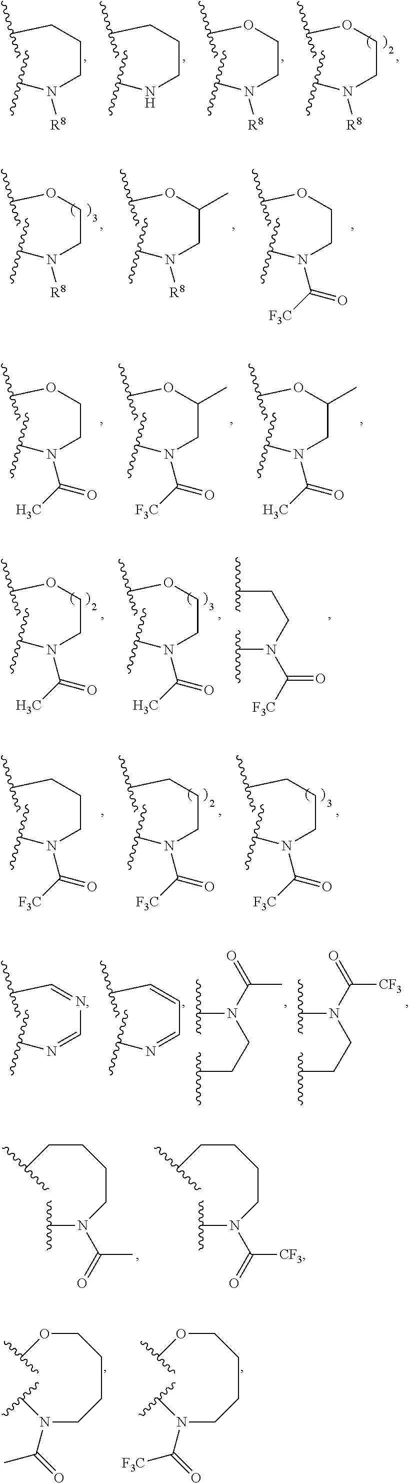

In certain embodiments, in the compound of Formula II, R³⁰ is selected from

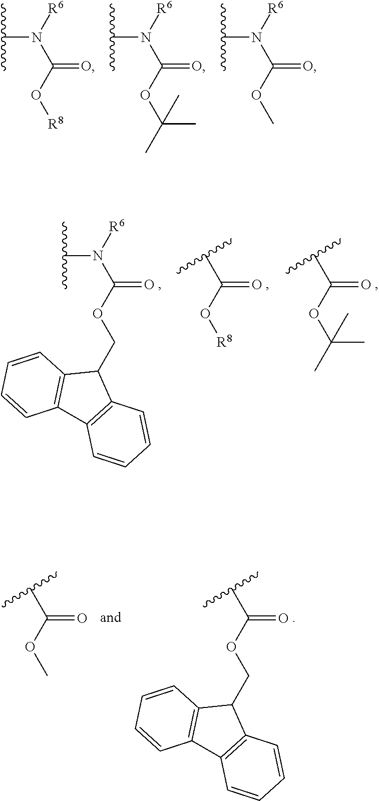

In certain embodiments, in the compound of Formula II, R²⁰⁰ is

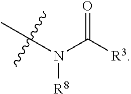

In certain embodiments, in the compound of Formula II, R²⁰⁰ is

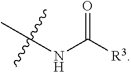

In certain embodiments, in the compound of Formula II, R²⁰⁰ is

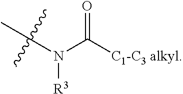

In certain embodiments, in the compound of Formula II, R²⁰⁰ is

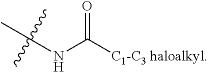

In certain embodiments, in the compound of Formula II, R²⁰⁰ is

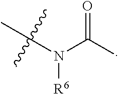

In certain embodiments, in the compound of Formula II, R²⁰⁰ is

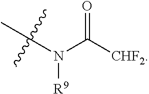

In certain embodiments, in the compound of Formula II, R²⁰⁰ is

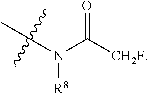

In certain embodiments, in the compound of Formula II, R²⁰⁰ is

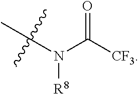

In certain embodiments, in the compound of Formula II, R²⁰⁰ is

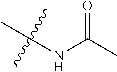 [SMILES: *NC(C)=O]

In certain embodiments, in the compound of Formula II, R²⁰⁰ is

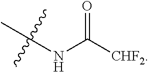 [SMILES: *NC(=O)C(F)F]

In certain embodiments, in the compound of Formula II, R²⁰⁰ is

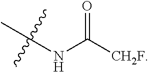

In certain embodiments, in the compound of Formula II, R²⁰⁰ is

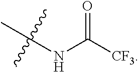 [SMILES: *NC(=O)C(F)(F)F]

Linkers

In non-limiting embodiments, in the compound of Formula II, Linker^{A} and Linker^{B} are independently selected from:

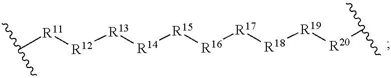

wherein:
R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, a divalent residue of a natural or unnatural amino acid, alkenyl, alkynyl, haloalkyl, alkoxy, and, heterocycle, heteroaryl, —CH₂CH₂—[O—(CH₂)₂]ₙ—O—, CH₂CH₂—[O—(CH₂)₂]ₙ—NR⁶—, —CH₂CH₂—[O—(CH₂)₂]ₙ—, -[—(CH₂)₂—O—]ₙ—, —[O—(CH₂)₂]ₙ—, —[O—CH(CH₃)C(O)]ₙ—, —[C(O)—CH(CH₃)—O]ₙ—,
—[O—CH₂C(O)]ₙ—, —[C(O)—CH₂—O]ₙ—, a divalent residue of a fatty acid, a divalent residue of an unsaturated or saturated mono- or di-carboxylic acid; each of which is optionally substituted with 1, 2, 3, or 4 substituents independently selected from R²¹;
n is independently selected at each instance from 0, 1, 2, 3, 4, 5, 6, 7, 8, 9, or 10; R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, heteroalkyl, and, heteroaryl, and heterocycle;
and the remaining variables are as defined herein.

In one embodiment, in the compound of Formula II, Linker^{A} is bond and Linker^{B} is

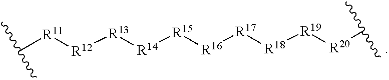

In one embodiment, in the compound of Formula II, Linker^{B} is bond and Linker^{A} is

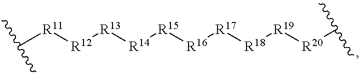

In one embodiment, in the compound of Formula II, a divalent residue of an amino acid is selected from

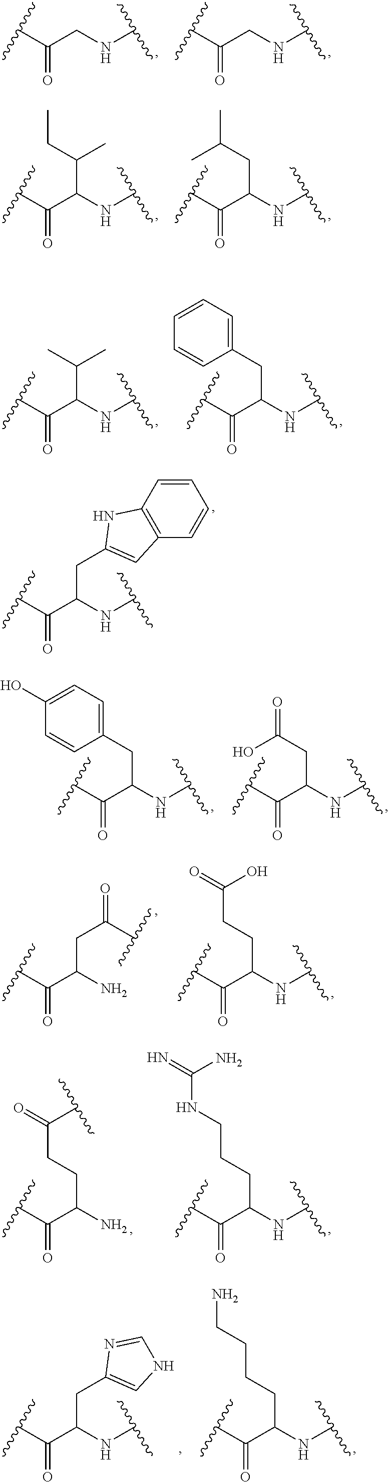

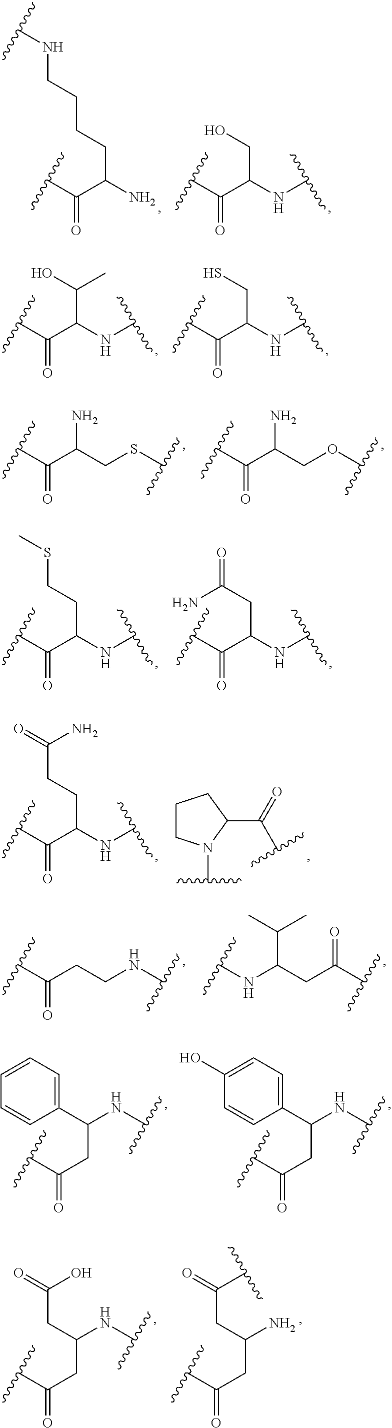

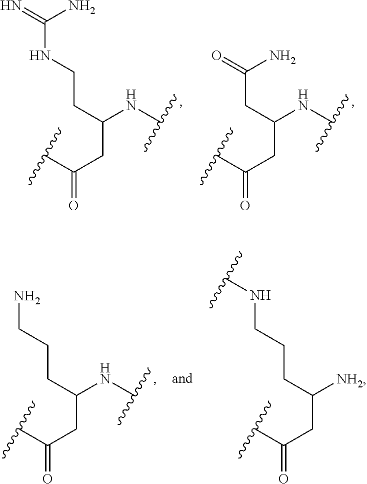 [SMILES: *NC(CCNC(=N)N)CC(*)=O ; *NC(CC(*)=O)CC(N)=O ; *NC(CCCN)CC(*)=O ; *NCCCC(N)CC(*)=O]

wherein the amino acid can be oriented in either direction and wherein the amino acid can be in the L- or D-form or a mixture thereof.

In one embodiment, in the compound of Formula II, a divalent residue of a dicarboxylic acid is generated from a nucleophilic addition reaction:

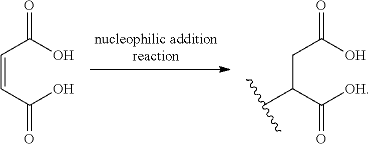 [SMILES: O=C(O)/C=C\C(=O)O ; *C(CC(=O)O)C(=O)O]

Non-limiting embodiments of a divalent residue of a dicarboxylic acid generated from a nucleophilic addition reaction include:

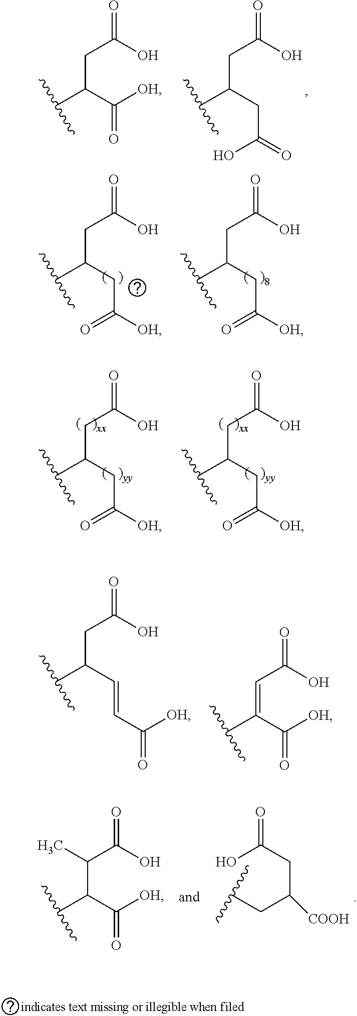

In one embodiment, in the compound of Formula II, a divalent residue of a dicarboxylic acid is generated from a condensation reaction:

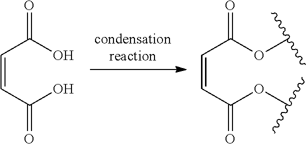 [SMILES: O=C(O)/C=C\C(=O)O ; *OC(=O)/C=C\C(=O)O*]

Non-limiting embodiments of a divalent residue of a dicarboxylic acid generated from a condensation include:

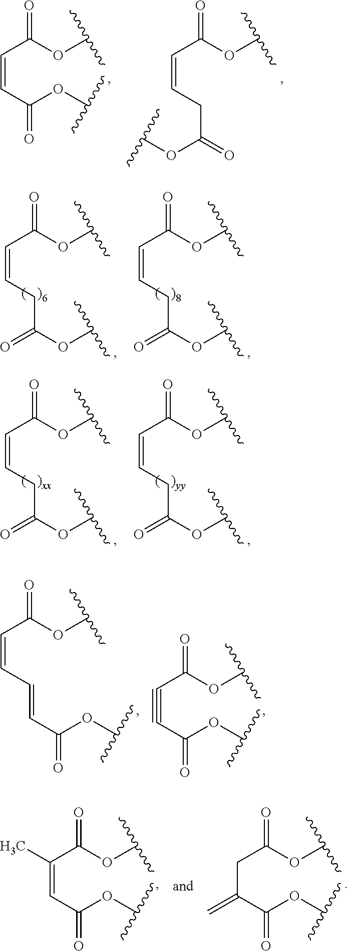

Non-limiting embodiments of a divalent residue of a saturated dicarboxylic acid include:

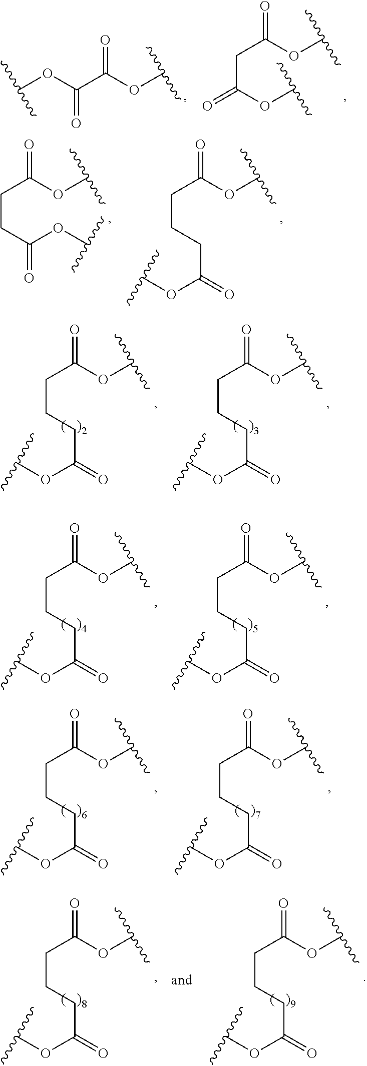

Non-limiting embodiments of a divalent residue of a saturated dicarboxylic acid include:

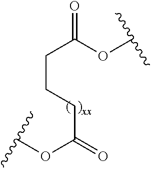 [SMILES: *OC(=O)CCCC(=O)O*]

Non-limiting embodiments of a divalent residue of a saturated monocarboxylic acid is selected from butyric acid (—OC(O)(CH₂)₂CH₂—), caproic acid (—OC(O)(CH₂)₄CH₂—), caprylic acid (—OC(O)(CH₂)₅CH₂—), capric acid (—OC(O)(CH₂)₈CH₂—), lauric acid (—OC(O)(CH₂)₁₀CH₂—), myristic acid (—OC(O)(CH₂)₁₂CH₂—), pentadecanoic acid (—OC(O)(CH₂)₁₃CH₂—), palmitic acid (—OC(O)(CH₂)₁₄CH₂—), stearic acid (—OC(O)(CH₂)₁₆CH₂—), behenic acid (—OC(O)(CH₂)₂₀CH₂—), and lignoceric acid (—OC(O)(CH₂)₂₂CH₂—);

Non-limiting embodiments of a divalent residue of a fatty acid include residues selected from linoleic acid, palmitoleic acid, vaccenic acid, paullinic acid, oleic acid, elaidic acid, gondoic acid, gadoleic acid, nervonic acid, myristoleic acid, and erucic acid:

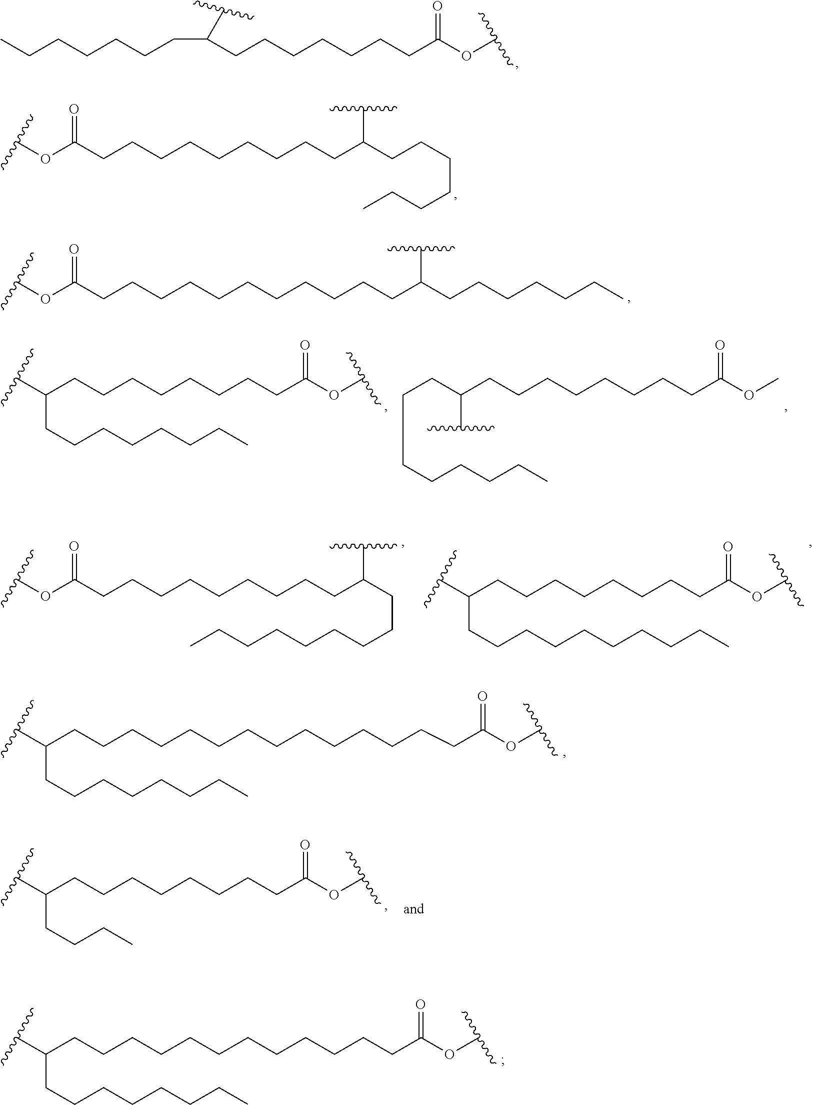

Non-limiting embodiments of a divalent residue of a fatty acid is selected from linoleic acid (—C(O)(CH₂)₇(CH)₂CH₂(CH)₂(CH₂)₄CH₂—), docosahexaenoic acid
(—C(O)(CH₂)₂(CHCHCH₂)₆CH₂—), eicosapentaenoic acid (—C(O)(CH₂)₃(CHCHCH₂)₅CH₂—), alpha-linolenic acid (—C(O)(CH₂)₇(CHCHCH₂)₃CH₂—) stearidonic acid
(—C(O)(CH₂)₄(CHCHCH₂)₄CH₂—), y-linolenic acid (—C(O)(CH₂)₄(CHCHCH₂)₃(CH₂)₃CH₂—), arachidonic acid (—C(O)(CH₂)₃,(CHCHCH₂)₄(CH₂)₄CH₂—), docosatetraenoic acid
(—C(O)(CH₂)₅(CHCHCH₂)₄(CH₂)₄CH₂—), palmitoleic acid (—C(O)(CH₂)₇CHCH(CH₂)₅CH₂—), vaccenic acid (—C(O)(CH₂)₉CHCH(CH₂)₅CH₂—), paullinic acid
(—C(O)(CH₂)₁₁CHCH(CH₂)₅CH₂—), oleic acid (—C(O)(CH₂)₇CHCH(CH₂)₇CH₂—), elaidic acid
(—C(O)(CH₂)₇CHCH(CH₂)₇CH₂—), gondoic acid (—C(O)(CH₂)₉CHCH(CH₂)₇CH₂—), gadoleic acid (—C(O)(CH₂)₇CHCH(CH₂)₉CH₂—), nervonic acid (—C(O)(CH₂)₁₃CHCH(CH₂)₃CH₂—), mead acid (—C(O)(CH₂)₃(CHCHCH₂)₃(CH₂)₆CH₂—), myristoleic acid (—C(O)(CH₂)₇CHCH(CH₂)₃CH₂—), and erucic acid (—C(O)(CH₂)₁₁CHCH(CH₂)₇CH₂—).

In certain embodiments, in the compound of Formula II, Linker^{C} is selected from:

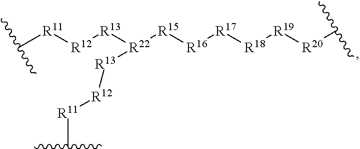

wherein:
R²² is independently at each occurrence selected from the group consisting of alkyl, —C(O)N—, —NC(O)—, —N—, —C(R²¹)—, —P(O)O—, —P(O)—, —P(O)(NR⁶R⁷)N—, alkenyl, haloalkyl, aryl, heterocycle, and heteroaryl, each of which is optionally substituted with 1, 2, 3, or 4 substituents independently selected from R²¹;
and the remaining variables are as defined herein.

In certain embodiments, in the compound of Formula II, Linker^{D} is selected from:

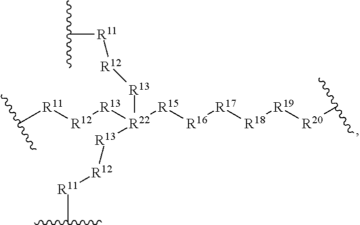

wherein:
R³² is independently at each occurrence selected from the group consisting of alkyl,
N⁺X—, —C—, alkenyl, haloalkyl, aryl, heterocycle, and heteroaryl, each of which is optionally substituted with 1, 2, 3, or 4 substituents independently selected from R²¹;
X— is an anionic group, for example Br— or Cl—; and
all other variables are as defined herein.

In certain embodiments, in the compound of Formula II, Linker^{A} is selected from:

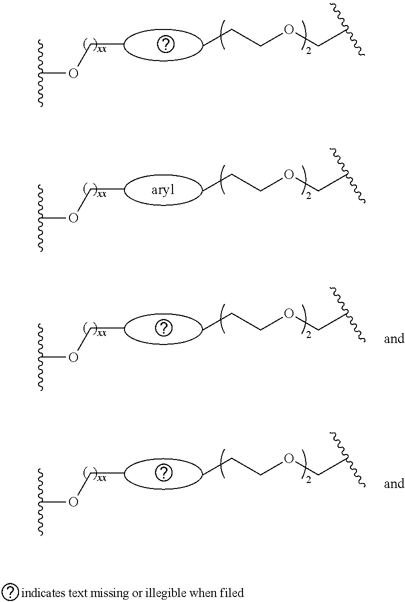

wherein each heteroaryl, heterocycle, cycloalkyl, and aryl can optionally be substituted with 1, 2, 3, or 4 of any combination of halogen, alkyl, haloalkyl, and, heteroaryl, heterocycle, or cycloalkyl, as allowed by valence.

In certain embodiments, in the compound of Formula II, Linker^{A} is selected from:

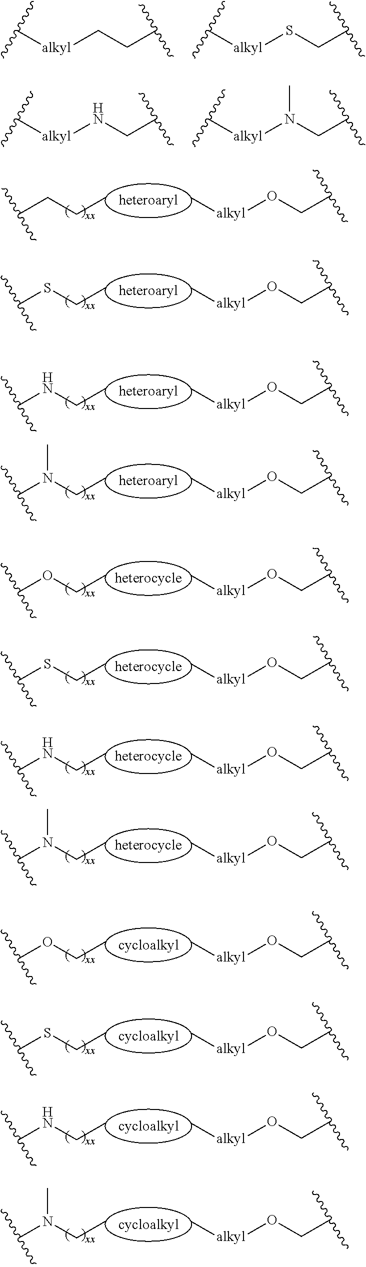

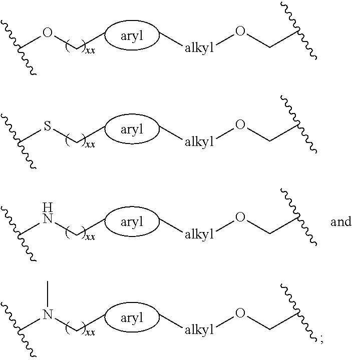

wherein each heteroaryl, heterocycle, cycloalkyl, and can optionally be substituted with 1, 2, 3, or 4 of any combination of halogen, alkyl, haloalkyl, aryl, heteroaryl, heterocycle, or cycloalkyl, as allowed by valence.

In certain embodiments, in the compound of Formula II, Linker B is selected from:

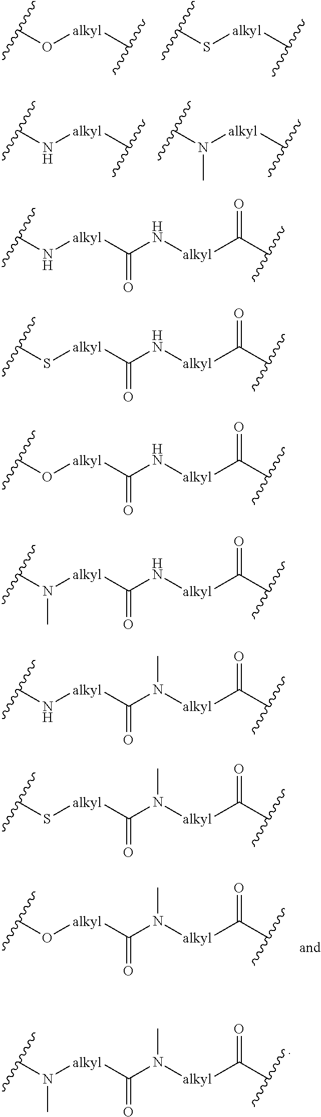

In certain embodiments, in the compound of Formula II, Linker B is selected from:

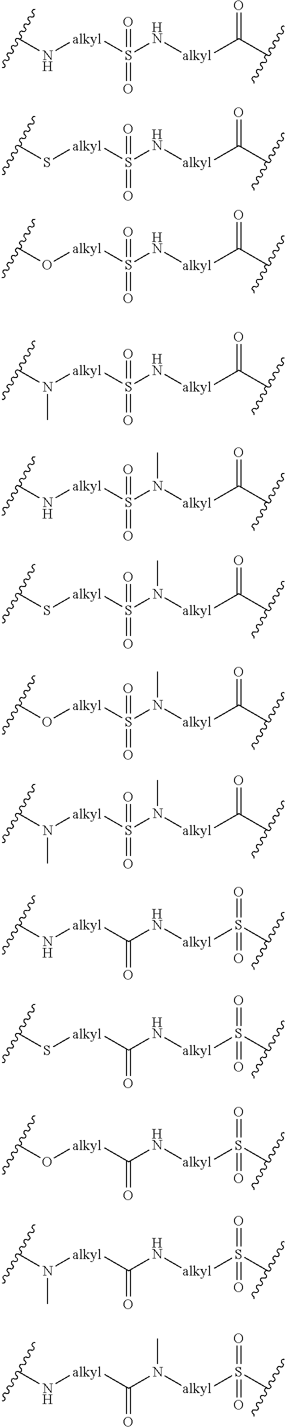

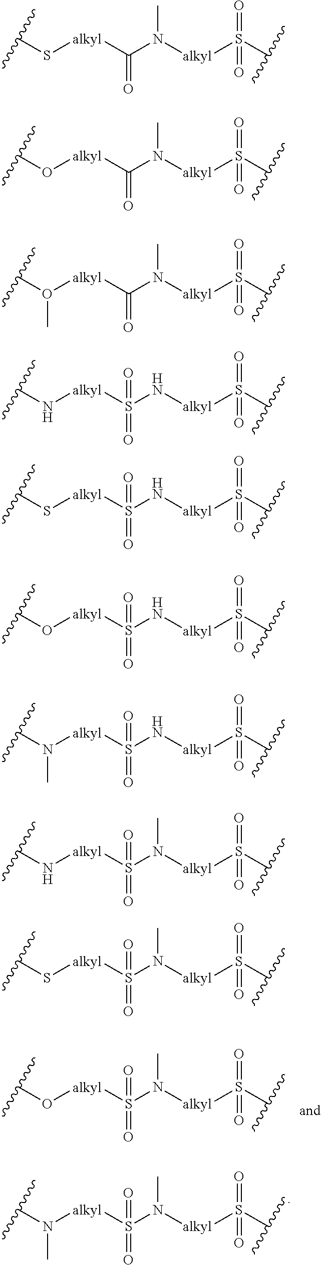

In certain embodiments, in the compound of Formula II, Linker^{B}, Linker^{C}, or Linker^{D} is selected from:

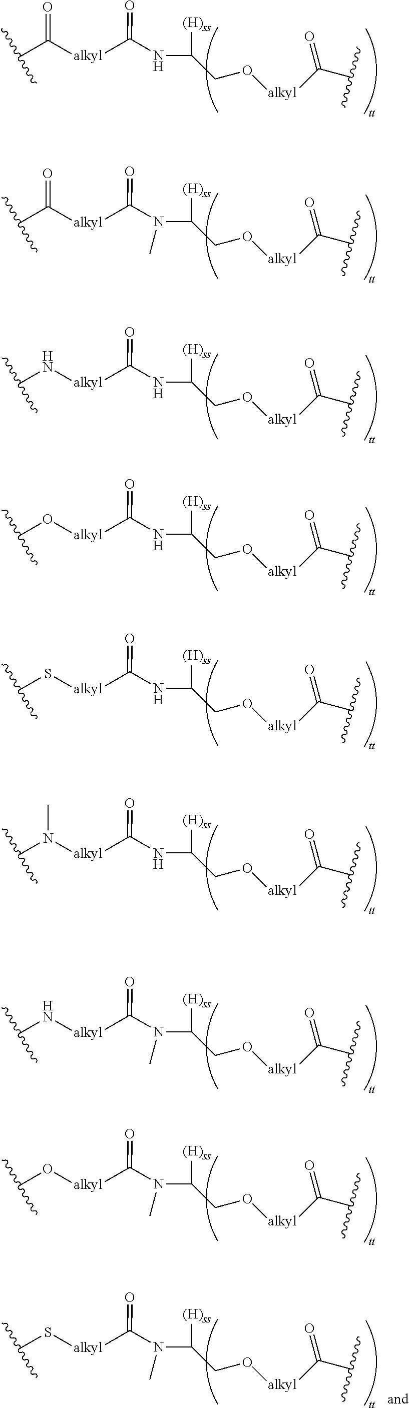

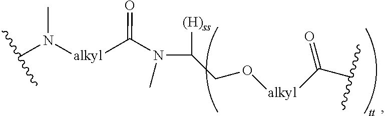

wherein tt is independently selected from 1, 2, or 3 and ss is 3 minus tt (3-tt).

In certain embodiments, in the compound of Formula II, Linker^{B}, Linker^{C}, or Linker^{D} is selected from:

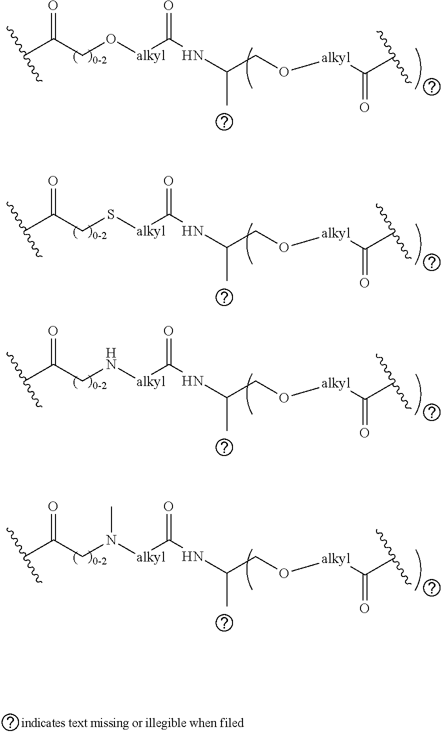

wherein tt and ss are as defined herein.

In certain embodiments, in the compound of Formula II, Linker^{B}, Linker^{C}, or Linker^{D} is selected from:

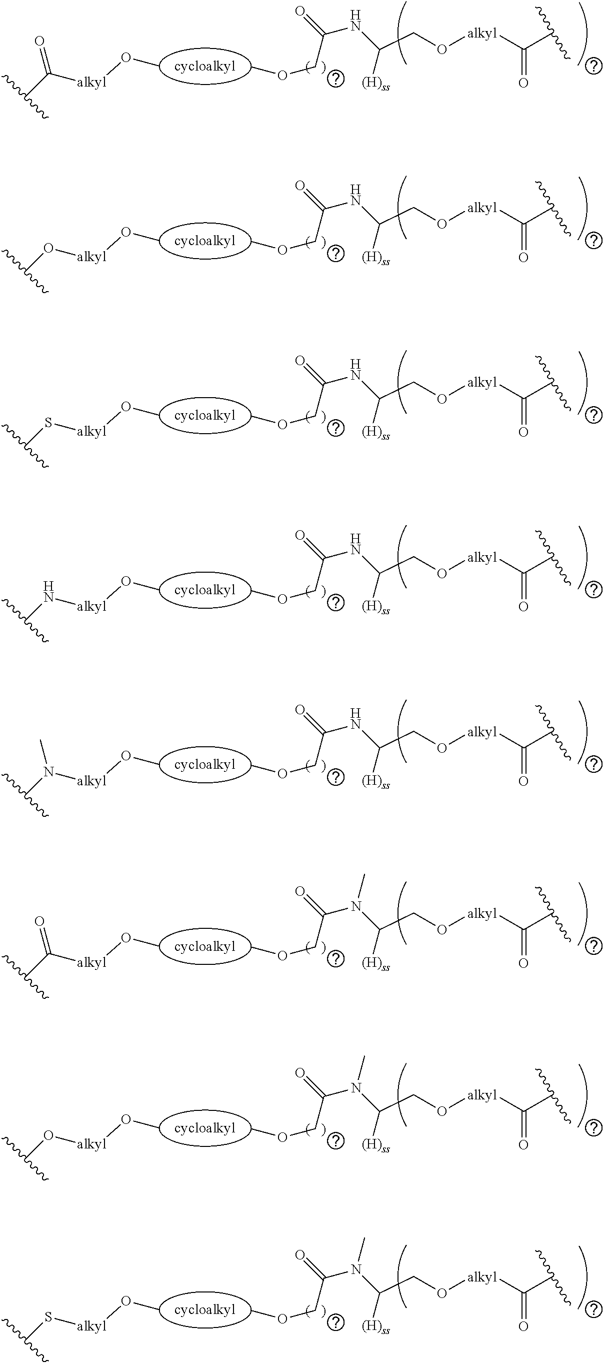

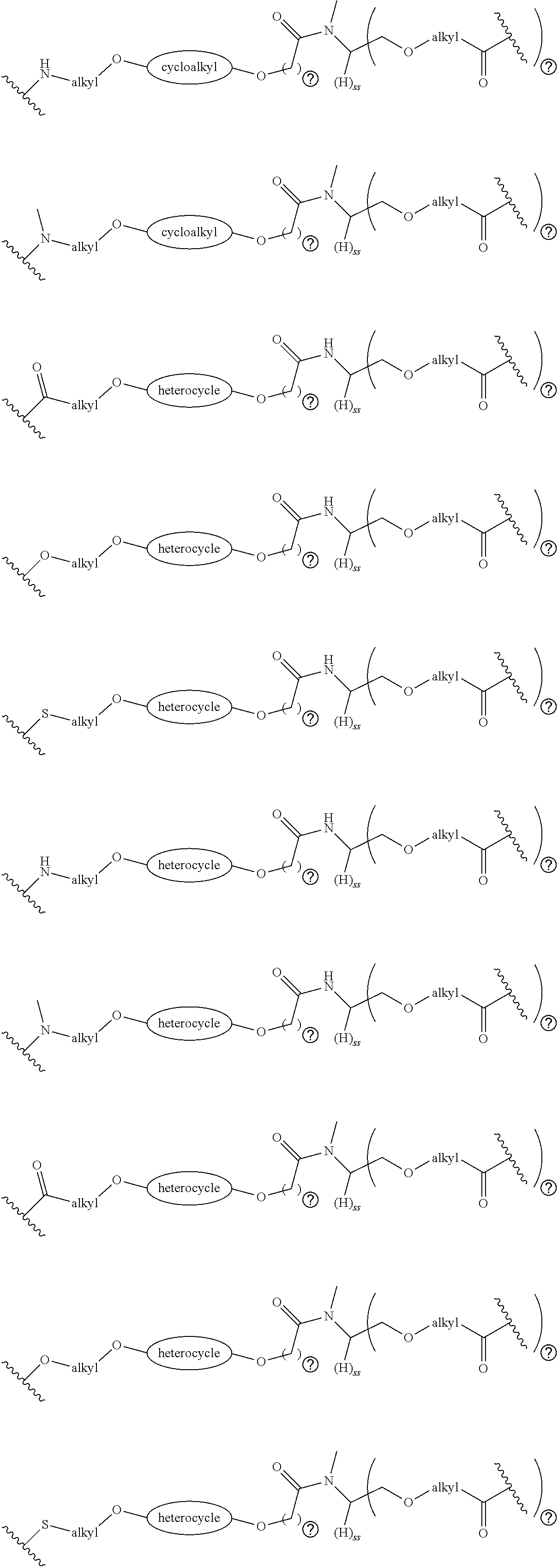

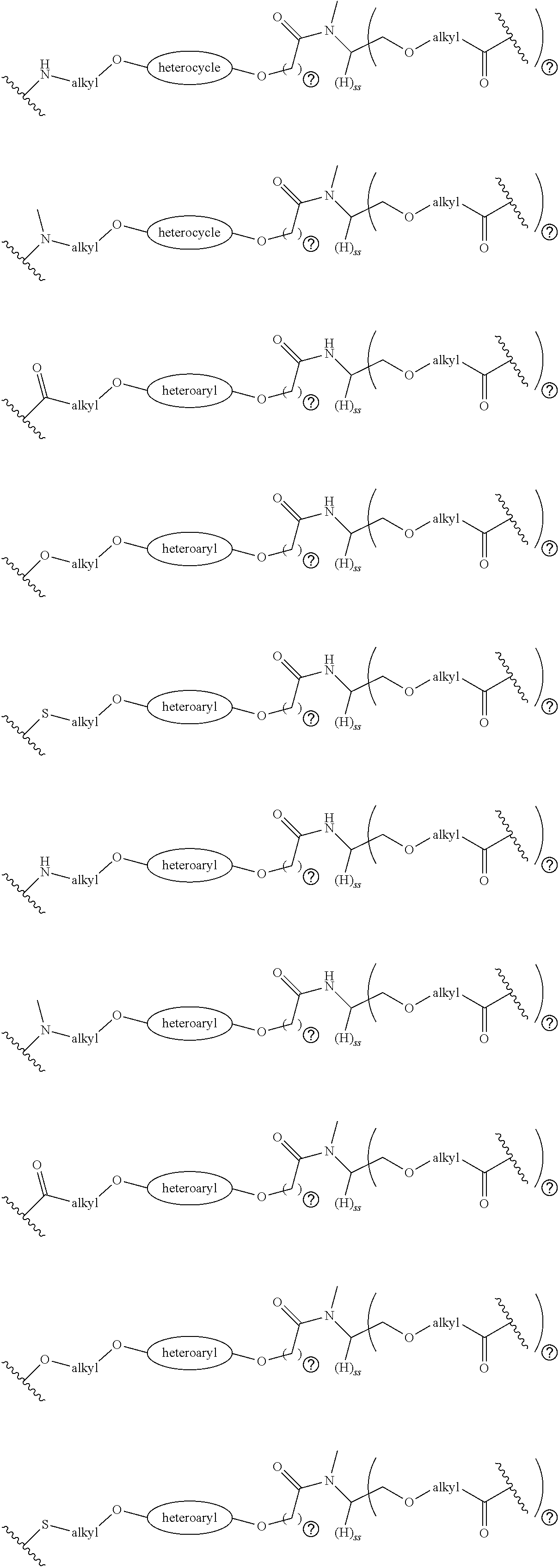

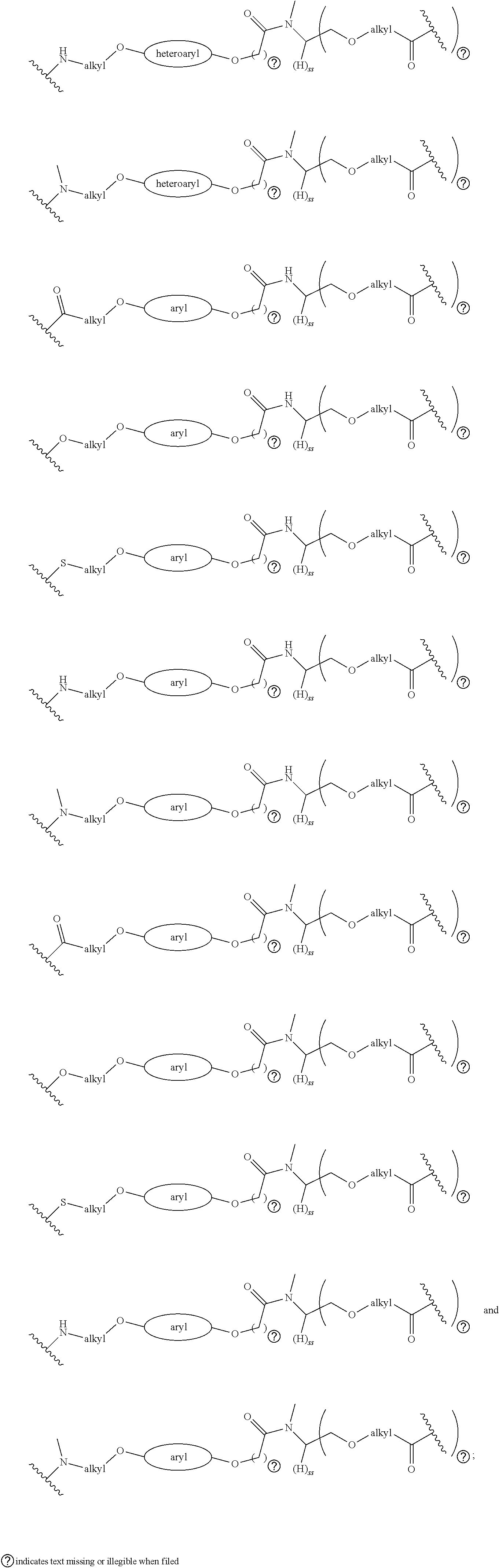

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence; and tt and ss are as defined herein.

In certain embodiments, in the compound of Formula II, Linker^{B}, Linker^{C}, or Linker^{D} is selected from:

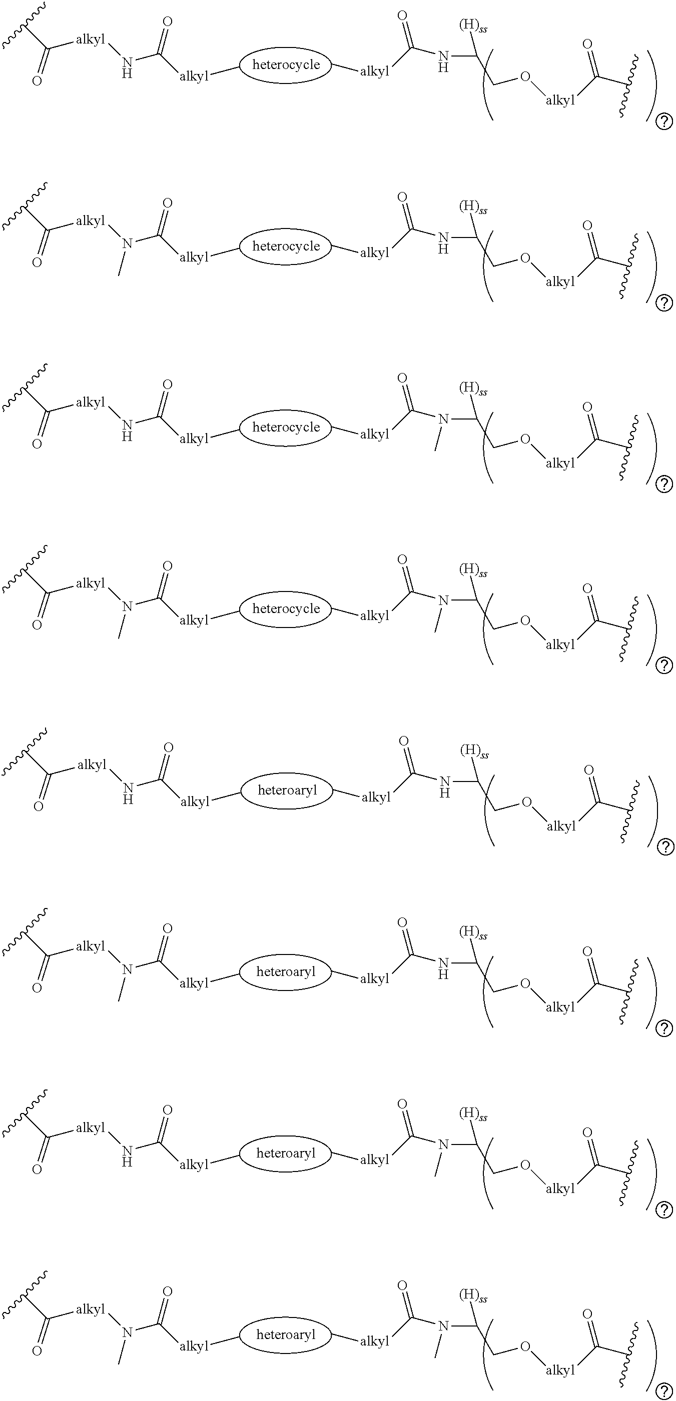

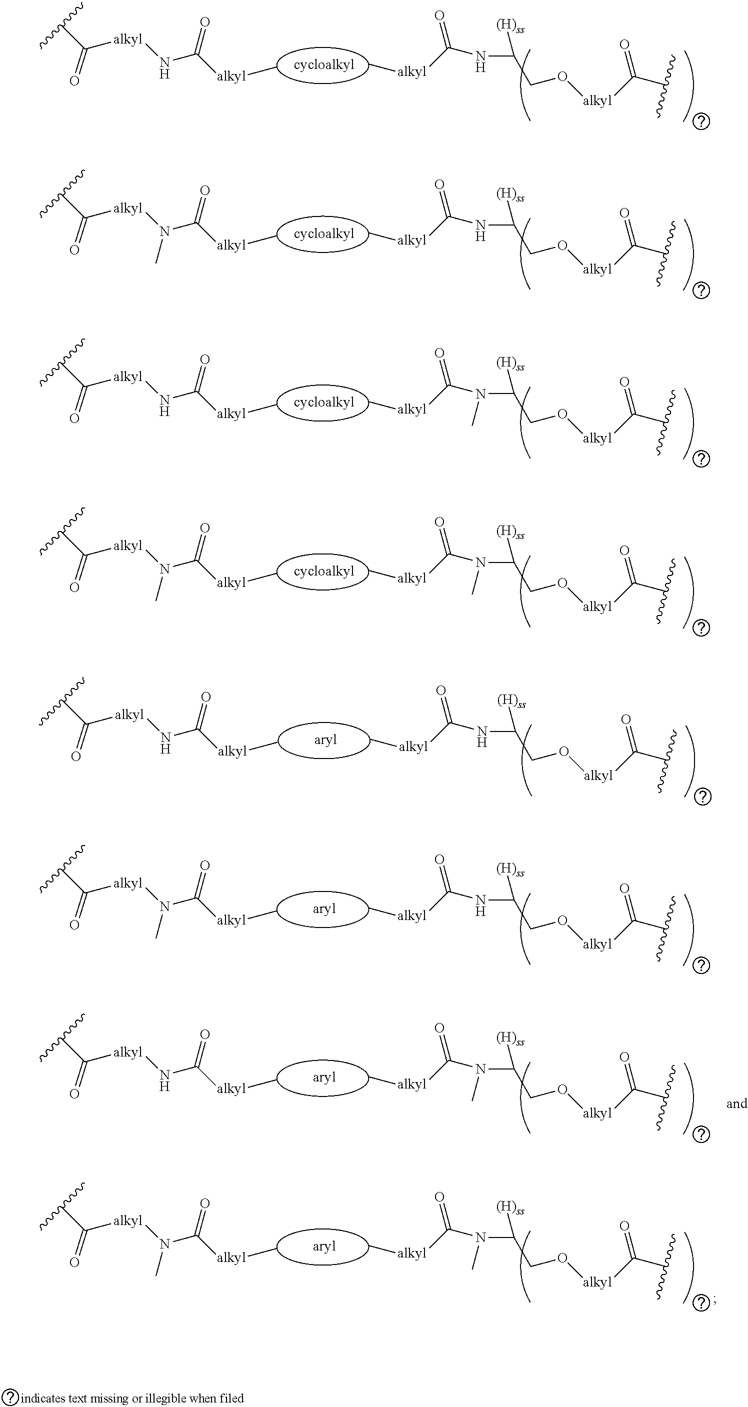

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence; and tt and ss are as defined herein.

In certain embodiments, in the compound of Formula II, Linker^{B}, Linker^{C}, or Linker^{D} is selected from:

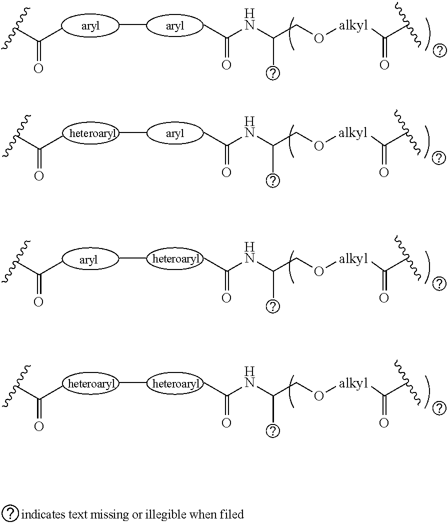

wherein each heteroaryl and aryl can optionally be substituted with 1, 2, 3, or 4 of any combination of halogen, alkyl, haloalkyl, aryl, heteroaryl, heterocycle, or cycloalkyl, as allowed by valence; and tt and ss are as defined herein.

In certain embodiments, in the compound of Formula II, Linker^{A} is selected from:

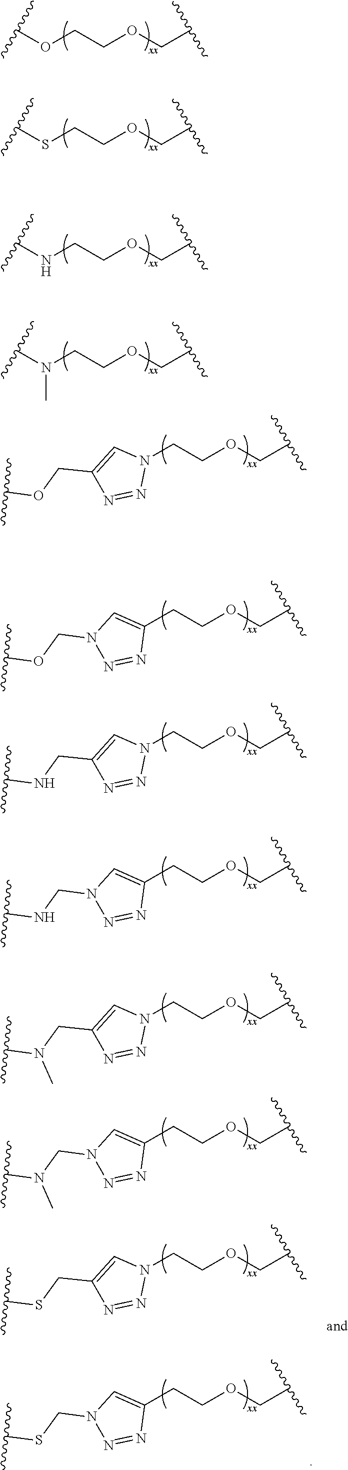

In certain embodiments in the compound of Formula II Linker^{A} is selected from:

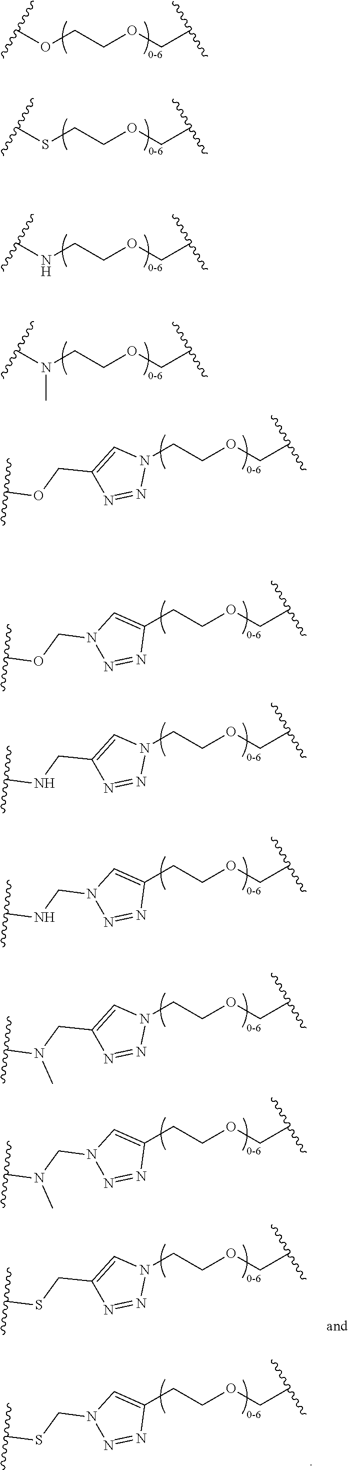

In certain embodiments, in the compound of Formula II, Linker^{A} is selected from:

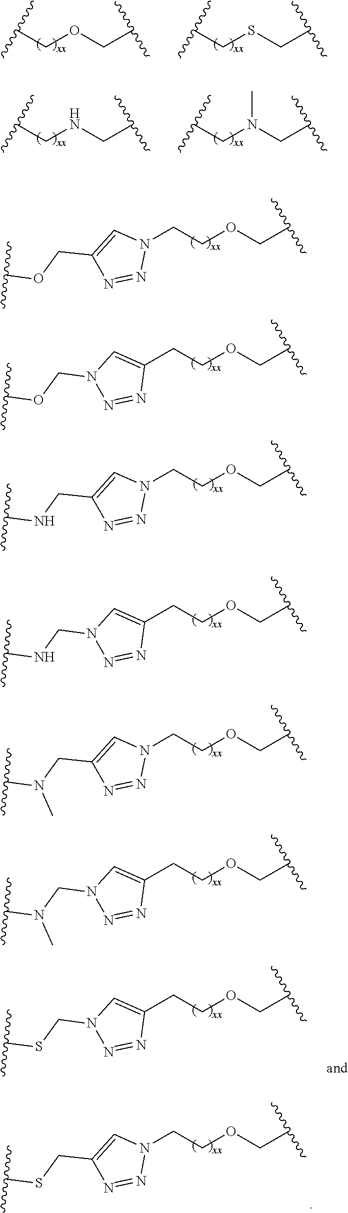

In certain embodiments, in the compound of Formula II, Linker^{A} is selected from:

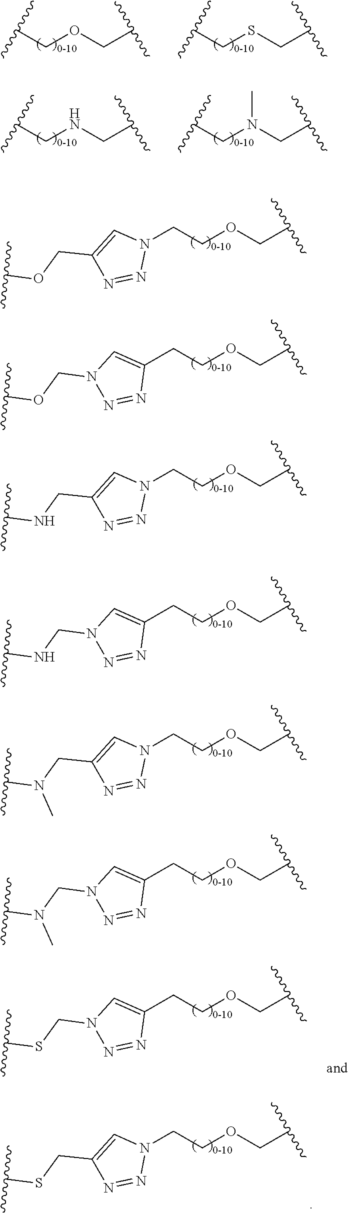

In certain embodiments in the compound of Formula II Linker^{B} is selected from:

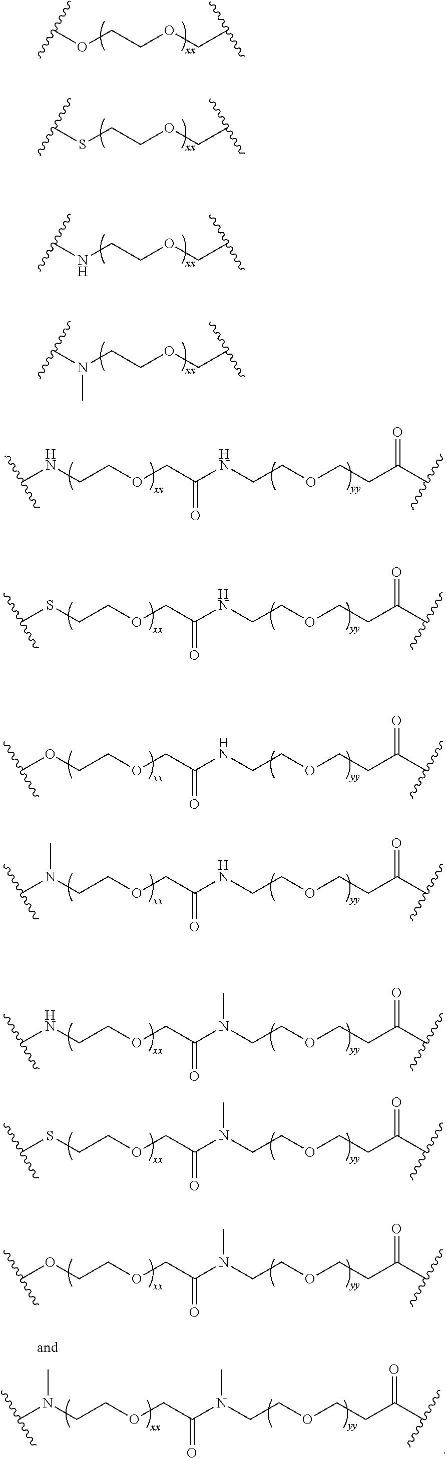

In certain embodiments, in the compound of Formula II, Linker^{B} is selected from:

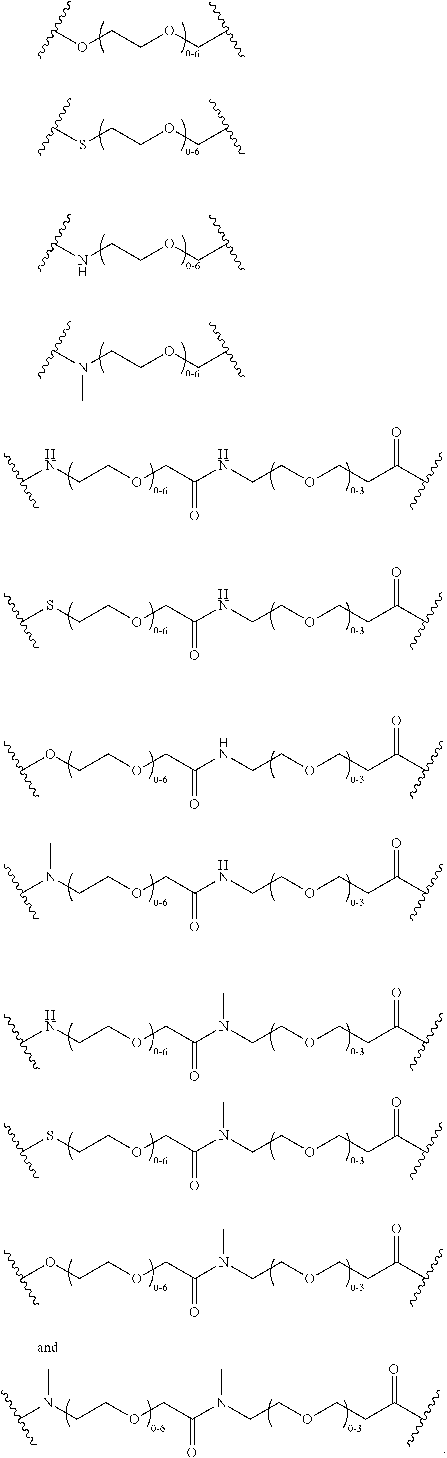

In certain embodiments, in the compound of Formula II, Linker^{B} is selected from:

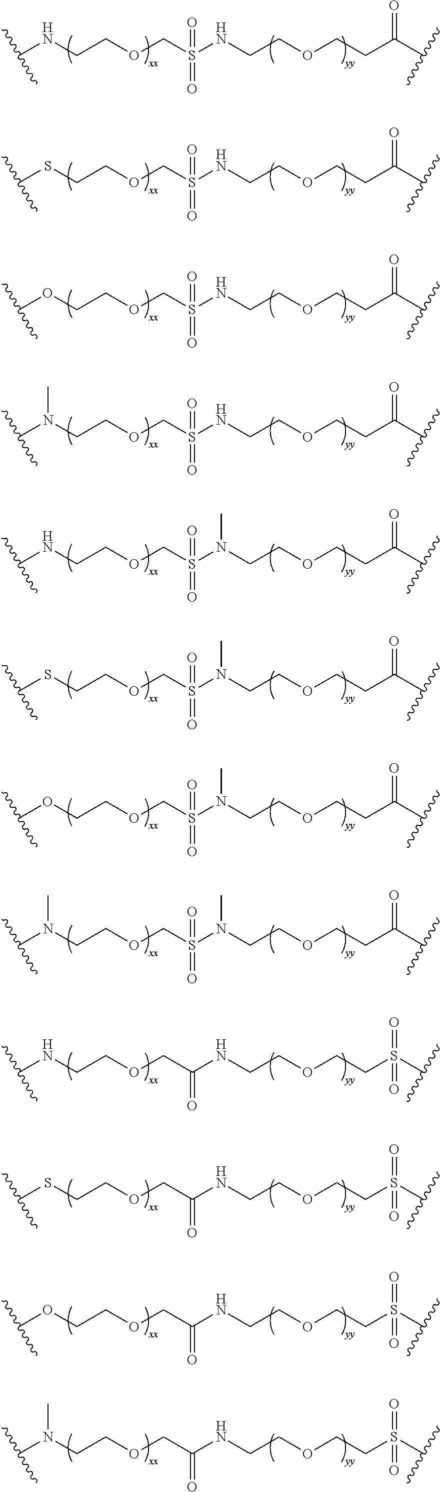

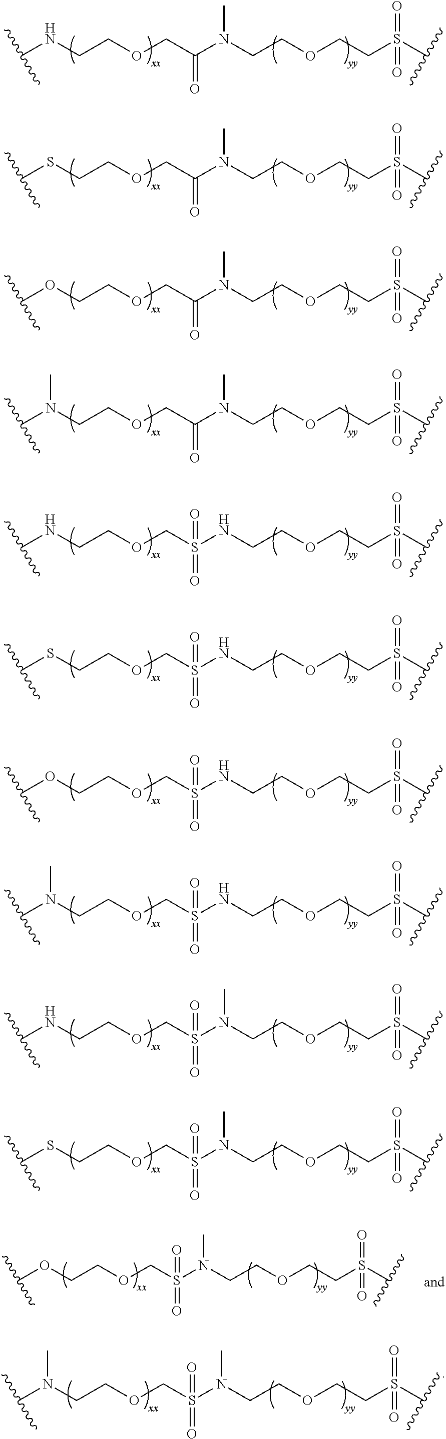

In certain embodiments, in the compound of Formula II, Linker^{B} is selected from:

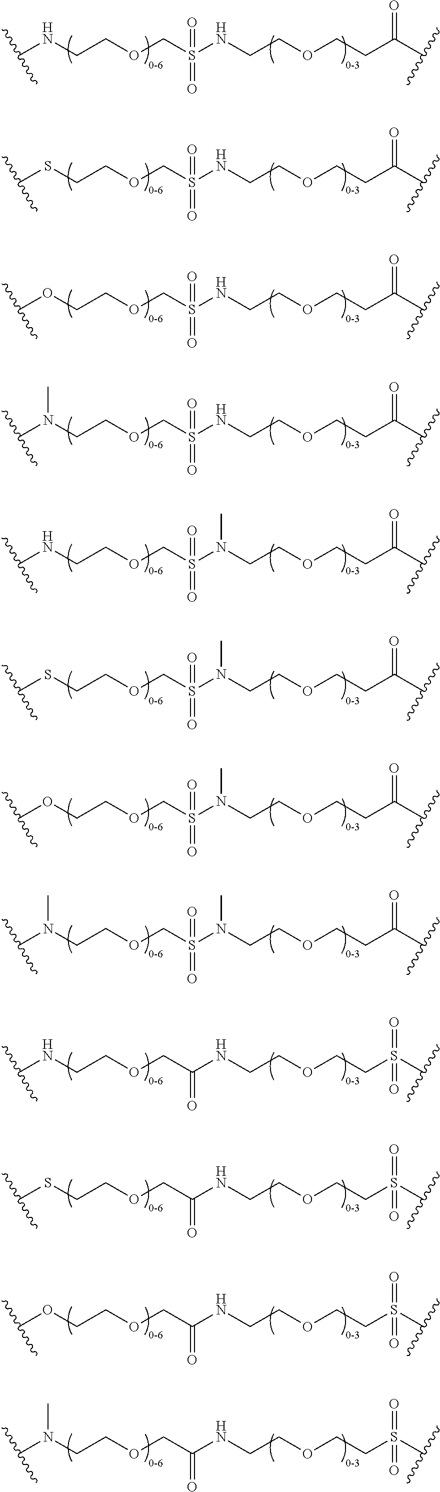

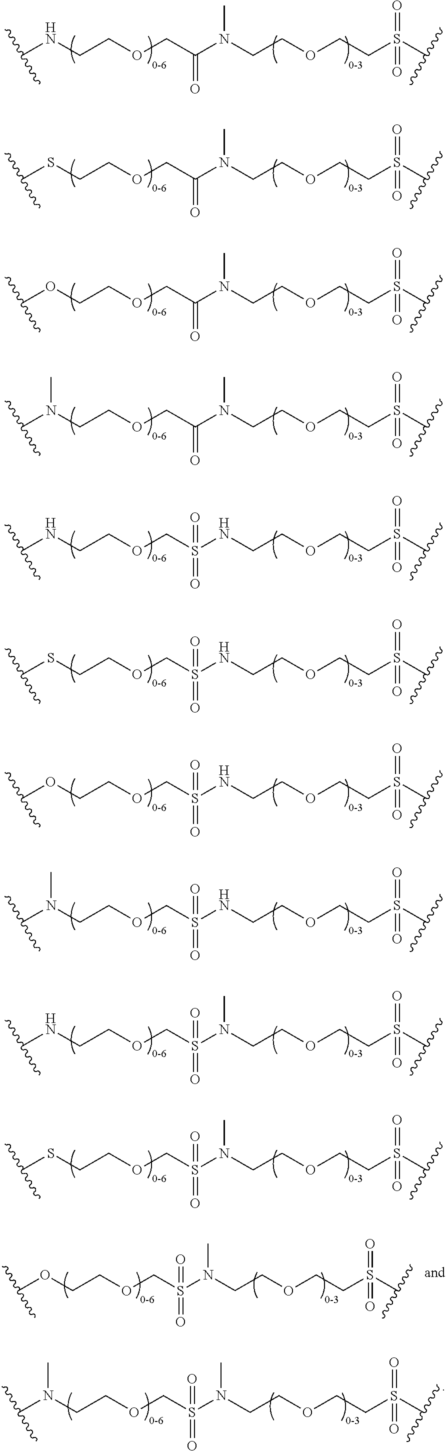

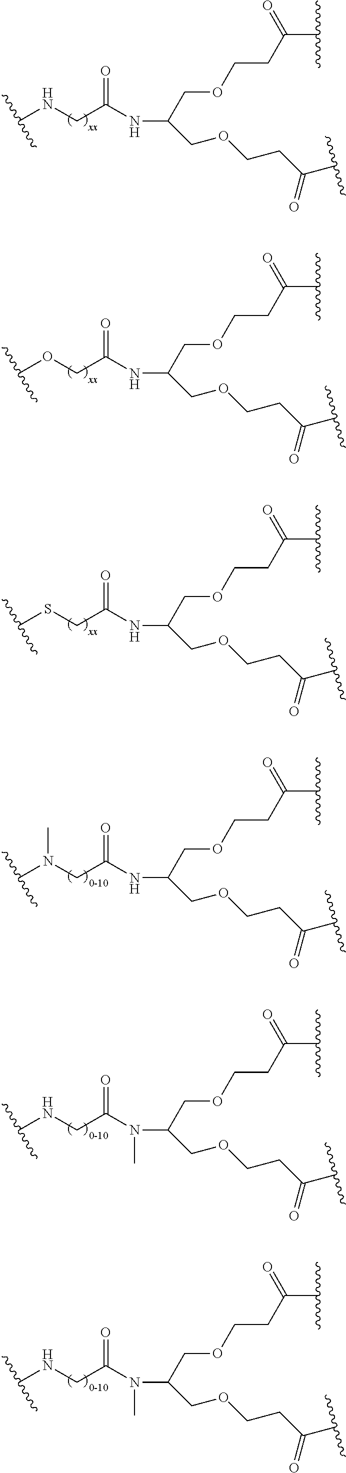

In certain embodiments, in the compound of Formula II, Linker^{C} is selected from:

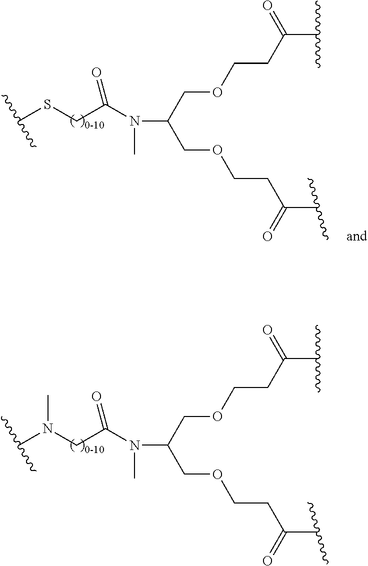 [SMILES: *SCC(=O)N(C)C(COCCC(*)=O)COCCC(*)=O ; *C(=O)CCOCC(COCCC(*)=O)N(C)C(=O)CN(*)C]

In certain embodiments, in the compound of Formula II, Linker^{C} is selected from:

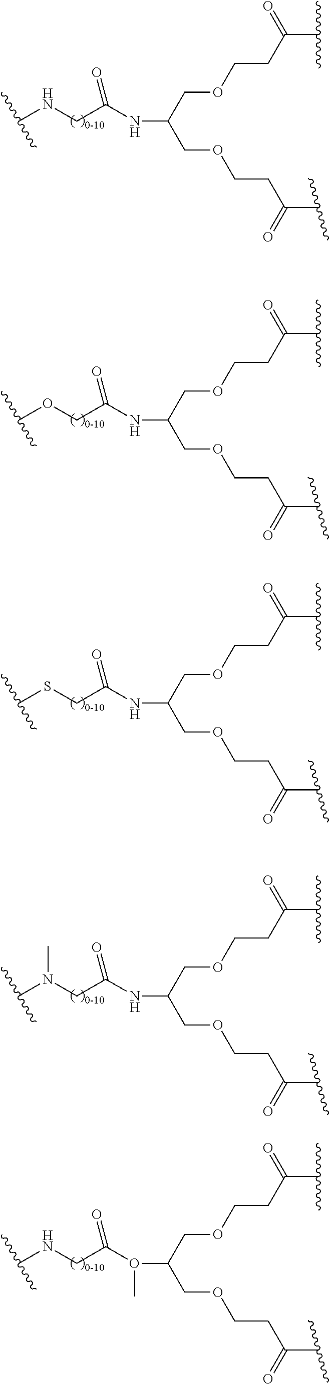

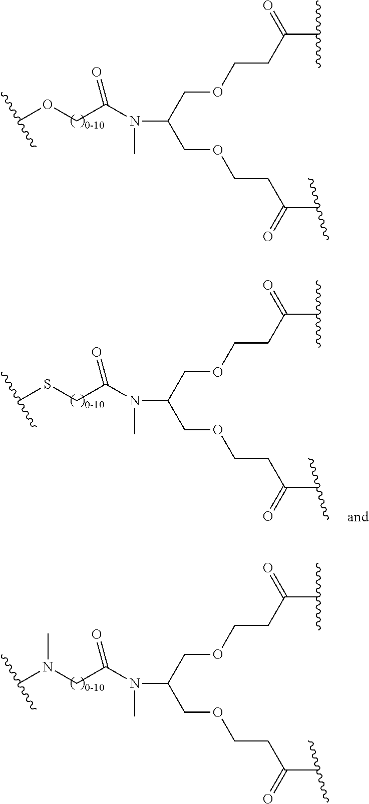 [SMILES: *OCC(=O)N(C)C(COCCC(*)=O)COCCC(*)=O ; *SCC(=O)N(C)C(COCCC(*)=O)COCCC(*)=O ; *C(=O)CCOCC(COCCC(*)=O)N(C)C(=O)CN(*)C]

In certain embodiments, in the compound of Formula II, Linker^{C} is selected from.

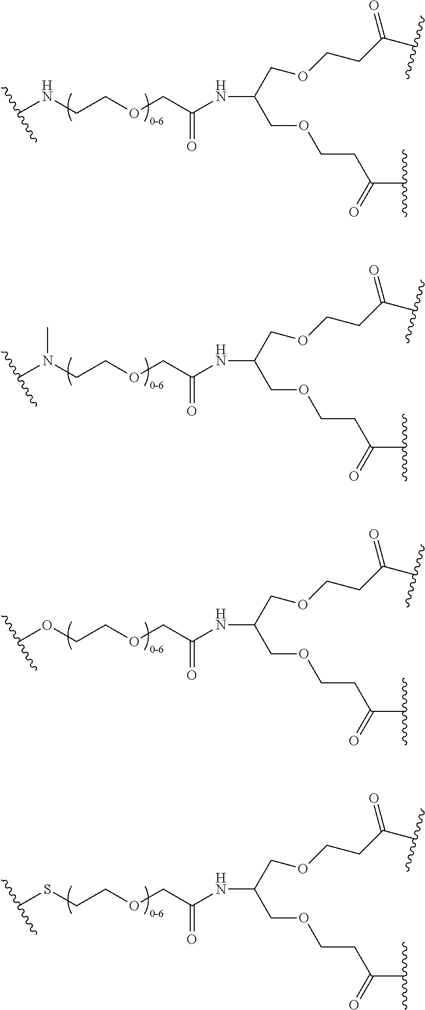

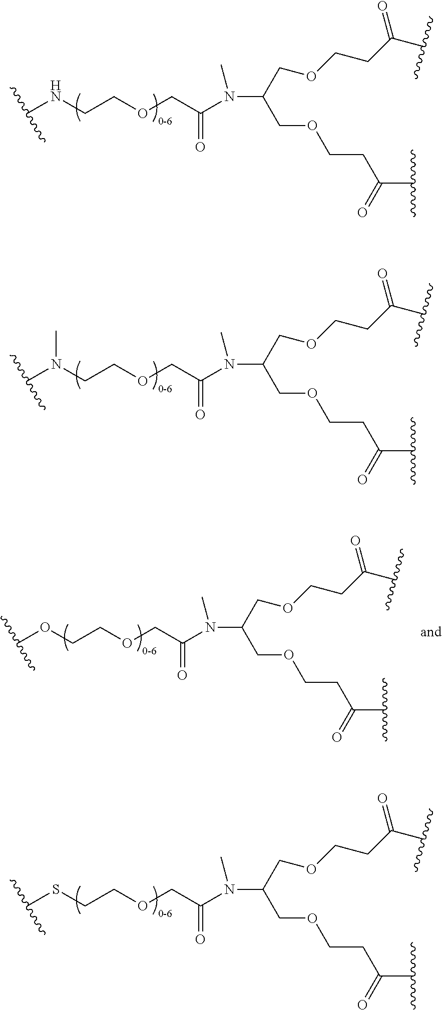

In certain embodiments, in the compound of Formula II, Linker^{C} is selected from:

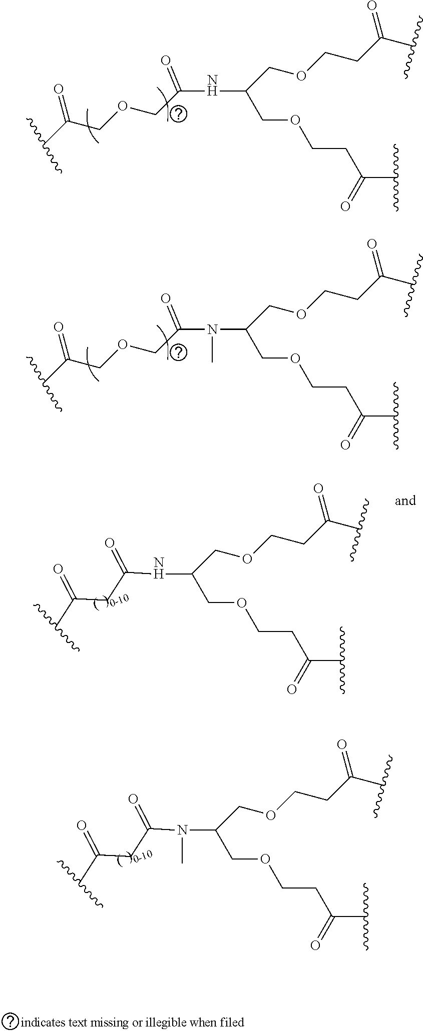

In certain embodiments, in the compound of Formula II, Linker^{C} is selected from:

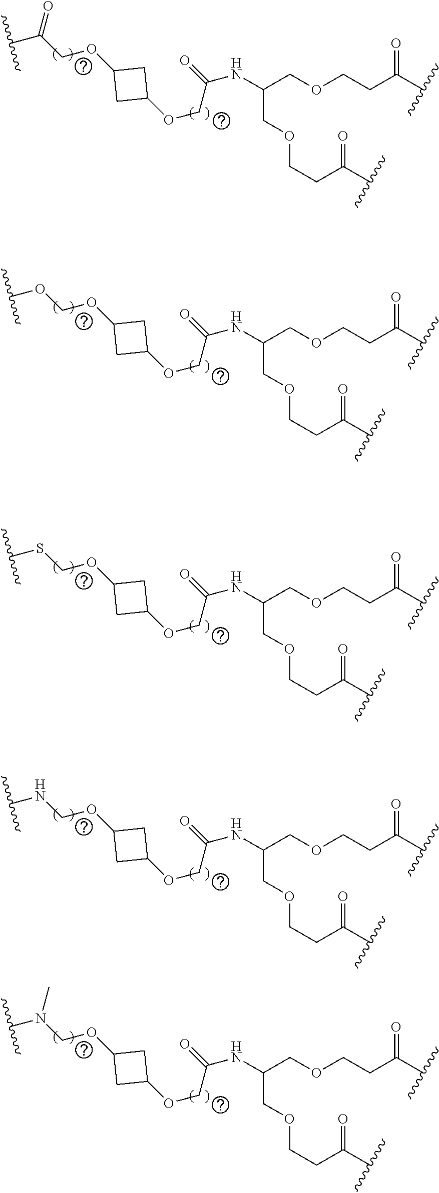

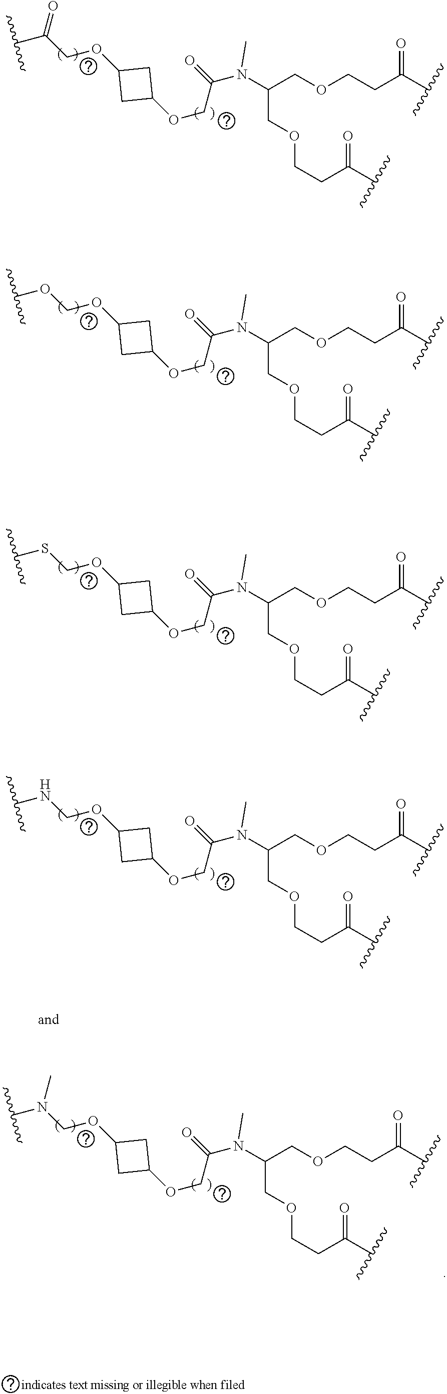

In certain embodiments, in the compound of Formula II, Linker^{C} is selected from:

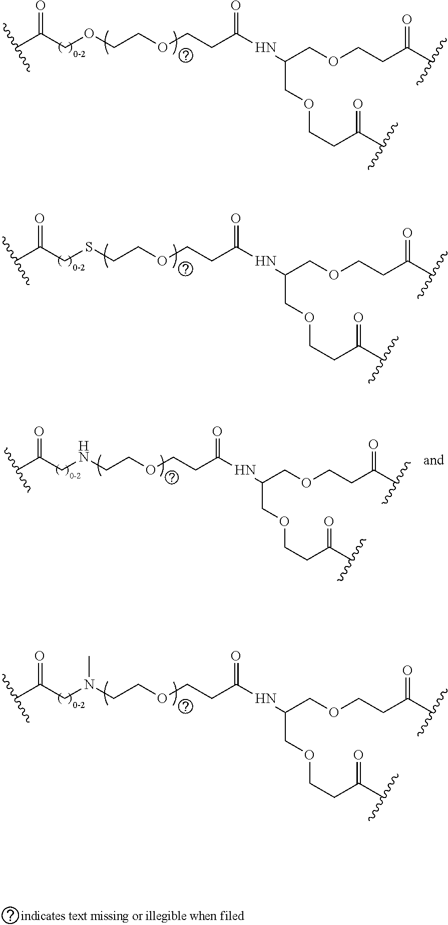

In certain embodiments, in the compound of Formula II, Linker^{C} is selected from:

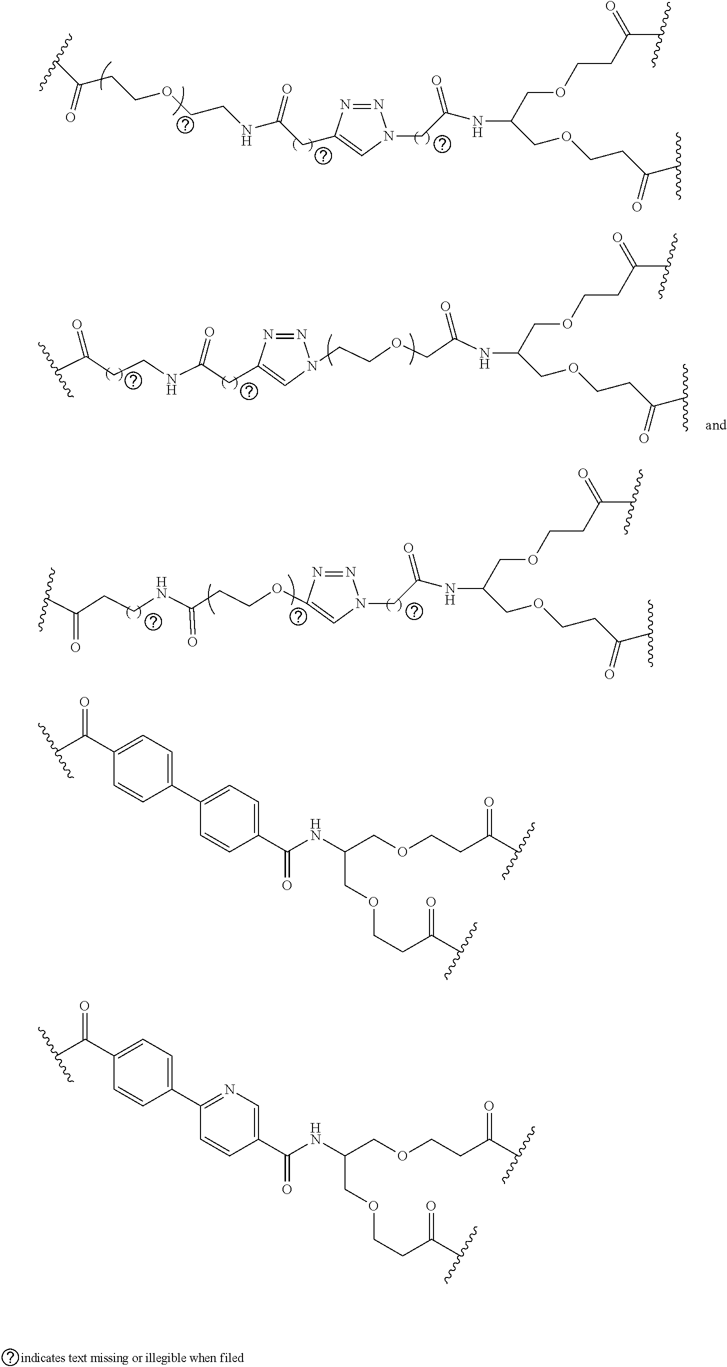

In certain embodiments, in the compound of Formula II, Linker^{C} is selected from:

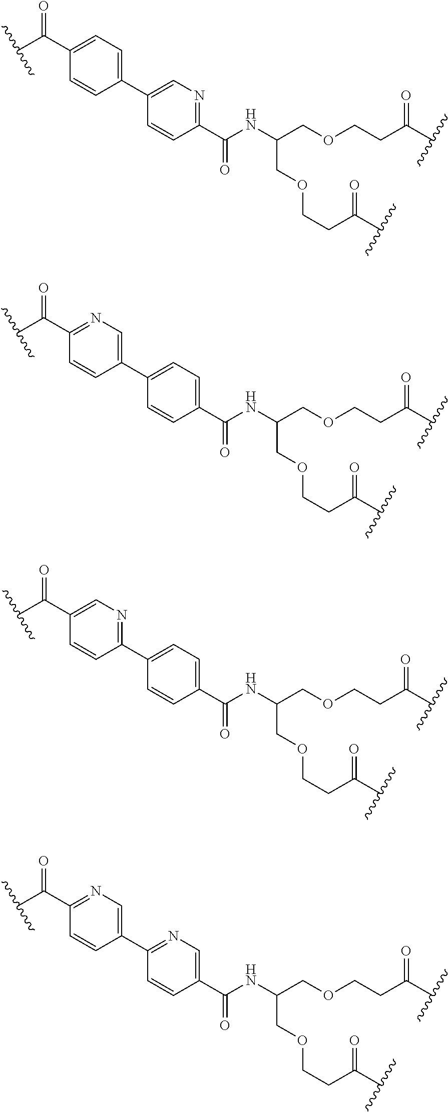 [SMILES: *C(=O)CCOCC(COCCC(*)=O)NC(=O)c1ccc(-c2ccc(C(*)=O)cc2)cn1 ; *C(=O)CCOCC(COCCC(*)=O)NC(=O)c1ccc(-c2ccc(C(*)=O)nc2)cc1 ; *C(=O)CCOCC(COCCC(*)=O)NC(=O)c1ccc(-c2ccc(C(*)=O)cn2)cc1 ; *C(=O)CCOCC(COCCC(*)=O)NC(=O)c1ccc(-c2ccc(C(*)=O)nc2)nc1]

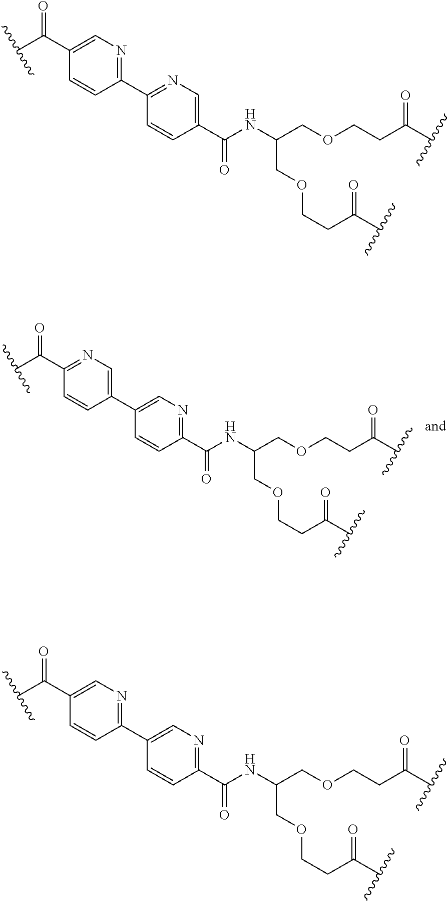 [SMILES: *C(=O)CCOCC(COCCC(*)=O)NC(=O)c1ccc(-c2ccc(C(*)=O)cn2)nc1 ; *C(=O)CCOCC(COCCC(*)=O)NC(=O)c1ccc(-c2ccc(C(*)=O)nc2)cn1 ; *C(=O)CCOCC(COCCC(*)=O)NC(=O)c1ccc(-c2ccc(C(*)=O)cn2)cn1]

In certain embodiments, in the compound of Formula II, Linker^{D} is selected from:

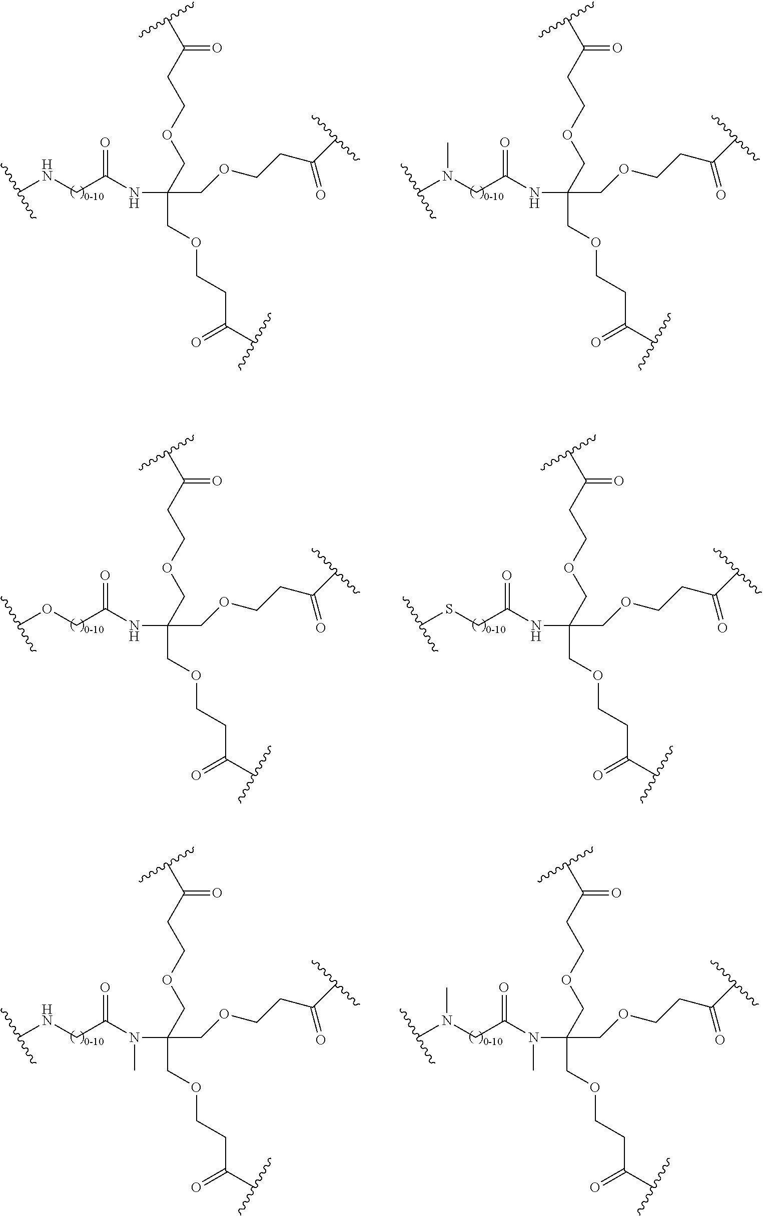 [SMILES: *NCC(=O)NC(COCCC(*)=O)(COCCC(*)=O)COCCC(*)=O ; *C(=O)CCOCC(COCCC(*)=O)(COCCC(*)=O)NC(=O)CN(*)C ; *OCC(=O)NC(COCCC(*)=O)(COCCC(*)=O)COCCC(*)=O ; *SCC(=O)NC(COCCC(*)=O)(COCCC(*)=O)COCCC(*)=O ; *NCC(=O)N(C)C(COCCC(*)=O)(COCCC(*)=O)COCCC(*)=O ; *C(=O)CCOCC(COCCC(*)=O)(COCCC(*)=O)N(C)C(=O)CN(*)C]

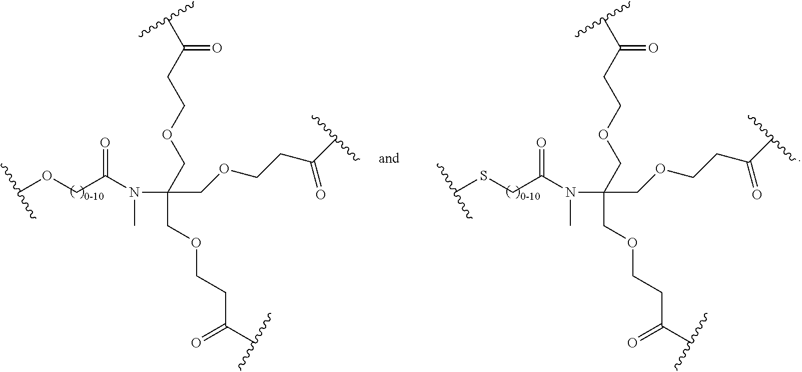 [SMILES: *OCC(=O)N(C)C(COCCC(*)=O)(COCCC(*)=O)COCCC(*)=O ; *SCC(=O)N(C)C(COCCC(*)=O)(COCCC(*)=O)COCCC(*)=O]

In certain embodiments, in the compound of Formula II, Linker^{D} is selected from.

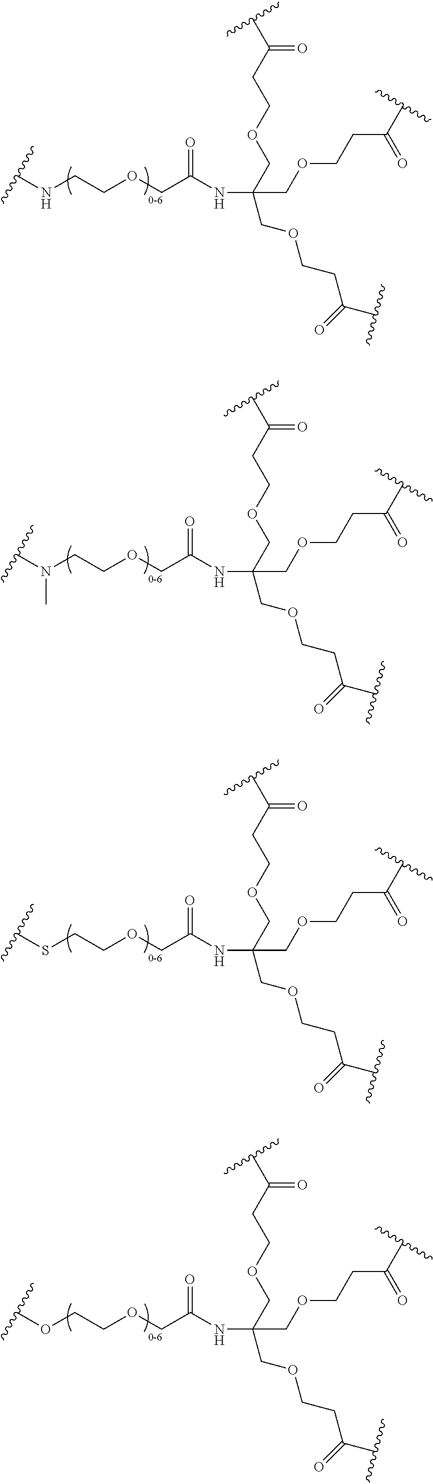

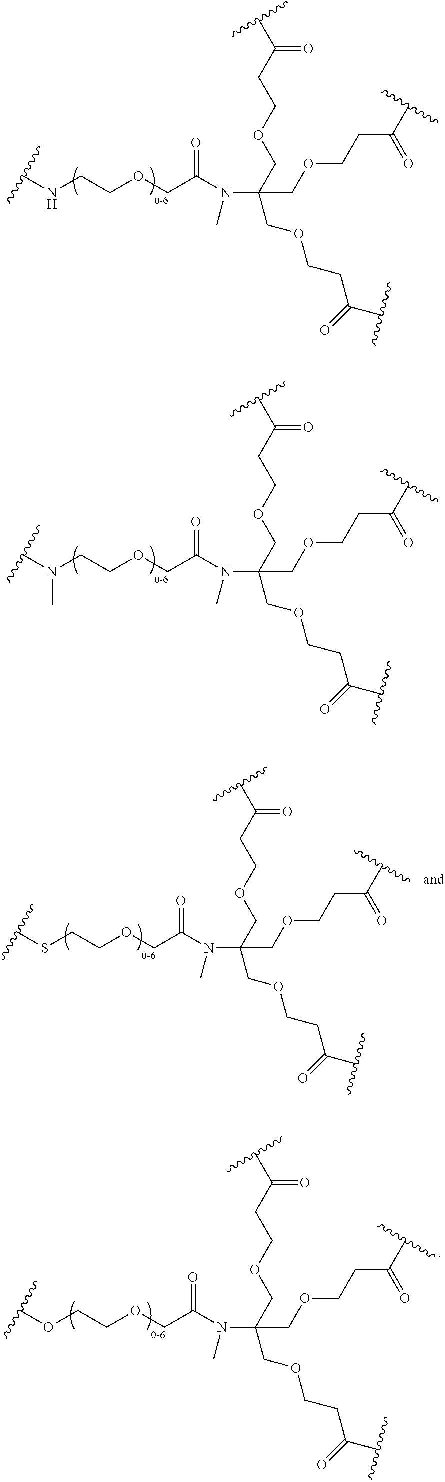

In certain embodiments, in the compound of Formula II, Linker^{D} is selected from:

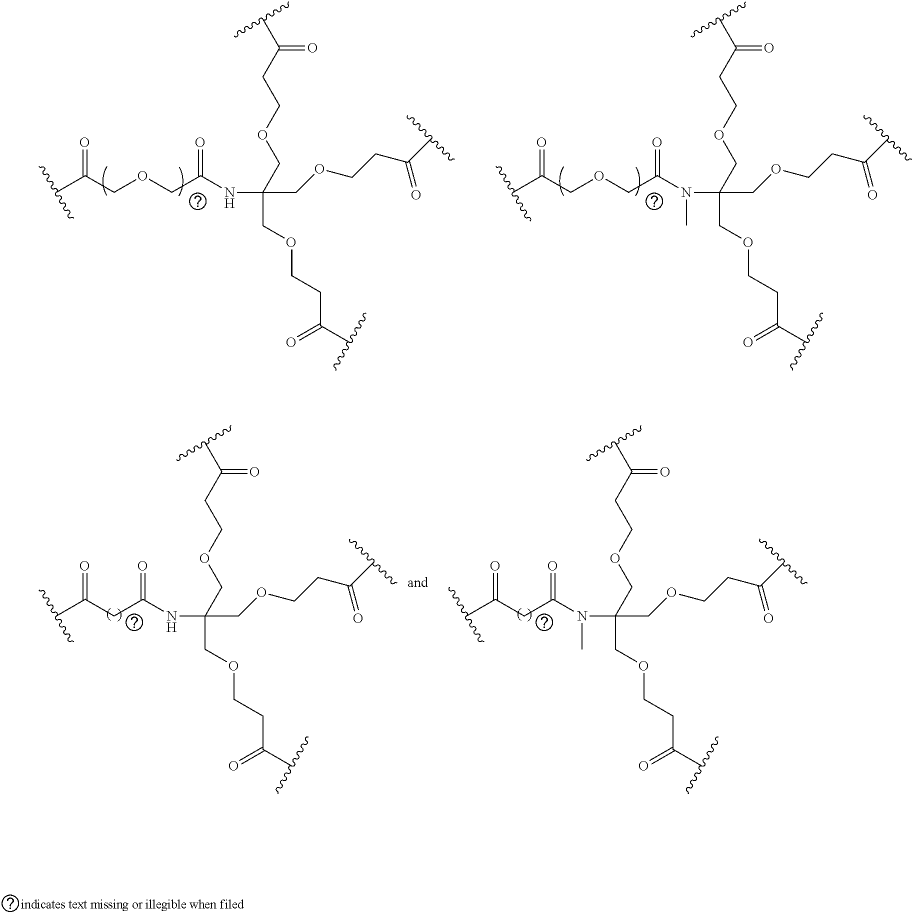

In certain embodiments, in the compound of Formula II, Linker^{D} is selected from:

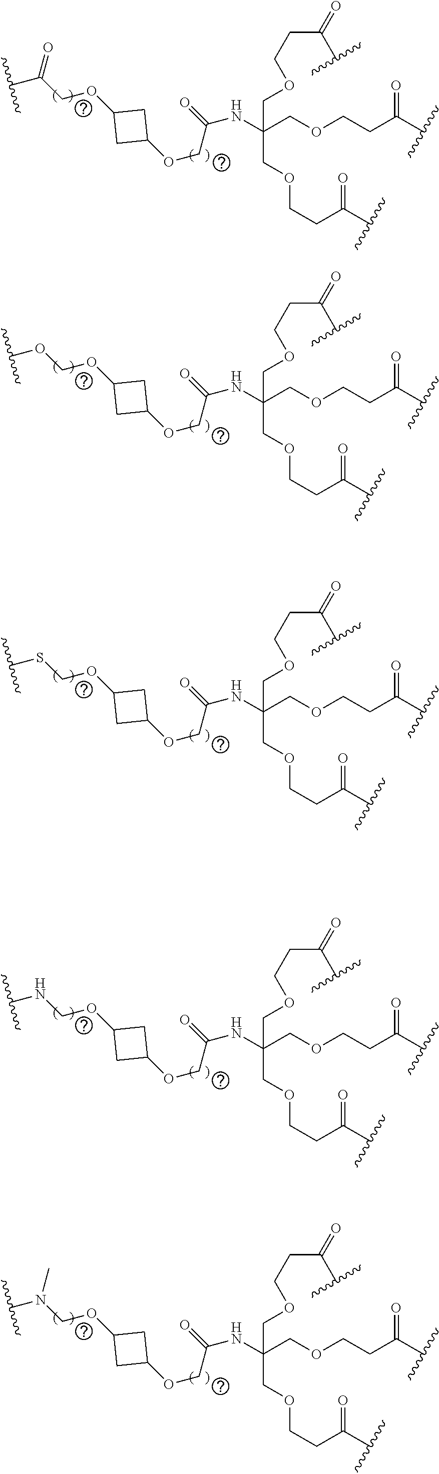

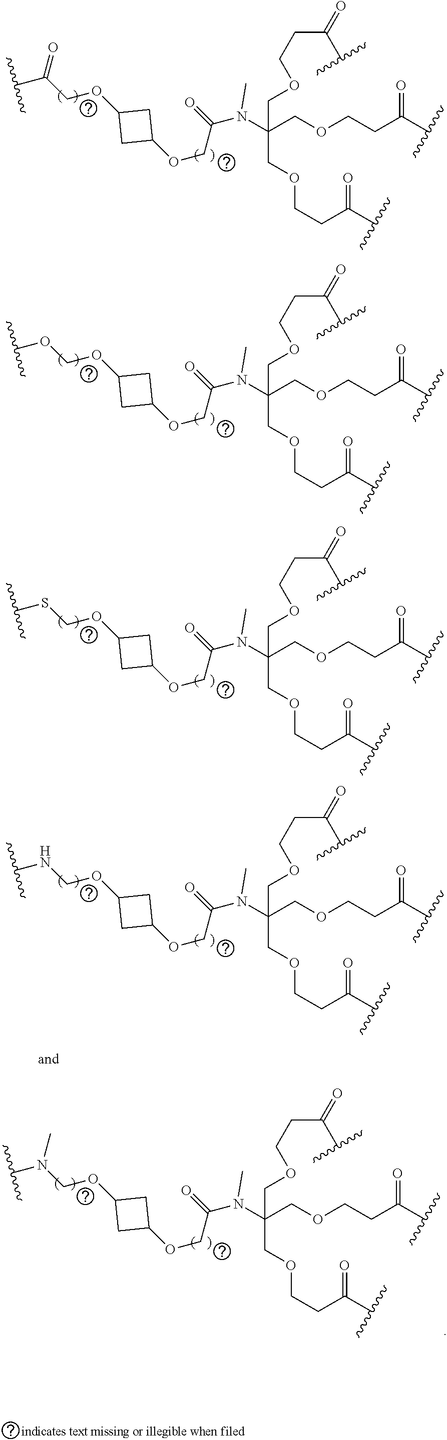

In certain embodiments, in the compound of Formula II, Linker^{D} is selected from:

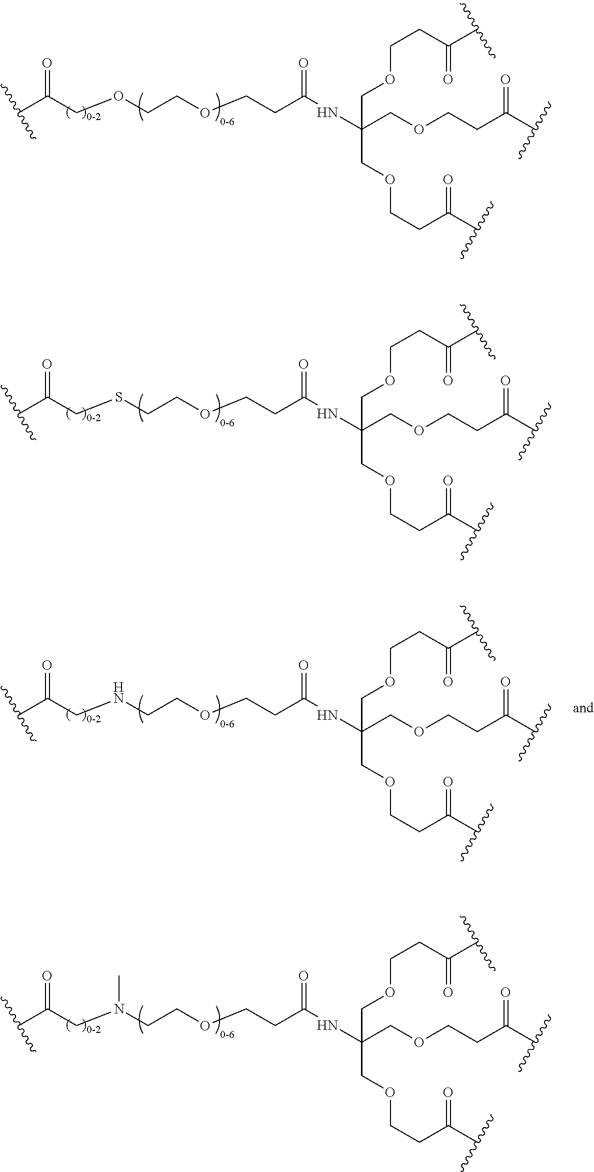

In certain embodiments, in the compound of Formula II, Linker^{D} is selected from:

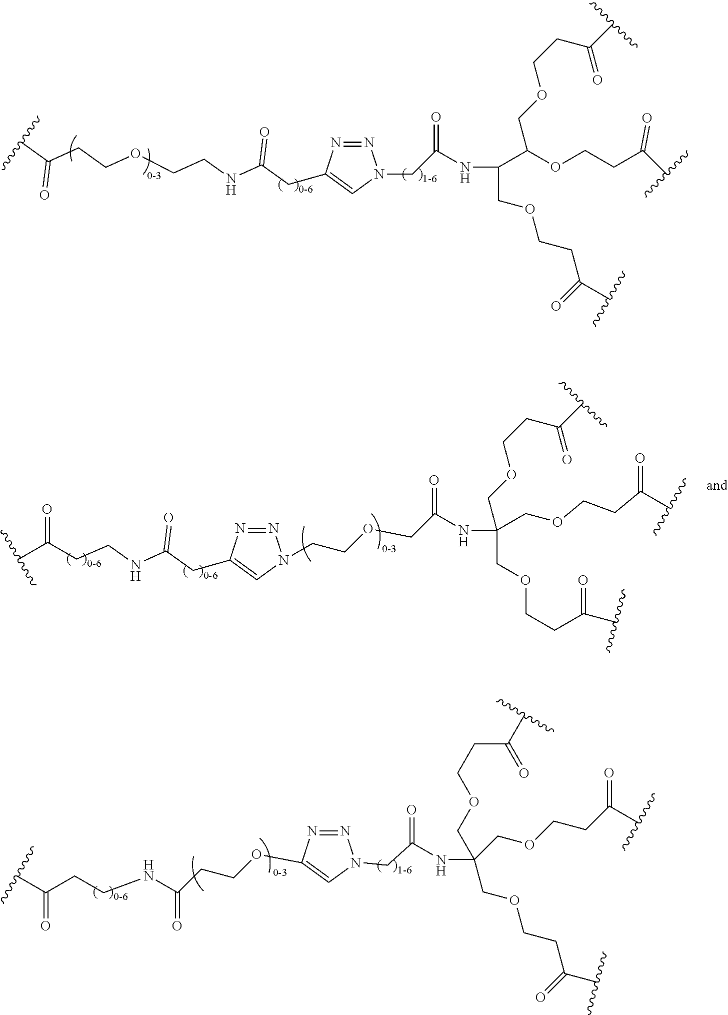

In certain embodiments, in the compound of Formula II, Linker^{D} is selected from:

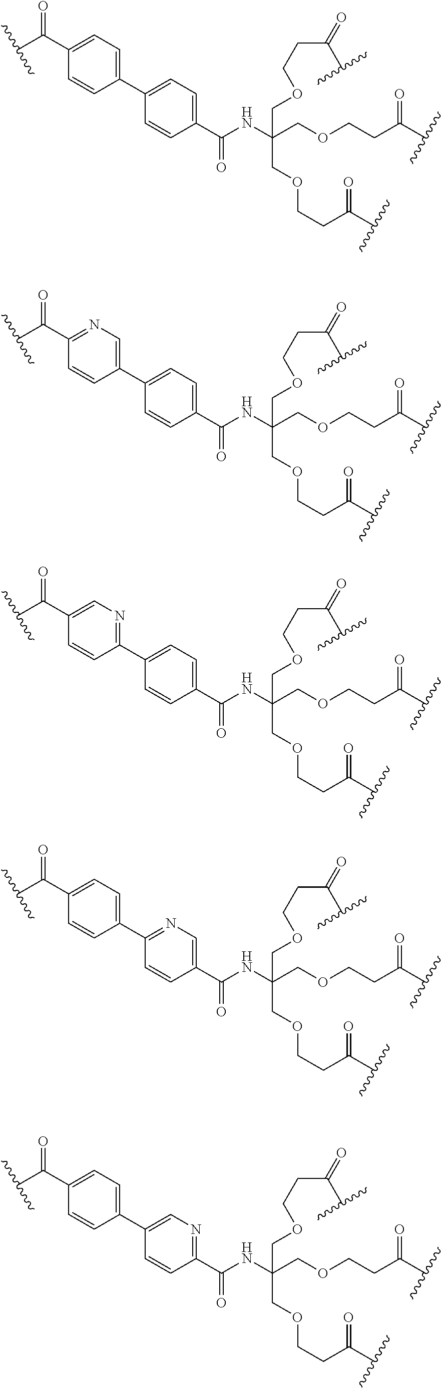 [SMILES: *C(=O)CCOCC(COCCC(*)=O)(COCCC(*)=O)NC(=O)c1ccc(-c2ccc(C(*)=O)cc2)cc1 ; *C(=O)CCOCC(COCCC(*)=O)(COCCC(*)=O)NC(=O)c1ccc(-c2ccc(C(*)=O)nc2)cc1 ; *C(=O)CCOCC(COCCC(*)=O)(COCCC(*)=O)NC(=O)c1ccc(-c2ccc(C(*)=O)cn2)cc1 ; *C(=O)CCOCC(COCCC(*)=O)(COCCC(*)=O)NC(=O)c1ccc(-c2ccc(C(*)=O)cc2)nc1 ; *C(=O)CCOCC(COCCC(*)=O)(COCCC(*)=O)NC(=O)c1ccc(-c2ccc(C(*)=O)cc2)cn1]

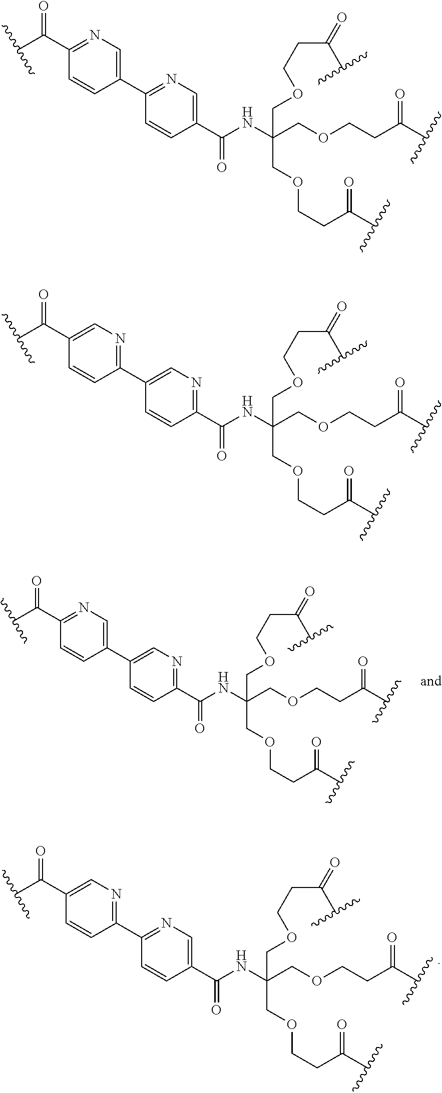 [SMILES: *C(=O)CCOCC(COCCC(*)=O)(COCCC(*)=O)NC(=O)c1ccc(-c2ccc(C(*)=O)nc2)nc1 ; *C(=O)CCOCC(COCCC(*)=O)(COCCC(*)=O)NC(=O)c1ccc(-c2ccc(C(*)=O)cn2)cn1 ; *C(=O)CCOCC(COCCC(*)=O)(COCCC(*)=O)NC(=O)c1ccc(-c2ccc(C(*)=O)nc2)cn1 ; *C(=O)CCOCC(COCCC(*)=O)(COCCC(*)=O)NC(=O)c1ccc(-c2ccc(C(*)=O)cn2)nc1]

In certain embodiments, in the compound of Formula II, the Linker^{A} is selected from

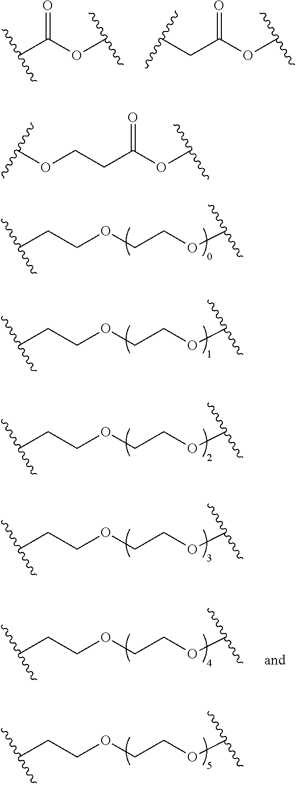

In certain embodiments, in the compound of Formula II, the Linker^{A} is selected from

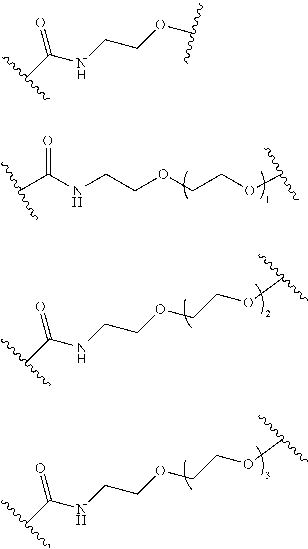

In certain embodiments in the compound of Formula II the Linker^{A} is selected from

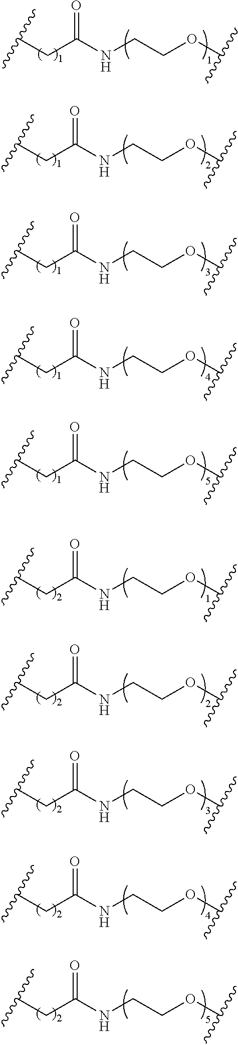

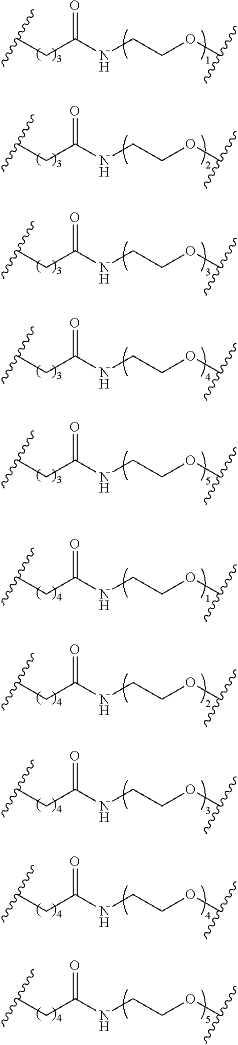

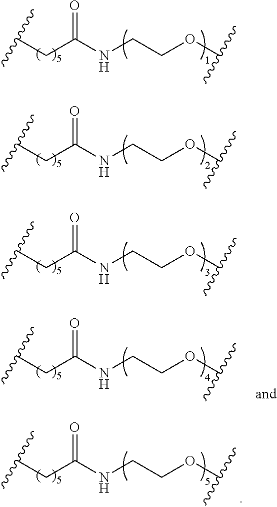

In certain embodiments, the Linker^{A} is selected from

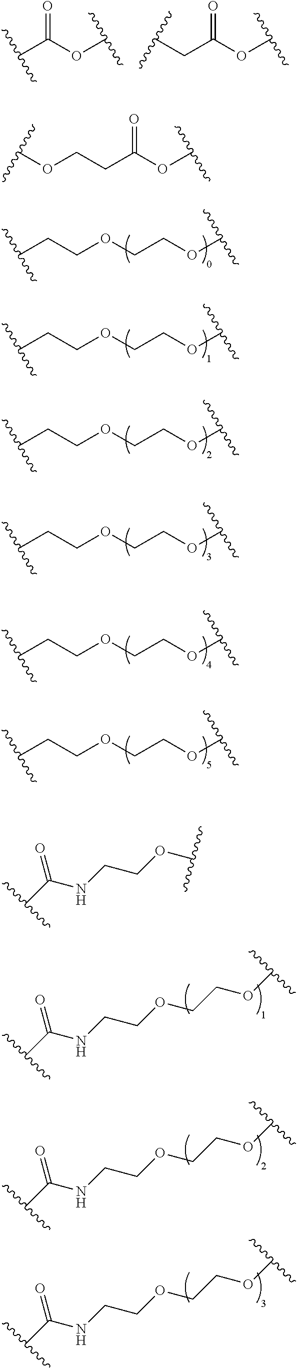

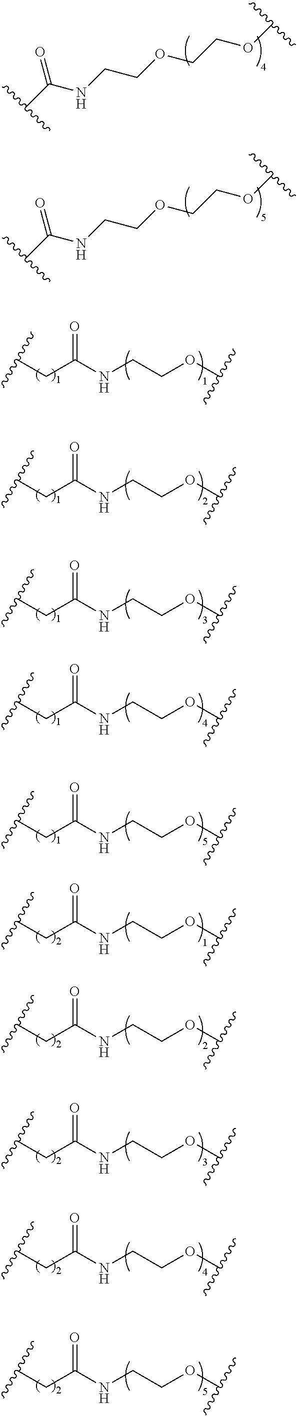

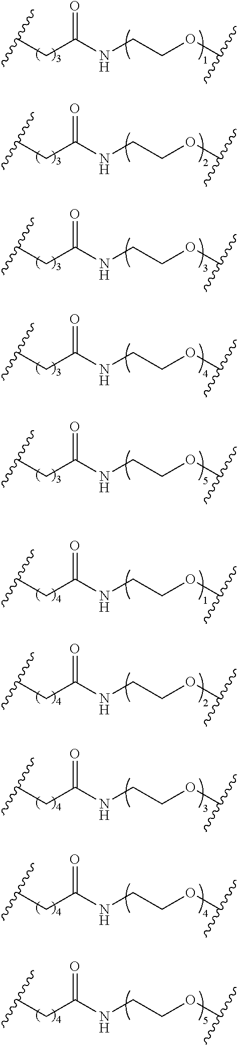

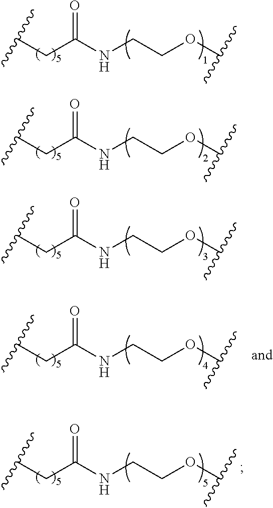

wherein each is optionally substituted with 1, 2, 3, or 4 substituents substituent selected from R²¹.

In certain embodiments, in the compound of Formula II, Linker^{A} is selected from:

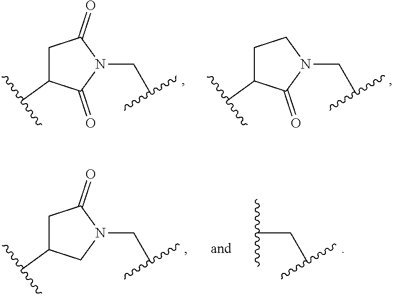

In certain embodiments, in the compound of Formula II, the Linker^{A} is selected from

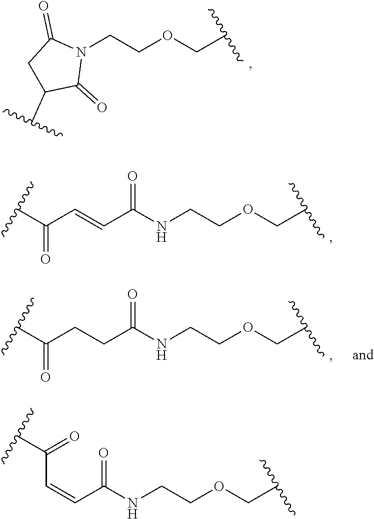 [SMILES: *COCCN1C(=O)CC(*)C1=O ; *COCCNC(=O)/C=C/C(*)=O ; *COCCNC(=O)CCC(*)=O ; *COCCNC(=O)/C=C\C(*)=O]

In certain embodiments, in the compound of Formula II, the Linker^{A} is selected from

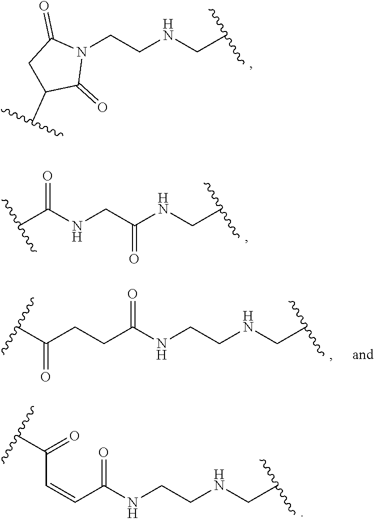

In certain embodiments in the compound of Formula II, the Linker^{A} is selected from

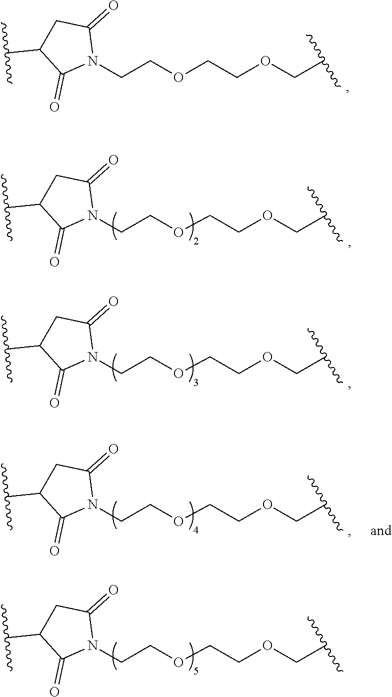

In certain embodiments, in the compound of Formula II the Linker^{A} is selected from

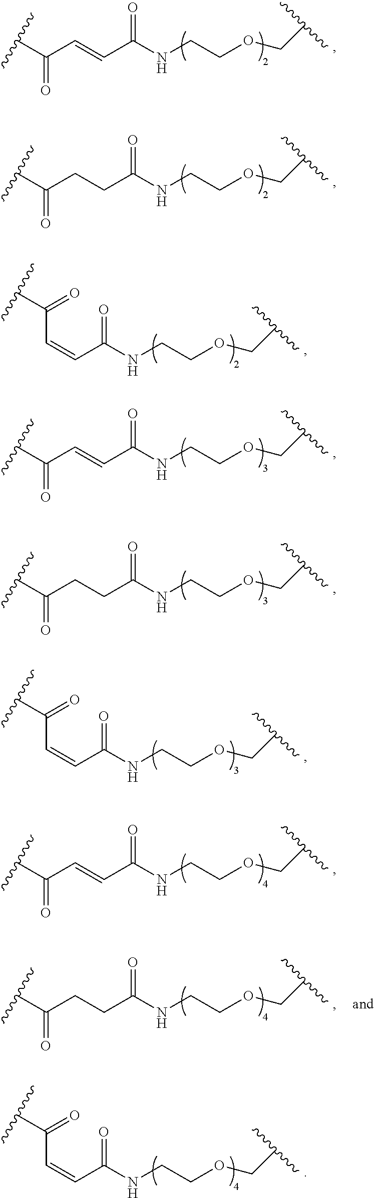

In certain embodiments, in the compound of Formula II, the Linker^{A} is selected from

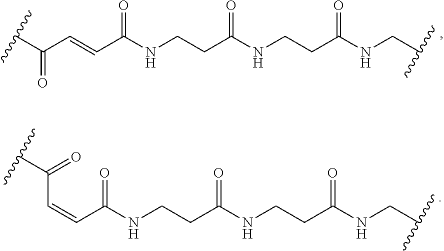 [SMILES: *CNC(=O)CCNC(=O)CCNC(=O)/C=C/C(*)=O ; *CNC(=O)CCNC(=O)CCNC(=O)/C=C\C(*)=O]

In certain embodiments, in the compound of Formula II, the Linker^{A} is selected from

In certain embodiments, in the compound of Formula II, the Linker^{A} is selected from

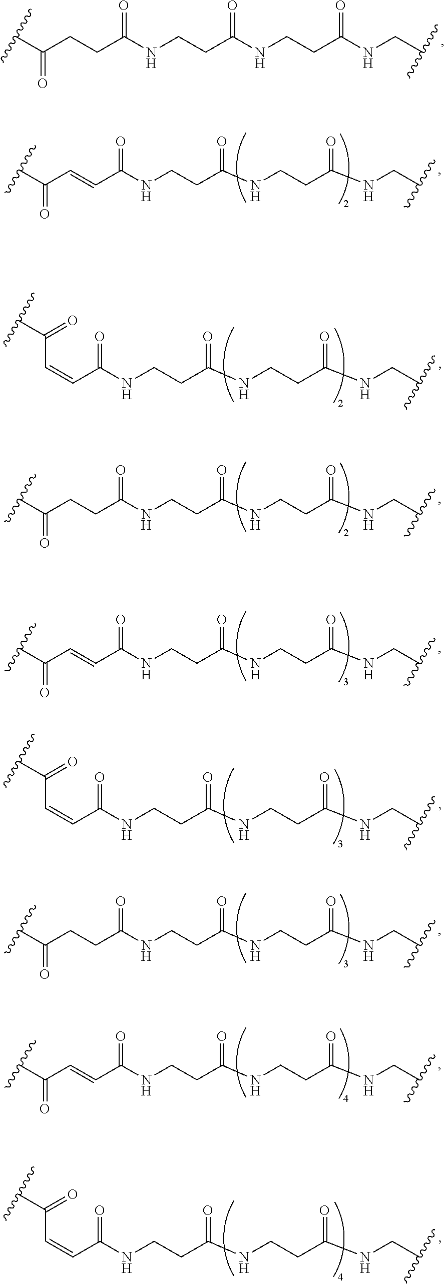

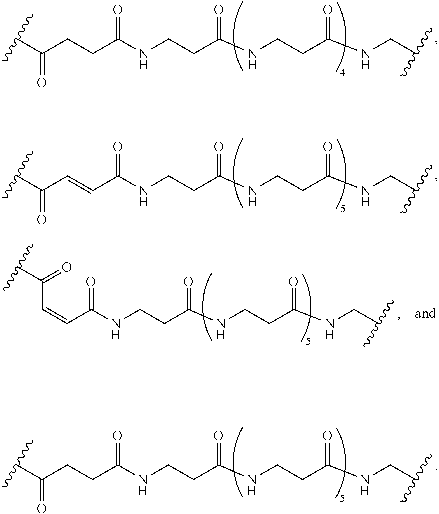

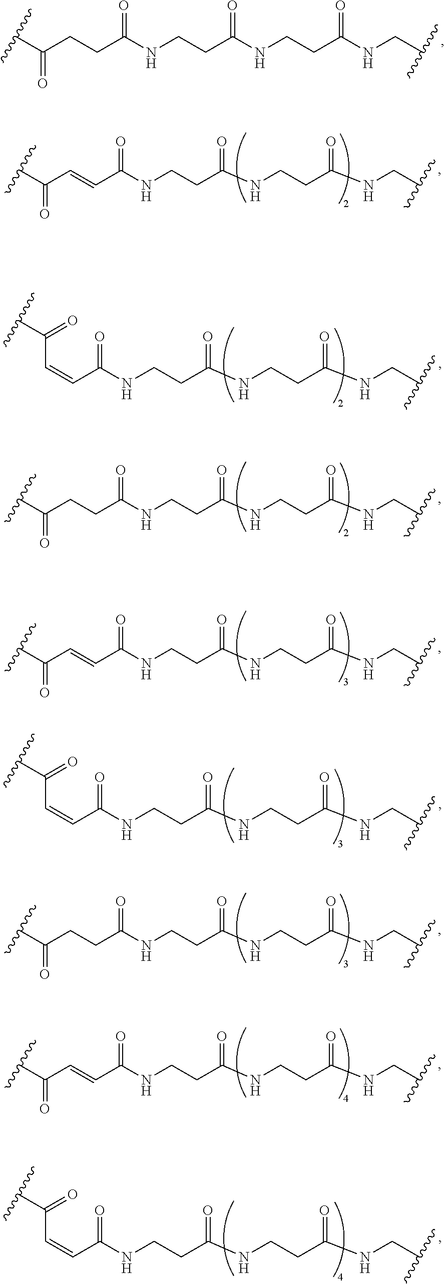

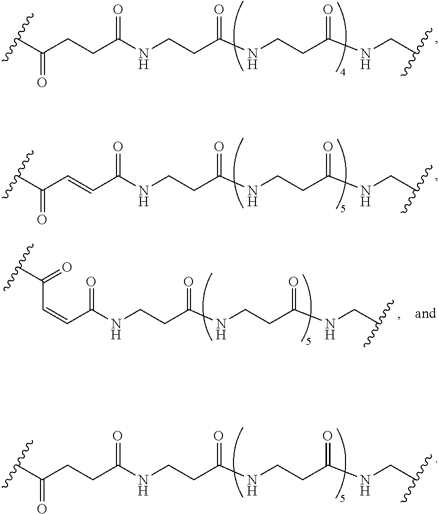

In certain embodiments in the compound of Formula II, the Linker^{A} is selected from

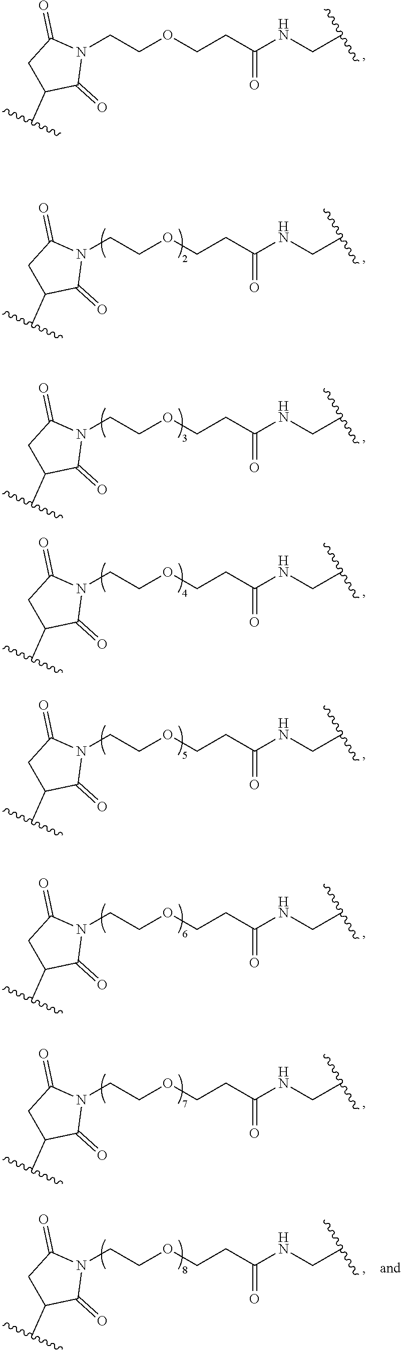

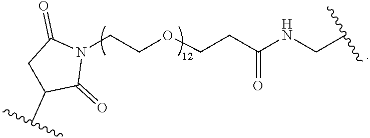

In certain embodiments, in the compound of Formula II, the Linker^{A} is selected from

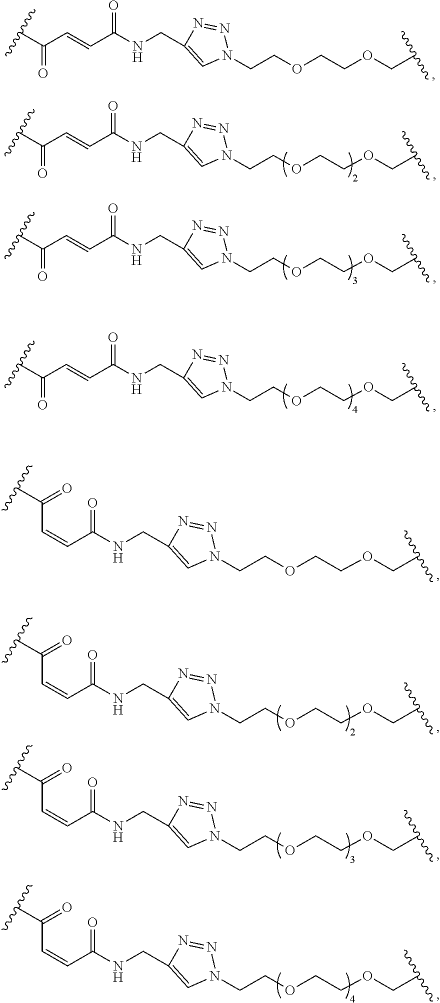

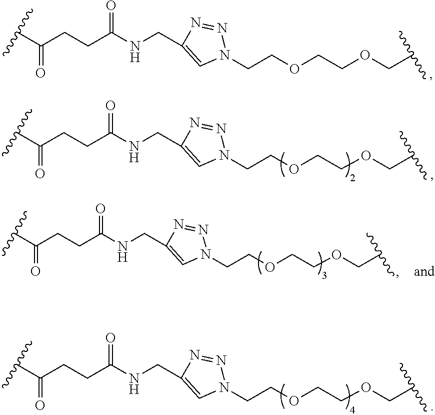

In certain embodiments, in the compound of Formula II, the Linker^{A} is selected from

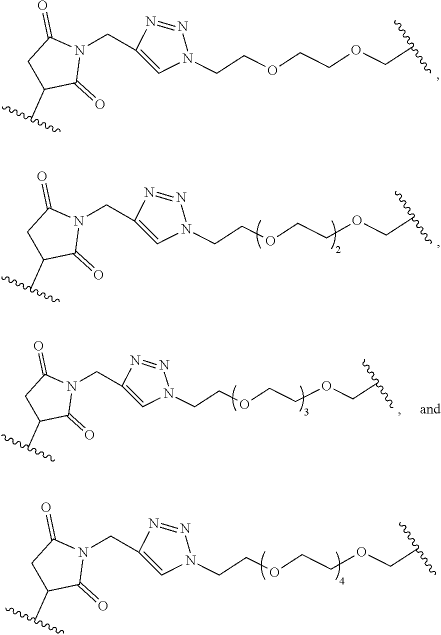

In certain embodiments, in the compound of Formula II, the Linker^{A} is selected from

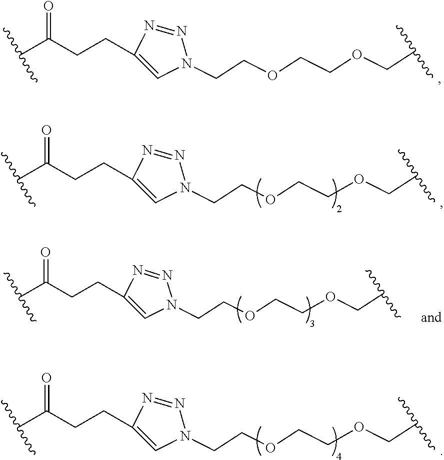

In certain embodiments, in the compound of Formula II, the Linker^{A} is selected from

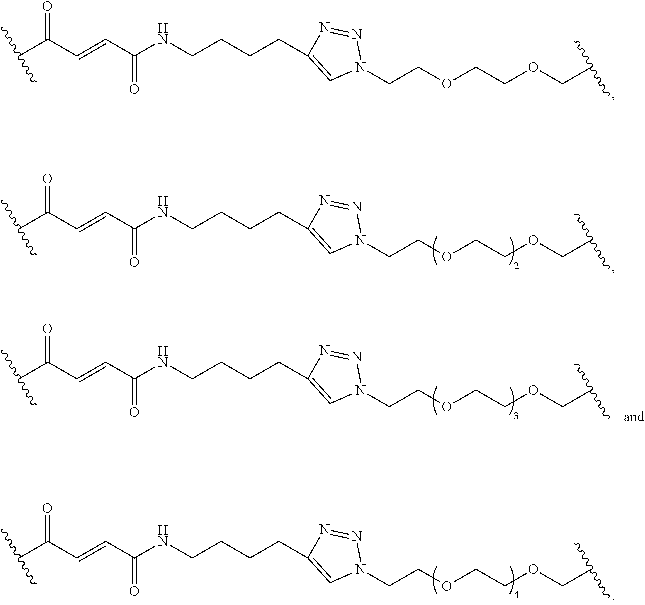

In certain embodiments in the compound of Formula II, the Linker^{A} is selected from

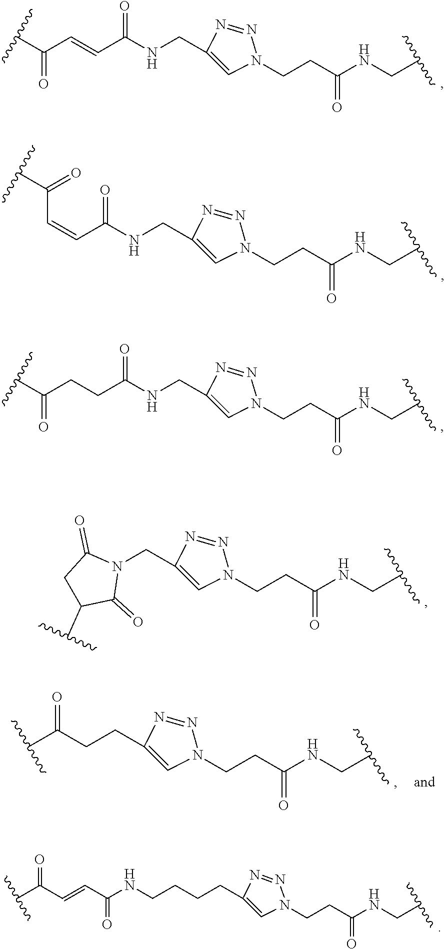 [SMILES: *CNC(=O)CCn1cc(CNC(=O)/C=C/C(*)=O)nn1 ; *CNC(=O)CCn1cc(CNC(=O)/C=C\C(*)=O)nn1 ; *CNC(=O)CCn1cc(CNC(=O)CCC(*)=O)nn1 ; *CNC(=O)CCn1cc(CN2C(=O)CC(*)C2=O)nn1 ; *CNC(=O)CCn1cc(CCC(*)=O)nn1 ; *CNC(=O)CCn1cc(CCCCNC(=O)/C=C/C(*)=O)nn1]

In certain embodiments in the compound of Formula II the Linker^{A} is selected from

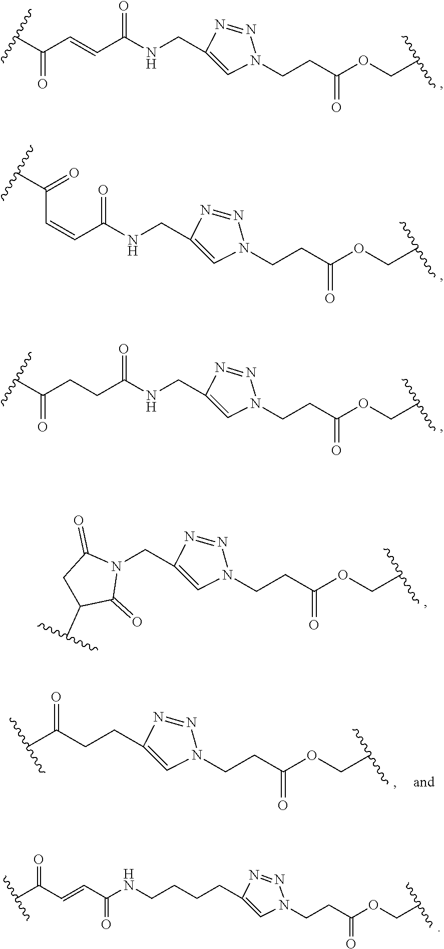 [SMILES: *COC(=O)CCn1cc(CNC(=O)/C=C/C(*)=O)nn1 ; *COC(=O)CCn1cc(CNC(=O)/C=C\C(*)=O)nn1 ; *COC(=O)CCn1cc(CNC(=O)CCC(*)=O)nn1 ; *COC(=O)CCn1cc(CN2C(=O)CC(*)C2=O)nn1 ; *COC(=O)CCn1cc(CCC(*)=O)nn1 ; *COC(=O)CCn1cc(CCCCNC(=O)/C=C/C(*)=O)nn1]

In certain embodiments in the compound of Formula II the Linker^{B} is selected from

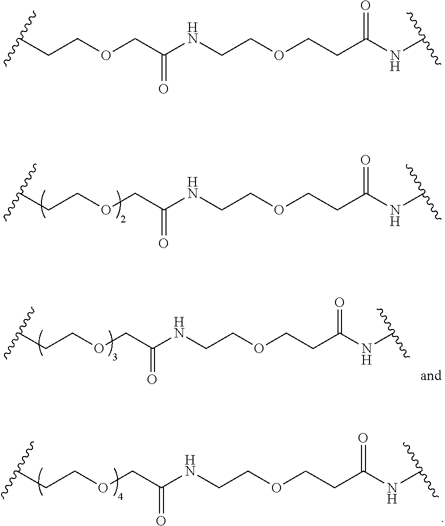

In certain embodiments, in the compound of Formula II, the Linker^{B} is selected from

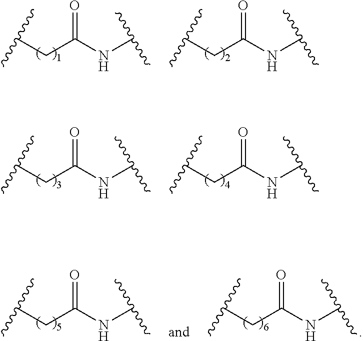

In certain embodiments, in the compound of Formula II, the Linker^{B} is selected from

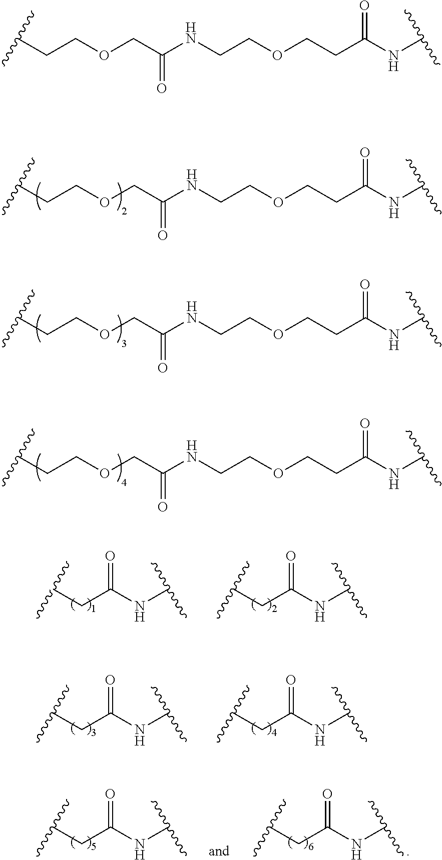

In certain embodiments, in the compound of Formula II, the Linker^{B} is selected from wherein each is optionally substituted with 1, 2, 3, or 4 substituents substituent selected from R²¹.

In certain embodiments, in the compound of Formula II Linker^{B} is selected from:

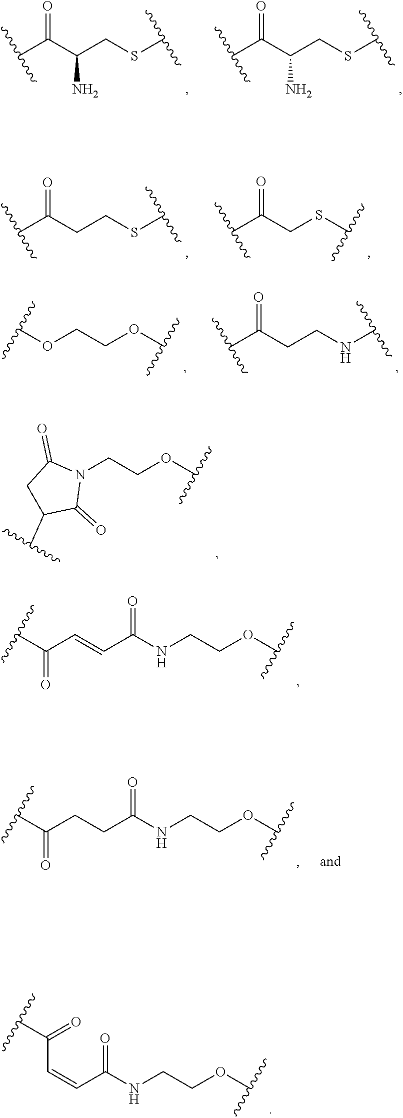

In certain embodiments, in the compound of Formula II, the Linker^{B} is selected from:

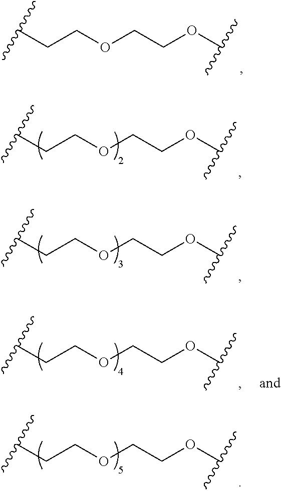

In certain embodiments, in the compound of Formula II, the Linker^{B} is selected from:

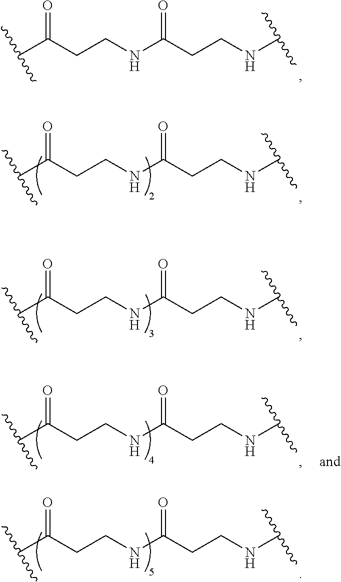

In certain embodiments, in the compound of Formula II, the Linker^{B} is selected from:

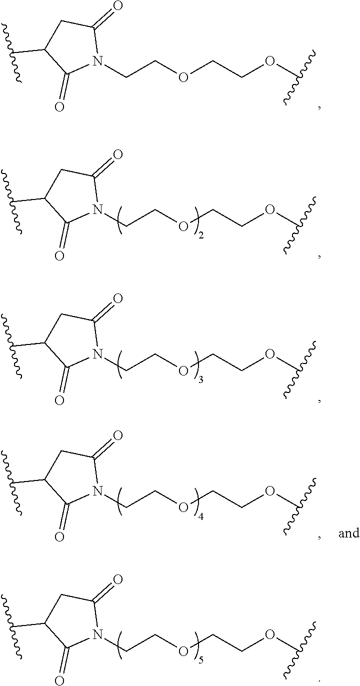

In certain embodiments, in the compound of Formula II, the Linker^{B} is selected from:

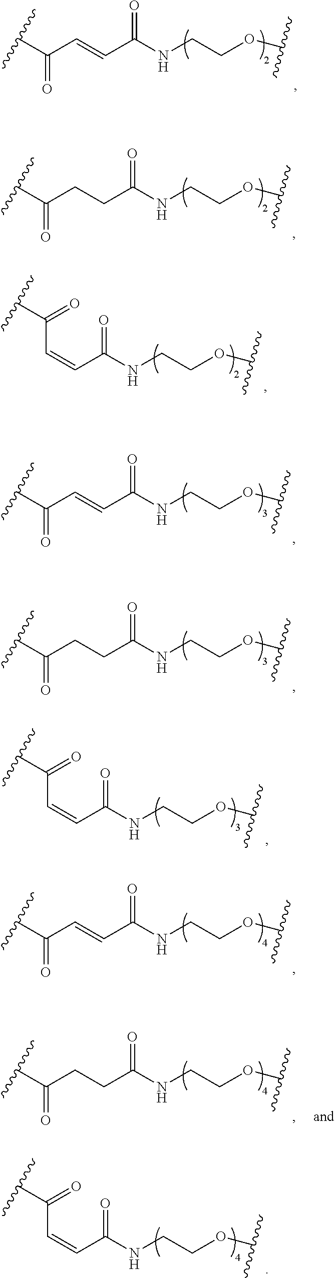

In certain embodiments, in the compound of Formula II, the Linker^{B} is selected from:

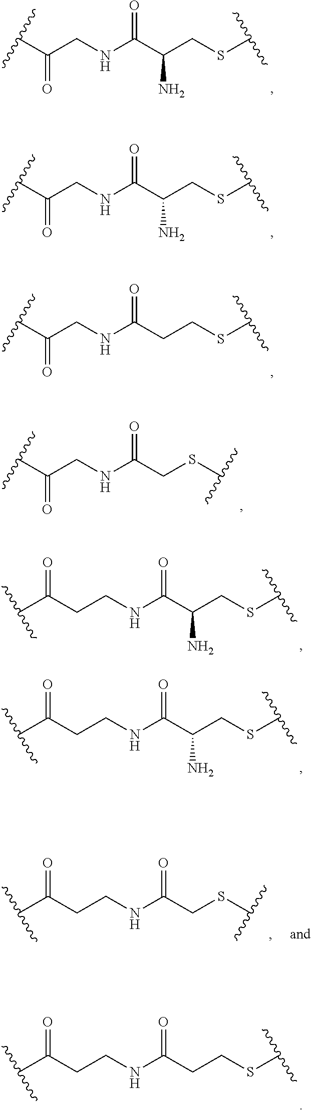

In certain embodiments, in the compound of Formula II, the Linker^{B} is selected from:

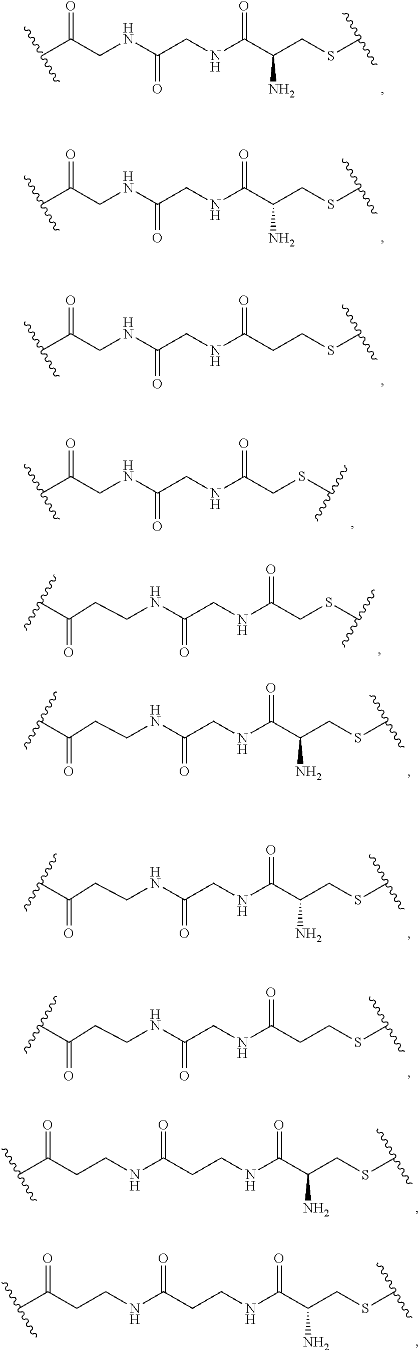

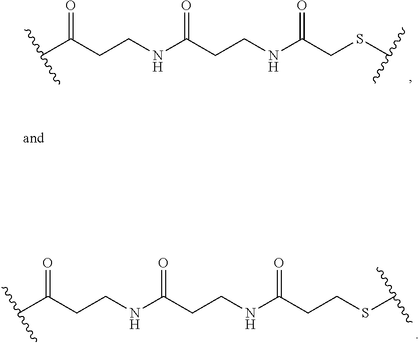 [SMILES: *SCC(=O)NCCC(=O)NCCC(*)=O ; *SCCC(=O)NCCC(=O)NCCC(*)=O]

In certain embodiments, in the compound of Formula II, the Linker^{B} is selected from:

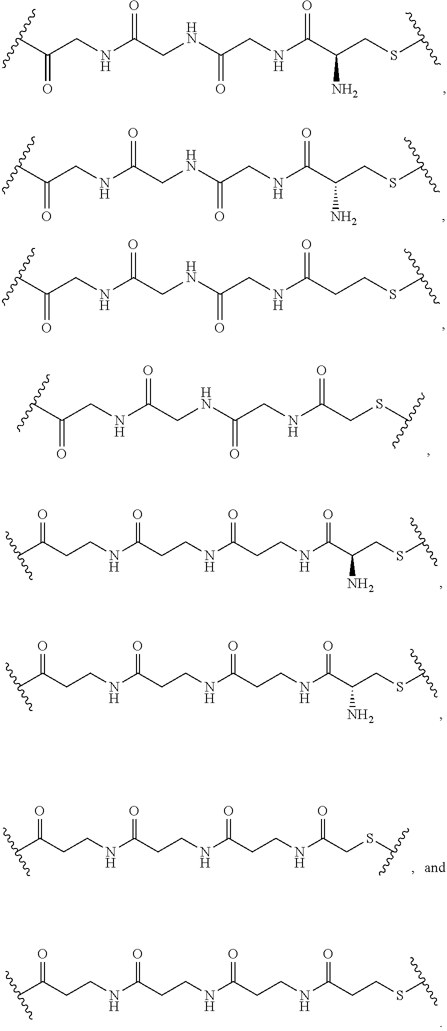

In certain embodiments in the compound of Formula II Linker^{B}-Linker^{A} is selected from:

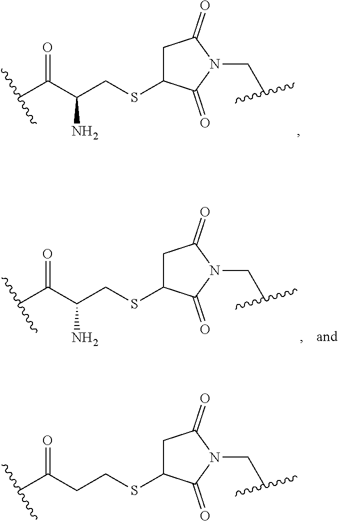 [SMILES: *CN1C(=O)CC(SC[C@@H](N)C(*)=O)C1=O ; *CN1C(=O)CC(SC[C@H](N)C(*)=O)C1=O ; *CN1C(=O)CC(SCCC(*)=O)C1=O]

In certain embodiments, in the compound of Formula II, Linker^{B}-Linker^{A} is selected from:

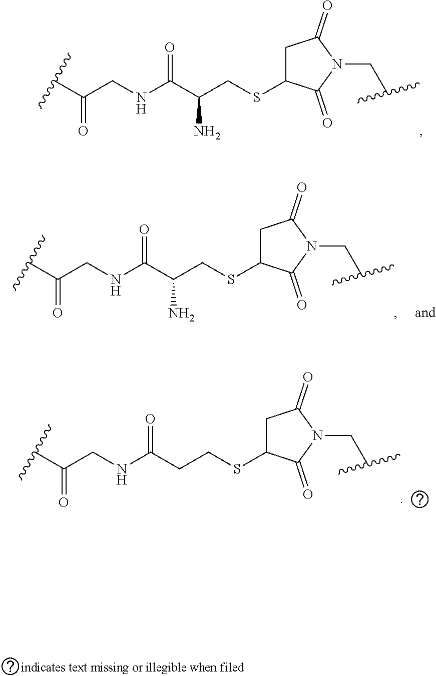 [SMILES: *CN1C(=O)CC(SC[C@@H](N)C(=O)NCC(*)=O)C1=O ; *CN1C(=O)CC(SC[C@H](N)C(=O)NCC(*)=O)C1=O ; *CN1C(=O)CC(SCCC(=O)NCC(*)=O)C1=O]

In certain embodiments, in the compound of Formula II, the Linker^{C} is selected from:

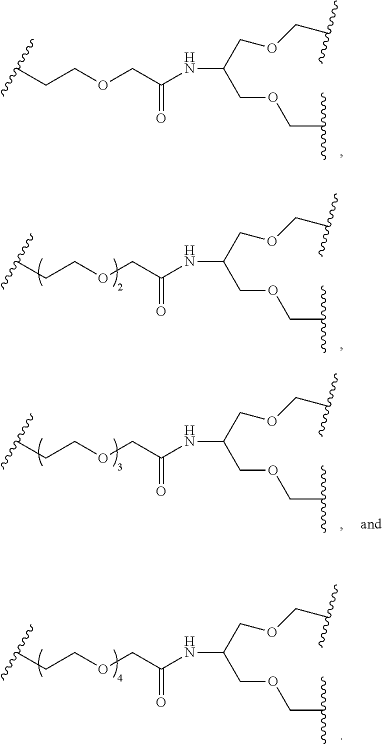

In certain embodiments, in the compound of Formula II, the Linker^{C} is selected from:

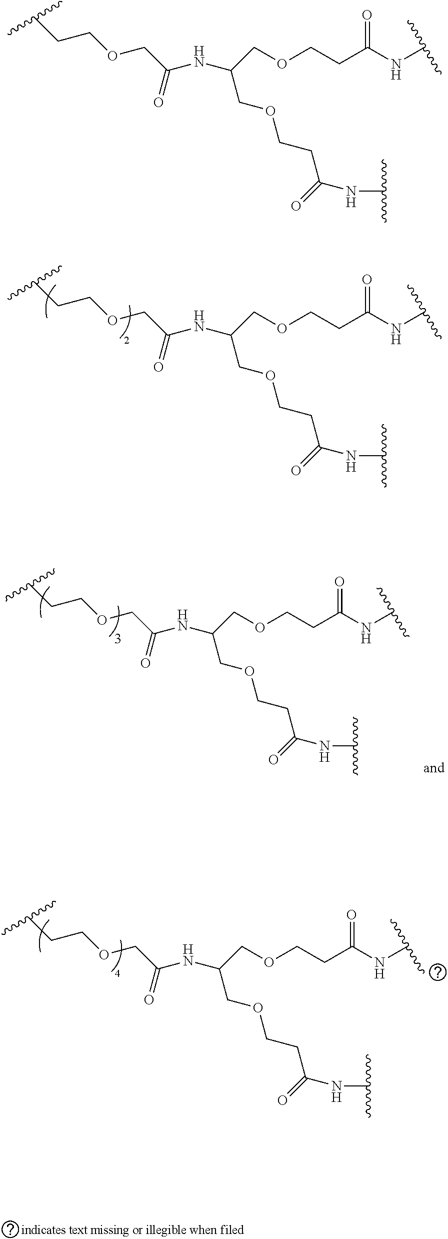

In certain embodiments, in the compound of Formula II, the Linker^{C} is selected from:

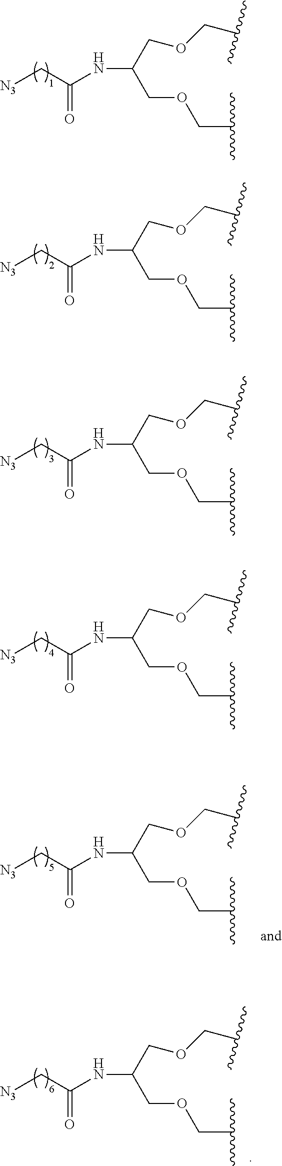

In certain embodiments in the compound of Formula II the Linker^{C} is selected from:

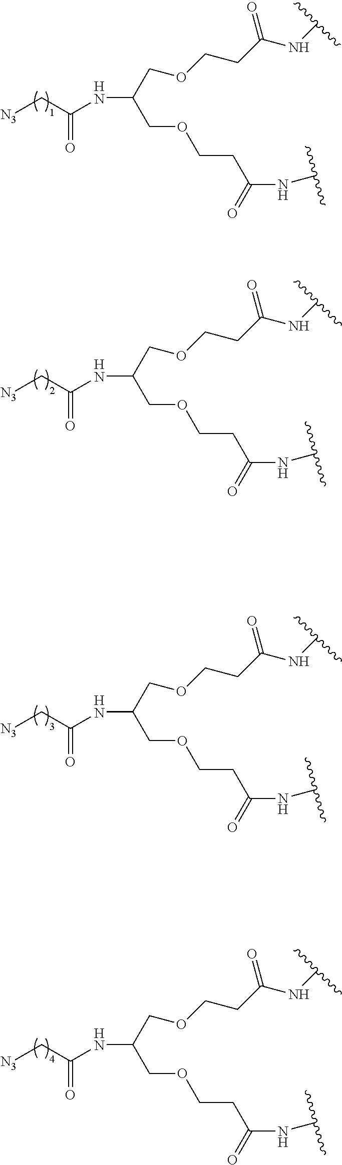

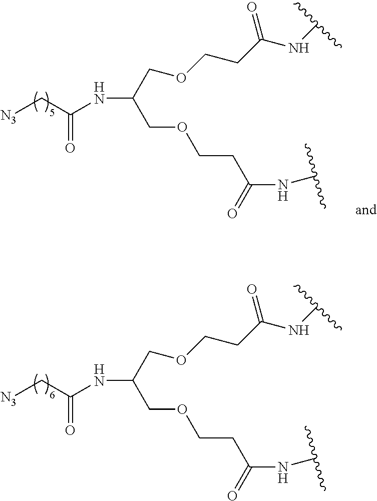

In certain embodiments, in the compound of Formula II, the Linker^{C} is selected from: II

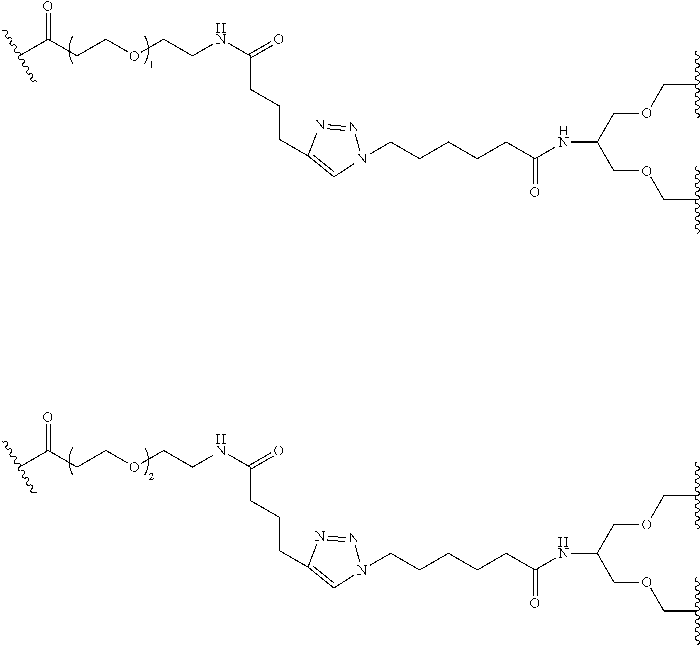

In certain embodiments in the compound of Formula II, the Linker^{C} is selected from:

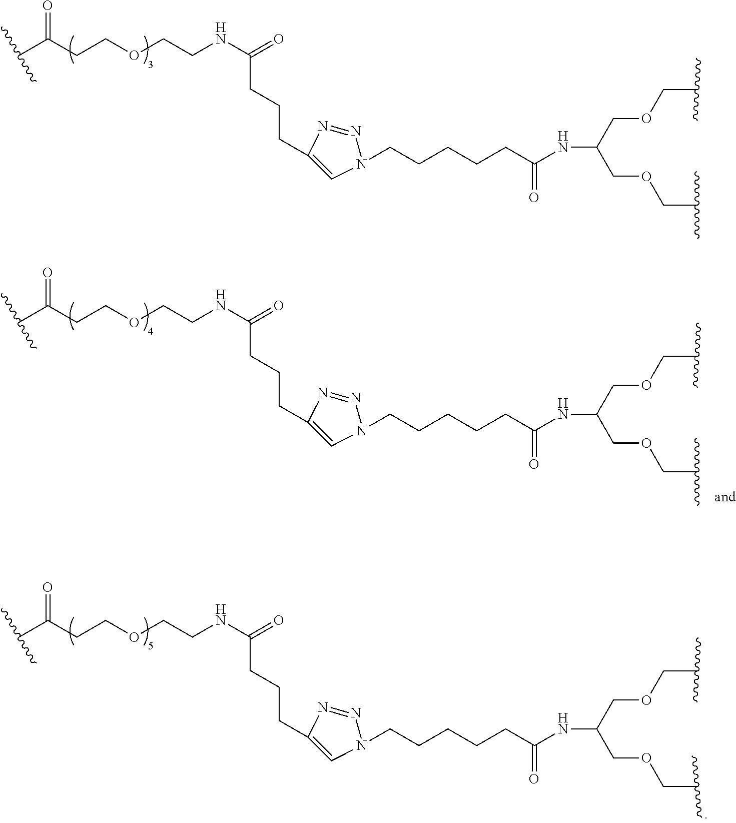

In certain embodiments, in the compound of Formula II, the Linker^{C} is selected from:

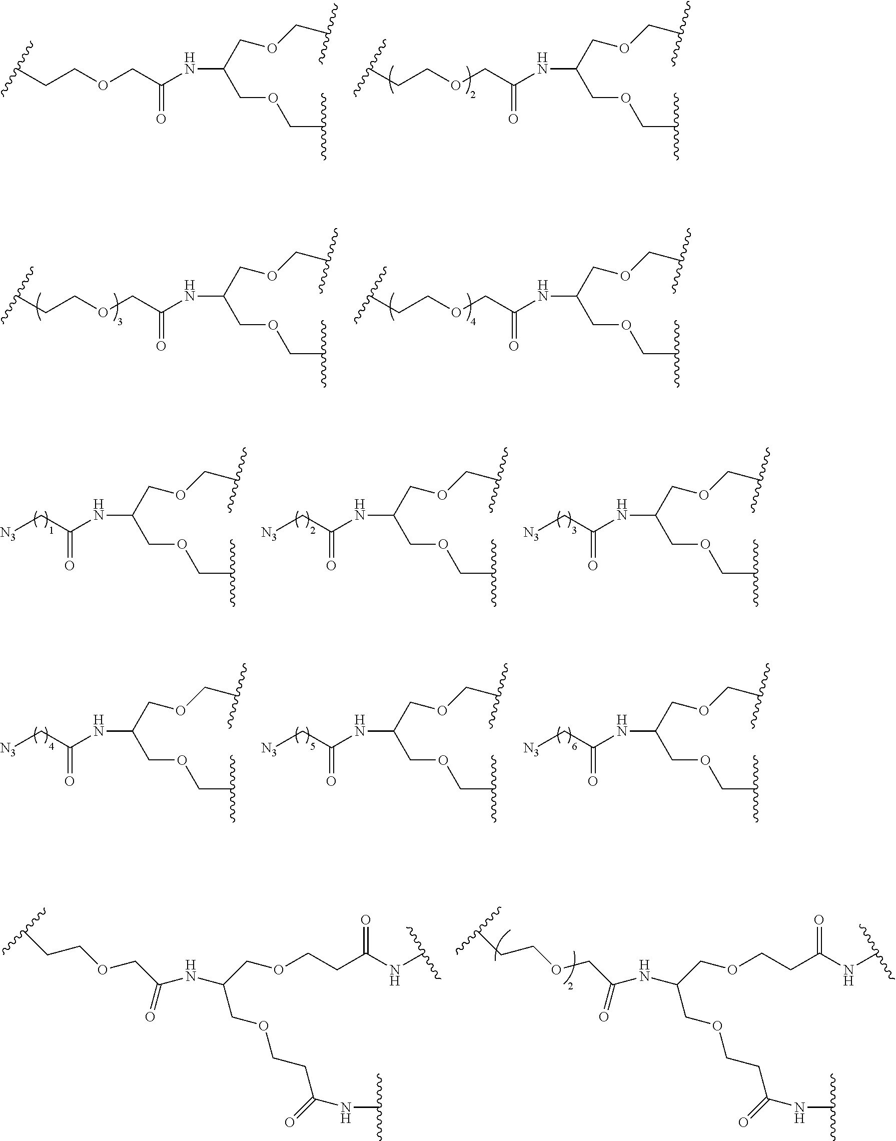

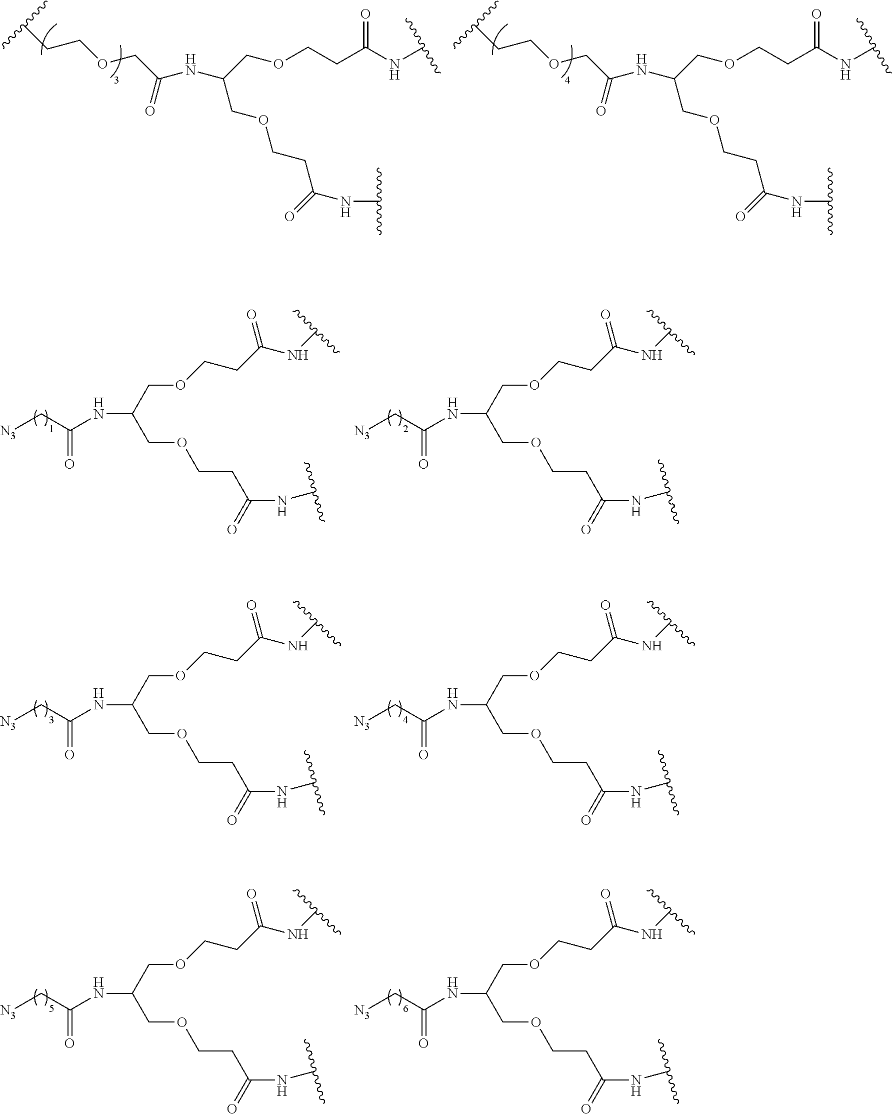

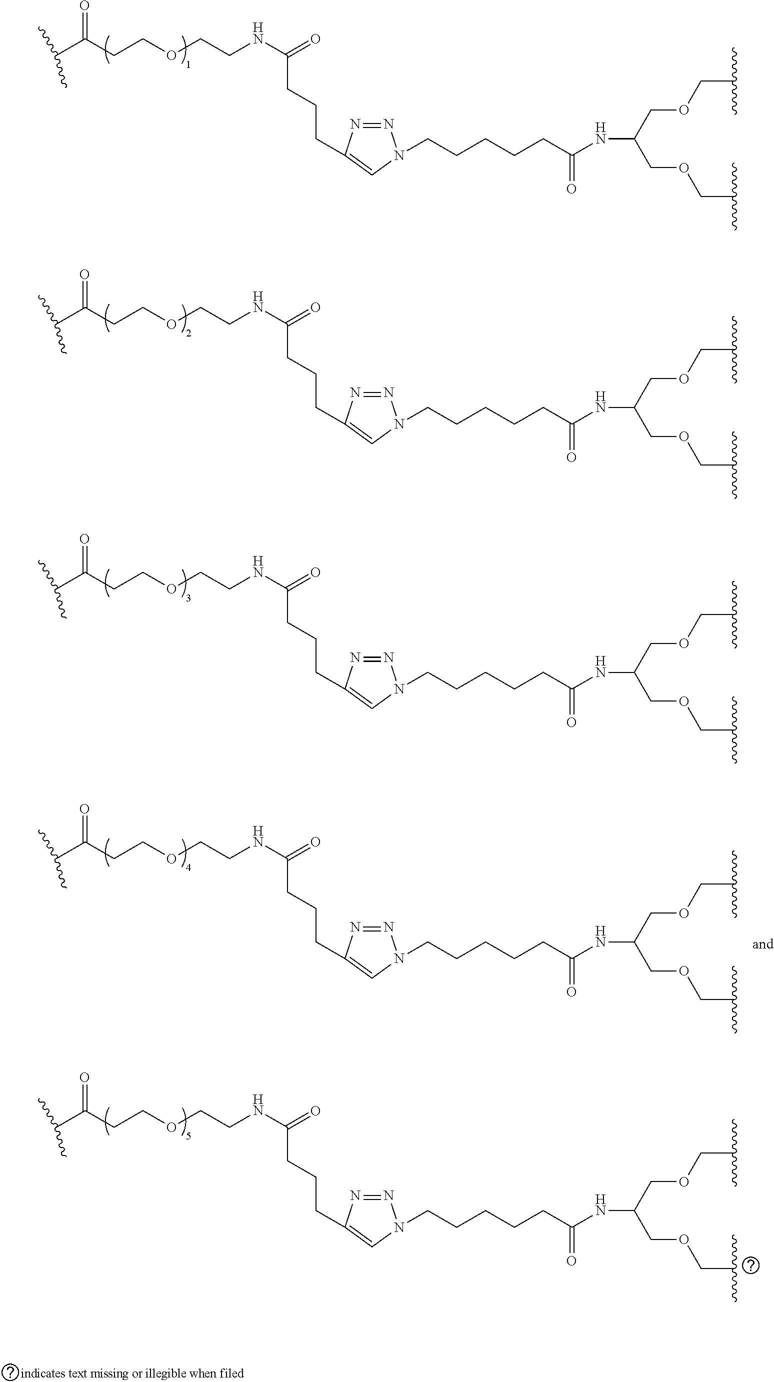

In certain embodiments, in the compound of Formula II, the Linker^{C} is selected from: wherein each is optionally substituted with 1, 2, 3, or 4 substituents substituent selected from R²¹.

In certain embodiments, in the compound of Formula II, the Linker^{C} is selected from:

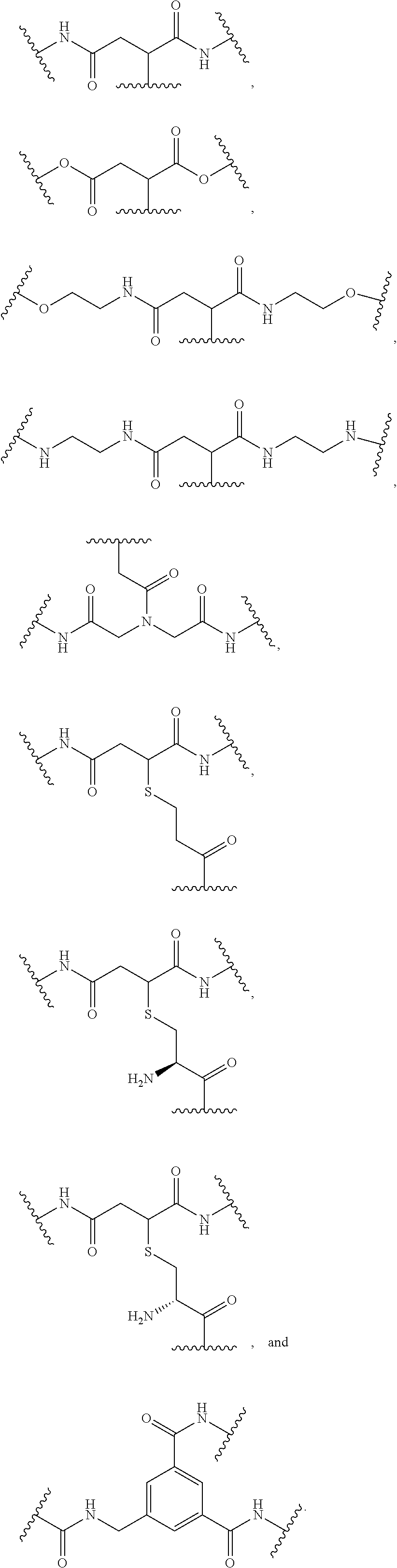

In certain embodiments, in the compound of Formula II, the Linker^{C} is selected from:

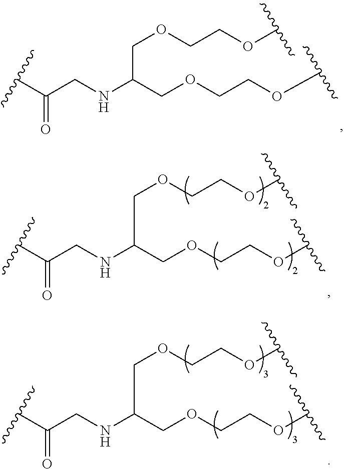

In certain embodiments, in the compound of Formula II, the Linker^{C} is selected from:

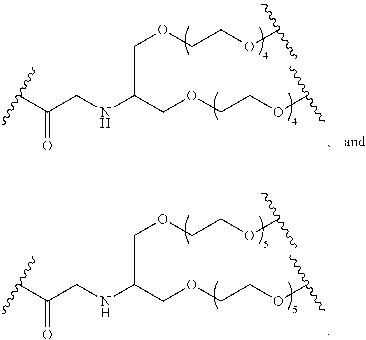

In certain embodiments, in the compound of Formula II, the Linker^{C} is selected from:

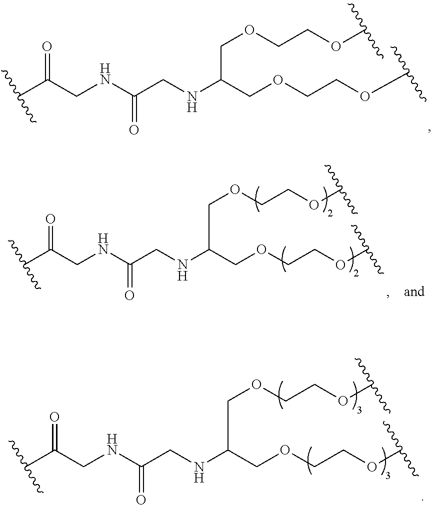

In certain embodiments, in the compound of Formula II, the Linker^{C} is selected from:

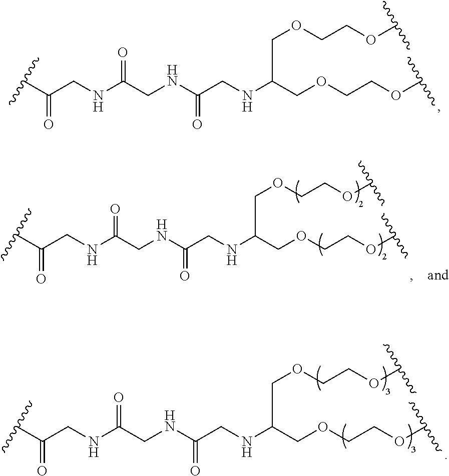

In certain embodiments, in the compound of Formula II, the Linker^{C} is selected from:

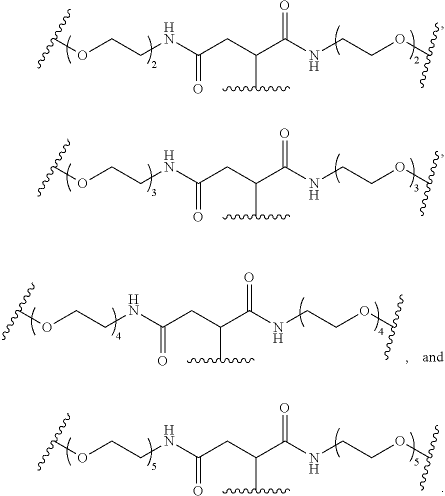

In certain embodiments, in the compound of Formula II, the Linker^{C} is selected from:

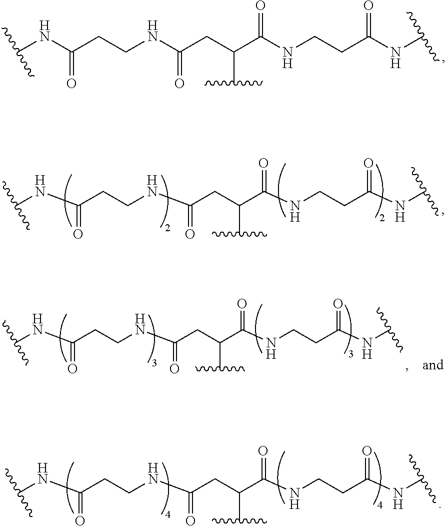

In certain embodiments, in the compound of Formula II, the Linker^{C} is selected from:

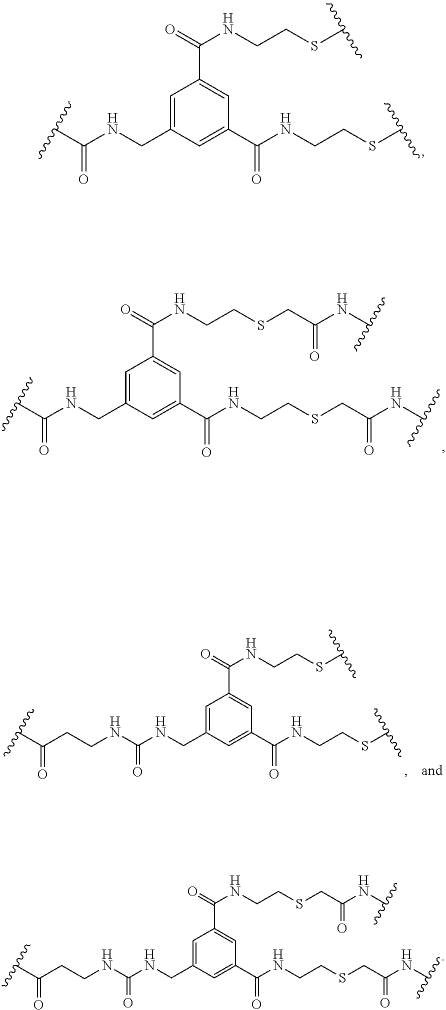 [SMILES: *SCCNC(=O)c1cc(CNC(*)=O)cc(C(=O)NCCS*)c1 ; *NC(=O)CSCCNC(=O)c1cc(CNC(*)=O)cc(C(=O)NCCSCC(=O)N*)c1 ; *SCCNC(=O)c1cc(CNC(=O)NCCC(*)=O)cc(C(=O)NCCS*)c1 ; *NC(=O)CSCCNC(=O)c1cc(CNC(=O)NCCC(*)=O)cc(C(=O)NCCSCC(=O)N*)c1]

In certain embodiments, in the compound of Formula II, Linker^{C}-(Linker^{A})₂ is selected from:

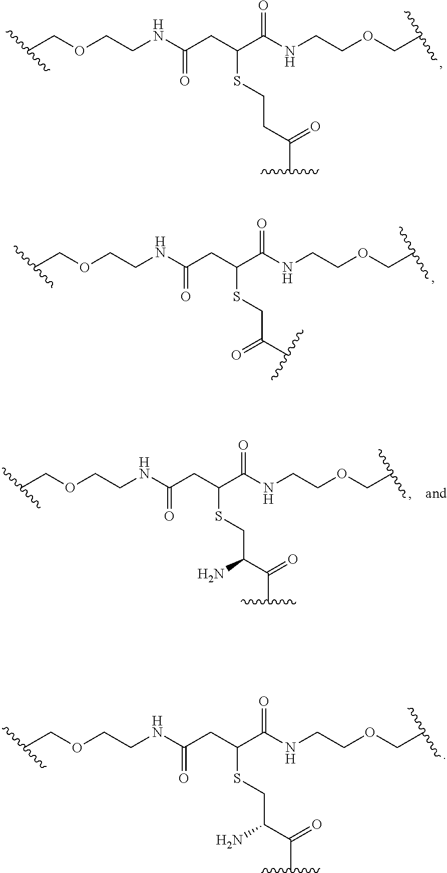 [SMILES: *COCCNC(=O)CC(SCCC(*)=O)C(=O)NCCOC* ; *COCCNC(=O)CC(SCC(*)=O)C(=O)NCCOC* ; *COCCNC(=O)CC(SC[C@H](N)C(*)=O)C(=O)NCCOC* ; *COCCNC(=O)CC(SC[C@@H](N)C(*)=O)C(=O)NCCOC*]

In certain embodiments, in the compound of Formula II, Linker^{C}-(Linker^{A})₂ is selected from:

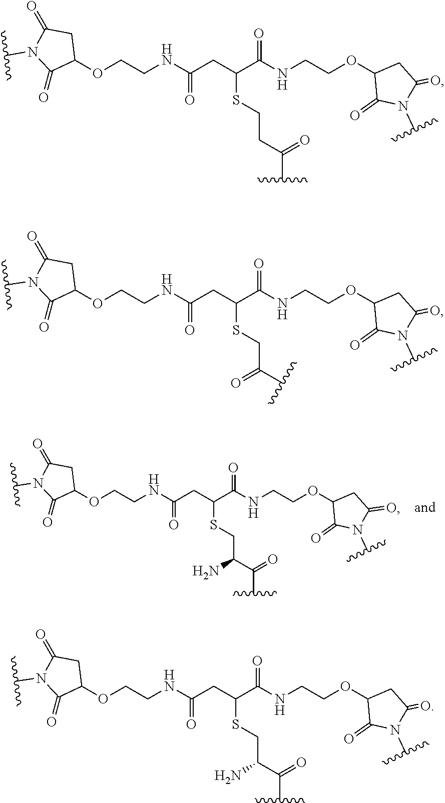 [SMILES: *C(=O)CCSC(CC(=O)NCCOC1CC(=O)N(*)C1=O)C(=O)NCCOC1CC(=O)N(*)C1=O ; *C(=O)CSC(CC(=O)NCCOC1CC(=O)N(*)C1=O)C(=O)NCCOC1CC(=O)N(*)C1=O ; *C(=O)[C@@H](N)CSC(CC(=O)NCCOC1CC(=O)N(*)C1=O)C(=O)NCCOC1CC(=O)N(*)C1=O ; *C(=O)[C@H](N)CSC(CC(=O)NCCOC1CC(=O)N(*)C1=O)C(=O)NCCOC1CC(=O)N(*)C1=O]

In certain embodiments, in the compound of Formula II, Linker^{C}-(Linker^{A})₂ is selected from:

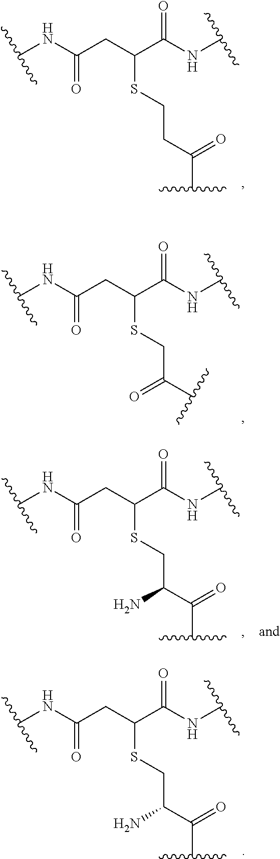

In certain embodiments, in the compound of Formula II, Linker^{C}-(Linker^{A})₂ is selected from:

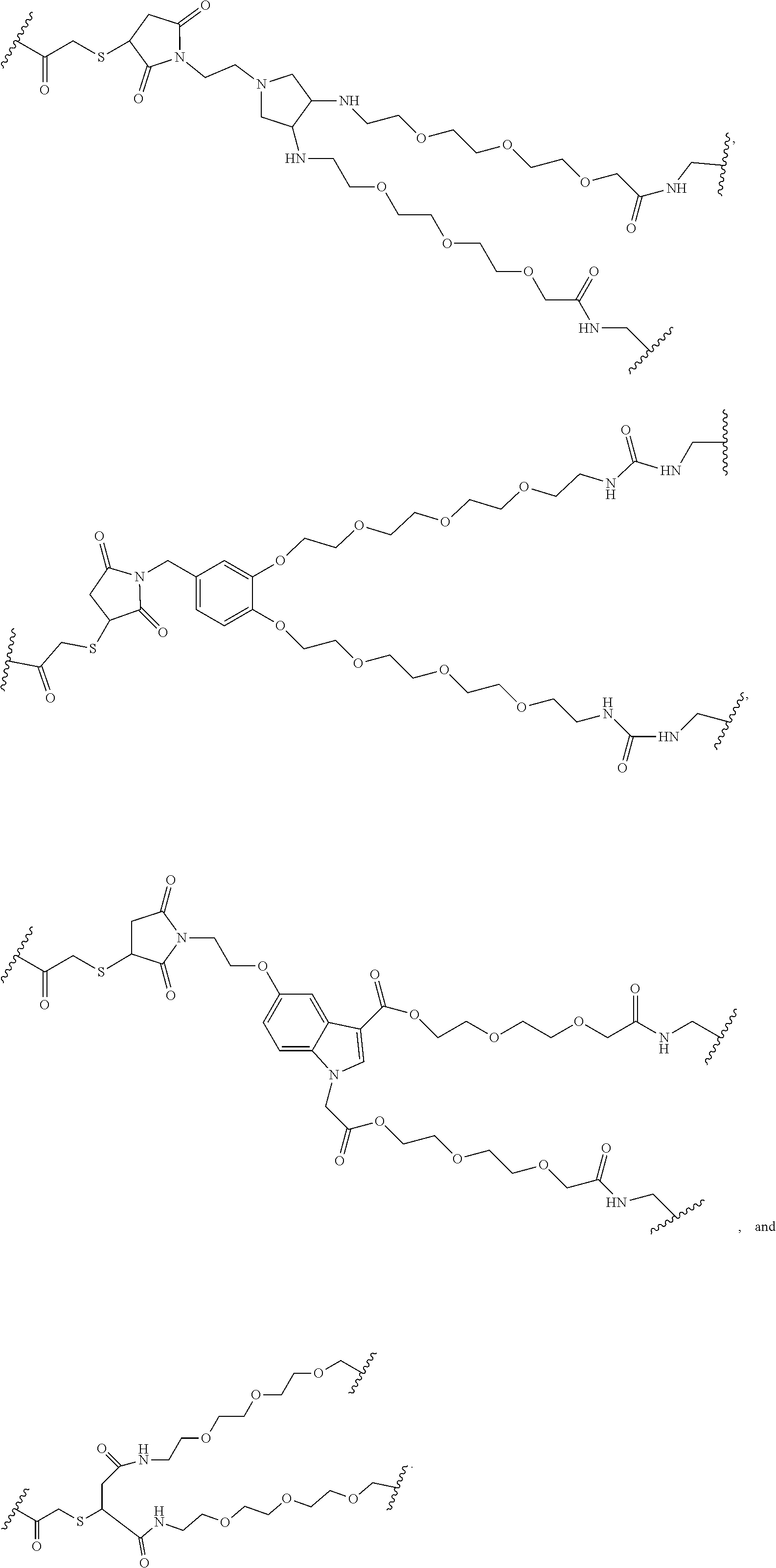 [SMILES: *CNC(=O)COCCOCCOCCNC1CN(CCN2C(=O)CC(SCC(*)=O)C2=O)CC1NCCOCCOCCOCC(=O)NC* ; *CNC(=O)NCCOCCOCCOCCOc1ccc(CN2C(=O)CC(SCC(*)=O)C2=O)cc1OCCOCCOCCOCCNC(=O)NC* ; *CNC(=O)COCCOCCOC(=O)Cn1cc(C(=O)OCCOCCOCC(=O)NC*)c2cc(OCCN3C(=O)CC(SCC(*)=O)C3=O)ccc21 ; *COCCOCCOCCNC(=O)CC(SCC(*)=O)C(=O)NCCOCCOCCOC*]

In certain embodiments, in the compound of Formula II, Linker^{D} is selected from:

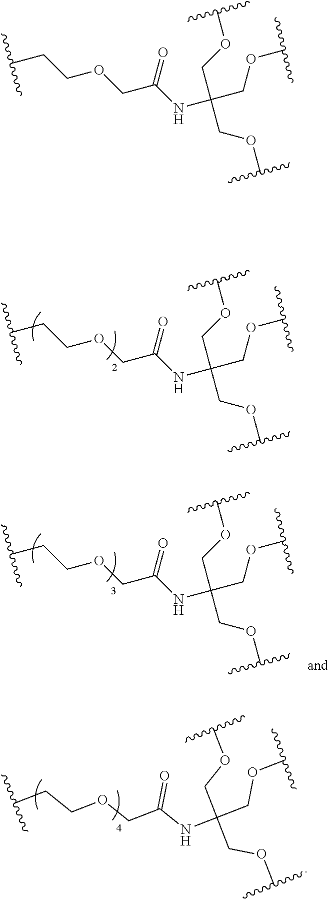

In certain embodiments, in the compound of Formula II, Linker^{D} is selected from:

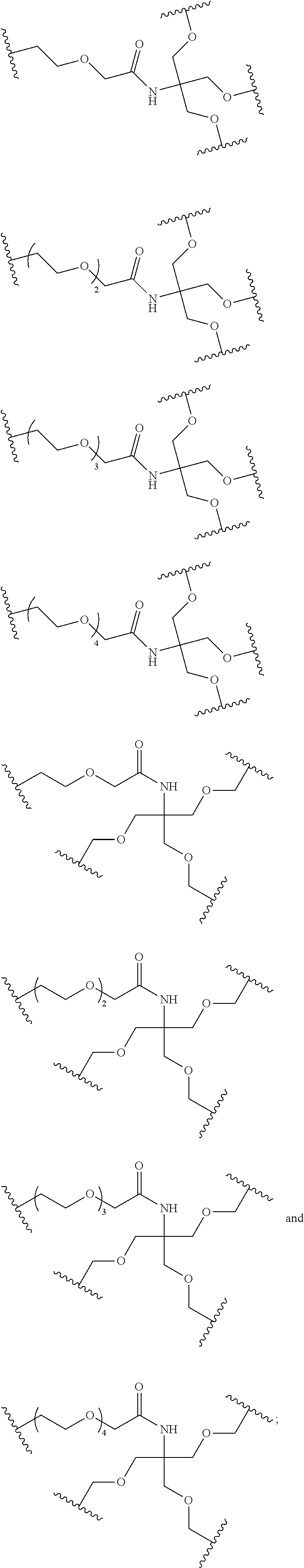

wherein each is optionally substituted with 1, 2, 3, or 4 substituents are selected from R²¹.

In certain embodiments, in the compound of Formula II, Linker^{B}-(Linker^{A}) is selected from

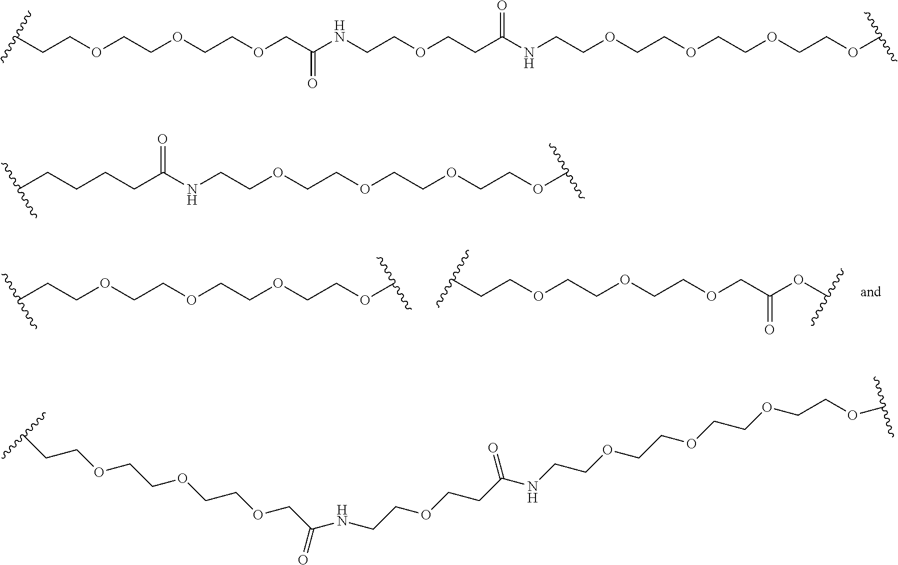 [SMILES: *CCOCCOCCOCC(=O)NCCOCCC(=O)NCCOCCOCCOCCO* ; *CCCCC(=O)NCCOCCOCCOCCO* ; *CCOCCOCCOCCO* ; *CCOCCOCCOCC(=O)O* ; *CCOCCOCCOCC(=O)NCCOCCC(=O)NCCOCCOCCOCCO*]

In certain embodiments, in the compound of Formula II, Linker^{C}-(Linker^{A}) is selected from

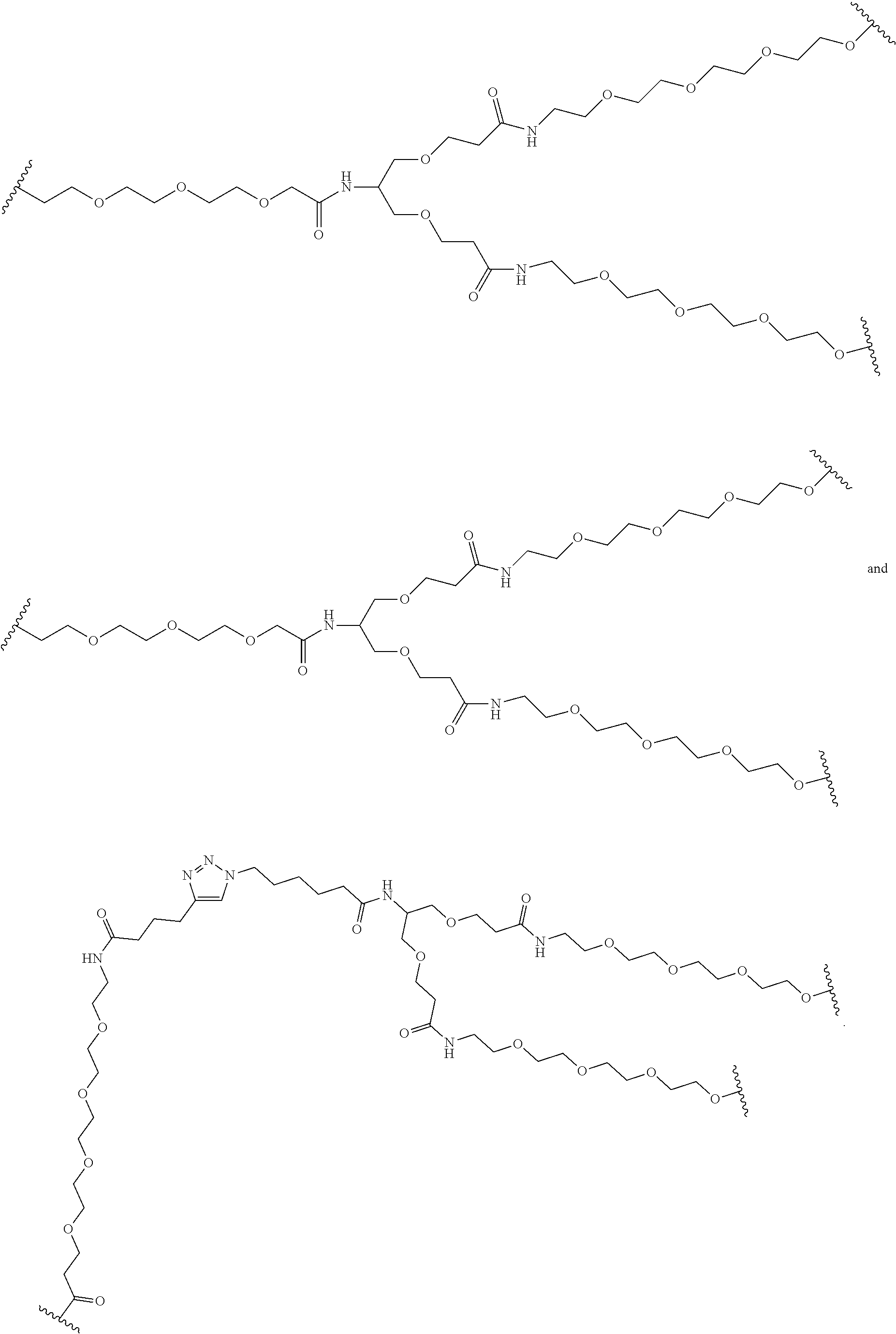 [SMILES: *CCOCCOCCOCC(=O)NC(COCCC(=O)NCCOCCOCCOCCO*)COCCC(=O)NCCOCCOCCOCCO* ; *CCOCCOCCOCC(=O)NC(COCCC(=O)NCCOCCOCCOCCO*)COCCC(=O)NCCOCCOCCOCCO* ; *OCCOCCOCCOCCNC(=O)CCOCC(COCCC(=O)NCCOCCOCCOCCO*)NC(=O)CCCCCn1cc(CCCC(=O)NCCOCCOCCOCCOCCC(*)=O)nn1]

In certain embodiments, in the compound of Formula II, Linker^{D}-(Linker^{A}) is selected from

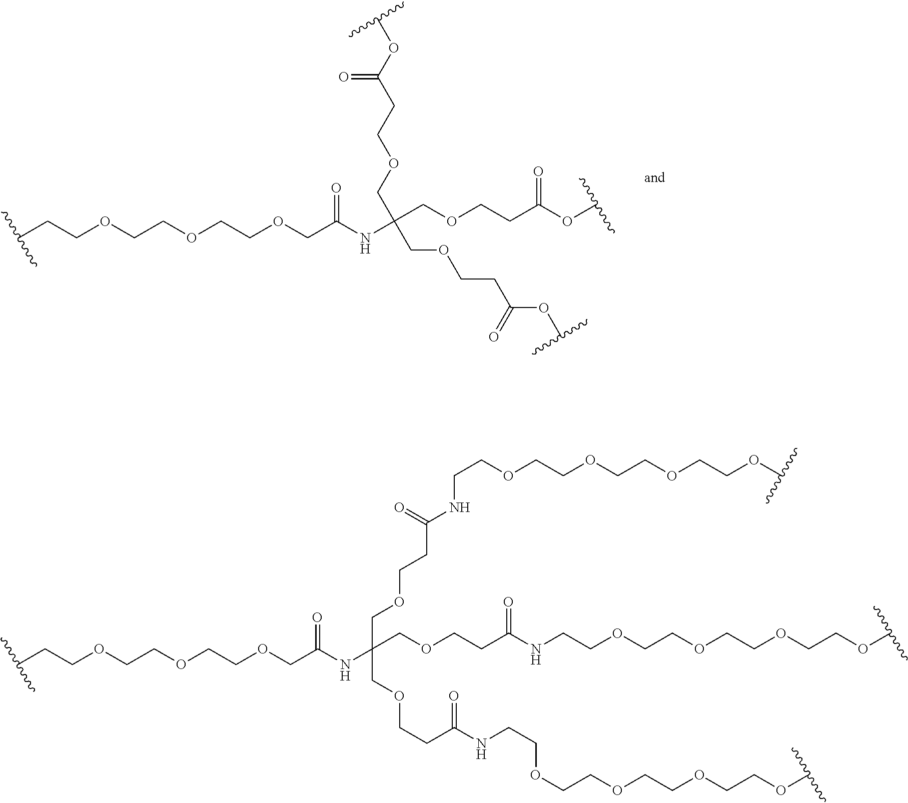 [SMILES: *CCOCCOCCOCC(=O)NC(COCCC(=O)O*)(COCCC(=O)O*)COCCC(=O)O* ; *CCOCCOCCOCC(=O)NC(COCCC(=O)NCCOCCOCCOCCO*)(COCCC(=O)NCCOCCOCCOCCO*)COCCC(=O)NCCOCCOCCOCCO*]

In various embodiments, R⁴ is independently selected at each occurrence from hydrogen, heteroalkyl, alkyl, haloalkyl, arylalkyl, heteroarylalkyl, alkenyl, alkynyl, aryl, heteroaryl, heterocycle, —OR⁶, —NR⁶R⁷, C(O)R³, S(O)R³, C(S)R³, and S(O)₂R³.

In various embodiments, in the compound of Formula II, R⁵ is independently selected from hydrogen, heteroalkyl,

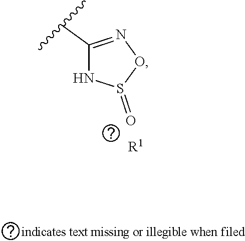

C₀-C₆alkyl-cyano, alkyl, alkenyl, alkynyl, haloalkyl, F, Cl, Br, I, aryl, arylalkyl, heteroaryl, heteroarylalkyl, heterocycle, heterocycloalkyl, haloalkoxy, —O-alkenyl, —O-alkynyl, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R⁸)—C(S)R³, C₀-C₆alkyl-N(R⁸)—S(O)₂R³ C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, and C₀-C₆alkyl-O—S(O)₂R³, each of which is optionally substituted with 1, 2, 3, or 4 substituents.

In various embodiments, in the compound of Formula II, R⁶ and R⁷ are independently selected at each occurrence from hydrogen, heteroalkyl, alkyl, arylalkyl, heteroaryl alkyl, alkenyl, alkynyl, and, haloalkyl, heteroaryl, heterocycle, -alkyl-OR⁸, -alkyl-NR⁸R⁹, C(O)R³, S(O)R³, C(S)R³, and S(O)₂R³.

In various embodiments, in the compound of Formula II, R⁸ and R⁹ are independently selected at each occurrence from hydrogen, heteroalkyl, alkyl, arylalkyl, heteroarylalkyl, alkenyl, alkynyl, aryl, heteroaryl, and heterocycle.

In various embodiments, the compound of Formula II has the structure of Formula II-A. In various embodiments, in the compound of Formula II-A, [TBM] and [LRP1BM] are as defined herein.

A compound of Formula II-A, having the structure:

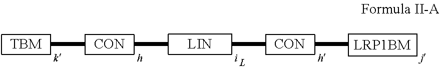

wherein:
[TBM] represents a Target binding motif comprising or consisting of:
   (a) a compound selected from:

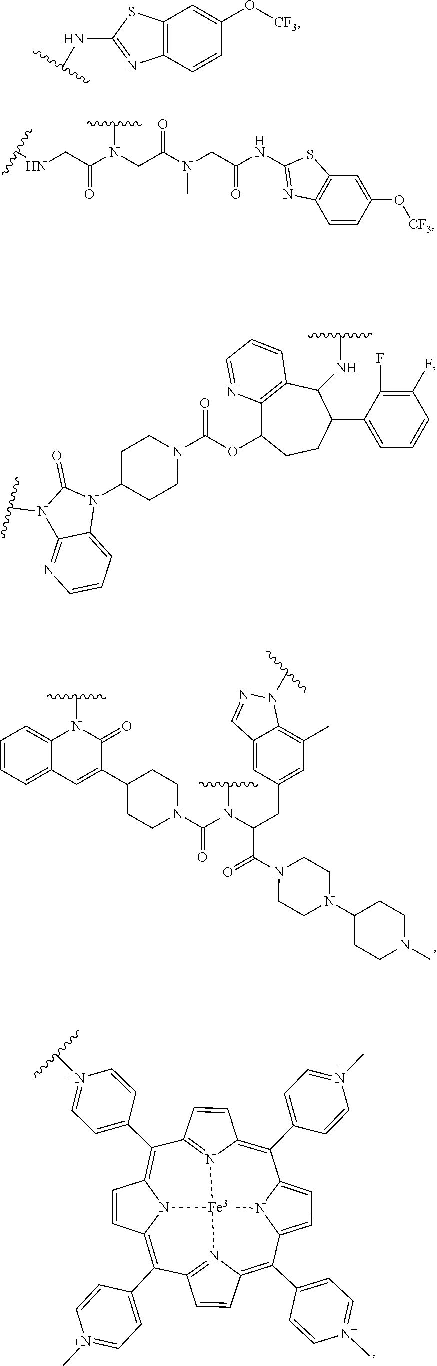

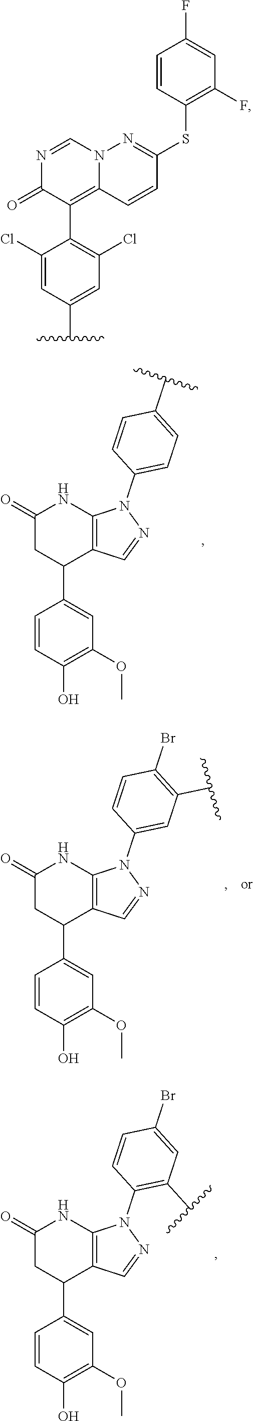 [SMILES: *c1cc(Cl)c(-c2c(=O)ncn3nc(Sc4ccc(F)cc4F)ccc23)c(Cl)c1 ; *c1ccc(-n2ncc3c2NC(=O)CC3c2ccc(O)c(OC)c2)cc1 ; *c1cc(-n2ncc3c2NC(=O)CC3c2ccc(O)c(OC)c2)ccc1Br ; *c1cc(Br)ccc1-n1ncc2c1NC(=O)CC2c1ccc(O)c(OC)c1]

or a derivative or prodrug thereof,
wherein
[symbol] indicates possible points of covalent attachment to a [Linker] or a [LRP1BM];
   (b) a compound of formula (I):

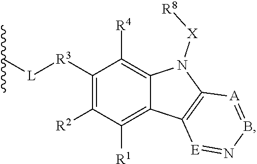

or a derivative or prodrug thereof,
wherein:
A is N or CR⁵;
B is N or CR⁶;
E is N or CR⁷;
L is a substituted or unsubstituted alkylene, substituted or unsubstituted alkenylene, substituted or unsubstituted alkynylene, substituted or unsubstituted carbocyclylene, substituted or unsubstituted heterocyclylene, substituted or unsubstituted arylene, substituted or unsubstituted heteroarylene, substituted or unsubstituted heteroalkylene, a bond, —O—, —NR^{A}—, —S—, —C(═O)—, —C(═O)O—, —C(═O)NR^{A}, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂—NR^{A}—, —NR^{A}S(O)₂—, or a combination thereof;
X is a bond or substituted or unsubstituted C₁₋₁₂ alkylene, wherein one or more carbon is optionally replaced with C(═O), O, S, SO₂, NH, or NC₁₋₆ alkyl optionally substituted with halogen, OH, or C₁₋₆ alkyl;
R⁸ is hydrogen, —N₃, alkynyl, OH, halogen, NH₂, N(C₁₋₆ alkyl)₂, aryl, heteroaryl, or a protecting group, wherein the aryl and heteroaryl are optionally substituted with halogen, SO₂, NH₂, or C₁₋₆ alkyl optionally substituted with halogen or C₃₋₈ cycloalkyl;
R³ is —(CH₂)ₙ—, —(CH₂)ₙ—C(═O), —(CH₂)ₙC(═O)—O—, (CH₂)ₙO—, -A-(CH₂)ₙ—O—, —(CH₂)ₙ-A-O—, -A-O—(CH₂)ₙ—(C═O)NR^{A}—, —(CH₂)ₙ—S—, -A-(CH₂)ₙS—, —(CH₂)ₙ-A-S—, -A-S—(CH₂)ₙ—(C═O)NR^{A}—, —(CH₂)ₙ—NR^{A}—, -A-(CH₂)ₙ—NR^{A}, —(CH₂)ₙ-A-NR^{A}—, —(CH₂)ₙ—(C═O)NR^{A}—, -A-(CH₂)ₙ—(C═O)NR^{A}—, —(CH₂)ₙ-A-(C═O)NR^{A}—, -A-NR^{A}—(CH₂)ₙ—(C═O)NR^{A}—, —(CH₂)ₙ—S(O)₂NR^{A}, -A-(CH₂)ₙ—S(O)₂NR^{A}—, or —(CH₂)ₙ-A-S(O)₂NR^{A}—;
each occurrence of R^{A} is independently selected from hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, substituted or unsubstituted aryl, substituted or unsubstituted heteroaryl, or a nitrogen protecting group when attached to a nitrogen atom, or two R^{A} groups are joined to form a substituted or unsubstituted heterocyclic ring;
each occurrence of A is independently selected from substituted or unsubstituted heterocyclylene, substituted or unsubstituted arylene, or substituted or unsubstituted heteroarylene;
R¹, R² and R⁴- R⁸ are each independently hydrogen, OH, halogen, NH₂, CH₃, SO₂, NO₂, a leaving group, a protecting group, aryl, heteroaryl, NHR¹², N(R¹²)₂ C₃₋₈ cycloalkyl, N(R¹²)₂heterocyclyl or —(CH₂)ₙ—R¹²;
R¹² is hydrogen, —CH₃, aryl, or heteroaryl; and
n is 0-12;
wherein one or more carbon of R¹-R⁷ is optionally replaced with C(═O)O, O, S, SO₂, NH, NH—C₁₋₆ alkyl, NC₁₋₆, alkyl, NH₂, or N(C₁₋₆ alkyl)₂; and
[symbol] indicates the point of covalent attachment to a [Linker] or a [LRP1BM];
   (c) a compound of formula (11):

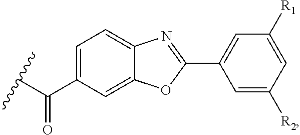

or a derivative or prodrug thereof,
wherein
R₁ and R₂ are each independently selected from hydrogen, N₃, alkynyl, OH, halogen, NH₂, N(C₁₋₆ alkyl)₂, C₁₋₆ alkyl, aryl, heteroaryl, NHR², N(R²)₂ C₃₋₈ cycloalkyl, N(R¹²)₂ heterocyclyl, or —(CH₂)ₙ—R¹²;
wherein the aryl and heteroaryl are optionally substituted with halogen, —SO₂, NO₂, —NH₂, or C₁₋₆ alkyl optionally substituted with halogen or C₃₋₈ cycloalkyl;
R¹² is hydrogen, —CH₃, aryl, or heteroaryl; and n is 0-12;
wherein one or more carbon of R¹ or R² is optionally replaced with C(═O), O, S, SO₂, NH, NH—C₁₋₆ alkyl, NC₁₋₆ alkyl, NH₂, or N(C₁₋₆ alkyl)₂; and
[symbol] indicates the point of covalent attachment to a [Linker] or a [LRP1BM];
   (d) a compound of formula (III):

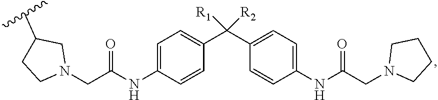

or a derivative or prodrug thereof,
wherein
R₁ is selected from benzene, phenyl, cyclohexyl, hydrogen, and CF₃;
R₂ is selected from hydrogen and CF₃; and
[symbol] indicates the point of covalent attachment to a [Linker] or a [LRP1BM];

(e) a compound of formula (IV):

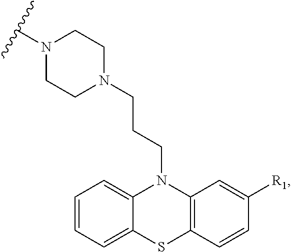

or a derivative or prodrug thereof,
wherein
R₁ is selected from hydrogen, Cl, OMe, SMe, and CF₃, and
[symbol] indicates the point of covalent attachment to a [Linker] or a [LRP1BM];
   (f) a compound of formula (V):

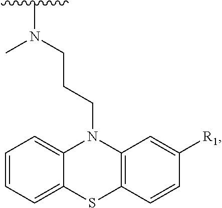

or a derivative or prodrug thereof,
wherein
R₁ is selected from hydrogen, Cl, OMe, SMe, and CF₃, and
[symbol] indicates the point of covalent attachment to a [Linker] or a [LRP1BM]; or
   (g) an amino acid sequence selected from:

SVWIWYE,
DVWIINKKLK,
MLRTKDLIWTLFFLGTAVS-NH₂,
MLRTKDLIWTLFFLGTAVS-KKRPKP-NH₂,
and
MLRTKDLIWTLFFLGTAVS-KKLVFF-NH₂;

[LRP1BM] represents a low density lipoprotein receptor-related protein 1 (LRP1) receptor binding motif comprising one of the following amino acid sequences:

TFFYGGSRGKRNNFKTEEYC-OH (or -NH₂),
TWPKHFDKHTFYSILKLGKH-OH,
EAKIEKHNHYQKK/C-NH₂,
EAKIEKHNHYQKQLEIAHEKLRK/C-NH₂,
R₈AKIEKHS₅HYQKK/C-NH₂,

wherein R₈ represents (R)-2-(7-octenyl)Ala-OH, S₅ represents (S)-2-(4-pentenyl)Ala-OH, and there is a hydrocarbon bridge between position 1 and 8,

LRKLRKRLLRDADDLLRKLRKRLLRDADDL-NH₂,
TEELRVRLASHLRKLRKRLL-NH₂,
Ac-VKFNKPFVFLNleIEQNTK-NH₂,

wherein Nle represents norleucine,

VKFNKPFVFLMIEQNTK,
TFFYGGCRGKRNNFKTEEYC-OH (or -NH₂),
TFFYGGSRGKRNNFRTEEYC-OH (or -NH₂),
TFFYGGSRGRRNNFRTEEYC-OH (or -NH₂),
cyeetkfnnrkGrsGGyfft-OH (or-NH₂),
TFFYGGCRAKRNNFKRAKY,
TFFYGGCRGKKNNFKRAKY,
PFFYGGCRGKRNNFKTEEY,
TFFYGGKRGKRNNFKTKEY,
TFFYGGCRGKRNNFKTKRY,
TFFYGGKRGKRNNFKTAEY,
TFFYGGKRGKRNNFKREKY,
RFKYGGCLGNKNNFLRLKY,
and
RFKYGGCLGNKNNYLRLKY,

wherein the underlined amino acids in the above sequences indicate that the amino acids may be present or absent and underlined K/C indicates that either K or C may be present; and
[LIN] is [LINKER] or [LINKER-2], each of which is a chemical moiety having a valency from 1 to 15, which covalently attaches to one or more [TBM] or [LRP1BM] groups, optionally through a [CON], wherein the [LIN] optionally itself contains one or more [CON] groups;
k′ is an integer ranging from 1 to 15;
j′ is an integer ranging from 1 to 15;
h and h′ are each independently an integer ranging from 0 to 15;
i_{L} is 0 to 15;
with the proviso that at least one of h, h′, and i_{L} is at least 1,
   or a salt, stereoisomer, or solvate thereof.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means.

Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from.

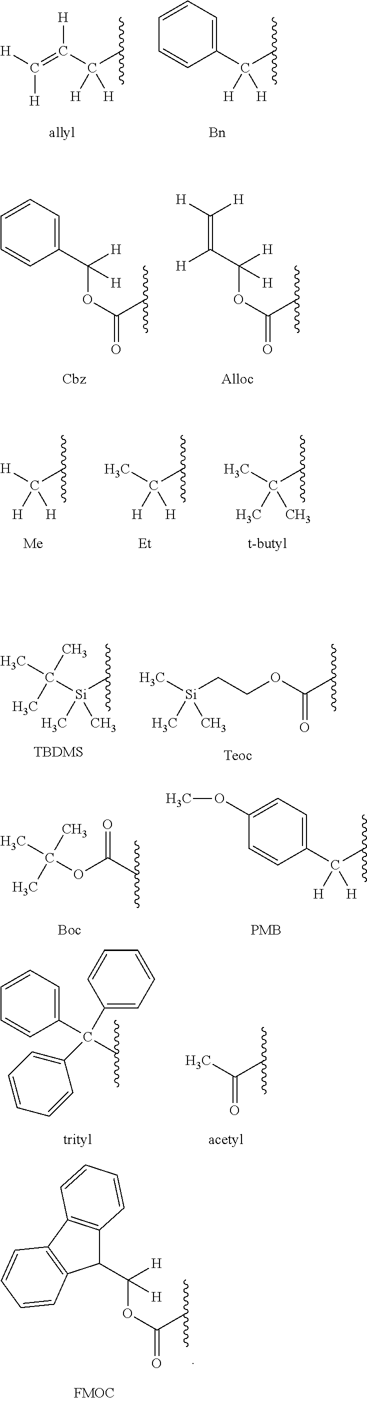

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Compositions

The compositions containing the compound(s) described herein include a pharmaceutical composition comprising at least one compound as described herein and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

### Methods of Treatment

In another aspect, the present disclosure relates to a method of treating, ameliorating, and/or preventing a disease or disorder in a subject, the method comprising administering to the subject a therapeutically effective amount of a compound of formula (I).

The disease or disorder can be any disease or disorder known to a person of skill in the art. Exemplary diseases or disorders include, but are not limited to, Addison's Disease, Autoimmune polyendodrine syndrome (APS) types 1, 2 and 3, autoimmune pancreatitis (AIP), diabetes mellitus type 1, autoimmune thyroiditis, Ord's thyroiditis, Grave's disease, autoimmune oophoritis, endometriosis, autoimmune orchitis, Sjogren's syndrome, autoimmune enteropathy, coeliac disease, Crohn's disease, microscopic colitis, ulcerative colitis, autophospholipid syndrome (APlS), aplastic anemia, autoimmune hemolytica anemia, autoimmune lymphoproliferative syndrome, autoimmune neutropenia, autoimmune thrombocytopenic purpura, cold agglutinin disease, essential mixed cryoglulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, eosinophilic fasciitis, Felty syndrome, AgG4-related disease, juvenile arthritis, Lyme disease (chronic), mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, rheumatoid arthritis, sarcoidosis, Schnitzler syndrome, systemic lupus erythematosus, undifferentiated connective tissue disease (UCTD), dermatomyositis, fibromyalgia, myositis, inclusion body myositis, myasthenia gravis, neuromyotonia, paraneoplastic cerebellar degeneration, polymyositis, acute disseminated encephalomyelitis (ADEM), acute motor axonic neuropathy, anti-NMDA receptor encephalitis, Balo concentric sclerosis, Bickerstaff's encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, multiple sclerosis, pattern II, Oshtoran Syndrome, Pediatric Autoimmune Neuropsychiatric Disorder Associated with Streptococcus (PANDAS), progressive inflammatory neuropathy, restless leg syndrome, stiff person syndrome, Syndenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease (AIED), Meniere's disease, Bechet's disease, Eosinophilic granulomatosis with polyangiitis (EGPA), giant cell arteritis, granulomatosis with polyangiitis (GPA), IgA vasculitis (IgAV), IgA nephropathy, Kawasaki's disease, leukocytoclastic vasculitis, lupus vasculitis, rheumatoid vasculitis, microscopic polyangiitis (MPA), polyarteritis nodosa (PAN), polymyalgia rheumatica, urticarial vasculitis, vasculitis, primary immune deficiency, chronic fatigue syndrome, complex regional pain syndrome, eosinophilic esophagitis, gastritis, interstitial lung disease, POEMS syndrome, Raynaud's syndrome, primary immunodeficiency, pyoderma gangrenosum, prostate cancer, metastatic prostate cancer, stomach cancer, colon cancer, rectal cancer, liver cancer, pancreatic cancer, lung cancer, breast cancer, cervix uteri cancer, corpus uteri cancer, ovary cancer, testis cancer, bladder cancer, renal cancer, brain/CNS cancer, head and neck cancer, throat cancer, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, esophagus, larynx, kidney cancer, lymphoma, inflammatory diseases of neurodegeneration, diseases of compromised immune response causing inflammation, chronic inflammatory diseases, hyperglycemic disorders, diabetes (I and II), pancreatic β-cell death and related hyperglycemic disorders, liver disease, renal disease, cardiovascular disease, muscle degeneration and atrophy, low grade inflammation, gout, silicosis, atherosclerosis and associated conditions, stroke and spinal cord injury, arteriosclerosis, Huntington's Disease (HD), Parkinson's Disease (PD), Amyotropic Lateral Sclerosis (ALS), multiple system atrophy (MSA), Alzheimer's Disease, Lewy body dementia, Multiple System Atrophy, spinal and bulbar muscular atrophy (Kennedy's disease), Tourette Syndrome, spinocerebellar ataxia (SCA) (e.g., Type 1 SCA1, Type 2 SCA2, Type 3 (Machado-Joseph disease) SCA3/MJD, Type 6 SCA6, Type 7 SCA7, Type 8 SCA8, Friedreich's Ataxia and Dentatorubral pallidoluysian atrophy DRPLA/Haw-River syndrome), schizophrenia, age associated memory impairment, autism, migraines, Rett syndrome, complex regional pain syndrome (CRPS), obsessive-compulsive disorder (OCD), attention-deficit disorder, bipolar disorder, depression, migraine via degradation of CGRP or CGRP receptor, ATTR amyloidosis, hereditary cerebral angiopathy, and combinations thereof.

In some embodiments, the disease or disorder is a neurological disease or disorder. Exemplary neurological diseases or disorders include, but are not limited to, Huntington's Disease (HD), Parkinson's Disease (PD), Amyotropic Lateral Sclerosis (ALS), multiple system atrophy (MSA), Alzheimer's Disease, Lewy body dementia, Multiple System Atrophy, spinal and bulbar muscular atrophy (Kennedy's disease), Tourette Syndrome, spinocerebellar ataxia (SCA) (e.g., Type 1 SCA1, Type 2 SCA2, Type 3 (Machado-Joseph disease) SCA3/MJD, Type 6 SCA6, Type 7 SCA7, Type 8 SCA8, Friedreich's Ataxia and Dentatorubral pallidoluysian atrophy DRPLA/Haw-River syndrome), schizophrenia, age associated memory impairment, autism, migraines, Rett syndrome, complex regional pain syndrome (CRPS), obsessive-compulsive disorder (OCD), attention-deficit disorder, bipolar disorder, depression, hereditary cerebral angiopathy, ATTR amyloidosis, and combinations thereof. In some embodiments, the neurological disease or disorder is Alzheimer's disease, migraine, hereditary cerebral angiopathy, or ATTR amyloidosis.

In some embodiments, the compound of formula (I) comprises any amyloid beta or extracellular tau binding motif disclosed elsewhere herein and the method treats, ameliorates, and/or prevents Alzheimer's disease in the subject. In other embodiments, the compound of formula (I) comprises any amyloid beta binding motif described elsewhere herein and the method treats, ameliorates, and/or prevents hereditary cerebral angiopathy in the subject. In other embodiments, the compound of formula (I) comprises any glutamate modulator described elsewhere herein and the method treats, ameliorates, and/or prevents Alzheimer's disease, OCD, SCA, CRPS, Rett syndrome, or a combination thereof in the subject. In other embodiments, the compound of formula (I) comprises any CGRP or CGRP receptor binding motif described elsewhere herein and the method treats, ameliorates, and/or prevents migraines in the subject. In other embodiments, the compound of formula (I) comprises any transthyretin binding motif described elsewhere herein and the method treats, ameliorates, and/or prevents ATTR amyloidosis in the subject. The methods described herein include administering to the subject a therapeutically effective amount of at least one compound described herein, which is optionally formulated in a pharmaceutical composition. In various embodiments, a therapeutically effective amount of at least one compound described herein present in a pharmaceutical composition is the only therapeutically active compound in a pharmaceutical composition. In certain embodiments, the method further comprises administering to the subject an additional therapeutic agent that treats the disease or disorder.

The additional therapeutic agent can be any therapeutic agent known to a person of skill in the art to treat, ameliorate, or prevent a disease or disorder. In some embodiments wherein the method comprises treating, ameliorating, and/or preventing Alzheimer's disease, the additional therapeutic agent is selected from the group consisting of Aricept (donepezil), Exelon (rivastigmine), Namenda (memantine), Namzaric (memantine and donepezil), Razadyne (galantamine), and combinations thereof.

In certain embodiments, administering the compound(s) described herein to the subject allows for administering a lower dose of the additional therapeutic agent as compared to the dose of the additional therapeutic agent alone that is required to achieve similar results in treating the disease or disorder in the subject. For example, in certain embodiments, the compound(s) described herein enhance(s) the activity of the additional therapeutic compound, thereby allowing for a lower dose of the additional therapeutic compound to provide the same effect.

In certain embodiments, the compound(s) described herein and the therapeutic agent are co-administered to the subject. In other embodiments, the compound(s) described herein and the therapeutic agent are coformulated and co-administered to the subject.

In certain embodiments, the subject is a mammal. In other embodiments, the mammal is a human.

### Combination Therapies

The compounds useful within the methods described herein can be used in combination with one or more additional therapeutic agents useful for treating the disease or disorder, and/or with an additional therapeutic agents that reduce or ameliorate the symptoms and/or side-effects of therapeutic agent used in the treatment of the disease or disorder. These additional therapeutic agents may comprise compounds that are commercially available or synthetically accessible to those skilled in the art. When the additional therapeutic agents useful for treating the disease or disorder are used, these additional therapeutic agents are known to treat, or reduce the symptoms of the disease or disorder.

In various embodiments, a synergistic effect is observed when a compound as described herein is administered with one or more additional therapeutic agents or compounds. A synergistic effect may be calculated, for example, using suitable methods such as, for example, the Sigmoid-Eₘₐₓ equation (Holford & Schemer, 1981, Clin. Pharmacokinet, 6:429-453), the equation of Loewe additivity (Loewe & Muischnek, 1926, Arch. Exp. Pathol Pharmacol, 114:313-326) and the median-effect equation (Chou & Talalay, 1984, Adv. Enzyme Regul, 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of the disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat the disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat the disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle.

The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In some embodiments, the compound(s) described herein are administered to a patient, alone or in combination with another pharmaceutical agent, using a sustained release formulation. In some embodiments, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of the disease or disorder in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents are considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXPERIMENTAL EXAMPLES

The invention is further described in detail by reference to the following experimental examples. These examples are provided for purposes of illustration only, and are not intended to be limiting unless so specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Bifunctional Molecules for Targeted Removal of Neurological Proteins

### Materials and Methods

### Synthetic Overview

Peptides are synthesized using standard Fmoc-based solid phase peptide synthesis, wherein Wang resin or CTC resin is used as the C-terminal carboxylic acid linker and Rink amide resin is used as the C-terminal amide linker. The terminal amino acid is deprotected using 20% piperidine in DMF and an coupled with a mixture of Fmoc-Amino Acid-OH, Oxyma, and diisopropylcarbodiimide in DMF. The peptide is capped in a solution of 9:1 pyridine:acetic anhydride.

### Results and Discussion

The present invention aims to treat neurological diseases by removing pathogenic proteins from the brain. Established protein degradation technologies target intracellular or extracellular circulating proteins whereas the present disclosure expands targeted protein degradation to extracellular neurological targets. As several neurological diseases arise from the accumulation and aggregation of pathogenic proteins, there are many opportunities to apply this protein degradation platform. Current treatment options, particularly for Alzheimer's disease, aim to improve symptoms without addressing the underlying cause or slowing disease progression.

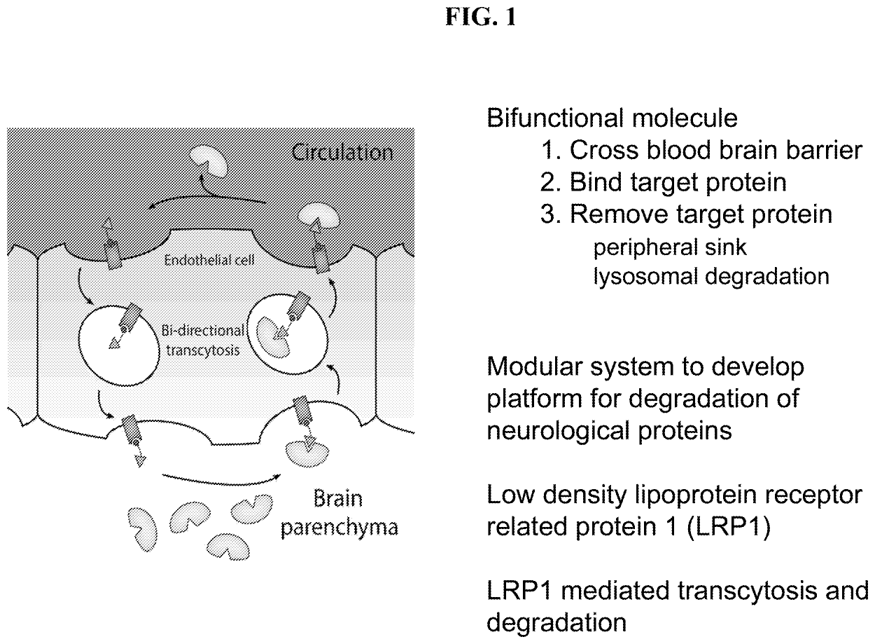

The present disclosure provides a bifunctional molecule comprised of a protein binding moiety coupled with the brain targeting peptide. Brain targeting is achieved via the low density lipoprotein receptor related protein 1 (LRP1). LRP1 is involved in endolysosomal trafficking, as well as receptor-mediated transcytosis across the blood brain barrier, suggesting that peptides targeting this receptor will be capable of both transport and degradation of target neurological proteins. Current efforts utilize the asialoglycoprotein receptor (ASGPr) in the liver for targeted degradation of extracellular proteins. However, since ASGPr is predominantly expressed on hepatocytes, it is effective for systemic extracellular targets, but inaccessible for selective degradation of neurological proteins. Alternatively, LRP1 is expressed in many tissues and implicated in both degradation and transcytosis across the blood-brain barrier. Ligands designed to target this receptor have facilitated receptor-mediated transcytosis across the blood-brain barrier of cargo ranging from small molecules to nanoparticles. Therefore, a ligand targeting LRP1 will expand targeted degradation to neurological protein targets (FIG. 1).

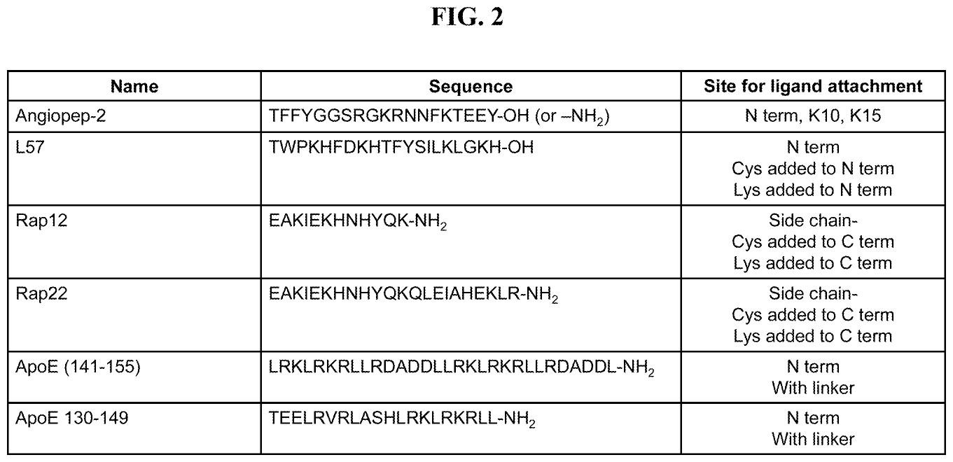

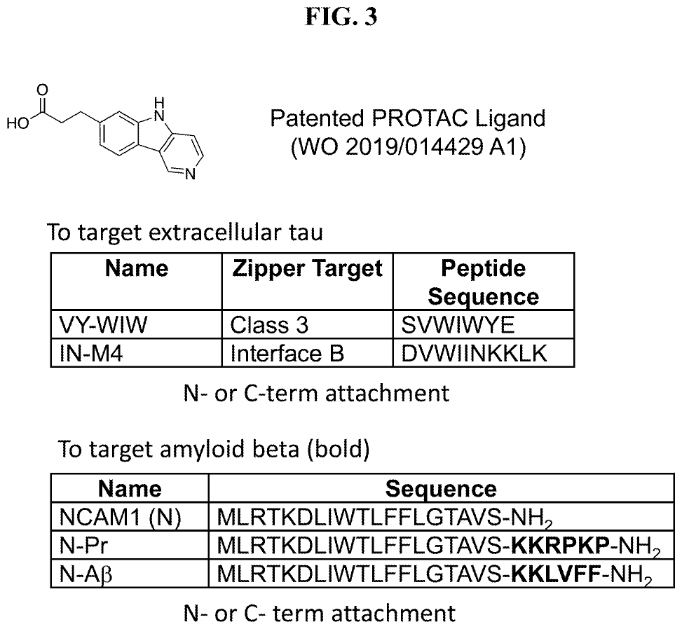

The bifunctional molecule uses an LRP1 binding motif to transport noncovalently bound cargo and has the general structure shown below, wherein the LRP1-binding motif is depicted in FIG. 2 and the Target binding motif is depicted in FIG. 3. The noncovalent nature the transport system allows for targeting endogenous proteins, thus redirecting protein trafficking. The bifunctional molecule expands protein degradation to extracellular neurological targets compared to current technologies that either target systemic proteins or intracellular targets. Additionally, this innovation expands targeted extracellular protein degradation to LRP1, which would be useful in disease states where ASGPr is downregulated.

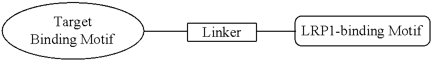

Furthermore, the novel bifunctional molecule allows for both transport and degradation of target neurological proteins instead of inhibiting these proteins. This allows for targeting the undruggable proteome through the use of any protein ligand instead of exclusively inhibitors. This approach also uses the cellular machinery for degrading extracellular proteins, resulting in permanent removal of the pathogenic species instead of temporary inhibition. The present disclosure also allows for a platform approach to the degradation and removal of pathogenic species from the brain. This synthetic peptide/small molecule combination involves a modular approach, which permits easy modification and optimization during platform development.

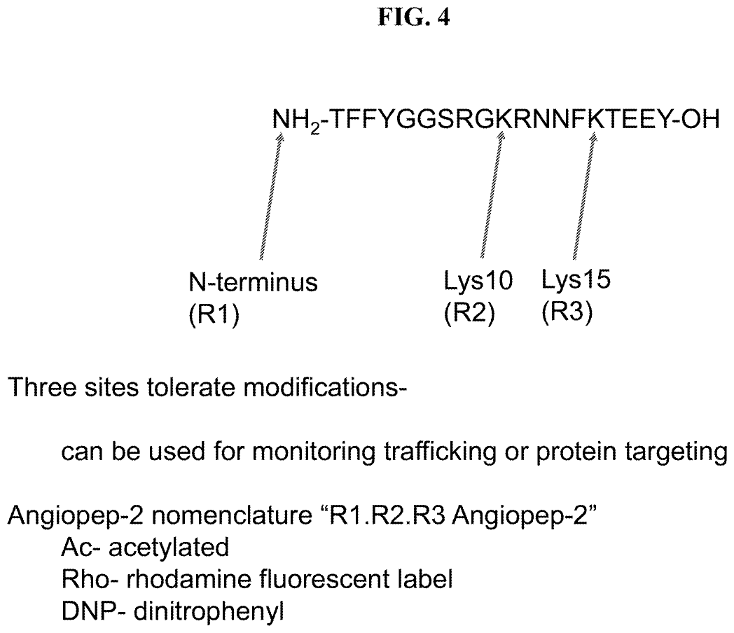

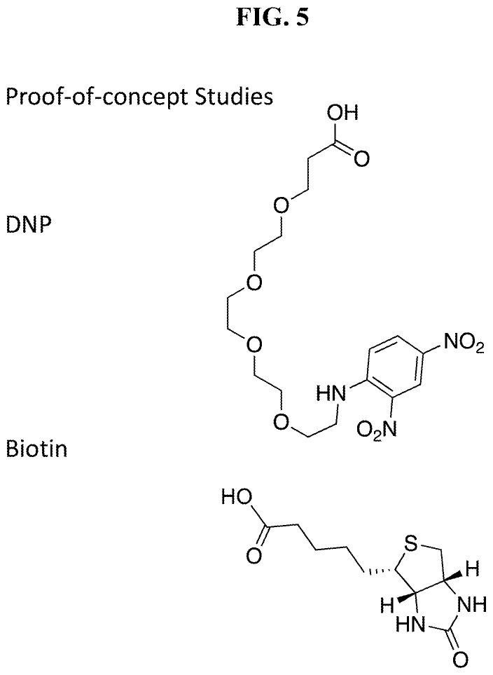

It has been demonstrated that Angiopep-2 is capable of transporting a noncovalently bound protein cargo into murine brain endothelial cells and astrocytes, allowing use of this peptide to target and redirect the trafficking of endogenous proteins. Therefore, it was decided to form a bifunctional molecule comprising a modified Angiopep-2 as the LRP1 binding motif, wherein Angiopep-2 was modified via acetylation and/or substitution with a rhodamine fluorescent label (FIG. 4). The modified Angiopep-2 was bonded to a biotin or ethoxylated dinitrophenyl Target binding motif for the use in the current proof of concept studies (FIG. 5).

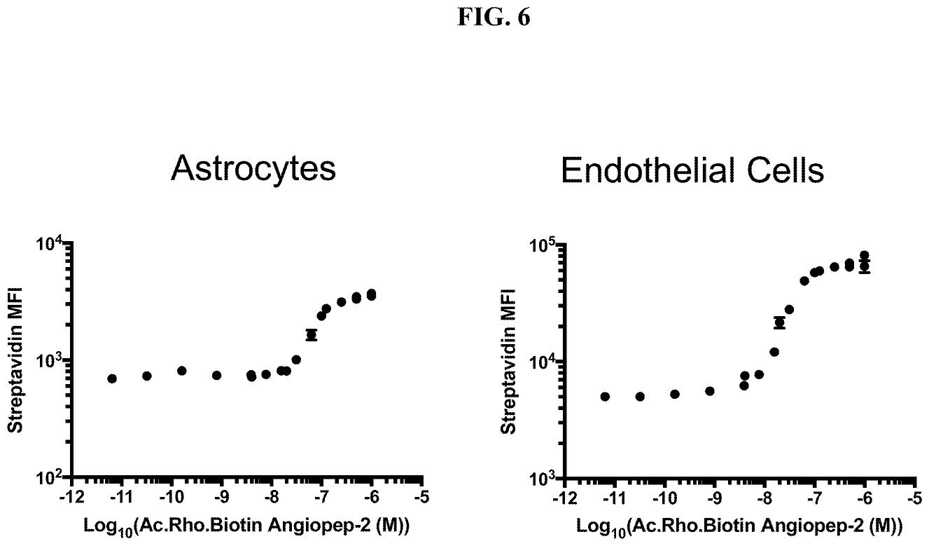

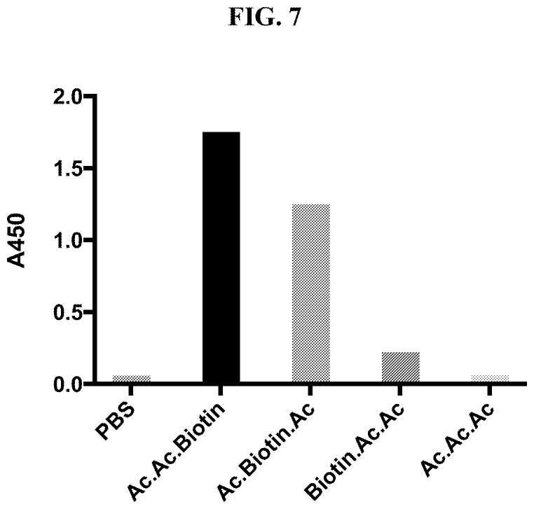

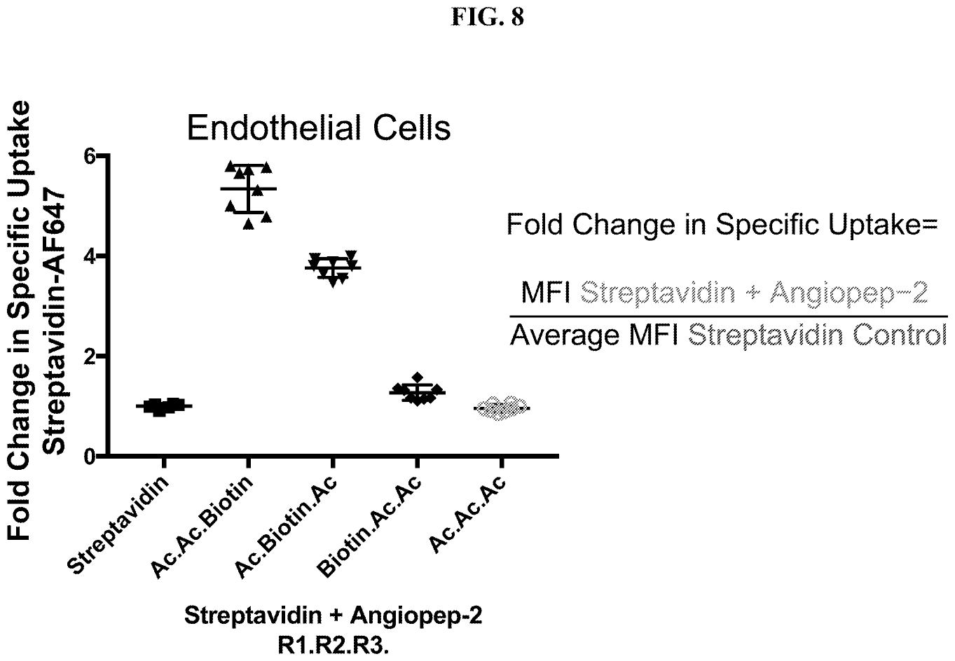

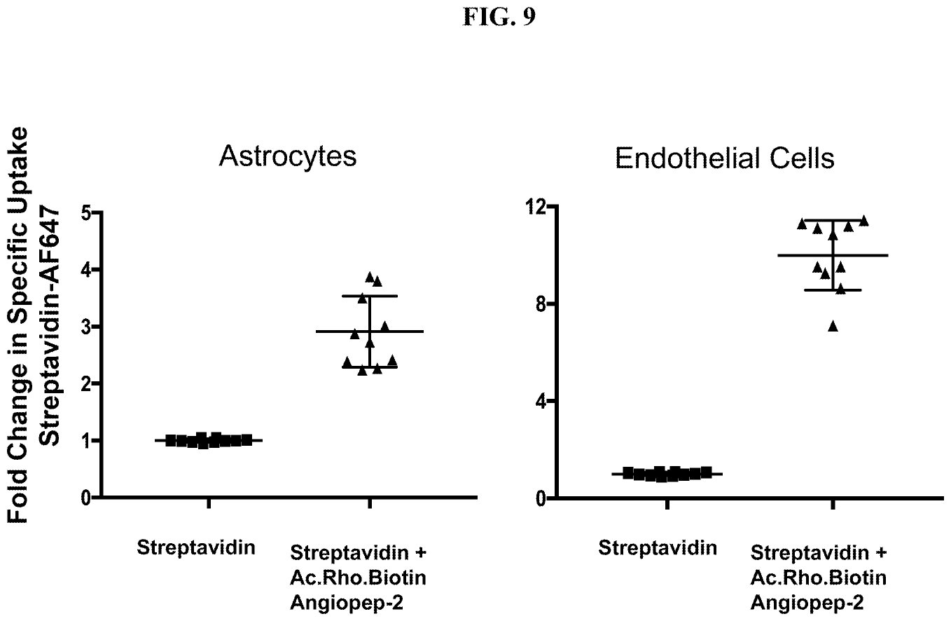

These studies demonstrated that the bifunctional molecule derived from Angiopep-2 can noncovalently transport streptavidin into murine brain endothelial cells and astrocytes. Specifically, the data herein show that biotinylated Angiopep-2 is capable of triggering endocytosis of streptavidin, displaying the capability of this peptide to facilitate transport of noncovalently bound cargo (FIGS. 6-9). FIG. 6 depicts the saturable level of target (streptavidin) uptake with increasing concentration of bifunctional molecule. FIG. 7 depicts ELISA studies demonstrating the interaction of LRP1BM-TBM (Angiopep-2-Biotin) with target protein Streptavidin. Cellular assay demonstrate LRP1BM-TBM (Angiopep-2-Biotin) mediated internalization of target protein Streptavidin in mouse brain endothelial cells (FIG. 8 and FIG. 9) as well as astrocytes (FIG. 9).

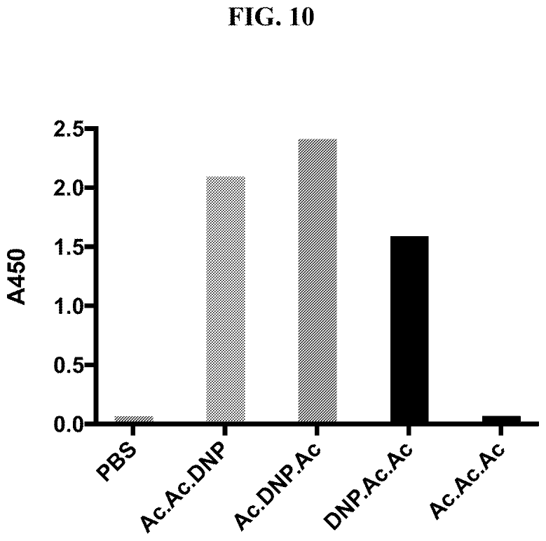

The trend seen in FIG. 8 correlates with the FIG. 7 binding results. FIG. 10 depicts ELISA studies demonstrating the interaction of LRP1BM-DNP (TBM) Angiopep-2 with target protein anti-DNP antibody. This data of the bifunctional molecule formed from Angiopep-2 and ethoxylated DNP molecule further demonstrates that DNP-modified Angiopep-2 binds anti-DNP antibody (FIG. 10). These findings represent a significant improvement over all previous uses of this peptide, which required covalent modification of the cargo with Angiopep-2.

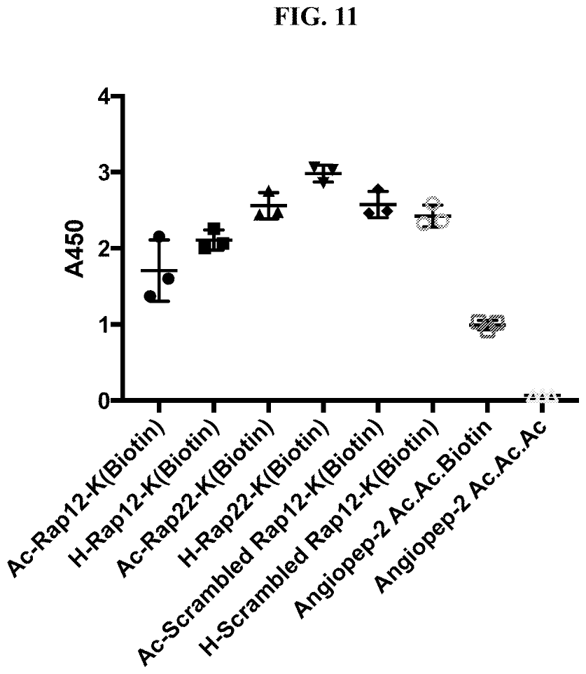

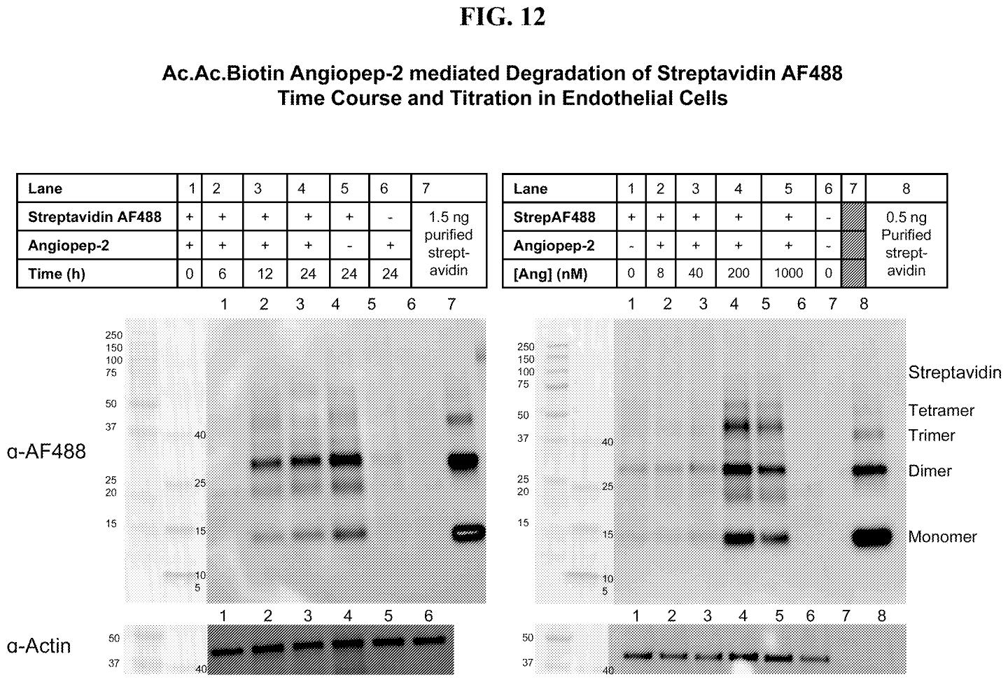

While the data herein demonstrate the potential of Angiopep 2 to facilitate both transcytosis and endolysosomal targeting, future work entails applying this platform to therapeutically relevant targets to evaluate the contribution of cargo size, valency, and mechanism of transport. Some studies have been done on other bifunctional molecules comprising an LRP1 binding motif depicted in FIG. 2 and a biotin Target binding motif, wherein these bifunctional molecules also noncovalently bind streptavidin (FIG. 11). Specifically, FIG. 11 depicts ELISA studies demonstrating the interaction of LRP1BM-Biotin (TBM) with target protein Streptavidin. FIG. 12 demonstrates the degradation of a target protein using an LRP1 binding motif.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

### ENUMERATED EMBODIMENTS

The following enumerated embodiments are provided, the numbering of which is not to be construed as designating levels of importance:

Embodiment 1 provides a compound of formula (I), or a salt, geometric isomer, stereoisomer, or solvate thereof:

[TBM]ₙ-[Linker]ₘ-[LRP1BM]ₒ (I),

wherein
m is an integer from 0 to 15;
n and o are each independently an integer from 1 to 15;
[TBM] represents a target binding motif comprising or consisting of:
   (a) a compound selected from:

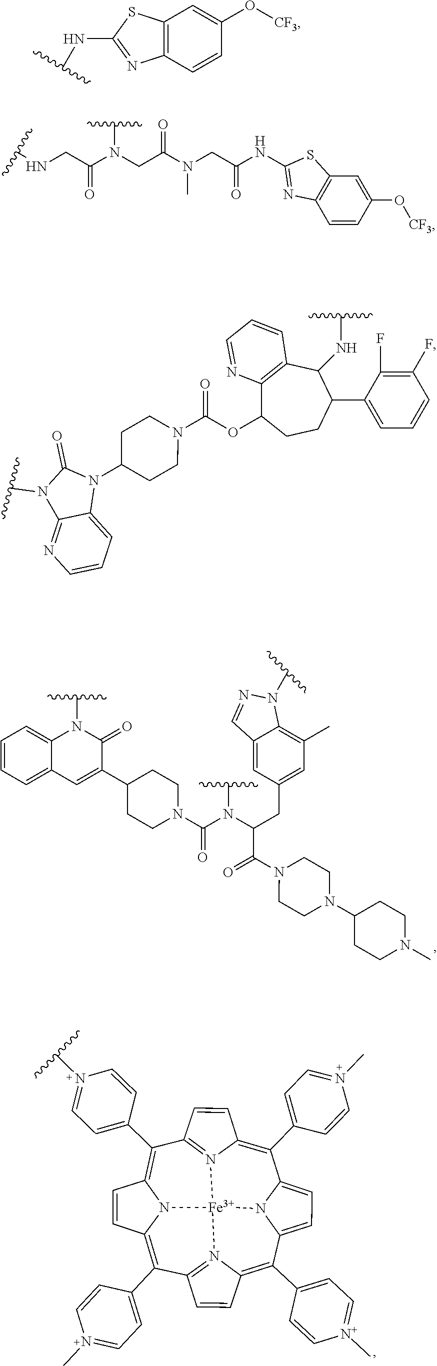

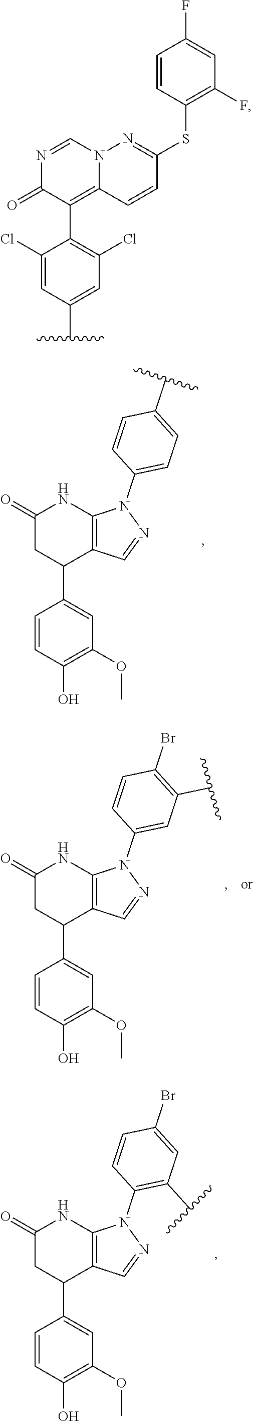 [SMILES: *c1cc(Cl)c(-c2c(=O)ncn3nc(Sc4ccc(F)cc4F)ccc23)c(Cl)c1 ; *c1ccc(-n2ncc3c2NC(=O)CC3c2ccc(O)c(OC)c2)cc1 ; *c1cc(-n2ncc3c2NC(=O)CC3c2ccc(O)c(OC)c2)ccc1Br ; *c1cc(Br)ccc1-n1ncc2c1NC(=O)CC2c1ccc(O)c(OC)c1]

or a derivative or prodrug thereof,
wherein
[symbol] indicates possible points of covalent attachment to a [Linker] or a [LRP1BM];
   (b) a compound of formula (I):

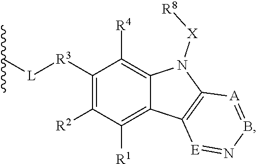

or a derivative or prodrug thereof,
wherein:
A is N or CR⁵;
B is N or CR⁶;
E is N or CR⁷;
L is a substituted or unsubstituted alkylene, substituted or unsubstituted alkenylene, substituted or unsubstituted alkynylene, substituted or unsubstituted carbocyclylene, substituted or unsubstituted heterocyclylene, substituted or unsubstituted arylene, substituted or unsubstituted heteroarylene, substituted or unsubstituted heteroalkylene, a bond, —O—, —NR^{A}—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination (hereof;
X is a bond or substituted or unsubstituted C₁₋₁₂ alkylene, wherein one or more carbon is optionally replaced with C(═O), O, S, SO₂, NH, or NC₁₋₆ alkyl optionally substituted with halogen, OH, or C₁₋₆ alkyl;
R⁸ is hydrogen, —N₃, alkynyl, OH, halogen, NH₂, N(C₁₋₆ alkyl)₂, aryl, heteroaryl, or a protecting group, wherein the aryl and heteroaryl are optionally substituted with halogen, SO₂, NH₂, or C₁₋₆ alkyl optionally substituted with halogen or C₃₋₈ cycloalkyl;
R³ is —(CH₂)ₙ, —(CH₂)ₙC(═O), —(CH₂)ₙ—C(═O)—O—, —(CH₂)ₙ—O—, -A-(CH₂)ₙ—O—, —(CH₂)ₙ-A-O—, -A-O—(CH₂)ₙ—(C═O)NR^{A}—, —(CH₂)ₙ—S—, -A-(CH₂)ₙ—S—, —(CH₂)ₙ-A-S—, -A-S—(CH₂)ₙ(C═O)NR^{A}—, —(CH₂)ₙ—NR^{A}—, -A-(CH₂)ₙ—NR^{A}—, —(CH₂)ₙ-A-NR^{A}—, —(CH₂)ₙ—(C═O)NR^{A}—, -A-(CH₂)ₙ—(C═O)NR^{A}—, —(CH₂)ₙ-A-C(═0)NR^{A}—, —NR^{A}—(CH₂)ₙ—(C═O)NR^{A}—, —(CH₂)ₙ—S(O)₂—NR_{A}—, -A-(CH₂)ₙ—S(O)₂NR^{A}—, or —(CH₂)ₙ-A-S(O)₂NR^{A}—;
each occurrence of R^{A} is independently selected from hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, substituted or unsubstituted aryl, substituted or unsubstituted heteroaryl, or a nitrogen protecting group when attached to a nitrogen atom, or two R^{A} groups are joined to form a substituted or unsubstituted heterocyclic ring;
each occurrence of A is independently selected from substituted or unsubstituted heterocyclylene, substituted or unsubstituted arylene, or substituted or unsubstituted heteroarylene;
R¹, R₂, and R⁴-R⁸ are each independently hydrogen, OH, halogen, NH₂, CH₃, SO₂, NO₂, a leaving group, a protecting group, aryl, heteroaryl, NHR¹², N(R¹²)₂ C₃₋₈ cycloalkyl, N(R¹²)₂ heterocyclyl, or —(CH₂)ₙ—R¹²;
R² is hydrogen, —CH₃, aryl, or heteroaryl; and
n is 0-12;
wherein one or more carbon of R¹-R⁷ is optionally replaced with C(═O), O, S, SO₂, NH, NH—C₁₋₆ alkyl, NC₁₋₆ alkyl, NH₂, or N(C₁₋₆ alkyl)₂; and
[symbol] indicates the point of covalent attachment to a [Linker] or a [LRP1BM];
   (c) a compound of formula (II):

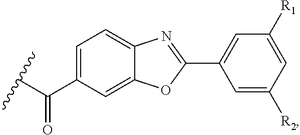

or a derivative or prodrug thereof,
wherein
R₁ and R₂ are each independently selected from hydrogen, N₃, alkynyl, OH, halogen, NH₂, N(C₁₋₆ alkyl)₂, C₁₋₆ alkyl, aryl, heteroaryl, NHR¹², N(R¹²)₂ C₃₋₈ cycloalkyl, N(R¹²)₂ heterocyclyl, or —(CH₁₂)—R¹²;
wherein the aryl and heteroaryl are optionally substituted with halogen, —SO₂, NO₂, —NH₂, or C₁₋₆ alkyl optionally substituted with halogen or C₃₋₈ cycloalkyl;
R¹² is hydrogen, —CH₃, aryl, or heteroaryl; and
n is 0-12;
wherein one or more carbon of R¹ or R² is optionally replaced with C(═O), O, S, SO₂, NH, NH—C₁₋₆ alkyl, NC₁₋₆ alkyl, NH₂, or N(C₁₋₆ alkyl)₂; and
[symbol] indicates the point of covalent attachment to a [Linker] or a [LRP1BM];
   (d) a compound of formula (III):

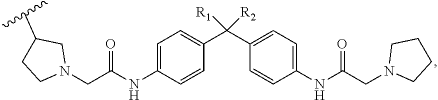

or a derivative or prodrug thereof,
wherein
R¹ is selected from benzene, phenyl, cyclohexyl, hydrogen, and CF₃;
R² is selected from hydrogen and CF₃; and
[symbol] indicates the point of covalent attachment to a [Linker] or a [LRP1BM];
   (e) a compound of formula (IV):

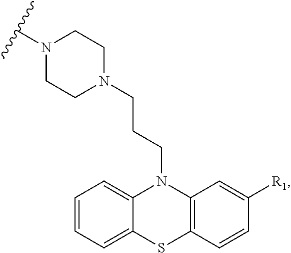

or a derivative or prodrug thereof,
wherein
R₁ is selected from hydrogen, Cl, OMe, SMe, and CF₃, and
[symbol] indicates the point of covalent attachment to a [Linker] or a [LRP1BM];
   (f) a compound of formula (V):

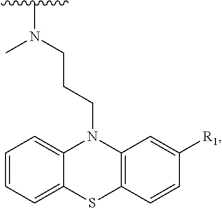

or a derivative or prodrug thereof,
wherein
R₁ is selected from hydrogen, Cl, OMe, SMe, and CF₃, and
[symbol] indicates the point of covalent attachment to a [Linker] or a [LRP1BM]; or
   (g) an amino acid sequence selected from:

SVWIWYE,
DVWIINKKLK,
MLRTKDLIWTLFFLGTAVS-NH₂,
MLRTKDLIWTLFFLGTAVS-KKRPKP-NH₂,
and
MLRTKDLIWTLFFLGTAVS-KKLVFF-NH₂;

[LRP1BM] represents a low density lipoprotein receptor-related protein 1 (LRP1) receptor binding motif comprising one of the following amino acid sequences:

TFFYGGSRGKRNNFKTEEYC-OH (or -NH₂),
TWPKHFDKHTFYSILKLGKH-OH,
EAKIEKHNHYQKK/C-NH₂,
EAKIEKHNHYQKQLEIAHEKLRK/C-NH₂,
R₈AKIEKHS₅HYQKK/C-NH₂,

wherein R₈ represents (R)-2-(7-octenyl)Ala-OH, S₅
represents (S)-2-(4-pentenyl)Ala-OH, and there is a hydrocarbon bridge between position 1 and 8,

LRKLRKRLLRDADDLLRKLRKRLLRDADDL-NH₂,
TEELRVRLASHLRKLRKRLL-NH₂,
Ac-VKFNKPFVFLNleIEQNTK-NH₂,

wherein Nle represents norleucine,

VKFNKPFVFLMIEQNTK,
TFFYGGCRGKRNNFKTEEYC-OH (or -NH₂),
TFFYGGSRGKRNNFRTEEYC-OH (or -NH₂),
TFFYGGSRGRRNNFRTEEYC-OH (or -NH₂),
cyeetkfnnrkGrsGGyfft-OH (or-NH₂),
TFFYGGCRAKRNNFKRAKY,
TFFYGGCRGKKNNFKRAKY,
PFFYGGCRGKRNNFKTEEY,
TFFYGGKRGKRNNFKTKEY,
TFFYGGCRGKRNNFKTKRY,
TFFYGGKRGKRNNFKTAEY,
TFFYGGKRGKRNNFKREKY,
RFKYGGCLGNKNNFLRLKY,
and
RFKYGGCLGNKNNYLRLKY,

wherein the underlined amino acids in the above sequences indicate that the amino acids may be present or absent and underlined K/C indicates that either K or C may be present; and
[Linker] represents a polyethylene glycol containing linker having 1-12 ethylene glycol residues, or [Linker] represents a Linking group comprising:
   (a) —CH₂CH₂(OCH₂CH₂)ₘOCH₂—, —(CH₂)ₘCH₂—, or —[N(R^{a})—CH(R^{b})(C═O)]ₘ—,
      or a polypropylene glycol or polypropylene-co-polyethylene glycol group containing 1-100 alkylene glycol units;
      wherein each R^{a} is independently H, C₁-C₃ alkyl, or C₁-C₆ alkanol, or combines with R^{b} to form a pyrrolidine or hydroxypyrroline group;
      wherein each R^{b} is independently selected from the group consisting of hydrogen, methyl, isopropyl, —CH(CH₃)CH₂CH₃, —CH₂CH(CH₃)₂, —(CH₂)₃-guanidine, —CH₂C(═O)NH₂, —CH₂C(═O)OH, —CH₂SH, —(CH₂)₂C(═O)NH₂, —(CH₂)₂C(═O)OH, —(CH₂)imidazole, —(CH₂)₄NH₂, —CH₂CH₂SCH₃, benzyl, —CH₂OH, —CH(OH)CH₃, —(CH₂)imidazole, or —(CH₂)phenol; and
      wherein m is an integer ranging from 1 to 15;
   (b) —[N(R′—(CH₂)₁₋₁₅—C(═O)]ₘ—, wherein R′ is H or a C₁-C₃ alkyl optionally substituted with 1-2 hydroxyl groups, and m is an integer ranging from 1 to 100;
   (c) —Z-D-Z′—, wherein:
      Z and Z′ are each independently a bond, —(CH₂)ᵢ—O—, —(CH₂)ᵢ—S—, —(CH₂)ᵢ—N(R)—,

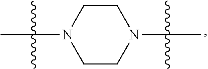 [SMILES: *N1CCN(*)CC1]

—(CH₂)ᵢ—C(R²)═C(R²)— (cis or trans), —(CH₂)ᵢ-≡-, or —Y—C(═O)—Y—,
each R is independently H, C₁-C₃ alkyl, or C₁-C₆ alkanol,
each R² is independently H or C₁-C₃ alkyl,
each Y is independently a bond, O, S, or N(R),
each i is independently 0 to 100,
D is a bond, —(CH₂)ᵢ—Y—C(═O)—Y—(CH₂)ᵢ—, —(CH₂)_{m′}—, or —[(CH₂)ₙ—X₁)]ⱼ—, with the proviso that Z, Z′, and D are not each simultaneously bonds;
X₁ is O, S, or N(R),
j is an integer ranging from 1 to 100,
m′ is an integer ranging from 1 to 100, n is an integer ranging from 1 to 100;
(d) —CH₂—(OCH₂CH₂)ₙ—CH₂—, —(CH₂CH₂O)_{n′}CH₂CH₂—, or —(CH₂CH₂CH₂O)ₙ—, wherein each n and n′ is independently an integer ranging from 1 to 25;
(e) -PEG-CON-PEG-, wherein each PEG is independently a polyethylene glycol group containing from 1-12 ethylene glycol residues and CON is selected from

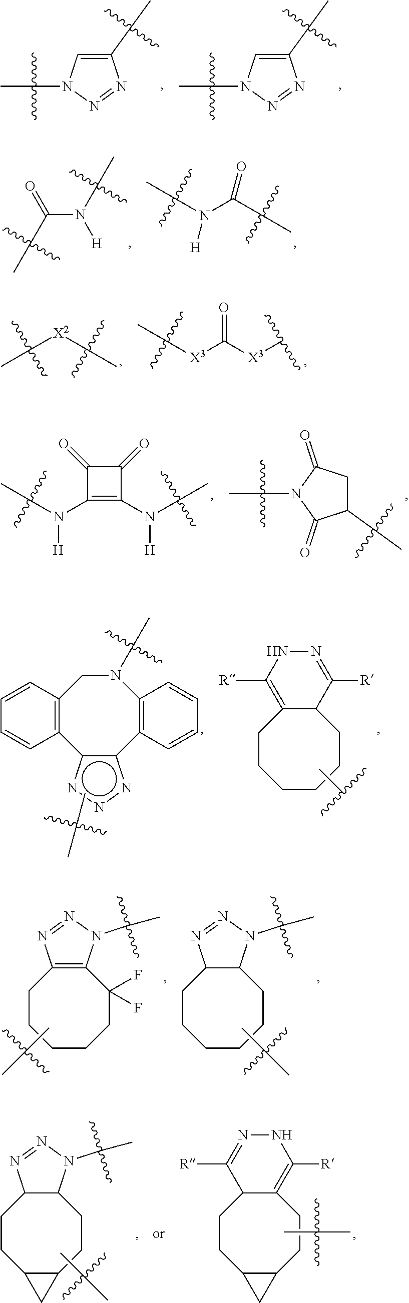

wherein R′ and R″ are each independently H, methyl, or a bond;
(f) -PEG-CON-PEG-, wherein each PEG is independently a polyethylene glycol group containing from 1-12 ethylene glycol residues and CON comprises a diamide structure selected from —C(═O)—N(R¹)—(CH₂)_{n″}—N(R¹)C(═O)—, —N(R¹)—C(═O)(CH₂)_{n″}—C(═O)N(R¹)—, or —N(R¹)—C(═O)(CH₂)_{n″}—N(R¹)C(═O)—, wherein each R¹ is independently H or C₁-C₃ alkyl, and n″ is independently an integer from 0 to 8;
(g) -PEG-CON-PEG-, wherein each PEG is independently a polyethylene glycol group containing from 1-12 ethylene glycol residues and CON comprises a structure

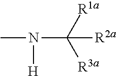

wherein:
   R^{1a}, R^{2a} and R^{3a} are each independently H, —(CH₂)_{M1}—, —(CH₂)_{M2}C(═O)_{M3}(NR⁴)_{M3}—(CH₂)_{M2}—, —(CH₂)_{M2}(NR⁴)_{M3}C(O)_{M3}—(CH₂)_{M2}—, or —(CH₂)_{M2}O—(CH₂)_{M1}—C(O)NR⁴—, with the proviso that R^{1a}, R^{2a} and R^{3a} are not simultaneously H;
   each M1 is independently 1, 2, 3, or 4; in certain embodiments, 1 or 2;
   each M2 is independently 0, 1, 2, 3, or 4; in certain embodiments, 0, 1 or 2;
   each M3 is independently 0 or 1; and
   each R⁴ is independently H, C₁-C₃ alkyl, C₁-C₆ alkanol, or —C(═O)(C₁-C₃ alkyl), with the proviso that M2, and M3 within the same
   R^{1a}, R^{2a} and R^{3a} cannot all be simultaneously 0;
(h) -PEG-CON-PEG-, wherein each PEG is independently a polyethylene glycol group containing from 1-12 ethylene glycol residues and CON comprises a structure:

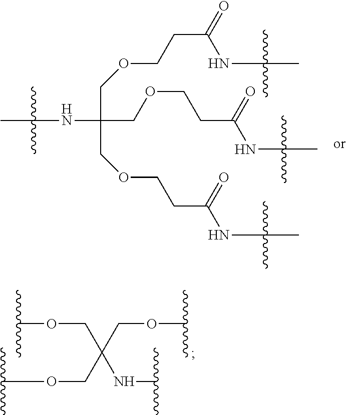 [SMILES: *NC(=O)CCOCC(COCCC(=O)N*)(COCCC(=O)N*)N* ; *NC(CO*)(CO*)CO*]

(i) a natural or an unnatural amino acid;
(j) [Gly-Gly-Gly-Gly-Ser]ₙ, where n is 1, 2, 3, 4, 5 or 6;
(k) [Ser-Ser-Ser-Ser-Gly]_{y}, where y is 1; or
(l) Ser-Gly-Ser-Ser-Ser-Ser-Gly-Ser-Ser-Ser-Ser-Gly-Ser.

Embodiment 2 provides the compound of embodiment 1, wherein the valence of the Linker is 1, 2, or 3.

Embodiment 3 provides the compound of any one of embodiments 1-2, wherein m is 1, 2, or 3.

Embodiment 4 provides the compound of any one of embodiments 1-3, wherein n and o are each independently 1, 2, or 3.

Embodiment 5 provides the compound of any one of embodiments 1-4, wherein the target binding motif binds noncovalently to an extracellular protein or a cell surface protein.

Embodiment 6 provides the compound of any one of embodiments 1-5, wherein the extracellular or cell surface protein comprises a calcitonin gene-related peptide (CGRP), a CGRP receptor, an N-methyl-D-aspartate (NMDA) receptor, myeloperoxidase (MPO), α-synuclein, IAPP, transthyretin, extracellular tau, amyloid precursor protein, a prion protein, or amyloid beta.

Embodiment 7 provides the compound of any one of embodiments 1-6, wherein the extracellular or cell surface protein comprises extracellular tau or amyloid beta.

Embodiment 8 provides the compound of any one of embodiments 1-7, wherein the extracellular or cell surface protein is found in the brain or the central nervous system.

Embodiment 9 provides the compound of any one of embodiments 1-8, wherein [TBM] is selected from

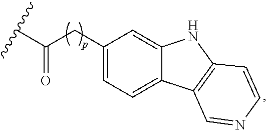

wherein p is an integer from 1-6; and

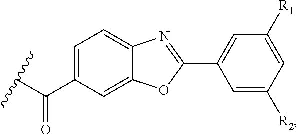

wherein R¹ and R² are each independently selected from F, Cl, Br, and I.

Embodiment 10 provides the compound of any one of embodiments 1-9, wherein the LRP1BM comprises the peptide of SEQ ID NO: 1.

Embodiment 11 provides the compound of any one of embodiments 1-10, wherein the C-terminal cysteine residue is absent from the peptide of SEQ ID NO: 1.

Embodiment 12 provides the compound of any one of embodiments 1-11, wherein the peptide of SEQ ID NO: 1 is attached to the Linker through its N-terminal tyrosine (Tyr1), Lys10, or Lys15.

Embodiment 13 provides a pharmaceutical composition comprising at least one pharmaceutically acceptable excipient and at least one compound of any one of embodiments 1-12.

Embodiment 14 provides the pharmaceutical composition of embodiment 13, further comprising another therapeutically active compound.

Embodiment 15 provides a method of treating, ameliorating, or preventing a disease or disorder in a subject, the method comprising:
administering a therapeutically effective amount of a composition comprising at least one compound of claim 1, or a salt, geometric isomer, stereoisomer, or solvate thereof.

Embodiment 16 provides the method of embodiment 15, wherein the disease or disorder is a neurological disease or disorder.

Embodiment 17 provides the method of embodiment 16, wherein the neurological disease or disorder is at least one of Huntington's Disease (HD), Parkinson's Disease (PD), Amyotropic Lateral Sclerosis (ALS), multiple system atrophy (MSA), Alzheimer's Disease, Lewy body dementia, Multiple System Atrophy, spinal and bulbar muscular atrophy (Kennedy's disease), Tourette Syndrome, spinocerebellar ataxia (SCA), schizophrenia, age associated memory impairment, autism, migraines, Rett syndrome, complex regional pain syndrome (CRPS), obsessive-compulsive disorder (OCD), attention-deficit disorder, bipolar disorder, hereditary cerebral angiopathy, ATTR amyloidosis, or depression.

Embodiment 18 provides the method of embodiment 16, wherein the neurological disease or disorder is Alzheimer's Disease.

Embodiment 19 provides the method of any one of embodiments 15-18, wherein the subject is further administered at least one additional therapeutic agent that treats, ameliorates, or prevents the disease or disorder.

Embodiment 20 provides the method of any one of embodiments 15-19, wherein the subject is a mammal.

Embodiment 21 provides the method of any one of embodiments 15-20, wherein the subject is a human.

Embodiment 22 provides the method of any one of embodiments 15-21, wherein the composition comprises at least one pharmaceutically acceptable carrier or excipient.

1. A compound of formula (I), or a salt, geometric isomer, stereoisomer, or solvate thereof:
[TBM]ₙ-[Linker]ₘ-[LRP1BM]ₒ (I),
wherein
m is 0, 1, 2, 3, 4, 5, 6, 7, 8, 9, 10, 11, 12, 13, 14, or 15;
n and o are each independently 0, 1, 2, 3, 4, 5, 6, 7, 8, 9, 10, 11, 12, 13, 14, or 15;
[TBM] represents a target binding motif comprising or consisting of:
(a) a compound selected from: 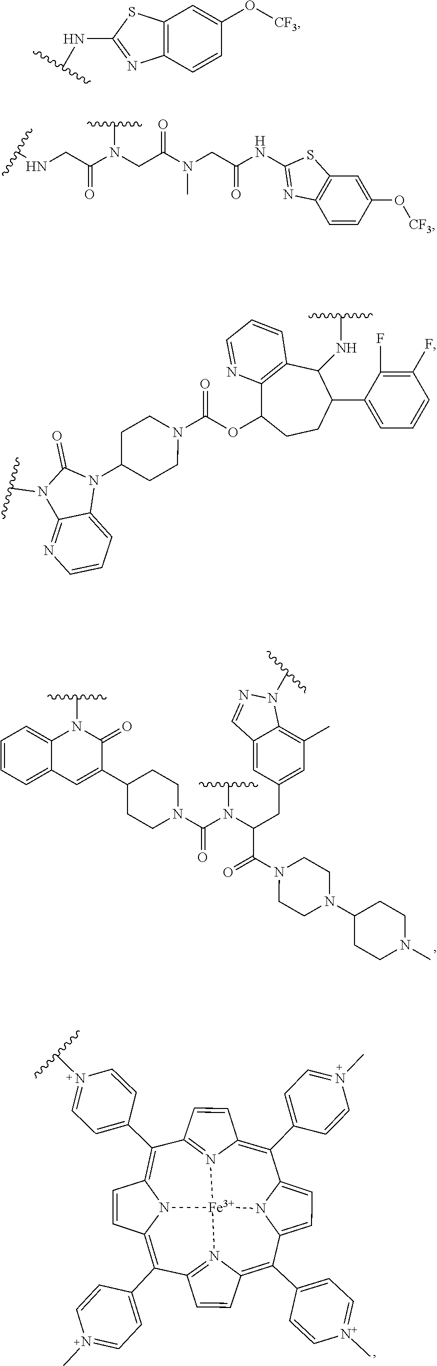 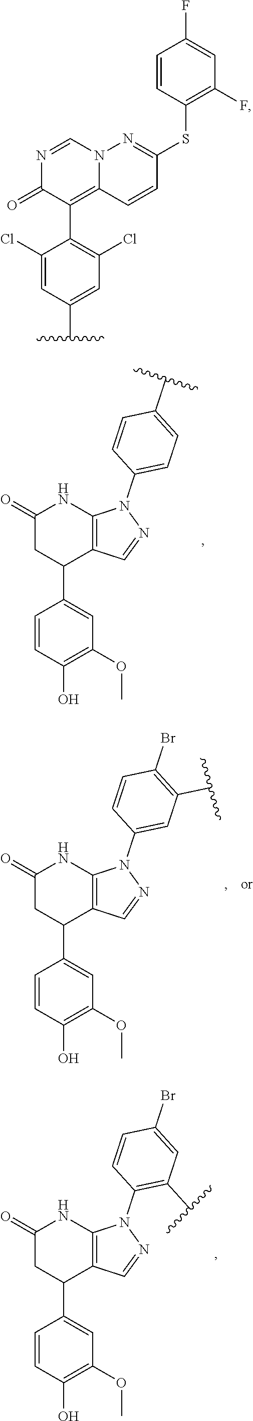 [SMILES: *c1cc(Cl)c(-c2c(=O)ncn3nc(Sc4ccc(F)cc4F)ccc23)c(Cl)c1 ; *c1ccc(-n2ncc3c2NC(=O)CC3c2ccc(O)c(OC)c2)cc1 ; *c1cc(-n2ncc3c2NC(=O)CC3c2ccc(O)c(OC)c2)ccc1Br ; *c1cc(Br)ccc1-n1ncc2c1NC(=O)CC2c1ccc(O)c(OC)c1]
or a derivative or prodrug thereof, wherein [symbol]
indicates possible points of covalent attachment to a [Linker] or a [LRP1BM];
(b) a compound of formula (I): 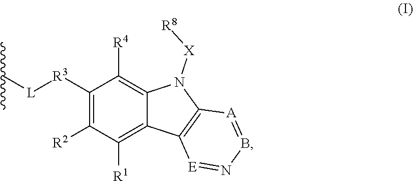
or a derivative or prodrug thereof,
wherein:
A is N or CR⁵;
B is N or CR⁶;
E is N or CR⁷;
L is a substituted or unsubstituted alkylene, substituted or unsubstituted alkenylene, substituted or unsubstituted alkynylene, substituted or unsubstituted carbocyclylene, substituted or unsubstituted heterocyclylene, substituted or unsubstituted arylene, substituted or unsubstituted heteroarylene, substituted or unsubstituted heteroalkylene, a bond, —O—, —NR^{A}—, —S—, —C(═O)—, —C(═O)O—, —C(═O)NR^{A}, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂—NR^{A}—, —NR^{A}S(O)₂—, or a combination thereof;
X is a bond or substituted or unsubstituted C₁₋₁₂ alkylene, wherein one or more carbon is optionally replaced with C(═O), O, S, SO₂, NH, or NC₁₋₆ alkyl optionally substituted with halogen, OH, or C₁₋₆ alkyl;
R⁸ is hydrogen, —N₃, alkynyl, OH, halogen, NH₂, N(C₁₋₆ alkyl)₂, aryl, heteroaryl, or a protecting group, wherein the aryl and heteroaryl are optionally substituted with halogen, SO₂, NH₂, or C₁₋₆ alkyl optionally substituted with halogen or C₃₋₈ cycloalkyl;
R³ is —(CH₂)ₙ—, —(CH₂)ₙ—C(═O), —(CH₂)ₙC(═O)—O—, (CH₂)ₙO—, -A-(CH₂)ₙ—O—, —(CH₂)ₙ-A-O—, -A-O—(CH₂)ₙ—(C═O)NR^{A}—, —(CH₂)ₙ—S—, -A-(CH₂)ₙS—, —(CH₂)ₙ-A-S—, -A-S—(CH₂)ₙ—(C═O)NR^{A}—, —(CH₂)ₙ—NR^{A}—, -A-(CH₂)ₙ—NR^{A}, —(CH₂)ₙ-A-NR^{A}—, —(CH₂)ₙ—(C═O)NR^{A}—, -A-(CH₂)ₙ—(C═O)NR^{A}—, —(CH₂)ₙ-A-(C═O)NR^{A}—, -A-NR^{A}—(CH₂)ₙ—(C═O)NR^{A}—, —(CH₂)ₙ—S(O)₂NR^{A}, -A-(CH₂)ₙ—S(O)₂NR^{A}—, or —(CH₂)ₙ-A-S(O)₂NR^{A}—;
each occurrence of R^{A} is independently selected from hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, substituted or unsubstituted aryl, substituted or unsubstituted heteroaryl, or a nitrogen protecting group when attached to a nitrogen atom, or two R^{A} groups are joined to form a substituted or unsubstituted heterocyclic ring;
each occurrence of A is independently selected from substituted or unsubstituted heterocyclylene, substituted or unsubstituted arylene, or substituted or unsubstituted heteroarylene;
R¹, R², and R⁴-R⁸ are each independently hydrogen, OH, halogen, NH₂, CH₃, SO₂, NO₂, a leaving group, a protecting group, aryl, heteroaryl, NHR¹², N(R¹²)₂C₃₋₈ cycloalkyl, N(R¹²)₂ heterocyclyl, or —(CH₂)ₙ—R¹²;
R¹² is hydrogen, —CH₃, aryl, or heteroaryl; and
n is 0, 1, 2, 3, 4, 5, 6, 7, 8, 9, 10, 11, or 12;
wherein one or more carbon of R¹-R⁷ is optionally replaced with C(═O), O, S, SO₂, NH, NH—C₁₋₆ alkyl, NC₁₋₆ alkyl, NH₂, or N(C₁₋₆ alkyl)₂; and [symbol]
indicates the point of covalent attachment to a [Linker] or a [LRP1BM];
(c) a compound of formula (II): 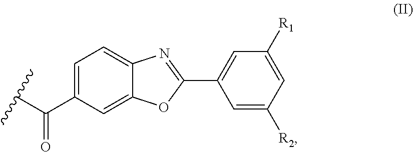
or a derivative or prodrug thereof,
wherein:
R₁ and R₂ are each independently selected from hydrogen, N₃, alkynyl, OH, halogen, NH₂, N(C₁₋₆ alkyl)₂, C₁₋₆ alkyl, aryl, heteroaryl, NHR¹², N(R¹²)₂, C₃₋₈ cycloalkyl, N(R¹²)₂ heterocyclyl, or —(CH₂)ₙ—R¹²;
wherein the aryl and heteroaryl are optionally substituted with halogen, —SO₂, NO₂, —NH₂, or C₁₋₆ alkyl optionally substituted with halogen or C₃₋₈ cycloalkyl;
each occurrence of R¹² is independently hydrogen, —CH₃, aryl, or heteroaryl; and
n is 0, 1, 2, 3, 4, 5, 6, 7, 8, 9, 10, 11, or 12;
wherein one or more carbon of R¹ or R² is optionally replaced with C(═O), O, S, SO₂, NH, NH—C₁₋₆ alkyl, NC₁₋₆ alkyl, NH₂, or N(C₁₋₆ alkyl)₂; and [symbol]
indicates the point of covalent attachment to a [Linker] or a [LRP1BM];
(d) a compound of formula (III): 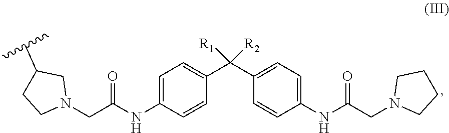
or a derivative or prodrug thereof,
wherein:
R₂ is selected from benzene, phenyl, cyclohexyl, hydrogen, and CF₃;
R₂ is selected from hydrogen and CF₃; and [symbol]
indicates the point of covalent attachment to a [Linker] or a [LRP1BM];
(e) a compound of formula (IV): 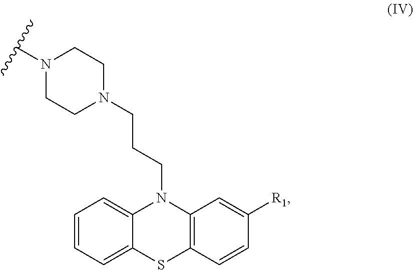
or a derivative or prodrug thereof,
wherein:
R₁ is selected from hydrogen, Cl, OMe, SMe, and CF₃, and [symbol]
indicates the point of covalent attachment to a [Linker] or a [LRP1BM];
(f) a compound of formula (V): 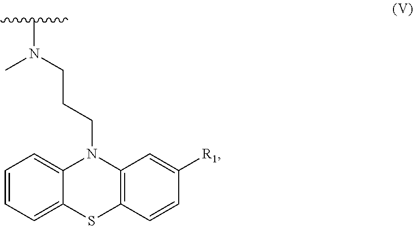
or a derivative or prodrug thereof,
wherein:
R₁ is selected from hydrogen, Cl, OMe, SMe, and CF₃, and [symbol]
indicates the point of covalent attachment to a [Linker] or a [LRP1BM]; or
(g) an amino acid sequence selected from:
SVWIWYE,
DVWIINKKLK,
MLRTKDLIWTLFFLGTAVS-NH₂,
MLRTKDLIWTLFFLGTAVS-KKRPKP-NH₂,
and
MLRTKDLIWTLFFLGTAVS-KKLVFF-NH₂;
[LRP1BM] represents a low density lipoprotein receptor-related protein 1 (LRP1) receptor binding motif comprising one of the following amino acid sequences:
TFFYGGSRGKRNNFKTEEYC-OH (or -NH₂),
TWPKHFDKHTFYSILKLGKH-OH,
EAKIEKHNHYQKK/C-NH₂,
EAKIEKHNHYQKQLEIAHEKLRK/C-NH₂,
R₈AKIEKHS₅HYQKK/C-NH₂,
wherein R₈ represents (R)-2-(7-octenyl)Ala-OH, S₅ represents (S)-2-(4-pentenyl)Ala-OH, and there is a hydrocarbon bridge between position 1 and 8,
LRKLRKRLLRDADDLLRKLRKRLLRDADDL-NH₂,
TEELRVRLASHLRKLRKRLL-NH₂,
Ac-VKFNKPFVFLNleIEQNTK-NH₂,
wherein Nle represents norleucine,
VKFNKPFVFLMIEQNTK,
TFFYGGCRGKRNNFKTEEYC-OH (or -NH₂),
TFFYGGSRGKRNNFRTEEYC-OH (or -NH₂),
TFFYGGSRGRRNNFRTEEYC-OH (or -NH₂),
cyeetkfnnrkGrsGGyfft-OH (or-NH₂),
TFFYGGCRAKRNNFKRAKY,
TFFYGGCRGKKNNFKRAKY,
PFFYGGCRGKRNNFKTEEY,
TFFYGGKRGKRNNFKTKEY,
TFFYGGCRGKRNNFKTKRY,
TFFYGGKRGKRNNFKTAEY,
TFFYGGKRGKRNNFKREKY,
RFKYGGCLGNKNNFLRLKY,
and
RFKYGGCLGNKNNYLRLKY,
wherein the underlined amino acids in the above sequences indicate that the amino acids may be present or absent and underlined K/C indicates that either K or C may be present; and
[Linker] represents a polyethylene glycol containing linker having 1, 2, 3, 4, 5, 6, 7, 8, 9, 10, 11, or 12 ethylene glycol residues, or
[Linker] represents a Linking group comprising:
(a) —CH₂CH₂(OCH₂CH₂)ₘOCH₂—, —(CH₂)ₘCH₂—, or —[N(R^{a})—CH(R^{b})(C═O)]ₘ—,
or a polypropylene glycol or polypropylene-co-polyethylene glycol group containing 1-100 alkylene glycol units;
wherein each R^{a} is independently H, C₁-C₃ alkyl, or C₁-C₆ alkanol, or combines with R^{b} to form a pyrrolidine or hydroxypyrroline group;
wherein each R^{b} is independently selected from the group consisting of hydrogen, methyl, isopropyl, —CH(CH₃)CH₂CH₃, —CH₂CH(CH₃)₂, —(CH₂)₃-guanidine, —CH₂C(═O)NH₂, —CH₂C(═O)OH, —CH₂SH, —(CH₂)₂C(═O)NH₂, —(CH₂)₂C(═O)OH, —(CH₂)imidazole, —(CH₂)₄NH₂, —CH₂CH₂SCH₃, benzyl, —CH₂OH, —CH(OH)CH₃, —(CH₂)imidazole, and —(CH₂)phenol; and
wherein m is 1, 2, 3, 4, 5, 6, 7, 8, 9, 10, 11, 12, 13, 14, or 15;
(b) —[N(R′—(CH₂)₁₋₁₅—C(═O)]ₘ—, wherein R′ is H or a C₁-C₃ alkyl optionally substituted with 1 or 2 hydroxyl groups, and m is an integer ranging from 1 to 100;
(c) —Z-D-Z′—, wherein:
Z and Z′ are each independently a bond, —(CH₂)ᵢ—O—, —(CH₂)ᵢ—S—, —(CH₂)ᵢ—N(R)—, 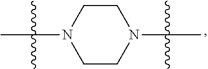 [SMILES: *N1CCN(*)CC1]
—(CH₂)ᵢ—C(R²)═C(R²)— (cis or trans), —(CH₂)ᵢ-≡-, or —Y—C(═O)—Y—,
each R is independently H, C₁-C₃ alkyl, or C₁-C₆ alkanol,
each R² is independently H or C₁-C₃ alkyl,
each Y is independently a bond, O, S, or N(R),
each i is independently an integer ranging from 0 to 100,
D is a bond, —(CH₂)ᵢ—Y—C(═O)—Y—(CH₂)ᵢ—, —(CH₂)_{m′}—, or —[(CH₂)ₙ—X₁)]ⱼ—, with the proviso that Z, Z′, and D are not each simultaneously bonds;
X₁ is O, S, or N(R),
j is an integer ranging from 1 to 100,
m′ is an integer ranging from 1 to 100,
n is an integer ranging from 1 to 100;
(d) —CH₂—(OCH₂CH₂)ₙ—CH₂—, —(CH₂CH₂O)_{n′}CH₂CH₂—, or —(CH₂CH₂CH₂O)ₙ—, wherein each n and n′ is independently 1, 2, 3, 4, 5, 6, 7, 8, 9, 10, 11, 12, 13, 14, 15, 16, 17, 18, 19, 20, 21, 22, 23, 24, or 25;
(e) -PEG-CON-PEG-, wherein each PEG is independently a polyethylene glycol group comprising 1, 2, 3, 4, 5, 6, 7, 8, 9, 10, 11, or 12 ethylene glycol residues and CON is selected from 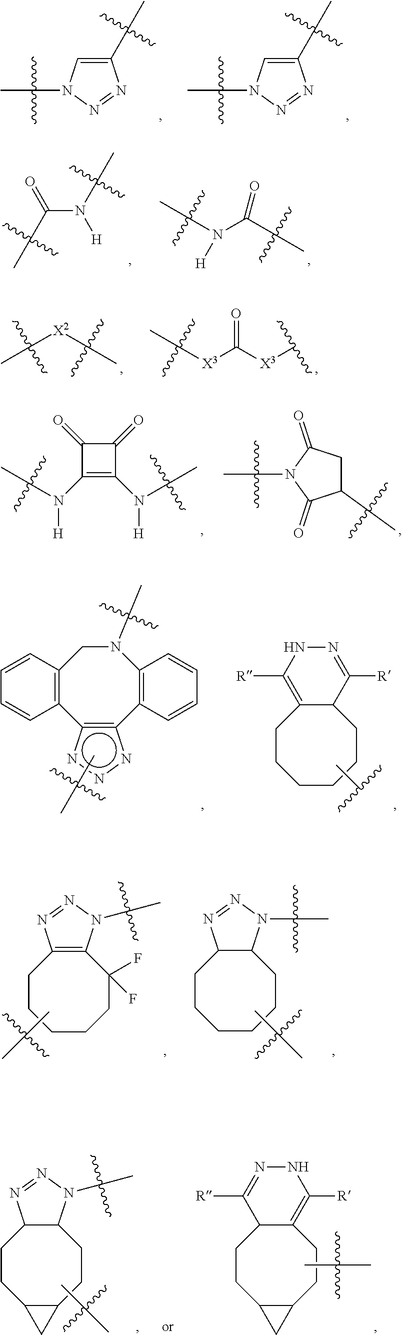
wherein R′ and R″ are each independently H, methyl, or a bond;
(f) -PEG-CON-PEG-, wherein each PEG is independently a polyethylene glycol group containing from 1, 2, 3, 4, 5, 6, 7, 8, 9, 10, 11, or 12 ethylene glycol residues and CON comprises a diamide structure selected from —C(═O)—N(R¹)—(CH₂)_{n″}—N(R¹)C(═O)—, —N(R¹)—C(═O)(CH₂)_{n″}—C(═O)N(R¹)—, or —N(R¹)—C(═O)(CH₂)_{n″}—N(R¹)C(═O)—, wherein each R¹ is independently H or C₁-C₃ alkyl, and n″ is independently 0, 1, 2, 3, 4, 5, 6, 7, or 8;
(g) -PEG-CON-PEG-, wherein each PEG is independently a polyethylene glycol group containing from 1, 2, 3, 4, 5, 6, 7, 8, 9, 10, 11, or 12 ethylene glycol residues and CON comprises a structure 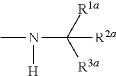
wherein:
R^{1a}, R^{2a} and R^{3a} are each independently H, —(CH₂)_{M1}—, —(CH₂)_{M2}C(═O)_{M3}(NR⁴)_{M3}—(CH₂)_{M2}—, —(CH₂)_{M2}(NR⁴)_{M3}C(O)_{M3}—(CH₂)_{M2}—, or —(CH₂)_{M2}O—(CH₂)_{M1}—C(O)NR⁴—, with the proviso that R^{1a}, R^{2a} and R^{3a} are not simultaneously H;
each M1 is independently 1, 2, 3, or 4;
each M2 is independently 0, 1, 2, 3, or 4;
each M3 is independently 0 or 1; and
each R⁴ is independently H, C₁-C₃ alkyl, C₁-C₆ alkanol, or —C(═O)(C₁-C₃ alkyl), with the proviso that M2, and M3 within the same R^{1a}, R^{2a} and R^{3a} cannot all be simultaneously 0;
(h) -PEG-CON-PEG-, wherein each PEG is independently a polyethylene glycol group containing from 1, 2, 3, 4, 5, 6, 7, 8, 9, 10, 11, or 12 ethylene glycol residues and CON comprises a structure: 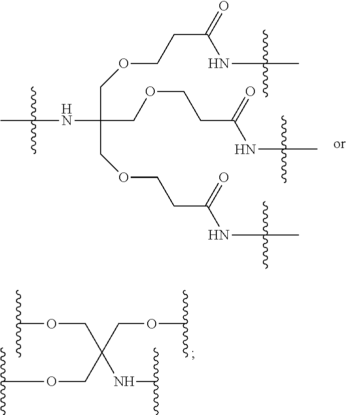 [SMILES: *NC(=O)CCOCC(COCCC(=O)N*)(COCCC(=O)N*)N* ; *NC(CO*)(CO*)CO*]
(i) a natural or an unnatural amino acid;
(j) [Gly-Gly-Gly-Gly-Ser]ₙ, where n is 1, 2, 3, 4, 5 or 6;
(k) [Ser-Ser-Ser-Ser-Gly]y, where y is equal to or greater than 1; or
(l) Ser-Gly-Ser-Ser-Ser-Ser-Gly-Ser-Ser-Ser-Ser-Gly-Ser.

2. The compound of claim 1, wherein the valence of the Linker is 1, 2, or 3.

3. The compound of claim 1, wherein m is 1, 2, or 3.

4. The compound of claim 1, wherein n and o are each independently 1, 2, or 3.

5. The compound of claim 1, wherein the target binding motif binds noncovalently to an extracellular protein or a cell surface protein.

6. The compound of claim 5, wherein the extracellular or cell surface protein comprises a calcitonin gene-related peptide (CGRP), a CGRP receptor, an N-methyl-D-aspartate (NMDA) receptor, myeloperoxidase (MPO), α-synuclein, IAPP, transthyretin, extracellular tau, amyloid precursor protein, a prion protein, or amyloid beta.

7. The compound of claim 5, wherein the extracellular or cell surface protein comprises extracellular tau or amyloid beta.

8. The compound of claim 5, wherein the extracellular or cell surface protein is found in the brain or the central nervous system.

9. The compound of claim 1, wherein [TBM] is selected from 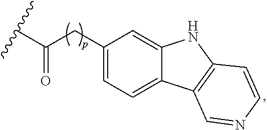
wherein p is 1, 2, 3, 4, 5, or 6; and 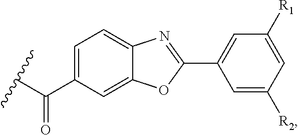
wherein R₁ and R₂ are each independently selected from F, Cl, Br, and I.

10. The compound of claim 1, wherein the LRP1BM comprises the peptide of SEQ ID NO: 1.

11. The compound of claim 10, wherein the C-terminal cysteine residue is absent from the peptide of SEQ ID NO: 1.

12. The compound of claim 11, wherein the peptide of SEQ ID NO: 1 is attached to the Linker through its N-terminal tyrosine (Tyr1), Lys10, or Lys15.

13. A pharmaceutical composition comprising at least one pharmaceutically acceptable excipient and at least one compound of claim 1.

14. The pharmaceutical composition of claim 13, further comprising another therapeutically active compound.

15. A method of treating, ameliorating, or preventing a disease or disorder in a subject, the method comprising:
administering a therapeutically effective amount of a composition comprising at least one compound of claim 1, or a salt, geometric isomer, stereoisomer, or solvate thereof.

16. The method of claim 15, wherein the disease or disorder is a neurological disease or disorder.

17. The method of claim 16, wherein the neurological disease or disorder is at least one of Huntington's Disease (HD), Parkinson's Disease (PD), Amyotropic Lateral Sclerosis (ALS), multiple system atrophy (MSA), Alzheimer's Disease, Lewy body dementia, Multiple System Atrophy, spinal and bulbar muscular atrophy (Kennedy's disease), Tourette Syndrome, spinocerebellar ataxia (SCA), schizophrenia, age associated memory impairment, autism, migraines, Rett syndrome, complex regional pain syndrome (CRPS), obsessive-compulsive disorder (OCD), attention-deficit disorder, bipolar disorder, hereditary cerebral angiopathy, ATTR amyloidosis, and depression.

18. The method of claim 16, wherein the neurological disease or disorder is Alzheimer's Disease.

19. The method of claim 15, wherein the subject is further administered at least one additional therapeutic agent that treats, ameliorates, or prevents the disease or disorder.

20. The method of claim 15, wherein the subject is a mammal.

21. The method of claim 15, wherein the subject is a human.

22. The method of claim 15, wherein the composition comprises at least one pharmaceutically acceptable carrier or excipient.
